(19) United States
(12) Patent Application Publication
(10) Pub. No.: US 2026/0327659 A1
(43) Pub. Date: Oct. 22, 2026

(54) PERITONEAL MACROPHAGES COMPRISING A NANOPARTICLE ENCAPSULATING A NUCLEIC ACID MOLECULE AND METHODS OF USE THEREOF

(71) Applicants: Alnylam Pharmaceuticals, Inc., Cambridge, MA (US); NORTHEASTERN UNIVERSITY, Boston, MA (US)

(72) Inventors: Dhaval Oza, Jamaica Plain, MA (US); Mansoor Amiji, Attleboro, MA (US)

(21) Appl. No.: 19/637,074

(22) Filed: Apr. 2, 2026

Related U.S. Application Data
(63) Continuation of application No. PCT/US2024/049520, filed on Oct. 2, 2024.
(60) Provisional application No. 63/542,218, filed on Oct. 3, 2023.

(51) Int. Cl. C12N 15/88; C12N 15/113

(52) U.S. Cl. CPC C12N 15/88; C12N 15/113; C12N 2310/14

(57) ABSTRACT

The present disclosure provides methods of delivering a nucleic acid molecule to a large peritoneal macrophage (LPM), by contacting a nanoparticle encapsulating the nucleic acid molecule with the LPM, thereby delivering the nucleic acid molecule to the LPM, and compositions generated by these methods. The disclosure also provides methods of delivering an LPM comprising a nanoparticle encapsulating the nucleic acid molecule to an injured tissue in a subject in need thereof, and methods of treating a disease in the subject, e.g., an inflammatory disease, an infectious disease, an autoimmune disease, or a cancer using these LPMs.

### RELATED APPLICATIONS

This application is a 35 § U.S.C. 111(a) continuation application which claims the benefit of priority to International Application No. PCT/US2024/049520, filed on Oct. 2, 2024, which, in turn, claims the benefit of priority to U.S. Provisional Application No. 63/542,218, filed on Oct. 3, 2023. The entire contents of each of the foregoing applications are expressly incorporated herein by reference.

### FEDERAL FUNDING LEGEND

This invention was made with government support under Grant Number R21-CA213114-01A1 awarded by the National Institutes of Health. The U.S. government has certain rights in the invention.

### FIELD OF THE DISCLOSURE

The present disclosure relates to peritoneal macrophages comprising a nanoparticle encapsulating a nucleic acid molecule, and methods of use thereof.

### BACKGROUND OF THE DISCLOSURE

Macrophages are one of the most heterogenous, multi-functional and versatile cells of the innate immune system. They reside in almost every mammalian tissue and have well-established roles of maintaining tissue homeostasis and monitoring tissue microenvironment for infection and tissue damage (Oza D and Amiji M M. In: Gupta S, Pathak Y V, editors. Macrophage Targeted Delivery Systems: Basic Concepts and Therapeutic Applications. 2022. p. 357-78; Nobs S P and Kopf M. Trends in Immunology. 2021; 42(6):495-507; Yona S and Gordon S. 2015; 6(328); and Teti G et al., Myeloid Cells in Health and Disease: American Society of Microbiology; 2017).

Tissue resident macrophages (TRMs) perform specialized functions and fulfil organ-specific roles reflected by their distinct transcriptomic profiles, plasticity, phenotype, and functionality across different tissues (Oza D and Amiji M M. 2022; Nobs S P and Kopf M. 2021; Yona S and Gordon S. 2015; Teti G et al., 2017; Gordon S and Plüddemann A. BMC Biology. 2017; 15(1):53; Jenkins S J and Allen J E. European journal of immunology. 2021; 51(8):1882-96; Cochain C et al., 2018; 122(12):1661-74; and Cox N et al., Annual review of immunology. 2021; 39:313-44). Based on their morphology, origin and phenotypic states, macrophages in the peritoneal cavity have been classified into monocyte-derived small peritoneal macrophages (SPMs) and tissue resident large peritoneal macrophages (LPMs), that are derived from embryogenic precursors (Cassado AdA et al., 2015; 6; and Okabe Y and Medzhitov R. Cell. 2014; 157). Novel findings about the behavior of LPMs in the context of acute tissue injuries have elucidated their tissue-specific functions and responses to injury stimuli (Okabe Y and Medzhitov R., 2014).

Despite being tissue-resident, LPMs have a unique migratory ability and can move to injured tissues within the abdominal cavity and impart wound healing properties (Parayath N N et al., Nano Letters. 2018; 18(6):3571-9; Honda M et al., Nature Communications. 2021; 12(1):7294; Ito T et al., Nature Communications. 2021; 12(1):2232; Wang J and Kubes P. Cell. 2016; 165(3):668-78; and Zindel J et al., 2021; 371(6533):eabe0595). While it has been speculated that LPMs are not necessarily “resident” and can migrate and infiltrate peritoneally located organs like the liver and intestines via an avascular route, it is not clear whether this phenomenon occurs more broadly across non-peritoneally located organs, and/or whether this is an inherent property of these unique TRM populations (Honda M et al., 2021; Ito T et al., 2021; and Wang J and Kubes P., 2016). Therefore, there remains a need for further exploration of migration of LPMs broadly across different tissues along with possible routes of migration to an injured tissue, particularly for organs distant from the peritoneal cavity, such as the lungs.

Macrophages are a very important therapeutic target considering their multiple vital roles in inflammatory diseases, autoimmune diseases, and cancer (Oza D and Amiji M M. 2022; Zhang C et al., 2021; 12; Xiao Y and Yu D. Pharmacology & Therapeutics. 2021; 221:107753; Wang H et al., Rotman Y. Cellular & Molecular Immunology. 2021; 18(1):73-91; and Tan Y et al., 2021; 11). However, despite significant progress with tissue-selective delivery with oligonucleotide therapies, there remain considerable roadblocks to selective delivery of therapeutic modalities to immune cells, such as LPMs (Setten R L et al., Nature Reviews Drug Discovery. 2019; 18(6):421-46; Wittrup A and Lieberman J. Nature Reviews Genetics. 2015; 16(9):543-52; Aigner A. 2019; 14(21):2777-82; and Roberts T C et al., Nature Reviews Drug Discovery. 2020; 19(10):673-94). Therefore, there is a need in the art for delivery of therapeutic modalities, such as nucleic acid molecules, to macrophages, e.g., LPMs.

### SUMMARY OF THE DISCLOSURE

The present disclosure meets this need in the art by providing methods of delivering a nucleic acid molecule to a large peritoneal macrophage (LPM), by contacting a nanoparticle (for example, a lipid nanoparticle (LNP)) encapsulating the nucleic acid molecule with the LPM, thereby delivering the nucleic acid molecule to the LPM, as well as compositions generated by these methods. The present disclosure successfully utilizes novel and effective methods of encapsulating a nucleic acid molecule (e.g., an siRNA) in a nanoparticle (e.g., an LNP) for methods of delivering the nanoparticle encapsulating the nucleic acid molecule to an LPM.

The methods of the disclosure are both simple, efficient and effective, and result in the production of an LPM comprising the nanoparticle encapsulating the nucleic acid molecule that can be used for a variety of therapeutic applications, disclosed herein, for example, delivering the nucleic acid molecule to an injured tissue, e.g., an injured extrperitoneal tissue, in a subject in need thereof; and/or methods of treatment of a disease, e.g., an inflammatory disease, an infectious disease, an autoimmune disease, or a cancer, in a subject in need thereof.

In one aspect, the disclosure provides a method of delivering a nucleic acid molecule to a large peritoneal macrophage (LPM), the meth-od comprising contacting a nanoparticle encapsulating the nucleic acid molecule with the LPM, thereby delivering the nucleic acid molecule to the LPM.

In some embodiments, the contacting the LPM with the nanoparticle encapsulating the nucleic acid molecule is performed in vivo.

In some embodiments, the contacting the LPM with the nanoparticle encapsulating the nucleic acid molecule is performed ex vivo.

In some embodiments, the LPM is a GATA6+ LPM.

In some embodiments, the nucleic acid molecule is selected from the group consisting of an oligonucleotide, a DNA, an RNA, a ribozyme, an aptamer, and a DNAzyme.

In some embodiments, the oligonucleotide is a single stranded oligonucleotide or a double stranded oligonucleotide.

In some embodiments, the DNA is selected from the group consisting of a genomic DNA (gDNA) and a copy DNA (cDNA).

In some embodiments, the ribozyme is selected from the group consisting of a hairpin ribozyme, a hammerhead ribozyme, a hepatitis delta virus ribozyme, a Varkud Satellite ribozyme, and a glmS ribozyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), and Toll Like Receptor 4 (TLR4).

In some embodiments, the nanoparticle is a lipid nanoparticle (LNP). In some embodiments, the LNP comprises cationic lipid C12-200, distearoylphosphatidylcholine (DSPC), cholesterol and Poly(ethylene) glycol (PEG)-C14. In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

In another aspect, the disclosure provides a method of delivering a nucleic acid molecule to an injured tissue in a subject in need thereof, the method comprising administering a nanoparticle encapsulating the nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, and allowing the LPM comprising the nanoparticle encapsulating the nucleic acid molecule to migrate to the injured tissue, thereby delivering the nucleic acid molecule to the injured tissue in the subject.

In some embodiments, the injured tissue is a non-peritoneal tissue. In some embodiments, the non-peritoneal tissue is a lung tissue.

In some embodiments, the lung tissue comprises an ablation or decrease in levels of tissue resident macrophages (TRMs) relative to an uninjured lung tissue. In some embodiments, the ablation or decrease in levels of the TRMs comprises an ablation or decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured lung tissue.

In some embodiments, the TRMs are alveolar macrophages.

In some embodiments, the LPM comprising the nanoparticle encapsulating the nucleic acid molecule migrates to the lung tissue in about 1 hour, about 2 hours, about 6 hours, about 12 hours, or about 24 hours after the ablation or decrease in levels of the TRMs in the lung tissue.

In some embodiments, the injured tissue is a peritoneal tissue. In some embodiments, the peritoneal tissue is a liver tissue.

In some embodiments, the liver tissue comprises an increase in levels of tissue resident macrophages (TRMs) relative to an uninjured liver tissue.

In some embodiments, the increase in levels of the TRMs comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the TRMs are F4/80+.

In some embodiments, the LPM comprising the nanoparticle encapsulating the nucleic acid molecule migrates to the liver tissue in about 1 hour, about 2 hours, about 6 hours, about 12 hours, or about 24 hours after the increase in levels of TRMs in the liver tissue.

In some embodiments, the serum of the subject comprises an increase in level of one or more enzymes selected from the group consisting of alanine transaminase (ALT), aspartate transaminase (AST), and bilirubin relative to serum of a subject with an uninjured liver tissue. In some embodiments, the increase in level of the one or more enzymes comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to the serum of a subject with an uninjured liver tissue.

In some embodiments, the liver tissue comprises an increase in level of pro-inflammatory macrophages relative to an uninjured liver tissue. In some embodiments, the increase in level of the pro-inflammatory macrophages comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the pro-inflammatory macrophages comprise one or more markers selected from the group consisting of iNOS-1, and TNF-a.

In some embodiments, the liver tissue comprises a decrease in level of anti-inflammatory macrophages relative to an uninjured liver tissue. In some embodiments, the decrease in level of the anti-inflammatory macrophages comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the anti-inflammatory macrophages comprise one or more markers selected from the group consisting of Arg-1, and CD206.

In some embodiments, the liver tissue comprises an increase in level of one or more pro-inflammatory cytokines selected from the group consisting of CXCL5, CCL11, CXCL1, IL-6, IL-9, IL-23, IL-28, CXCL10, CCL7, CCL3 and CCL5 relative to an uninjured liver tissue. In some embodiments, the increase in level of the one or more pro-inflammatory cytokines comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the liver tissue comprises a decrease in level of one or more anti-inflammatory cytokines selected from the group consisting of IL-4, and IL-10 relative to an uninjured liver tissue. In some embodiments, the decrease in level of the one or more anti-inflammatory cytokines comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the LPM comprising the nanoparticle encapsulating the nucleic acid molecule migrates to the injured tissue via systemic circulation in the subject.

In some embodiments, the migration of the LPM comprising the nanoparticle encapsulating the nucleic acid molecule to the injured tissue is detected by Diffuse in vivo Flow Cytometry (DiFC).

In some embodiments, the administering of the nanoparticle encapsulating the nucleic acid molecule to the subject is performed intraperitoneally.

In some embodiments, the LPM is a GATA6+ LPM.

In some embodiments, the nucleic acid molecule is selected from the group consisting of an oligonucleotide, a DNA, an RNA, a ribozyme, an aptamer, and a DNAzyme.

In some embodiments, the oligonucleotide is a single stranded oligonucleotide or a double stranded oligonucleotide.

In some embodiments, the DNA is selected from the group consisting of a genomic DNA (gDNA) and a copy DNA (cDNA).

In some embodiments, the ribozyme is selected from the group consisting of a hairpin ribozyme, a hammerhead ribozyme, a hepatitis delta virus ribozyme, a Varkud Satellite ribozyme, and a glmS ribozyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), and Toll Like Receptor 4 (TLR4).

In some embodiments, the nanoparticle is a lipid nanoparticle (LNP).

In some embodiments, the LNP comprises cationic lipid C12-200, distearoylphosphatidylcholine (DSPC), cholesterol and Poly(ethylene) glycol (PEG)-C14.

In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

In another aspect, the disclosure provides a method of treating a disease in a subject in need thereof, the method comprising administering a nanoparticle encapsulating a nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, and allowing the LPM comprising the nanoparticle encapsulating the nucleic acid molecule to mi-grate to an injured tissue, thereby treating the disease in the subject.

In some embodiments, the disease is selected from the group consisting of an inflammatory disease, an infectious disease, an autoimmune disease, and a cancer.

In some embodiments, the inflammatory disease is selected from the group consisting of drug induced liver injury, peritoneal adhesion, inflammatory bowel disease, acute respiratory distress syndrome (ARDS), severe acute respiratory syndrome (SARS), idiopathic pulmonary fibrosis (IPF), a chronic inflammatory disease, an inflammatory bone disease, an inflammatory lung disease, a chronic obstructive airway disease, Behcet's disease, an inflammatory diseases of the eye, a chronic inflammatory diseases of the gums, tuberculosis, leprosy, an inflammatory disease of the kidney, an inflammatory disease of the skin, an inflammatory disease of the central nervous system, a chronic demyelinating diseases of the nervous system, infectious meningitis, encephalomyelitis, Parkinson's disease, Huntington's disease, amyotrophic lateral sclerosis, a viral or autoimmune encephalitis, immune-complex vasculitis, systemic lupus erythematosus, an inflammatory disease of the heart, preeclampsia, schizophrenia, chronic liver failure, brain trauma, spinal cord trauma, and endometriosis.

In some embodiments, the infectious disease is a disease caused by a bacteria, a virus, or a fungi.

In some embodiments, the infectious disease is selected from the group consisting of COVID-19, viral hepatitis, tetanus, typhoid fever, diphtheria, syphilis, bacterial vaginosis, Trichomonas vaginalis, meningitis, urinary tract infection, bacterial gastroenteritis, impetigo, cellulitis, pneumonia, lyme disease, and leprosy.

In some embodiments, the infectious disease is an infection associated with one or more pathogens selected from the group consisting of coronavirus, Mycobacterium tuberculosis, Streptococcus, Pseudomonas, Shigella, Campylobacter, Salmonella, Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, and Vibrio cholera.

In some embodiments, the autoimmune disease is selected from the group consisting of a rheumatologic autoimmune disease, a gastrointestinal autoimmune disease, a liver autoimmune disease, vasculitis, a renal autoimmune disease, a dermatological autoimmune disease, a hematologic autoimmune disease, atherosclerosis, uveitis, an ear autoimmune disease, Raynaud's syndrome, an autoimmune endocrine disease, and a disease associated with organ transplantation.

In some embodiments, the cancer is selected from the group consisting of hepatocellular carcinoma, acute lymphocytic cancer, acute myeloid leukemia, alveolar rhabdomyosarcoma, bladder cancer, bone cancer, brain cancer, breast cancer, cancer of the anus, anal canal cancer, rectum cancer, cancer of the eye, cancer of the intrahepatic bile duct, cancer of the joints, cancer of the neck, gallbladder cancer, pleura cancer, nose cancer, nasal cavity cancer, middle ear cancer, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumor, liver cancer, lung cancer, lymphoma, malignant mesothelioma, mastocytoma, melanoma, multiple myeloma, nasopharynx cancer, nonHodgkin lymphoma, ovarian cancer, pancreatic cancer, peritoneum cancer, omentum cancer, mesentery cancer, pharynx cancer, prostate cancer, colorectal cancer, renal cancer, skin cancer, small intestine cancer, soft tissue cancer, solid tumor, stomach cancer, testicular cancer, thyroid cancer, ureter cancer, and urinary bladder cancer.

In some embodiments, the injured tissue is a non-peritoneal tissue. In some embodiments, the non-peritoneal tissue is a lung tissue.

In some embodiments, the lung tissue comprises an ablation or decrease in levels of tissue resident macrophages (TRMs) relative to an uninjured lung tissue. In some embodiments, the ablation or decrease in levels of the TRMs comprises an ablation or decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured lung tissue.

In some embodiments, the TRMs are alveolar macrophages.

In some embodiments, the LPM comprising the nanoparticle encapsulating the nucleic acid molecule migrates to the lung tissue in about 1 hour, about 2 hours, about 6 hours, about 12 hours, or about 24 hours after the ablation or decrease in levels of the TRMs in the lung tissue.

In some embodiments, the injured tissue is a peritoneal tissue. In some embodiments, the peritoneal tissue is a liver tissue.

In some embodiments, the liver tissue comprises an increase in levels of tissue resident macrophages (TRMs) relative to an uninjured liver tissue. In some embodiments, the increase in levels of the TRMs comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the TRMs are F4/80+.

In some embodiments, the LPM comprising the nanoparticle encapsulating the nucleic acid molecule migrates to the liver tissue in about 1 hour, about 2 hours, about 6 hours, about 12 hours, or about 24 hours after the increase in levels of TRMs in the liver tissue.

In some embodiments, the serum of the subject comprises an increase in level of one or more enzymes selected from the group consisting of alanine transaminase (ALT), aspartate transaminase (AST), and bilirubin relative to serum of a subject with an uninjured liver tissue. In some embodiments, the increase in level of the one or more enzymes comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to the serum of a subject with an uninjured liver tissue.

In some embodiments, the liver tissue comprises an increase in level of pro-inflammatory macrophages relative to an uninjured liver tissue. In some embodiments, the increase in level of the pro-inflammatory macrophages comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the pro-inflammatory macrophages comprise one or more markers selected from the group consisting of iNOS-1, and TNF-α.

In some embodiments, the liver tissue comprises a decrease in level of anti-inflammatory macrophages relative to an uninjured liver tissue. In some embodiments, the decrease in level of the anti-inflammatory macrophages comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the anti-inflammatory macrophages comprise one or more markers selected from the group consisting of Arg-1, and CD206.

In some embodiments, the liver tissue comprises an increase in level of one or more pro-inflammatory cytokines selected from the group consisting of CXCL5, CCL11, CXCL1, IL-6, IL-9, IL-23, IL-28, CXCL10, CCL7, CCL3 and CCL5 relative to an uninjured liver tissue. In some embodiments, the increase in level of the one or more pro-inflammatory cytokines comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the liver tissue comprises a decrease in level of one or more anti-inflammatory cytokines selected from the group consisting of IL-4, and IL-10 relative to an uninjured liver tissue. In some embodiments, the decrease in level of the one or more anti-inflammatory cytokines comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the LPM comprising the nanoparticle encapsulating the nucleic acid molecule migrates to the injured tissue via systemic circulation in the subject.

In some embodiments, the migration of the LPM comprising the nanoparticle encapsulating the nucleic acid molecule to the injured tissue is detected by Diffuse in vivo Flow Cytometry (DiFC).

In some embodiments, the administering of the nanoparticle encapsulating the nucleic acid molecule to the subject is performed intraperitoneally.

In some embodiments, the LPM is a GATA6+ LPM.

In some embodiments, the nucleic acid molecule is selected from the group consisting of an oligonucleotide, a DNA, an RNA, a ribozyme, an aptamer, and a DNAzyme.

In some embodiments, the oligonucleotide is a single stranded oligonucleotide or a double stranded oligonucleotide.

In some embodiments, the DNA is selected from the group consisting of a genomic DNA (gDNA) and a copy DNA (cDNA).

In some embodiments, the ribozyme is selected from the group consisting of a hairpin ribozyme, a hammerhead ribozyme, a hepatitis delta virus ribozyme, a Varkud Satellite ribozyme, and a glmS ribozyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), and Toll Like Receptor 4 (TLR4).

In some embodiments, the nanoparticle is a lipid nanoparticle (LNP).

In some embodiments, the LNP comprises cationic lipid C12-200, distearoylphosphatidylcholine (DSPC), cholesterol and Poly(ethylene) glycol (PEG)-C14.

In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

In one aspect, the disclosure provides a method of detecting migration of a large peritoneal macrophage (LPM) comprising a nanoparticle encapsulating a nucleic acid molecule to an injured tissue in a subject, the method comprising administering the nanoparticle encapsulating the nucleic acid molecule to the subject, allowing the nanoparticle encapsulating the nucleic acid molecule to contact the LPM in the subject, thereby generating the LPM comprising the nanoparticle encapsulating the nucleic acid molecule, and allowing the LPM comprising the nanoparticle encapsulating the nucleic acid molecule to migrate to the injured tissue, thereby detecting the migration of the LPM comprising the nanoparticle encapsulating the nucleic acid molecule to the injured tissue in the subject.

In some embodiments, the LPM comprising the nanoparticle encapsulating the nucleic acid molecule migrates to the injured tissue via systemic circulation in the subject.

In some embodiments, detecting the migration of the LPM comprising the nanoparticle encapsulating the nucleic acid molecule to the injured tissue in the subject is performed by Diffuse in vivo Flow Cytometry (DiFC).

In some embodiments, the administering of the nanoparticle encapsulating the nucleic acid molecule to the subject is performed intraperitoneally.

In some embodiments, the DiFC is performed about 0.5 hour, about 1 hour, about 2 hours, about 3 hours, about 6 hours, about 12 hours, about 24 hours or about 48 hours after the administering of the nanoparticle encapsulating the nucleic acid molecule to the subject.

In some embodiments, the nucleic acid molecule is labeled with a cy5.5 fluorophore.

In some embodiments, the nucleic acid molecule is selected from the group consisting of an oligonucleotide, a DNA, an RNA, a ribozyme, an aptamer, and a DNAzyme.

In some embodiments, the oligonucleotide is a single stranded oligonucleotide or a double stranded oligonucleotide.

In some embodiments, the DNA is selected from the group consisting of a genomic DNA (gDNA) and a copy DNA (cDNA).

In some embodiments, the ribozyme is selected from the group consisting of a hairpin ribozyme, a hammerhead ribozyme, a hepatitis delta virus ribozyme, a Varkud Satellite ribozyme, and a glmS ribozyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), and Toll Like Receptor 4 (TLR4).

In some embodiments, the siRNA comprises at least one modified nucleotide.

In some embodiments, the at least one modified nucleotide is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In some embodiments, the nanoparticle is a lipid nanoparticle (LNP).

In some embodiments, the LNP comprises cationic lipid C12-200, distearoylphosphatidylcholine (DSPC), cholesterol and Poly(ethylene) glycol (PEG)-C14.

In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

In another aspect, the disclosure provides a composition comprising a large peritoneal macrophage (LPM) comprising a nanoparticle encapsulating a nucleic acid molecule.

In some embodiments, the LPM is a GATA6+ LPM.

In another aspect, the disclosure provides a pharmaceutical composition comprising the composition(s) described herein, and a pharmaceutically acceptable carrier.

In some embodiments, the nucleic acid molecule is selected from the group consisting of an oligonucleotide, a DNA, an RNA, a ribozyme, an aptamer, and a DNAzyme.

In some embodiments, the oligonucleotide is a single stranded oligonucleotide or a double stranded oligonucleotide.

In some embodiments, the DNA is selected from the group consisting of a genomic DNA (gDNA) and a copy DNA (cDNA).

In some embodiments, the ribozyme is selected from the group consisting of a hairpin ribozyme, a hammerhead ribozyme, a hepatitis delta virus ribozyme, a Varkud Satellite ribozyme, and a glmS ribozyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), and Toll Like Receptor 4 (TLR4).

In some embodiments, the siRNA comprises at least one modified nucleotide.

In some embodiments, the at least one modified nucleotide is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In some embodiments, the nanoparticle is a lipid nanoparticle (LNP).

In some embodiments, the LNP comprises cationic lipid C12-200, distearoylphosphatidylcholine (DSPC), cholesterol and Poly(ethylene) glycol (PEG)-C14.

In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

### BRIEF DESCRIPTION OF THE DRAWINGS

FIG. 1A depicts a schematic representation of robust delivery of LNPs (e.g., C12-200 LNPs) encapsulating a nucleic acid molecule (e.g., cy5.5 labeled siRNA) to an LPM.

FIG. 1B depicts representative flow cytometry gating and analysis of cy5.5 Mean Fluorescence Intensity (MFI) in peritoneal CD11bhi F4/80 hi macrophages obtained from the respective treatment groups. Cells were pre-gated on size and viability. Data is representative of one sample from an n=4 per treatment group.

FIG. 1C depicts representative histograms depicting the average cy5.5 MFI from an n=4 of respective treatment groups.

FIG. 1D depicts quantification of MFI of cy5.5 in LPMs with the indicated groups. n=4 for all groups.

FIG. 1E depicts representative immunofluorescence images of siRNA-cy5.5 (red) uptake in GATA6 positive (orange) PMs after isolating peritoneal lavage 6 h and 24 h post intraperitoneal siRNA-cy5.5 administration. PBS-control mice were treated for 24 h. Scale bars, 100 μm.

FIG. 1F depicts representative immunofluorescence images of siRNA-cy5.5 (red) uptake in GATA6 positive (orange) PMs after isolating peritoneal lavage 6 h and 24 h post intraperitoneal siRNA-cy5.5 administration. PBS-control mice were treated for 24 h. Scale bars, 100 μm.

FIG. 1G depicts quantification of cy5.5 intensity of isolated GATA6+ LPMs in all the treatment groups. n=4 for all groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 1H depicts quantification of intensity of HA-PEI encapsulated cy5.5 labeled siRNA are taken up by LPMs at 6 h and 24 h. n=3 for all groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 1I depicts representative flow cytometry histograms of cy5.5 MFI within the F4/80 CD11b gated LPM population from the isolated peritoneal lavage from HA-PEI and C12-200 encapsulated siRNA-cy5.5 treated mice.

FIG. 1J depicts cy5.5 average MFI as calculated from the histograms. n=3 for all groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 2A depicts a schematic representation of detection of circulating LPMs labeled with siRNA-cy5.5 from peritoneal lavage by flow cytometry and DiFC scan in a phantom mouse.

FIG. 2B depicts representative flow cytometry gating and analysis of cy5.5 MFI in peritoneal CD11bhi F4/80 hi macrophages obtained from the respective treatment groups along with histogram of Flash Red 3 (FR3) microsphere MFI. Cells were pre-gated on size and viability. Data is representative of one sample from an n=3 per treatment group except for the FR3 microsphere that was run as itself.

FIG. 2C depicts comparative histograms of average cy5.5 MFI from the peritoneal CD11bhi F4/80 hi macrophages for the respective treatment groups compared to the FR3 microspheres.

FIG. 2D depicts quantification of MFI of cy5.5 in LPMs with the indicated groups. n=3 for all groups.

FIG. 2E depicts a schematic representation of the DiFC ‘phantom mouse’ study post 6 h treatment with siRNA-cy5.5 encapsulated in C12-200 formulation.

FIG. 2F depicts representative graphs of DiFC scans depicted as number of peaks detected over time from one sample per group from an n=3/group with the indicated treatment groups. Each peak (red circles) represents a circulating PM labeled with siRNA-cy5.5 in the peritoneal lavage F4/80+ cells, depicted as signal versus time.

FIG. 2G depicts quantification of mean peak amplitude of all the peaks measured over time depicting the intensity of labeled circulating PMs as detected by DiFC.

FIG. 2H depicts quantification of mean circulating LPMs detected per minute as scanned by DiFC in the phantom mouse model. n=3 for all the treatment groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 3A depicts a schematic representation of depletion of alveolar macrophages (AMs) after administration of intranasal clodronate.

FIG. 3B depicts flow cytometry analysis of CD11bhi F4/80 hi large resident macrophages in the isolated broncho-alveolar lavage fluid (BALF) samples. Cells were pre-gated on size and viability. Data is representative of one sample from an n=4 per treatment group.

FIG. 3C depicts quantification of flow cytometry analysis depicting a percentage of CD11bhi F4/80 hi macrophages in the BALF samples from respective treatment groups. n=4 for all other groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 3D depicts representative immunofluorescence images of cy5 labeled (red) siRNA labeled GATA6+ PMs (orange) in the isolated BALF cells with the indicated treatment groups. n=3 for all the groups. Scale bars, 200 m.

FIG. 3E depicts quantification of GATA6+ cells from the immunocytochemistry staining and analysis of isolated BALF cells with the indicated treatment groups.

FIG. 3F depicts quantification of cy5 intensity of isolated BALF cells in all the treatment groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 4A depicts a DiFC design for mouse scanning and detection of LPM in systemic circulation.

FIG. 4B depicts representative flow cytometry gating and analysis of cy5 MFI in peritoneal CD11bhi F4/80 hi macrophages obtained from the respective treatment 78 groups along with histogram of Flash Red 3 (FR3) microsphere MFI. Cells were pre-gated on size and viability. Data is representative of one sample from an n=3 per treatment group except for the FR3 microsphere that was run as itself.

FIG. 4C depicts comparative histograms of average cy5 MFI from the peritoneal CD11bhi F4/80 hi macrophages for the respective treatment groups compared to the FR3 microspheres.

FIG. 4D depicts quantification of MFI of cy5 in LPMs with the indicated groups. n=3 for all groups.

FIG. 4E depicts a study schematics for the DiFC ‘phantom mouse’ study post 6 h treatment with siRNA-cy5 encapsulated in C12-200 formulation.

FIG. 4F depicts representative graphs of DiFC scans depicted as number of peaks detected over time from one sample per group from an n=3/group with the indicated treatment groups. Each peak (red circles)79 represents a circulating PM labeled with siRNA-cy5 in the peritoneal lavage F4/80+ cells, depicted as signal versus time.

FIG. 4G depicts quantification of mean peak amplitude of all the peaks measured over time depicting the intensity of labeled circulating PMs as detected by DiFC.

FIG. 4H depicts quantification of mean circulating LPMs detected per minute as scanned by DiFC in the phantom mouse model. n=3 for all the treatment groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 5A depicts a schematics of DiFC design for mouse scanning and detection of LPM in systemic circulation.

FIG. 5B depicts a schematic representation of DiFC mediated scanning and detection of circulating macrophages at different time points post 12 hours of clodronate clodronate+siRNA-cy5.5 administration.

FIG. 5C depicts representative graphs of DiFC scans depicted as number of peaks detected over 600 seconds from one mouse per group from an n=4/group with the indicated treatments. Graphs are representative snapshots of a 10 minutes scan period from a total scanning time of 45 minutes per mouse. Each peak (arrowhead) represents a circulating cell labeled with siRNA-cy5.5 in systemic circulation, depicted as signal versus time.

FIG. 5D depicts quantification of mean circulating macrophages per minute, as detected by DiFC after quantifying ‘matched cellular peaks’ from a total scan time of 45 minutes. n=4 for all the treatment groups. Data has been represented as Mean+/−SEM, *p<0.05, **p<0.01, ***p<0.001, **** p<0.0001, ns not significant. P values were calculated with an ordinary one-way ANOVA followed by Dunnett's multiple comparison test, with a single pooled variance.

FIG. 6A depicts a schematic representation of detection of circulating LPMs in whole blood PBMCs upon clodronate-induced AM depletion.

FIG. 6B depicts representative graphs of DiFC scans depicted as number of peaks detected over 600 sec from one mouse per group from an n=4/group with the indicated treatments. Graphs are representative snapshots of a 10 mm scan period from a total scanning time of 45 min per mouse. Each peak (arrowhead) represents a circulating cell labeled with siRNA-cy5 in systemic circulation, depicted as signal versus time.

FIG. 6C depicts quantification of mean circulating macrophages per min as detected by DiFC after quantifying ‘matched cellular peaks’ from a total scan time of 45 mm. n=4 for all the treatment groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 6D depicts representative flow cytometry gating strategy to assess percentage of cy5.5+F4/80 hi CD11bhi macrophage population in whole blood PBMCs.

FIG. 6E depicts representative flow cytometry analysis of F4/80 hi CD11bhi macrophage population along with histograms of cy5.5+ cells within this population. Cells were pre-gated on size and viability. Data is representative of one sample from an n=4 per treatment group.

FIG. 6F depicts quantification of F4/80+CD11b+ macrophage population from the flow cytometry analysis of blood PBMCs for the respective treatment groups.

FIG. 6G depicts quantification of cy5.5+ within the CD11bhi F4/80 hi gated macrophage population with the indicated treatment groups. n=4 for all the groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 7A depicts representative immunofluorescence images of GATA6+ LPMs (yellow) in the isolated whole blood PBMC lymphocytes with the indicated treatment groups. n=4 for all the groups. Scale bars, 200 μm.

FIG. 7B depicts quantification of GATA6+ cells from the immunocytochemistry staining and analysis of isolated whole blood PBMC lymphocytes with the indicated treatment groups. n=4 for all groups. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 8A depicts the development of a mouse model of AILI, as observed from circulating biomarkers and immunohistochemical (IHC) evaluation of the liver.

FIGS. 8B-8D depicts circulating liver injury biomarkers from serum chemistry post APAP injection. B) Serum ALT C) Serum AST D) Serum total bilirubin. Data has been represented as Mean+/−SEM *p<0.05 for saline vs respective groups. P values were calculated with an ordinary one-way ANOVA. N=4 per group.

FIG. 8E depicts histopathological evaluation of liver injury from H&E staining (40×).

FIG. 9 depicts immunohistochemistry (IHC) staining of macrophage-specific protein F4/80, which reveals an increase in F4/80+ macrophages from 6 to 48 hours post APAP injury.

FIG. 10 depicts IHC staining of LPM specific nuclear GATA6 protein, which reveals infiltration of GATA6+ LPMs to the liver upon AILI.

FIG. 11 depicts that systemically circulating rare labeled macrophages were detected by DiFC after inciting acute liver injury.

FIG. 12 depicts the circulating and secreted pro-inflammatory and anti-inflammatory cytokine profile in peritoneal lavage and liver post APAP injection, which reveals a robust pro-inflammatory response to AILI.

FIG. 13A depicts the gene expression analysis of canonical macrophage markers in LPMs, which reveals a pro-inflammatory macrophage phenotype upon AILI.

FIG. 13B depicts the gene expression analysis of canonical macrophage markers in the liver, which reveals a pro-inflammatory macrophage phenotype upon AILI.

FIG. 14 depicts that a robust in vitro silencing of HMGB1, NFKB1 and TLR4 in primary mouse LPMs was observed with C12-200 encapsulated modified siRNAs.

FIG. 15A depicts that HMGB1 silencing led to protection from LPS-induced inflammation in primary mouse LPMs. In vitro silencing of NF-κB, TLR-4 and HMGB1 gene expression in LPMs treated with LPS. Study design and relative mRNA expression of NF-κB1, TLR-4 and HMGB1, normalized to the PBS-treated group. All the qPCR data was first normalized to a geo-mean of 2 housekeeping genes GAPDH and PPIB before being represented at mRNA expression relative to the respective control siRNA groups.

FIG. 15B depicts gene expression analysis by qPCR of pro-inflammatory macrophage markers iNOS-1, TNF-α; and anti-inflammatory macrophage markers Arg1 and IL-10. Data has been represented as Mean+/−SEM *p<0.05, **p<0.01, ***p<0.001, **** p<0.0001, ns not significant. P values were calculated with an ordinary one-way ANOVA followed by Dunnett's multiple comparison test, with a single pooled variance.

FIG. 16 depicts a schematic representation of the strategy for mitigating AILI and inflammation.

FIG. 17 is a panel of bar graphs depicting in vivo silencing of HMGB1 mRNA expression in LPMs administered at 6 hours and 24 hours intraperitoneal injection and intravenous injection.

FIG. 18A is a panel of bar graphs depicting relative mRNA expression of macrophage pro-inflammatory markers in LPMs.

FIG. 18B is a panel of bar graphs depicting relative mRNA expression of macrophage pro-inflammatory markers in liver.

FIG. 19A is a panel of bar graphs depicting relative mRNA expression of macrophage anti-inflammatory markers in LPMs.

FIG. 19B is a panel of bar graphs depicting relative mRNA expression of macrophage anti-inflammatory markers in liver.

FIG. 20A is a panel of bar graph depicting quantification of percentage GATA6-expressing cells within the isolated liver NPCs in all the treatment groups.

FIG. 20B is a panel of bar graphs depicting relative mRNA expression of iNOS1.

FIG. 20C is a panel of bar graphs depicting Arg1 relative to expression in saline control groups. mRNA expression was derived from quantitative polymerase chain reaction (qPCR) after normalizing expression with an average of housekeeping genes PPIA and GAPDH.

FIG. 20D is a panel of bar graphs depicting secreted pro-inflammatory cytokine levels of TNF-α and IFN-γ in pg/ml.

FIG. 20E is a panel of bar graphs depicting secreted anti-inflammatory cytokine levels of IL-4 and IL-10 in pg/ml. n=4 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 21 shows representative graphs of DiFC scans depicted as number of peaks detected over 600 seconds from one mouse per group from an n=4/group with the indicated treatments. Graphs are representative snapshots of a 10 minutes scan period from a single mouse from a total scanning time of 45 minutes per mouse. Each peak (arrowhead) represents a circulating cell labeled with siRNA-Cy5 (C12-200) in systemic circulation, depicted as signal versus time. n=4 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 22A shows flow cytometry analysis showing representative dot plots of F4/80 hi CD11bhi macrophage population of respective treatment groups in isolated PBMCs.

FIG. 22B shows representative histograms of Cy5+ cells within the F4/80 hi CD11bhi macrophage population. Cells were pre-gated on size and viability. Data is representative of one sample from an n=4 per treatment group.

FIG. 22C shows quantification of overall percentage of F4/80 hi CD11bhi in whole blood PBMCs with the indicated treatment groups.

FIG. 22D shows quantification of percentage Cy5+ cells within the F4/80 hi CD11bhi gated macrophage population with the indicated treatment groups.

FIG. 22E shows quantification of percentage GATA6-expressing cells within the isolated whole blood PBMC lymphocytes with the treatment groups. n=4 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 23A is a panel of bar graphs depicting the relative mRNA expression of HMGB1, depicted as percentage mRNA remaining relative to expression in PBS-treated controls.

FIG. 23B is a panel of bar graphs depicting the relative mRNA expression of NF-κB1, depicted as percentage mRNA remaining relative to expression in PBS-treated controls.

FIG. 23C is a panel of bar graphs depicting the relative mRNA expression of TLR-4, depicted as percentage mRNA remaining relative to expression in PBS-treated controls. mRNA expression was derived from quantitative polymerase chain reaction (qPCR) after normalizing expression with an average of housekeeping genes PPIA and GAPDH.

FIG. 23D is a panel of bar graphs depicting the relative mRNA expression of iNOS1 and TNF-α relative to expression in saline control groups.

FIG. 23E is a panel of bar graphs depicting the relative mRNA expression of Arg1 and IL-10 relative to expression in saline control groups.

FIG. 23F is a panel of bar graphs depicting secreted pro-inflammatory cytokine levels of TNF-α and IFN-γ in pg/ml.

FIG. 23G is a panel of bar graphs depicting secreted anti-inflammatory cytokine levels of IL-4 and IL-10 in pg/ml. n=4 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 24A shows relative mRNA expression of HMGB1 depicted as percentage mRNA remaining relative to expression in gLuc (control) siRNA at 6 hours, 24 hours and 48 hours post siRNA-Cy5 (C12-200) administration in both GLPMs and liver. mRNA expression was derived from quantitative polymerase chain reaction (qPCR) after normalizing expression with an average of housekeeping genes PPIA and GAPDH. n=4 for all groups.

FIG. 24B shows flow cytometry analysis showing representative dot plots of F4/80 hi CD11bhi macrophage population of respective treatment groups in isolated liver NPCs.

FIG. 24C shows representative histograms of Cy5+ cells within the F4/80 hi CD11bhi macrophage population. Cells were pre-gated on size and viability. Data is representative of one sample from an n=4 per treatment group.

FIG. 24D shows quantification of overall percentage of F4/80 hi CD11bhi in liver NPCs with the indicated treatment groups.

FIG. 24E shows quantification of percentage Cy5+ cells within the F4/80 hi CD11bhi gated macrophage population with the indicated treatment groups.

FIG. 24F shows quantification of percentage siRNA-Cy5 (C12-200) carrying GATA6-expressing cells within the isolated liver NPCs in all the treatment groups. n=4 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 25A shows relative mRNA expression of HMGB1 depicted as percentage mRNA remaining relative to expression in gLuc (control) siRNA at 6 hours, 24 hours and 48 hours post siRNA-Cy5 (C12-200) administration in both GLPMs and liver. mRNA expression was derived from quantitative polymerase chain reaction (qPCR) after normalizing expression with an average of housekeeping genes PPIA and GAPDH. n=3 for all groups.

FIG. 25B is a panel of bar graphs depicting levels of serum ALT (depicted on a log 10 scale), serum AST (depicted on a log 10 scale) and serum TBil in pg/ml for the respective treatment groups.

FIG. 25C shows quantification of hepatocellular necrosis based on the histopathological scoring depicted as percentage area of necrosis from the total field observed for the respective treatment groups. n=3 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 26A is a panel of bar graphs depicting relative mRNA expression of iNOS1 and TNF-α.

FIG. 26B is a panel of bar graphs depicting Arg1 and IL-10 in HMGB1 siRNA and NAC-treated groups relative to expression in the gLuc (control) siRNA treated group in GLPMs.

FIG. 26C is a panel of bar graphs depicting secreted pro-inflammatory cytokine levels of TNF-α and IFN-γ in pg/ml in liver NPCs.

FIG. 26D is a panel of bar graphs depicting secreted anti-inflammatory cytokine levels of IL-4 and IL-10 in pg/ml. n=4 for all groups in liver NPCs. N=3 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

FIG. 27A shows relative mRNA expression of HMGB1 depicted as percentage mRNA remaining relative to expression in PBS control at 6 hours, 24 hours and 48 hours post siRNA-GalNAc administration in whole liver. mRNA expression was derived from quantitative polymerase chain reaction (qPCR) after normalizing expression with an average of housekeeping genes PPIA and GAPDH. n=3 for all groups.

FIG. 27B is a panel of bar graphs depicting levels of serum ALT (depicted on a log 10 scale), serum AST (depicted on a log 10 scale), and serum TBil in pg/ml for the respective treatment groups.

FIG. 27C shows quantification of hepatocellular necrosis based on the histopathological scoring depicted as percentage area of necrosis from the total field observed for the respective treatment groups. n=3 for all groups. Data has been represented as Mean+/−SD *p<0.05, **p<0.01, ***p<0.001, **** p<0.0001, ns not significant. P values were calculated with an ordinary one-way ANOVA followed by Dunnett's multiple comparison test, with a single pooled variance.

### DETAILED DESCRIPTION OF THE DISCLOSURE

The present disclosure provides methods of delivering a nucleic acid molecule to a large peritoneal macrophage (LPM), by contacting a nanoparticle (for example, a lipid nanoparticle (LNP)) encapsulating the nucleic acid molecule with the LPM, thereby delivering the nucleic acid molecule to the LPM. The present disclosure successfully utilizes an effective approach of encapsulating a nucleic acid molecule (e.g., an siRNA) in a nanoparticle (e.g., an LNP) for methods of delivering the nanoparticle encapsulating the nucleic acid molecule to the LPM. Compositions generated by these methods are also provided by the present disclosure as are methods of using these compositions.

The methods of the disclosure are both simple, efficient and effective, and result in the production of an LPM comprising the nanoparticle encapsulating the nucleic acid molecule that can be used for a variety of therapeutic applications, for example, delivering the nucleic acid molecule to an injured tissue, e.g., an injured extrperitoneal tissue, in a subject in need thereof. LPMs have a unique ability to migrate to peritoneally located injured tissues and impart wound healing properties. The present disclosure surprisingly demonstrates for the very first time that these LPMs (e.g., GATA6+ LPMS), migrate and infiltrate non-peritoneal tissues, such as the lungs, thereby allowing the use of LPMs as a novel therapeutic tool for treatment of a disease, for example, an inflammatory disease, an infectious disease, an autoimmune disease, or a cancer in a subject in need thereof.

Accordingly, in another aspect the present disclosure provides a method of delivering a nucleic acid molecule to an injured tissue, e.g., an injured extrperitoneal tissue, in a subject in need thereof. The method comprises administering a nanoparticle (for example, an LNP) encapsulating the nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, allowing the LPM comprising the nanoparticle encapsulating the nucleic acid molecule to migrate to the injured tissue, thereby delivering the nucleic acid molecule to the injured tissue in the subject.

The present disclosure also provides methods of therapeutically utilizing LPMs as delivery vehicles to carry the nanoparticle (for example, an LNP) encapsulated nucleic acid modalities to treat a disease, e.g., an inflammatory disease, an infectious disease, an autoimmune disease, or a cancer, in a subject in need thereof. Accordingly, in another aspect the present disclosure provides a method of treating a disease in a subject in need thereof, the method comprising administering a nanoparticle (for example, an LNP) encapsulating a nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, allowing the LPM comprising the nanoparticle encapsulating the nucleic acid molecule to migrate to an injured tissue and deliver the nucleic acid molecule to the injured tissue, thereby treating the disease in the subject.

In another aspect, the present disclosure provides a method of detecting migration of a large peritoneal macrophage (LPM) comprising a nanoparticle (for example, an LNP) encapsulating a nucleic acid molecule to an injured tissue in a subject, the method comprising administering the nanoparticle encapsulating the nucleic acid molecule to the subject, allowing the nanoparticle encapsulating the nucleic acid molecule to contact the LPM in the subject, thereby generating the LPM comprising the nanoparticle encapsulating the nucleic acid molecule, allowing the LPM comprising the nanoparticle encapsulating the nucleic acid molecule to migrate to the injured tissue and deliver the nucleic acid molecule to the injured tissue, thereby detecting the migration of the LPM comprising the nanoparticle encapsulating the nucleic acid molecule to the injured tissue in the subject. Therefore, the present disclosure broadens the opportunity to develop nucleic acid molecule therapies targeted to LPMs without the need to remove them from the body and engineer them ex vivo; and utilizes these cells as a delivery modality.

The following detailed description discloses how to make and use the present disclosure. In the following description, for purposes of explanation, specific numbers, materials, and configurations are set forth in order to provide a thorough understanding of the disclosure. It will be apparent, however, to one having ordinary skill in the art that the disclosure may be practiced without these specific details. In some instances, well-known features may be omitted or simplified so as not to obscure the present disclosure. Furthermore, reference in the specification to phrases such as “one embodiment” or “an embodiment” mean that a particular feature, structure, or characteristic described in connection with the embodiment is included in at least one embodiment of the disclosure. The appearances of phrases such as “in one embodiment” in various places in the specification are not necessarily all referring to the same embodiment.

### I. DEFINITIONS

In order that the present disclosure may be more readily understood, certain terms are first defined. In addition, it should be noted that whenever a value or range of values of a parameter are recited, it is intended that values and ranges intermediate to the recited values are also intended to be part of this disclosure. Unless otherwise specified, each of the following terms have the meaning set forth in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

The term “large peritoneal macrophage (LPM)” is used herein to refer to tissue-resident macrophages (TRMs) of the peritoneal cavity that are formed during embryonic stages (Cassado AdA et al., 2015; and Okabe Y and Medzhitov R. 2014; each of which is incorporated in its entirety herein by reference). LPMs provide the first line of defense against life-threatening pathologies of the peritoneal cavity, such as abdominal sepsis, peritoneal metastatic tumor growth, or peritoneal injuries caused by trauma, or abdominal surgery. Apart from their primary phagocytic function, reminiscent of primitive defense mechanisms sustained by coelomocytes in the coelomic cavity of invertebrates, LPMs fulfill an essential homeostatic function by achieving an efficient clearance of apoptotic cells, that is crucial for the maintenance of self-tolerance. LPMs have a unique migratory ability and can move to injured tissues within the abdominal cavity and impart wound healing properties (Parayath N N et al., 2018; Honda M et al., 2021; Ito T et al., 2021; Wang J and Kubes P. 2016; and Zindel J et al., 2021; each of which is incorporated in its entirety herein by reference). In some embodiments, the LPMs are GATA6+ LPMs.

The term “tissue resident macrophage (TRM)” is used herein to refer to macrophages that perform specialized functions and fulfil organ-specific roles reflected by their distinct transcriptomic profiles, plasticity, phenotype, and functionality across different tissues (Oza D and Amiji M M. 2022; Nobs S P and Kopf M. 2021; Yona S and Gordon S. 2015; Teti G et al., 2017; Gordon S and Plüddemann A. 2017; Jenkins S J and Allen J E. 2021; Cochain C et al., 2018; and Cox N et al., 2021; each of which is incorporated in its entirety herein by reference). In particular, TRMs are a heterogeneous population of immune cells that fulfill tissue-specific and niche-specific functions (Davies et al., Nat Immunol. 2013 October; 14(10): 986-995; incorporated in its entirety herein by reference). These functions include, but are not limited to, dedicated homeostatic functions, such as clearance of cellular debris and iron processing, to central roles in tissue immune surveillance, response to infection and the resolution of inflammation. There also exists a marked heterogeneity in the origins of tissue macrophages that arise from hematopoietic versus self-renewing embryo-derived populations. In some embodiments, the TRMs are LPMs. In some embodiments, the TRMs are alveolar macrophages. In some embodiments, the TRMs are F4/80+ macrophages.

The term “Diffuse in vivo Flow Cytometry (DiFC)” is used herein to refer to a technique of flow cytometry used for enumerating fluorescently labeled circulating cells noninvasively in the bloodstream. In particular, DiFC comprises use of laser-induced fluorescence and highly scattered photons to detect moving cells and fluorescent sensors in relatively large, deeply seated blood vessels. DiFC is non-invasive and does not require drawing blood, and can be performed continuously for extended periods of time and/or can be repeated at multiple timepoints to resolve the kinetics of the migration. Further, DiFC can be used to count events as they pass through systemic circulation in a live subject in real time (Tan X et al., 2019; and Pera V et al., 2017; each of which is incorporated in its entirety herein by reference). In some embodiments, DiFC is used for detecting fluorescent cells in blood, e.g., peripheral blood. In some embodiments, DiFC is used for detecting circulating tumor cells in blood, e.g., in a model of hematogenous metastasis. In some embodiments, DiFC is performed about 0.5 hour, about 1 hour, about 2 hours, about 3 hours, about 6 hours, about 12 hours, about 24 hours or about 48 hours after administering a nanoparticle encapsulating a nucleic acid molecule to a subject.

The term “injured tissue” is used herein to have an ordinary meaning in the art, and includes any and all types of damage to an organ, tissue and/or body part. The injured tissue may comprise any injury or measurable damage resulting from, for example, mechanical forces (i.e., trauma), cuts, tears, lacerations, drugs, toxicants, endotoxin, ischemia, and/or genetic abnormalities. In some embodiments, the injured tissue is a non-peritoneal tissue, e.g., a lung tissue. In some embodiments, the injured tissue is a peritoneal tissue, e.g., a liver tissue or an intestine tissue.

As used herein, “CD45,” refers to the well known gene that encodes for the CD45 protein, a member of the protein tyrosine phosphatase (PTP) family. CD45 is a type I transmembrane protein that is present in various isoforms on all differentiated hematopoietic cells (except erythrocytes and plasma cells). CD45 has been shown to be an essential regulator of T- and B-cell antigen receptor signalling. It functions through either direct interaction with components of the antigen receptor complexes via its extracellular domain (a form of co-stimulation), or by activating various Src family kinases required for the antigen receptor signaling via its cytoplasmic domain. CD45 also suppresses JAK kinases, and so functions as a negative regulator of cytokine receptor signaling. CD45 is also known as Protein Tyrosine Phosphatase Receptor Type C; PTPRC; T200; GP180; LCA; Receptor-Type Tyrosine-Protein Phosphatase C; CD45 Antigen, L-CA; Protein Tyrosine Phosphatase, Receptor Type, C Polypeptide; T200 Leukocyte Common Antigen; Leukocyte-Common Antigen; Leukocyte Common Antigen; T200 Glycoprotein; EC 3.1.3.48; IMD105; CD45R; B220; or LY5.

The sequence of a human CD45 mRNA transcript can be found at, for example, GenBank Accession No.s NM_001267798.2, NM_002838.5, or NM_080921.4. Additional examples of CD45 mRNA sequences are readily available through publicly available databases, e.g., GenBank, UniProt, OMIM, and the Macaca genome project web site. Further information on CD45 can be found, for example, at www.ncbi.nlm.nih.gov/gene/?term=CD45.

As used herein, “HMGB1,” refers to the well known gene that encodes for the high mobility group box 1 (HMGB1) protein, also known as high-mobility group protein 1 (HMG-1) and amphoterin. HMGB1 is a DAMP (damage-associated molecular patterns) which is a key inducer of sterile inflammation. DAMPs like HMGB1 following sterile inflammation can bind to TLR4 expressed on macrophages and amplify the innate immune response. HMGB1 is among the most important chromatin proteins. In the nucleus HMGB1 interacts with nucleosomes, transcription factors, and histones. This nuclear protein organizes the DNA and regulates transcription. After binding, HMGB1 bends the DNA, which facilitates the binding of other proteins. HMGB1 also supports transcription of many genes in interactions with many transcription factors. It also interacts with nucleosomes to loosen packed DNA and remodel the chromatin. Contact with core histones changes the structure of nucleosomes. The presence of HMGB1 in the nucleus depends on posttranslational modifications. When the protein is not acetylated, it stays in the nucleus, but hyperacetylation on lysine residues causes it to translocate into the cytosol. HMGB1 has been shown to play an important role in helping the RAG endonuclease form a paired complex during V(D)J recombination. HMGB1 is also known as High Mobility Group Box; SBP-1; HMG3; HMG1; High-Mobility Group (Nonhistone Chromosomal) Protein; Sulfoglucuronyl Carbohydrate Binding Protein; High Mobility Group Protein B1; High Mobility Group Protein 1; DKFZp686A04236; Amphoterin; HMG-1; or High-Mobility Group Box 1.

The sequence of a human HMGB1 mRNA transcript can be found at, for example, GenBank Accession No.s NM_001313892.2, NM_001313893.1, NM_001363661.2, NM_001370339.1, NM_001370340.1, NM_001370341.1, or NM_002128.7. Additional examples of HMGB1 mRNA sequences are readily available through publicly available databases, e.g., GenBank, UniProt, OMIM, and the Macaca genome project web site. Further information on HMGB1 can be found, for example, at www.ncbi.nlm.nih.gov/gene/?term=HMGB1.

As used herein, “NFkB1,” refers to the well known gene that encodes for the Nuclear factor NF-kappa-B p105 subunit protein. NFkB1 protein is a 105 kD protein which can undergo cotranslational processing by the 26S proteasome to produce a 50 kD protein. The 105 kD protein is a Rel protein-specific transcription inhibitor and the 50 kD protein is a DNA binding subunit of the NF-kappaB (NF-kB) protein complex. NF-kB is a transcription factor that is activated by various intra- and extra-cellular stimuli such as cytokines, oxidant-free radicals, ultraviolet irradiation, and bacterial or viral products. NF-kB represents a family of transcription factors regulating a large array of genes involved in immune and inflammatory responses. Activated NF-kB translocates into the nucleus and stimulates the expression of genes involved in a wide variety of biological functions; over 200 known genes are targets of NF-kB in various cell types, under specific conditions. Inappropriate activation of NF-kB has been associated with a number of inflammatory diseases while persistent inhibition of NF-kB leads to inappropriate immune cell development or delayed cell growth. NFkB1 is also known as Nuclear Factor Kappa B Subunit 1; Nuclear Factor Of Kappa Light Polypeptide Gene Enhancer In B-Cells 1; Nuclear Factor NF-Kappa-B P105 Subunit; NF-KappaB; NFKB-P50; NFkappaB; NF-KB1; KBF1; DNA-Binding Factor KBF1; EBP-1; P105; P50; Nuclear Factor Kappa-B DNA Binding Subunit; Nuclear Factor NF-Kappa-B P50 Subunit; NF-Kappabeta; NF-Kappa-B1; NFKB-P105; CVID12; or NF-KB.

The sequence of a human NFkB1 mRNA transcript can be found at, for example, GenBank Accession No.s NM_001165412.2, NM_001319226.2, NM_001382625.1, NM_001382626.1, NM_001382627.1, NM_001382628.1, or NM_003998.4. Additional examples of NFkB1 mRNA sequences are readily available through publicly available databases, e.g., GenBank, UniProt, OMIM, and the Macaca genome project web site. Further information on NFkB1 can be found, for example, at www.ncbi.nlm.nih.gov/gene/?term=NFKB1.

As used herein, “TLR4,” refers to the well known gene that encodes for the Toll-like receptor 4 protein, a transmembrane protein, member of the toll-like receptor family, which belongs to the pattern recognition receptor (PRR) family. Its activation leads to an intracellular signaling pathway NF-KB and inflammatory cytokine production which is responsible for activating the innate immune system. TLR4 expressing cells are myeloid (erythrocytes, granulocytes, macrophages) rather than lymphoid (T-cells, B-cells, NK cells). Most myeloid cells also express high levels of CD14, which facilitates activation of TLR4 by lipopolysaccharide (LPS). TLR4 belongs to the family of pattern recognition receptors and recognizes many DAMPs and PAMPs that are selectively expressed on innate immune cells like macrophages and dendritic cells. TLR4 helps prime the macrophages for creating an inflammatory response. TLR4 is most well known for recognizing LPS, a component present in many Gram-negative bacteria (e.g., Neisseria spp.) and selected Gram-positive bacteria. Its ligands also include several viral proteins, polysaccharide, and a variety of endogenous proteins such as low-density lipoprotein, beta-defensins, and heat shock protein. Palmitic acid and lauric acid are also TLR4 agonists, and chronic inflammatory responses via cytokine release can result from high dietary intake of these nutrients. However, unsaturated omega-3 and omega-6 fatty acids serve as TLR4 antagonists and can negate the inflammation caused by a high-fat diet. TLR4 has also been designated as CD284 (cluster of differentiation 284). The molecular weight of TLR4 is approximately 95 kDa. TLR4 is also known as Toll Like Receptor 4; HToll; Toll-Like Receptor 4; ARMD10; CD284; TLR-4; Toll Like Receptor 4 Protein; Homolog Of Drosophila Toll; CD284 Antigen; or TOLL.

The sequence of a human TLR4 mRNA transcript can be found at, for example, GenBank Accession No.s NM_003266.4, NM_138554.5, or NM_138557.3. Additional examples of TLR4 mRNA sequences are readily available through publicly available databases, e.g., GenBank, UniProt, OMIM, and the Macaca genome project web site. Further information on TLR4 can be found, for example, at www.ncbi.nlm.nih.gov/gene/?term=TLR4.

The entire contents of each of the foregoing GenBank Accession numbers and the Gene database numbers are incorporated herein by reference as of the date of filing this application.

As used herein, “target sequence” refers to a contiguous portion of the nucleotide sequence of an mRNA molecule formed during the transcription of a gene (e.g., CD45, HMGB1, NFkB1, TLR4, and/or) including mRNA that is a product of RNA processing of a primary transcription product. In one embodiment, the target portion of the sequence will be at least long enough to serve as a substrate for siRNA-directed cleavage at or near that portion of the nucleotide sequence of an mRNA molecule formed during the transcription of the gene.

The target sequence may be from about 19-36 nucleotides in length, e.g., about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety.

The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

The term “nucleic acid” is used herein to refer to any nucleic acid molecule including, but not limited to, a DNA (e.g., a gDNA, or a cDNA), an oligonucleotide (e.g., a single stranded oligonucleotide or a double stranded oligonucleotide), an RNA (e.g., a sense RNA, an antisense RNA, an mRNA, a tRNA, a rRNA, a small interfering RNA (siRNA), a double-stranded RNA (a dsRNA), a short hairpin RNA (shRNA), a piwi-interacting RNAs (piRNA), a micro RNA (miRNA), a small nucleolar RNA (snoRNA), or a small nuclear RNA (snRNAs)), a ribozyme, an aptamer, and/or a DNAzyme. The nucleic acid molecules described herein include non-naturally occurring or modified forms of nucleic acid molecules, as well as naturally occurring forms of nucleic acid molecules.

The terms “siRNA,” “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. The siRNA directs the sequence-specific degradation of mRNA through a process known as RNA interference (RNAi). The siRNA modulates, e.g., inhibits, the expression of a gene, e.g., CD45, High Mobility Group Box 1 (HMGB1), Nuclear factor-kB1 (NFkB1), or Toll Like Receptor 4 (TLR4), in a cell, e.g., an LPM or a cell of an injured tissue, within a subject, such as a human subject.

In one embodiment, an siRNA of the disclosure includes a single stranded RNA that interacts with a target RNA sequence, e.g., a CD45, a HMGB1, a NFkB1, or a TLR4 target mRNA sequence, to direct the cleavage of the target RNA. Without wishing to be bound by theory it is believed that long double stranded RNA introduced into cells is broken down into siRNA by a Type III endonuclease known as Dicer (Sharp et al., (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (siRNA) generated within a cell and which promotes the formation of a RISC complex to effect silencing of the target gene, i.e., a CD45, a HMGB1, a NFkB1, or a TLR4 gene.

In certain embodiments, the siRNA may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “siRNA” for use in the compositions, uses, and methods of the disclosure is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a CD45, a HMGB1, a NFkB1, or a TLR4 RNA. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this specification, an siRNA may include ribonucleotides with chemical modifications; an siRNA may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a RNA molecule, are encompassed by an “siRNA” or “RNAi” for the purposes of this specification and claims.

In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 19 to 36 base pairs in length, e.g., about 19-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, 3, 4, 5, 6, 7, 8, 9, 10, 20, 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, an RNA of the disclosure is a dsRNA, each strand of which comprises 19-23 nucleotides, that interacts with a target RNA sequence, e.g., a CD45, a HMGB1, a NFkB1, a TLR4, or, to direct cleavage of the target RNA.

In some embodiments, an RNA of the disclosure is a dsRNA of 24-30 nucleotides that interacts with a target RNA sequence, e.g., a CD45, a HMGB1, a NFkB1, a TLR4, or a gLuc target mRNA sequence, to direct the cleavage of the target RNA.

As used herein, the term “nucleotide overhang” refers to at least one unpaired nucleotide that protrudes from the duplex structure of a double stranded RNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotides, e.g., a 1, 2, 3, 4, 5, 6, 7, 8, 9, or 10 nucleotide, overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′ end of the antisense strand of the duplex. In certain embodiments, an extended overhang is present on the 5′ end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the disclosure include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an RNA, e.g., a dsRNA, which includes a region that is substantially complementary to a target sequence, e.g., a CD45, a HMGB1, a NFkB1, a TLR4, or a gLuc mRNA.

As used herein, the term “region of complementarity” refers to the region on the antisense strand that is substantially complementary to a sequence, for example a target sequence, e.g., a CD45, a HMGB1, a NFkB1, a TLR4, or a gLuc nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the RNA, e.g., dsRNA. In some embodiments, a double stranded RNA agent of the disclosure includes a nucleotide mismatch in the antisense strand. In some embodiments, the antisense strand of the double stranded RNA agent of the disclosure includes no more than 4 mismatches with the target mRNA, e.g., the antisense strand includes 4, 3, 2, 1, or 0 mismatches with the target mRNA. In some embodiments, the antisense strand double stranded RNA agent of the disclosure includes no more than 4 mismatches with the sense strand, e.g., the antisense strand includes 4, 3, 2, 1, or 0 mismatches with the sense strand. In some embodiments, a double stranded RNA agent of the disclosure includes a nucleotide mismatch in the sense strand. In some embodiments, the sense strand of the double stranded RNA agent of the disclosure includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the dsRNA. In another embodiment, the nucleotide mismatch is, for example, in the 3′-terminal nucleotide of the dsRNA agent. In some embodiments, the mismatch(s) is not in the seed region.

An RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a CD45, a HMGB1, a NFkB1, a TLR4, or a gLuc gene, generally does not contain any mismatch within the central 13 nucleotides. The methods described herein or methods known in the art can be used to determine whether an RNAi agent containing a mismatch to a target sequence is effective in inhibiting the expression of a CD45, a HMGB1, a NFkB1, a TLR4, or a gLuc gene. Consideration of the efficacy of RNAi agents with mismatches in inhibiting expression of a CD45, a HMGB1, a NFkB1, a TLR4, or a gLuc gene is important, especially if the particular region of complementarity in a CD45, a HMGB1, a NFkB1, a TLR4, or a gLuc gene is known to have polymorphic sequence variation within the population.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50 °C or 70 °C for 12-16 hours followed by washing (see, e.g., “Molecular Cloning: A Laboratory Manual, Sambrook, et al., (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucleotides or polynucleotides, such as the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a CD45, a HMGB1, a NFkB1, a TLR4, or a gLuc gene). For example, a polynucleotide is complementary to at least a part of a CD45, a HMGB1, a NFkB1, a TLR4, or a gLuc mRNA if the sequence is substantially complementary to a non-interrupted portion of an mRNA encoding a CD45, a HMGB1, a NFkB1, a TLR4, or a gLuc gene.

Accordingly, in some embodiments, the antisense polynucleotides disclosed herein are fully complementary to the target CD45, HMGB1, NFKB1, TLR4, or gLuc sequence. In other embodiments, the antisense polynucleotides disclosed herein are substantially complementary to the target CD45, HMGB1, NFKB1, TLR4, or gLuc sequence and comprise a contiguous nucleotide sequence.

In other embodiments, the antisense polynucleotides disclosed herein are substantially complementary to the target CD45, HMGB1, NFKB1, TLR4, or gLuc sequence and comprise a contiguous nucleotide sequence which is at least about 80% complementary over its entire length to any one of the sense strand nucleotide sequences of any one of CD45, HMGB1, NFKB1, TLR4, or gLuc sequence, such as about 85%, about 90%, about 91%, about 92%, about 93%, about 94%, about 95%, about 96%, about 97%, about 98%, about 99%, or 100% complementary.

In one embodiment, an RNAi agent of the disclosure includes a sense strand that is substantially complementary to an antisense polynucleotide which, in turn, is the same as a target CD45, HMGB1, NFKB1, TLR4, or gLuc sequence, and wherein the sense strand polynucleotide comprises a contiguous nucleotide sequence which is at least about 80% complementary over its entire length to the equivalent region of the nucleotide sequence of CD45, HMGB1, NFKB1, TLR4, or gLuc sequence, such as about 85%, about 90%, about 91%, about 92%, about 93%, about 94%, about 95%, about 96%, about 97%, about 98%, about 99%, or 100% complementary.

In some embodiments, an iRNA of the disclosure includes a sense strand that is substantially complementary to an antisense polynucleotide which, in turn, is complementary to a target CD45, HMGB1, NFKB1, TLR4, or gLuc sequence, and wherein the sense strand polynucleotide comprises a contiguous nucleotide sequence which is at least about 80% complementary over its entire length to any one of the antisense strand nucleotide sequences in any one of any one of CD45, HMGB1, NFKB1, TLR4, or gLuc sequence, such as about 85%, about 90%, about 91%, about 92%, about 93%, about 94%, about 95%, about 96%, about 97%, about 98%, about 99%, or 100% complementary.

In general, an “siRNA” or “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In certain embodiments of the instant disclosure, inclusion of a deoxy-nucleotide if present within an RNAi agent can be considered to constitute a modified nucleotide.

In an aspect of the disclosure, an agent for use in the methods and compositions of the disclosure is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

The term “nanoparticle” as used herein indicates a composite structure of nanoscale dimensions. In particular, nanoparticles are typically particles of a size in the range of from about 1 to about 1000 nm, and are usually spherical although different morphologies are possible depending on the nanoparticle composition. The portion of the nanoparticle contacting an environment external to the nanoparticle is generally identified as the surface of the nanoparticle. The size limitation of nanoparticles can be restricted to two dimensions, and can include composite structure having a diameter from about 1 to about 1000 nm, where the specific diameter depends on the nanoparticle composition and on the intended use of the nanoparticle according to the experimental design. For example, nanoparticles to be used in several therapeutic applications have a size of about 200 nm or a diameter from about 1 to about 100 nm. The term “targeted nanoparticle” denotes a nanoparticle that is conjugated to a targeting agent or ligand. Additional desirable properties of the nanoparticle, such as surface charges and steric stabilization, can also vary in view of the specific application of interest. Nanoparticle dimensions and properties can be detected by techniques well-known in the art. Exemplary techniques to detect particles dimensions include, but are not limited to, dynamic light scattering (DLS) and a variety of microscopies such at transmission electron microscopy (TEM) and atomic force microscopy (AFM). Exemplary techniques to detect particle morphology include, but are not limited, to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person. In some embodiments, the nanoparticle is a lipid nanoparticle. In some embodiments, the nanoparticle is a polymeric nanoparticle.

The term “lipid nanoparticle” or “LNP” is a vesicle comprising a lipid layer encapsulating a pharmaceutically active molecule, such as a nucleic acid molecule or a plasmid from which the nucleic acid molecule is transcribed. In some embodiments, the nucleic acid molecule is selected from the group consisting of a small interfering (siRNA), a double stranded siRNA (dsRNA), a single stranded siRNA (ssRNAi), a microRNA (miRNA), and an antisense oligonucleotide molecule. In some embodiments, the LNP comprises cationic lipid C12-200, distearoylphosphatidylcholine (DSPC), cholesterol and Poly(ethylene) glycol (PEG)-C14. C12-200 comprising LNP formulations are further described in U.S. Provisional Ser. No. 61/175,770, filed May 5, 2009; and International Application No. PCT/US2010/33777, filed May 5, 2010; the entire contents of each of which are hereby incorporated herein by reference. Additional examples of LNPs that may be used in the present disclosure are described in, for example, U.S. Pat. Nos. 6,858,225, 6,815,432, 8,158,601, and 8,058,069, the entire contents of each of which are hereby incorporated herein by reference.

The term “polymeric nanoparticle” as used herein refers to a nanoparticle comprising one or more polymers. The term “polymer” as used herein indicates a large molecule composed of repeating structural units typically connected by covalent chemical bonds. A suitable polymer may be a linear and/or branched, and can take the form of a homopolymer or a co-polymer. If a co-polymer is used, the co-polymer may be a random copolymer or a branched co-polymer. Exemplary polymers comprise water-dispersible and in particular water soluble polymers. For example, suitable polymers include, but are not limited to polysaccharides, polyesters, polyamides, polyethers, polycarbonates, polyacrylates, etc. For therapeutic and/or pharmaceutical uses and applications, the polymer should have a low toxicity profile and in particular that are not toxic or cytotoxic. Suitable polymers include polymers having a molecular weight of about 500,000 or below. In particular, suitable polymers can have a molecular weight of about 100,000 and below.

The term “cationic lipid” includes those lipids having one or two fatty acid or fatty aliphatic chains and an amino acid containing head group that may be protonated to form a cationic lipid at physiological pH. In some embodiments, a cationic lipid is referred to as an “amino acid conjugate cationic lipid.”

The term “biodegradable cationic lipid” refers to a cationic lipid having one or more biodegradable groups located in the mid- or distal section of a lipidic moiety (e.g., a hydrophobic chain) of the cationic lipid. The incorporation of the biodegradable group(s) into the cationic lipid results in faster metabolism and removal of the cationic lipid from the body following delivery of the active pharmaceutical ingredient to a target area.

The phrase “contacting a nanoparticle encapsulating a nucleic acid molecule with a large peritoneal macrophage (LPM),” or “allowing a nanoparticle encapsulating a nucleic acid molecule to contact an LPM”, and variations thereof, as used herein, include contacting an LPM by any possible means. Contacting a nanoparticle encapsulating a nucleic acid molecule with an LPM includes contacting the LPM with the nanoparticle encapsulating the nucleic acid molecule in vivo; Contacting a nanoparticle encapsulating a nucleic acid molecule with an LPM includes contacting the LPM with the nanoparticle encapsulating the nucleic acid molecule ex vivo; or contacting the LPM with the nanoparticle encapsulating the nucleic acid molecule in vitro. The contacting may be done directly or indirectly. Thus, for example, the nanoparticle encapsulating the nucleic acid molecule may be put into physical contact with the LPM by the individual performing the method, or alternatively, the nanoparticle encapsulating the nucleic acid molecule may be put into a situation that will permit or cause it to subsequently come into contact with the LPM.

Contacting an LPM ex vivo or in vitro may be done, for example, by incubating the LPM with the nanoparticle encapsulating the nucleic acid molecule. Contacting an LPM in vivo may be done, for example, by injecting the nanoparticle encapsulating the nucleic acid molecule into or near the tissue where the LPM is located, or by injecting the nanoparticle encapsulating the nucleic acid molecule into another area, e.g., the bloodstream or the subcutaneous space, such that the nanoparticle encapsulating the nucleic acid molecule will subsequently reach the tissue where the LPM to be contacted is located. For example, the nanoparticle encapsulating the nucleic acid molecule may contain or be coupled to a ligand, that directs the nanoparticle encapsulating the nucleic acid molecule to a site of interest, e.g., a peritoneal tissue or a non-peritoneal tissue. Combinations of in vitro, ex vivo and in vivo methods of contacting are also possible. For example, an LPM may also be contacted in vitro or ex vivo with a nanoparticle encapsulating the nucleic acid molecule and subsequently transplanted into a subject.

In certain embodiments, contacting an LPM with a nanoparticle encapsulating the nucleic acid molecule includes “introducing” or “delivering the nanoparticle encapsulating the nucleic acid molecule into the LPM” by facilitating or effecting uptake or absorption into the LPM. Absorption or uptake of a nanoparticle encapsulating the nucleic acid molecule can occur through unaided diffusion or active cellular processes, or by auxiliary agents or devices. Introducing a nanoparticle encapsulating the nucleic acid molecule into an LPM may be in vitro, ex vivo or in vivo. For example, for in vivo introduction, the nanoparticle encapsulating the nucleic acid molecule can be injected into a tissue site or administered systemically. In vitro introduction into an LPM includes methods known in the art such as electroporation and lipofection. Further approaches are described herein below or are known in the art.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In an embodiment, the subject is a human, such as a human being treated or assessed for a disease or disorder that would benefit from reduction in CD45, HMGB1, NFKB1, TLR4, or gLuc expression; a human at risk for a disease or disorder that would benefit from reduction in CD45, HMGB1, NFKB1, TLR4, or gLuc expression; a human having a disease or disorder that would benefit from reduction in CD45, HMGB1, NFKB1, TLR4, or gLuc expression; or human being treated for a disease or disorder that would benefit from reduction in CD45, HMGB1, NFKB1, TLR4, or gLuc expression as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result, such as reducing at least one sign or symptom of a disease selected from the group consisting of an inflammatory disease, an infectious disease, an autoimmune disease, and a cancer; or a CD45, a HMGB1, a NFkB1, a TLR4, or a gLuc-associated disease or disorder in a subject. Treatment also includes a reduction of one or more sign or symptoms associated with unwanted CD45, HMGB1, NFKB1, TLR4, or gLuc expression; diminishing the extent of unwanted CD45, HMGB1, NFKB1, TLR4, or gLuc activation or stabilization; amelioration or palliation of unwanted CD45, HMGB1, NFKB1, TLR4, or gLuc activation or stabilization. “Treatment” can also mean prolonging survival as compared to expected survival in the absence of treatment.

The term “lower” in the context of the level of CD45, HMGB1, NFKB1, TLR4, or gLuc in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of CD45, HMGB1, NFKB1, TLR4, or gLuc in a subject is a decrease to a level accepted as within the range of normal for an individual without such disorder. In certain embodiments, “lower” is the decrease in the difference between the level of a marker or symptom for a subject suffering from a disease and a level accepted within the range of normal for an individual. The term “lower” can also be used in association with normalizing a symptom of a disease or condition, i.e., decreasing the difference between a level in a subject suffering from a CD45, a HMGB1, a NFkB1, a TLR4, or a gLuc-associated disorder towards or to a level in a normal subject not suffering from a CD45, a HMGB1, a NFkB1, a TLR4, or a gLuc-associated disorder. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder or condition thereof, may be treated or ameliorated by a reduction in expression of a CD45, a HMGB1, a NFkB1, a TLR4, or a gLuc gene, refers to a reduction in the likelihood that a subject will develop a symptom associated with such a disease, disorder, or condition, e.g., a symptom of a CD45, a HMGB1, a NFkB1, a TLR4, or a gLuc-associated disorder, e.g., an inflammatory disease, an infectious disease, an autoimmune disease, and/or a cancer. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

The term “inflammatory disease” is used herein to refer to a disease, disorder, or condition characterized by having inflammation or an inflammatory component of body tissue. Inflammation may be localized or systemic. Inflammatory diseases notably include drug induced liver injury; peritoneal adhesions; inflammatory bowel disease; acute respiratory distress syndrome (ARDS); severe acute respiratory syndrome (SARS); idiopathic pulmonary fibrosis (IPF); hepatitis; graft rejection including skin graft rejection; chronic inflammatory diseases of the joint including arthritis, rheumatoid arthritis, osteoarthritis, acute gouty arthritis, and inflammatory bone diseases (e.g., associated with increased bone resorption); inflammatory lung diseases such as asthma, adult respiratory distress syndrome, and chronic obstructive airway disease; Behcet's disease; inflammatory diseases of the eye including corneal dystrophy, trachoma, onchocerciasis, uveitis, sympathetic ophthalmitis and endophthalmitis; chronic inflammatory diseases of the gums including gingivitis and periodontitis; tuberculosis; leprosy; inflammatory diseases of the kidney including uremic complications, glomerulonephritis and nephrosis; inflammatory disorders of the skin including scleroderma, psoriasis and eczema; inflammatory diseases of the central nervous system, including chronic demyelinating diseases of the nervous system, infectious meningitis, encephalomyelitis, Parkinson's disease, Huntington's disease, amyotrophic lateral sclerosis and viral or autoimmune encephalitis; autoimmune disorders, immune-complex vasculitis; systemic lupus erythematosus (SLE); and inflammatory diseases of the heart such as cardiomyopathy, coronary thrombosis, ischemic heart disease, hypercholesterolemia, atherosclerosis; as well as various other diseases with significant inflammatory components, including preeclampsia, schizophrenia, chronic liver failure, brain and spinal cord trauma, or endometriosis.

In some embodiments, the inflammatory disease is selected from the group consisting of arthritis, rheumatoid arthritis, osteoarthritis, acute gout arthritis, an inflammatory bone disease; an inflammatory lung disease, preferably asthma, adult respiratory distress syndrome, or chronic obstructive airway disease; Behcet's disease; an inflammatory disease of the eye preferably corneal dystrophy, trachoma, onchocerciasis, uveitis, sympathetic ophthalmitis or endophthalmitis; a chronic inflammatory disease of the gums preferably gingivitis or periodontitis; tuberculosis; leprosy; an inflammatory disease of the kidney preferably a uremic complication, glomerulonephritis or nephrosis; an inflammatory disorder of the skin preferably psoriasis; a chronic demyelinating diseases of the nervous system; infectious meningitis; encephalomyelitis; Parkinson's disease; Huntington's disease; amyotrophic lateral sclerosis; an immune-complex vasculitis; systemic lupus erythematosus (SLE); an inflammatory disease of the heart, preferably cardiomyopathy, coronary thrombosis, ischemic heart disease, hypercholesterolemia, or atherosclerosis; preeclampsia; schizophrenia; chronic liver failure, or brain or spinal cord trauma; endometriosis. Preferably, said inflammatory skin disease is selected from among acne (e.g., acne vulgaris or acne conglobate), rosacea, psoriasis, eczema, atopic dermatitis, scleroderma, seborrheic dermatitis, boils, carbuncles, pemphigus, cellulitis, Grover's disease, hidradenitis suppurativa, lichen planus, or any other inflammatory skin disease described herein. Said inflammatory bone disease is preferably selected from among osteoporosis, periodontal disease, ankylosing spondylitis, osteoarthritis, Paget's disease, Lumbar disc herniation (LDH, including e.g., bulging disc, protruded disc, extruded disc, and sequestrated disc), or rheumatoid arthritis, or any other bone disease in which inflammation mediates bone loss or inflammatory bone disease described herein.

The term “infectious disease” is used herein to refer to any infection, disease or condition that can be caused by an organism such as a bacteria, a virus, a fungi or any other pathogenic microbial agent. In some embodiments, the infectious disease is selected from the group consisting of COVID-19, viral hepatitis, tetanus, typhoid fever, diphtheria, syphilis, bacterial vaginosis, Trichomonas vaginalis, meningitis, urinary tract infection, bacterial gastroenteritis, impetigo, cellulitis, pneumonia, lyme disease, and leprosy. In some embodiments, the infectious disease is an infection associated with one or more pathogens selected from the group consisting of coronavirus, Mycobacterium tuberculosis, Streptococcus, Pseudomonas, Shigella, Campylobacter, Salmonella, Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, and Vibrio cholera.

The term “autoimmune disease” is used herein to refer to any disease resulting from an immune response against a self-tissue or tissue component, including both self-antibody responses and cell-mediated responses. In some embodiments, the autoimmune disease is a condition that results from, or is aggravated by, the production of antibodies, e.g., by B cells, that are reactive with normal body tissues and antigens. In some embodiments, the autoimmune disease is one that involves secretion of an autoantibody that is specific for an epitope from a self-antigen (e.g., a nuclear antigen). The term autoimmune disease, as used herein, encompasses organ-specific autoimmune diseases, in which an autoimmune response is directed against a single tissue, and non-organ specific autoimmune diseases, in which an autoimmune response is directed against a component present in several or many organs throughout the body. Autoimmune diseases notably include rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases, such as diabetes.

In some embodiments, the autoimmune disease is selected from the group consisting of rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases such as diabetes.

In some embodiments, the rheumatologic autoimmune disease is selected from the group consisting of rheumatoid arthritis such as acute arthritis, chronic rheumatoid arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, chronic progredient arthritis, arthritis deformans, chronic primary polyarthritis, reactive arthritis, and ankylosing spondylitis, Sjogren's syndrome, scleroderma, lupus such as SLE and lupus nephritis, polymyositis/cryoglobulinemia dermatomyositis, antiphospholipid antibody syndrome, and psoriatic arthritis.

In some embodiments, the gastrointestinal and liver autoimmune disease is selected from the group consisting of autoimmune gastritis and pernicious anemia, autoimmune hepatitis, primary biliary cirrhosis, primary sclerosing cholangitis, and celiac disease.

In some embodiments, the vasculitis is selected from the group consisting of ANCA-associated vasculitis, Churg-Strauss vasculitis, Wegener's granulomatosis, and polyarteritis.

In some embodiments, the renal autoimmune diseases is selected from the group consisting of glomerulonephritis, syndrome Goodpasture, and Berger's disease.

In some embodiments, the dermatological autoimmune diseases is selected from the group consisting of psoriasis such as plaque psoriasis, guttate psoriasis, pustular psoriasis, and psoriasis of the nails, urticaria such as chronic allergic urticaria and chronic idiopathic urticaria, pemphigus vulgaris, bullous pemphigoid, lupus erythematosus, inflammatory hyperproliferative skin diseases, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non specific dermatitis, primary irritant contact dermatitis, and atopic dermatitis, eczema including allergic or atopic eczema, asteatotic eczema, dyshidrotic eczema, and vesicular palmoplantar eczema.

In some embodiments, the hematologic autoimmune diseases is selected from the group consisting of thrombocytopenic purpura, thrombotic thrombocytopenic purpura, post-transfusion purpura, and autoimmune hemolytic anemia.

In some embodiments, the autoimmune ear diseases is selected from the group consisting of inner ear disease and hearing loss.

In some embodiments, the autoimmune diseases associated with organ transplantation is selected from the group consisting of graft rejection and Graft vs Host disease (GvHD). In some embodiments, the organ transplant is selected from the group consisting of blood transplant, bone marrow transplant, stem cell transplant, kidney transplant, pancreas transplant, liver transplant, orthotopic liver transplant, lung transplant, heart transplant, intestine transplant, small intestine transplant, large intestine transplant, thymus transplant, allograft stem cells transplant, allograft of lesser intensity, bone transplant, tendon transplant, cornea transplant, skin transplant, cardiac valves transplant, veins transplant, arteries transplant, blood vessels transplant, stomach transplant, and testicle transplant.

In some embodiments, the autoimmune endocrine diseases is selected from the group consisting of juvenile onset (Type 1) diabetes mellitus, including pediatric insulin-dependent diabetes mellitus (IDDM), adult onset diabetes mellitus (Type II diabetes), autoimmune diabetes, idiopathic diabetes insipidus, and diseases related to diabetes (such as diabetic retinopathy, diabetic nephropathy, diabetic large-artery disorder) Addison's disease, and autoimmune thyroid disease (such as Graves' disease, Hashimoto's thyroiditis, subacute thyroiditis, idiopathic hypothyroidism).

In some embodiments, the autoimmune disease is selected from the group consisting of atherosclerosis, uveitis, and Raynaud's syndrome.

The term “cancer” used herein to refer to diseases caused by uncontrolled cell division, growth of cells in additional sites, and/or hyperproliferation of cells whose loss of normal controls results in unregulated growth, lack of differentiation, local tissue invasion, and/or metastasis. In some embodiments, the cancer is selected from the group consisting of hepatocellular carcinoma, acute lymphocytic cancer, acute myeloid leukemia, alveolar rhabdomyosarcoma, bladder cancer, bone cancer, brain cancer, breast cancer, cancer of the anus, anal canal cancer, rectum cancer, cancer of the eye, cancer of the intrahepatic bile duct, cancer of the joints, cancer of the neck, gallbladder cancer, pleura cancer, nose cancer, nasal cavity cancer, middle ear cancer, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumor, liver cancer, lung cancer, lymphoma, malignant mesothelioma, mastocytoma, melanoma, multiple myeloma, nasopharynx cancer, nonHodgkin lymphoma, ovarian cancer, pancreatic cancer, peritoneum or omentum, cancer, mesentery cancer, pharynx cancer, prostate cancer, colorectal cancer, renal cancer, skin cancer, small intestine cancer, soft tissue cancer, solid tumor, stomach cancer, testicular cancer, thyroid cancer, ureter cancer, and urinary bladder cancer. As used herein, the term “tumor” refers to an abnormal growth of cells or tissues, e.g., of malignant type or benign type.

“Therapeutically effective amount,” as used herein, is intended to include the amount of an RNAi agent that, when administered to a subject having a CD45, a HMGB1, a NFkB1, a TLR4, or a gLuc-associated disorder, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having a CD45, a HMGB1, a NFkB1, a TLR4, or a gLuc-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired effect at a reasonable benefit/risk ratio applicable to any treatment. The iRNA employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

### II. METHODS OF THE DISCLOSURE

The present disclosure provides methods of delivering a nucleic acid molecule to a large peritoneal macrophage (LPM), by contacting a nanoparticle encapsulating the nucleic acid molecule with the LPM, thereby delivering the nucleic acid molecule to the LPM. The methods of the disclosure are both simple, efficient and effective, and result in the production of an LPM comprising the nanoparticle encapsulating the nucleic acid molecule that can be used for a variety of therapeutic applications, for example, delivering the nucleic acid molecule to a site of interest, e.g., an injured tissue, in a subject in need thereof. The present disclosure also provides methods of therapeutically utilizing LPMs as delivery vehicles to carry the nanoparticle encapsulated nucleic acid modalities to treat a disease, e.g., an inflammatory disease, an infectious disease, an autoimmune disease, or a cancer, in a subject in need thereof.

In some embodiments, the nucleic acid molecule is selected from the group consisting of an oligonucleotide, a DNA, an RNA, a ribozyme, an aptamer, and a DNAzyme.

In some embodiments, the nanoparticle is a lipid nanoparticle (LNP). In some embodiments, the LNP comprises cationic lipid C12-200, distearoylphosphatidylcholine (DSPC), cholesterol and Poly(ethylene) glycol (PEG)-C14.

In some embodiments, the nanoparticle is a polymeric nanoparticle (LNP).

### A. Large Peritoneal Macrophages (LPMs)

In certain embodiments of the disclosure, there are disclosed methods and compositions for producing a large peritoneal macrophage (LPM) comprising a nanoparticle encapsulating a nucleic acid molecule.

In one aspect, the disclosure provides methods of delivering a nucleic acid molecule to a large peritoneal macrophage (LPM), the method comprising contacting a nanoparticle (e.g., a lipid nanoparticle (LNP)) encapsulating the nucleic acid molecule with the LPM, thereby delivering the nucleic acid molecule to the LPM.

In some embodiments, the contacting the LPM with the nanoparticle encapsulating the nucleic acid molecule is performed in vivo. In some embodiments, the contacting the LPM with the nanoparticle encapsulating the nucleic acid molecule is performed ex vivo.

The term “large peritoneal macrophage (LPM)” is used herein to refer to tissue-resident macrophages (TRMs) of the peritoneal cavity that are formed during embryonic stages (Cassado AdA et al., 2015; and Okabe Y and Medzhitov R. 2014; each of which is incorporated in its entirety herein by reference). LPMs provide the first line of defense against life-threatening pathologies of the peritoneal cavity, such as abdominal sepsis, peritoneal metastatic tumor growth, or peritoneal injuries caused by trauma, or abdominal surgery. Apart from their primary phagocytic function, reminiscent of primitive defense mechanisms sustained by coelomocytes in the coelomic cavity of invertebrates, LPMs fulfill an essential homeostatic function by achieving an efficient clearance of apoptotic, that is crucial for the maintenance of self-tolerance. LPMs have a unique migratory ability and can move to injured tissues within the abdominal cavity and impart wound healing properties (Parayath N N et al., 2018; Honda M et al., 2021; Ito T et al., 2021; Wang J and Kubes P. 2016; and Zindel J et al., 2021; each of which is incorporated in its entirety herein by reference).

In some embodiments, the LPMs are positive for the zinc-finger ranscription factor GATA6, i.e., they are GATA6+ LPMs.

LPMs have a unique ability to migrate to peritoneally located injured tissues and impart wound healing properties. The present disclosure surprisingly demonstrates for the very first time that these LPMs (e.g., GATA6+ LPMS) migrate and infiltrate non-peritoneal tissues (e.g., lungs) for example, following depletion of tissue resident macrophages (e.g., alveolar macrophages (AMs)).

In some embodiments, the LPMs comprising the nanoparticle encapsulating the nucleic acid molecule migrate to an injured tissue in a subject in need thereof, and serve as a delivery vehicle for delivering the nucleic acid molecule to a cell of the injured tissue in the subject.

In some embodiments, the injured tissue is a non-peritoneal tissue. In some embodiments, non-peritoneal tissue is a lung tissue.

In some embodiments, the administering of the nanoparticle encapsulating the nucleic acid molecule to the subject is performed intraperitoneally.

In some embodiments, the nucleic acid molecule is selected from the group consisting of an oligonucleotide, a DNA, an RNA, a ribozyme, an aptamer, and a DNAzyme.

### B. Nanoparticles of the Disclosure

Nucleic acid molecules of the disclosure can be encapsulated within nanoparticles, such as lipid nanoparticles or polymeric nanoparticles. For example, nucleic acid molecules, e.g., siRNAs, of the disclosure may be fully encapsulated in a lipid formulations, e.g., a LNP. In some embodiments, the LNP forms a SPLP, pSPLP, SNALP, or other nucleic acid-lipid particles.

The term “nanoparticle” as used herein indicates a composite structure of nanoscale dimensions. In particular, nanoparticles are typically particles of a size in the range of from about 1 to about 1000 nm, and are usually spherical although different morphologies are possible depending on the nanoparticle composition. The portion of the nanoparticle contacting an environment external to the nanoparticle is generally identified as the surface of the nanoparticle. The size limitation of nanoparticles can be restricted to two dimensions, and can include composite structure having a diameter from about 1 to about 1000 nm, where the specific diameter depends on the nanoparticle composition and on the intended use of the nanoparticle according to the experimental design. For example, nanoparticles to be used in several therapeutic applications have a size of about 200 nm or a diameter from about 1 to about 100 nm. The term “targeted nanoparticle” denotes a nanoparticle that is conjugated to a targeting agent or ligand. Additional desirable properties of the nanoparticle, such as surface charges and steric stabilization, can also vary in view of the specific application of interest. Nanoparticle dimensions and properties can be detected by techniques well-known in the art. Exemplary techniques to detect particles dimensions include, but are not limited to, dynamic light scattering (DLS) and a variety of microscopies such at transmission electron microscopy (TEM) and atomic force microscopy (AFM). Exemplary techniques to detect particle morphology include, but are not limited, to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person. In some embodiments, the nanoparticle is a lipid nanoparticle. In some embodiments, the nanoparticle is a polymeric nanoparticle.

The term “lipid nanoparticle” or “LNP” is a vesicle comprising a lipid layer encapsulating a pharmaceutically active molecule, such as a nucleic acid molecule or a plasmid from which the nucleic acid molecule is transcribed. In some embodiments, the nucleic acid molecule is selected from the group consisting of a small interfering (siRNA), a double stranded siRNA (dsRNA), a single stranded siRNA (ssRNAi), a microRNA (miRNA), and an antisense oligonucleotide molecule. In some embodiments, the LNP comprises cationic lipid C12-200, distearoylphosphatidylcholine (DSPC), cholesterol and Poly(ethylene) glycol (PEG)-C14. C12-200 comprising LNP formulations are further described in U.S. Provisional Ser. No. 61/175,770, filed May 5, 2009; and International Application No. PCT/US2010/33777, filed May 5, 2010; the entire contents of each of which are hereby incorporated herein by reference. Additional examples of LNPs that may be used in the present disclosure are described in, for example, U.S. Pat. Nos. 6,858,225, 6,815,432, 8,158,601, and 8,058,069, the entire contents of each of which are hereby incorporated herein by reference.

The term “polymeric nanoparticle” as used herein refers to a nanoparticle comprising one or more polymers. The term “polymer” as used herein indicates a large molecule composed of repeating structural units typically connected by covalent chemical bonds. A suitable polymer may be a linear and/or branched, and can take the form of a homopolymer or a co-polymer. If a co-polymer is used, the co-polymer may be a random copolymer or a branched co-polymer. Exemplary polymers comprise water-dispersible and in particular water soluble polymers. For example, suitable polymers include, but are not limited to polysaccharides, polyesters, polyamides, polyethers, polycarbonates, polyacrylates, etc. For therapeutic and/or pharmaceutical uses and applications, the polymer should have a low toxicity profile and in particular that are not toxic or cytotoxic. Suitable polymers include polymers having a molecular weight of about 500,000 or below. In particular, suitable polymers can have a molecular weight of about 100,000 and below.

As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to siRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure.

In some embodiments, the LNP comprises a cationic lipid.

The cationic lipid can be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleoyl-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid can comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the LNP includes 40% 2, 2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidylethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C)₈. The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the lipidoid ND98-4HCl (MW 1487) (see U.S. patent application Ser. No. 12/056,230, filed Mar. 26, 2008, which is incorporated herein by reference), Cholesterol (Sigma-Aldrich), and PEG-Ceramide C16 (Avanti Polar Lipids) can be used to prepare lipid-dsRNA nanoparticles (i.e., LNP01 particles).

disclosuredisclosure In some embodiments, suitable cationic lipids include one or more biodegradable groups. The biodegradable group(s) include one or more bonds that may undergo bond breaking reactions in a biological environment, e.g., in an organism, organ, tissue, cell, or organelle. Functional groups that contain a biodegradable bond include, for example, esters, dithiols, and oximes. Biodegradation can be a factor that influences the clearance of the compound from the body when administered to a subject. Biodegredation can be measured in a cell based assay, where a formulation including a cationic lipid is exposed to cells, and samples are taken at various time points. The lipid fractions can be extracted from the cells and separated and analyzed by LC-MS. From the LC-MS data, rates of biodegradation (e.g., as t½ values) can be measured. the cationic lipid comprises a biodegradable group.

In one embodiment, a cationic lipid of any of the embodiments described herein has an in vivo half life (t½) (e.g., in the liver, spleen or plasma) of less than about 3 hours, such as less than about 2.5 hours, less than about 2 hours, less than about 1.5 hours, less than about 1 hour, less than about 0.5 hour or less than about 0.25 hours. The cationic lipid preferably remains intact, or has a half-life sufficient to form a stable lipid nanoparticle which effectively delivers the desired active pharmaceutical ingredient (e.g., a nucleic acid) to its target but thereafter rapidly degrades to minimize any side effects to the subject. For instance, in mice, the cationic lipid preferably has a t½ in the spleen of from about 1 to about 7 hours.

In another embodiment, a cationic lipid of any of the embodiments described herein containing a biodegradable group or groups has an in vivo half life (t½) (e.g., in the liver, spleen or plasma) of less than about 10% (e.g., less than about 7.5%, less than about 5%, less than about 2.5%) of that for the same cationic lipid without the biodegrable group or groups.

In certain embodiments, the cationic lipid is

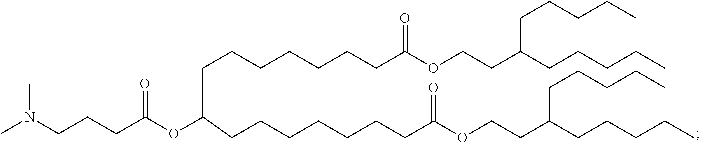 [SMILES: CCCCCC(CCCCC)CCOC(=O)CCCCCCCC(CCCCCCCC(=O)OCCC(CCCCC)CCCCC)OC(=O)CCCN(C)C]

In certain embodiments, the dsRNA agents of the disclosure are formulated with a cationic lipid, e.g.,

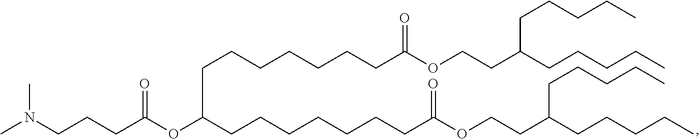 [SMILES: CCCCCC(CCCCC)CCOC(=O)CCCCCCCC(CCCCCCCC(=O)OCCC(CCCCC)CCCCC)OC(=O)CCCN(C)C]

distearoylphosphatidylcholine (DSPC), cholesterol (Chol), and 1,2-Dimyristoyl-rac-glycero-3-methoxypolyethylene glycol (PEG-DMG). In one embodiment, the ratio of

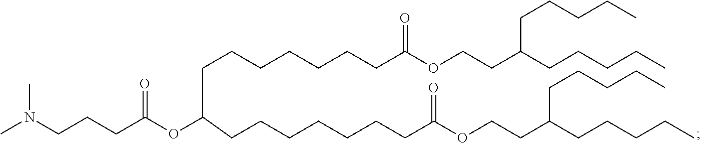 [SMILES: CCCCCC(CCCCC)CCOC(=O)CCCCCCCC(CCCCCCCC(=O)OCCC(CCCCC)CCCCC)OC(=O)CCCN(C)C]

DSPC:Chol:PEG-DMG is about 50:12:36:2, respectively.

Included in the present disclosure is the free form of the cationic lipids described herein, as well as pharmaceutically acceptable salts and stereoisomers thereof. The cationic lipid can be a protonated salt of the amine cationic lipid. The term “free form” refers to the amine cationic lipids in non-salt form. The free form may be regenerated by treating the salt with a suitable dilute aqueous base solution such as dilute aqueous NaOH, potassium carbonate, ammonia and sodium bicarbonate. The pharmaceutically acceptable salts of the instant cationic lipids can be synthesized from the cationic lipids of this disclosure which contain a basic or acidic moiety by conventional chemical methods. Generally, the salts of the basic cationic lipids are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the cationic lipids of this disclosure include non-toxic salts of the cationic lipids of this disclosure as formed by reacting a basic instant cationic lipids with an inorganic or organic acid. For example, non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, and trifluoroacetic (TFA).

When the cationic lipids of the present disclosure are acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, and zinc. In one embodiment, the base is selected from ammonium, calcium, magnesium, potassium and sodium. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N′-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, and tromethamine.

It will also be noted that the cationic lipids of the present disclosure may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

### C. Nucleic Acid Molecules of the Disclosure

The present disclosure provides nucleic acid molecules, for example, encapsulated in a nanoparticle described herein, for delivery to an LPM. In some embodiments, the LPM further delivers the nucleic acid molecule to an injured tissue in a subject in need thereof.

In some embodiments, the nucleic acid molecule is selected from the group consisting of an oligonucleotide, a DNA, an RNA, a ribozyme, an aptamer, and a DNAzyme.

In some embodiments, the nucleic acid molecule is an oligonucleotide. In some embodiments, the nucleic acid molecule is a DNA. In some embodiments, the nucleic acid molecule is an RNA. In some embodiments, the nucleic acid molecule is a ribozyme. In some embodiments, the nucleic acid molecule is an aptamer. In some embodiments, the nucleic acid molecule is a DNAzyme.

In some embodiments, one or more of the nucleic acid molecules disclosed herein can be used for altering gene expression, for example, by effecting a disruption in a gene, such as a knock-out, insertion, missense or frameshift mutation, such as biallelic frameshift mutation, deletion of all or part of the gene, e.g., one or more exon or portion therefore, and/or knock-in. For example, the one or more of the nucleic acid molecules disclosed herein can be used in combination with sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof. Methods to employ one or more of the nucleic acid molecules of the present disclosure in conjunction with sequence-specific or targeted nucleases (e.g., ZFN, TALENs and/or CRISPR/Cas systems) would be apparent to one of skill in the art.

Additionally, the one or more of the nucleic acid molecules disclosed herein can be used for facilitating alteration in gene expression, for example, by using antisense techniques, such as by RNA interference (RNAi).

In some embodiments, the oligonucleotide is a single stranded oligonucleotide or a double stranded oligonucleotide. In some embodiments, the oligonucleotide is a single stranded oligonucleotide. In some embodiments, the oligonucleotide is a double stranded oligonucleotide

In some embodiments, the DNA is selected from the group consisting of a genomic DNA (gDNA) and a copy DNA (cDNA). In some embodiments, the DNA is a genomic DNA (gDNA). In some embodiments, the DNA is a copy DNA (cDNA).

In some embodiments, the nucleic acid molecule is a ribozyme. Ribozymes (ribonucleic acid enzymes) are RNA molecules that have the ability to catalyze specific biochemical reactions, including RNA splicing in gene expression, similar to the action of protein enzymes. The most common activities of natural or in vitro evolved ribozymes are the cleavage (or ligation) of RNA and DNA and peptide bond formation. For example, the smallest ribozyme known (GUGGC-3′) can aminoacylate a GCCU-3′ sequence in the presence of PheAMP. Within the ribosome, ribozymes function as part of the large subunit ribosomal RNA to link amino acids during protein synthesis. Ribozymes can be used in a variety of RNA processing reactions, including RNA splicing, viral replication, and transfer RNA biosynthesis.

In some embodiments, the ribozyme is selected from the group consisting of a hairpin ribozyme, a hammerhead ribozyme, a hepatitis delta virus ribozyme, a Varkud Satellite ribozyme, and a glmS ribozyme. In some embodiments, the ribozyme is a hairpin ribozyme. In some embodiments, the ribozyme is a hammerhead ribozyme. In some embodiments, the ribozyme is a hepatitis delta virus ribozyme. In some embodiments, the ribozyme is a Varkud Satellite ribozyme. In some embodiments, the ribozyme is a glmS ribozyme.

In some embodiments, the nucleic acid molecule is an aptamer. Aptamers are short sequences of artificial DNA, RNA, XNA, or peptide that bind a specific target molecule, or family of target molecules. They exhibit a range of affinities (KD in the pM to M range), with variable levels of off-target binding and are sometimes classified as chemical antibodies. Aptamers and antibodies can be used in combination in any of the applications described herein.

Aptamers are based on a specific oligomer sequence of 20-100 bases and 3-20 kDa. In some embodiments, the aptamers of the disclosure comprise chemical modifications for functional enhancements or compatibility with larger engineered molecular systems. DNA, RNA, XNA, and peptide aptamer chemistries can each offer distinct profiles in terms of shelf stability, durability in serum or in vivo, specificity and sensitivity, cost, ease of generation, amplification, and characterization, and familiarity to users. Typically, DNA- and RNA-based aptamers exhibit low immunogenicity, are amplifiable via Polymerase Chain Reaction (PCR), and have complex secondary structure and tertiary structure. DNA- and XNA-based aptamers exhibit superior shelf stability. XNA-based aptamers can introduce additional chemical diversity to increase binding affinity or greater durability in serum or in vivo.

In some embodiments, the aptamers target small molecules, heavy metal ions, larger ligands such as proteins, and/or whole cells. Such targets include, but are not limited to, lysozyme, thrombin, human immunodeficiency virus trans-acting responsive element (HIV TAR), hemin, interferon γ, vascular endothelial growth factor (VEGF), prostate specific antigen (PSA), dopamine, and/or the non-classical oncogene-heat shock factor 1 (HSF1). Aptamers can also be utilized against cancer cells, prions, bacteria, and viruses. Viral targets of aptamers include influenza A and B viruses, Respiratory syncytial virus (RSV), SARS coronavirus (SARS-CoV), and SARS-CoV-2.

In some embodiments, the nucleic acid molecule is a DNAzyme. DNAzyme also called DNA enzymes, Deoxyribozymes, or catalytic DNA, are DNA oligonucleotides that are capable of performing a specific chemical reaction, often but not always catalytic. This is similar to the action of other biological enzymes, such as proteins or ribozymes. DNAzyme should not be confused with DNA aptamers which are oligonucleotides that selectively bind a target ligand, but do not catalyze a subsequent chemical reaction.

The most abundant class of DNAzyme are ribonucleases, which catalyze the cleavage of a ribonucleotide phosphodiester bond through a transesterification reaction, forming a 2′3′-cyclic phosphate terminus and a 5′-hydroxyl terminus. For example, a DNA molecule with sequence 5′-GGAGAACGCGAGGCAAGGCTGGGAGAAATGTGGATCACGATT-3′ acts as a DNAzyme that uses light to repair a thymine dimer, using serotonin as cofactor.

Several studies have shown the usage of DNAzymes to inhibit influenza A and B virus replication in host cells (Kumar B et al., 2018. Archives of Virology. 163 (4): 831-844; and Asha K et al., 2018. Journal of Clinical Medicine. 8 (1): 6; each of which is incorporated in its entirety herein by reference). In some embodiments, the DNAzymes are used to inhibit the replication of influenza A and B virus, SARS coronavirus (SARS-CoV), Respiratory syncytial virus (RSV), human rhinovirus 14 and HCV.

In some embodiments, the nucleic acid molecule is an RNA. Ribozymes In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the RNA is a sense RNA. In some embodiments, the RNA is an antisense RNA. In some embodiments, the RNA is a messenger RNA (mRNA). In some embodiments, the RNA is a transfer RNA (tRNA). In some embodiments, the RNA is a ribosomal RNA (rRNA). In some embodiments, the RNA is a small interfering RNA (siRNA). In some embodiments, the RNA is a double-stranded RNA (dsRNA). In some embodiments, the RNA is a short hairpin RNA (shRNA). In some embodiments, the RNA is a piwi-interacting RNA (piRNA). In some embodiments, the RNA is a micro RNA (miRNA). In some embodiments, the RNA is a small nucleolar RNA (snoRNA). In some embodiments, the RNA is a small nuclear RNA (snRNA). In some embodiments, the RNA is a guide RNA (gRNA).

In some embodiments, the nucleic acid molecule is an siRNA. In some embodiments, the siRNA inhibits the expression of a CD45, a HMGB1, a NFkB1, a TLR4, or a gLuc gene in an LPM and/or a cell of an injured tissue disclosed herein, such as an LPM or a cell of an injured tissue within a subject, e.g., a mammal, such as a human. The siRNA includes an antisense strand having a region of complementarity which is complementary to at least a part of an mRNA formed in the expression of a CD45, a HMGB1, a NFkB1, a TLR4, or a gLuc gene. In some embodiments, the region of complementarity is about 19-30 nucleotides in length (e.g., about 30, 29, 28, 27, 26, 25, 24, 23, 22, 21, 20, or 19 nucleotides in length).

Upon contact with a cell, such as an LPM, expressing the CD45, HMGB1, NFKB1, TLR4, or gLuc gene, the siRNA inhibits the expression of the CD45, HMGB1, NFKB1, TLR4, or gLuc gene (e.g., a human, CD45, HMGB1, NFKB1, TLR4, or gLuc gene) by at least about 50% as assayed by, for example, a PCR or branched DNA (bDNA)-based method, or by a protein-based method, such as by immunofluorescence analysis, using, for example, western blotting or flow cytometric techniques. In certain embodiments, inhibition of expression is determined by the qPCR method provided in the examples herein with the siRNA at, e.g., a 10 nM concentration, in an appropriate organism cell line provided therein. In certain embodiments, inhibition of expression in vivo is determined by knockdown of the human gene in a rodent expressing the human gene, e.g., a mouse or an AAV-infected mouse expressing the human target gene, e.g., when administered as single dose, e.g., at 3 mg/kg at the nadir of RNA expression.

In some embodiments, the nucleic acid molecule is a dsRNA. A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a CD45, a HMGB1, a NFkB1, a TLR4, or a gLuc gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22,18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA.

A dsRNA as described herein can further include one or more single-stranded nucleotide overhangs, e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the disclosure may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the disclosure can be prepared using solution-phase or solid-phase organic synthesis or both.

In an aspect, a dsRNA of the disclosure includes at least two nucleotide sequences, a sense sequence and an anti-sense sequence. In this aspect, one of the two sequences is complementary to the other of the two sequences, with one of the sequences being substantially complementary to a sequence of an mRNA generated in the expression of a CD45, a HMGB1, a NFkB1, a TLR4, or a gLuc gene.

In certain embodiments, the substantially complementary sequences of the dsRNA are contained on separate oligonucleotides. In other embodiments, the substantially complementary sequences of the dsRNA are contained on a single oligonucleotide.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al., (2005) Nat Biotech 23:222-226).

### D. Modified Nucleic Acid Molecules of the Disclosure

In certain embodiments, the nucleic acid molecule, e.g., a siRNA or a dsRNA, of the disclosure is un-modified, and does not comprise, e.g., chemical modifications or conjugations known in the art and described herein. In other embodiments, the nucleic acid molecule, e.g., a siRNA or a dsRNA, of the disclosure, is chemically modified to enhance stability or other beneficial characteristics. In certain embodiments of the disclosure, substantially all of the nucleotides of the nucleic acid molecule of the disclosure are modified. In other embodiments of the disclosure, all of the nucleotides of the nucleic acid molecule or substantially all of the nucleotides of the nucleic acid molecule are modified, i.e., not more than 5, 4, 3, 2, or 1 unmodified nucleotides are present in a strand of the nucleic acid molecule.

The nucleic acid molecule featured in the disclosure can be synthesized or modified by methods well established in the art, such as those described in “Current protocols in nucleic acid chemistry,” Beaucage, S. L. et al., (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of nucleic acid molecules useful in the embodiments described herein include, but are not limited to nucleic acid molecules, e.g., RNAs containing modified backbones or no natural internucleoside linkages. Nucleic acid molecule having modified backbones include, among others, those that do not have a phosphorus atom in the backbone. For the purposes of this specification, and as sometimes referenced in the art, modified nucleic acid molecule, such as dsRNAs that do not have a phosphorus atom in their internucleoside backbone can also be considered to be oligonucleosides. In some embodiments, a modified nucleic acid molecule will have a phosphorus atom in its internucleoside backbone.

Modified backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the disclosure, the nucleic acid molecules, such as dsRNA agents of the disclosure are in a free acid form. In other embodiments of the disclosure, the dsRNA agents of the disclosure are in a salt form. In one embodiment, the dsRNA agents of the disclosure are in a sodium salt form. In certain embodiments, when the dsRNA agents of the disclosure are in the sodium salt form, sodium ions are present in the agent as counterions for substantially all of the phosphodiester and/or phosphorothiotate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the disclosure are in the sodium salt form, sodium ions are present in the agent as counterions for all of the phosphodiester and/or phosphorothiotate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Suitable nucleic acid molecule, e.g., siRNA, mimetics are contemplated for use in methods provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as O—P(O)(OH)—OCH2-.

Modified RNAs can also contain one or more substituted sugar moieties. The RNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)₋ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative US patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

A nucleic acid molecule, e.g., an siRNA, of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxythimidine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2 °C (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In some embodiments, an RNAi agent of the disclosure can also be modified to include one or more bicyclic sugar moieties. A “bicyclic sugar” is a furanosyl ring modified by a ring formed by the bridging of two carbons, whether adjacent or non-adjacent. A “bicyclic nucleoside” (“BNA”) is a nucleoside having a sugar moiety comprising a ring formed by bridging two carbons, whether adjacent or non-adjacent, of the sugar ring, thereby forming a bicyclic ring system. In certain embodiments, the bridge connects the 4′-carbon and the 2′-carbon of the sugar ring, optionally, via the 2′-acyclic oxygen atom. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge.

A locked nucleoside can be represented by the structure (omitting stereochemistry),

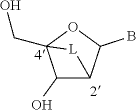

wherein B is a nucleobase or modified nucleobase and L is the linking group that joins the 2′-carbon to the 4′-carbon of the ribose ring. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C1-C12 alkyl, or a nitrogen protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and U.S. patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

The RNA of an iRNA can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge (i.e., L in the preceding structure). In one embodiment, a constrained ethyl nucleotide is in the S conformation referred to herein as “S-cEt.”

An siRNA of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, U.S. Patent Publication No. 2013/0190383; and PCT publication WO 2013/036868, the entire contents of each of which are hereby incorporated herein by reference.

In some embodiments, an siRNA of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3”-phosphate, inverted base dT(idT) and others. Disclosure of this modification can be found in PCT Publication No. WO 2011/005861.

Other modifications of the nucleotides of an siRNA of the disclosure include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

1. Modified siRNAs Comprising Motifs of the Disclosure

In certain aspects of the disclosure, the double stranded RNA agents of the disclosure include agents with chemical modifications as disclosed, for example, in WO2013/075035, the entire contents of each of which are incorporated herein by reference. As shown herein and in WO2013/075035, one or more motifs of three identical modifications on three consecutive nucleotides may be introduced into a sense strand or antisense strand of a dsRNA agent, particularly at or near the cleavage site. In some embodiments, the sense strand and antisense strand of the dsRNA agent may otherwise be completely modified. The introduction of these motifs interrupts the modification pattern, if present, of the sense or antisense strand. The dsRNA agent may be optionally conjugated with a GalNAc derivative ligand, for instance on the sense strand.

More specifically, when the sense strand and antisense strand of the double stranded RNA agent are completely modified to have one or more motifs of three identical modifications on three consecutive nucleotides at or near the cleavage site of at least one strand of a dsRNA agent, the gene silencing activity of the dsRNA agent was observed.

Accordingly, the disclosure provides double stranded RNA agents capable of inhibiting the expression of a target gene (i.e., CD45, HMGB1, NFKB1, TLR4, or gLuc gene) in vivo. The RNAi agent comprises a sense strand and an antisense strand. Each strand of the RNAi agent may be, for example, 17-30 nucleotides in length, 25-30 nucleotides in length, 27-30 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as “dsRNAi agent.” The duplex region of a dsRNAi agent may be, for example, the duplex region can be 27-30 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 19, 20, 21, 22, 23, 24, 25, 26, and 27 nucleotides in length.

In certain embodiments, the dsRNAi agent may contain one or more overhang regions or capping groups at the 3′-end, 5′-end, or both ends of one or both strands. The overhang can be, independently, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In certain embodiments, the overhang regions can include extended overhang regions as provided above. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In certain embodiments, the nucleotides in the overhang region of the dsRNAi agent can each independently be a modified or unmodified nucleotide including, but no limited to 2′-sugar modified, such as, 2′-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand, or both strands of the dsRNAi agent may be phosphorylated. In some embodiments, the overhang region(s) contains two nucleotides having a phosphorothioate between the two nucleotides, where the two nucleotides can be the same or different. In some embodiments, the overhang is present at the 3′-end of the sense strand, antisense strand, or both strands. In some embodiments, this 3′-overhang is present in the antisense strand. In some embodiments, this 3′-overhang is present in the sense strand.

The dsRNAi agent may contain only a single overhang, which can strengthen the interference activity of the RNAi, without affecting its overall stability. For example, the single-stranded overhang may be located at the 3′-end of the sense strand or, alternatively, at the 3′-end of the antisense strand. The RNAi may also have a blunt end, located at the 5′-end of the antisense strand (i.e., the 3′-end of the sense strand) or vice versa. Generally, the antisense strand of the dsRNAi agent has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In certain embodiments, the dsRNAi agent is a double blunt-ended of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, and 13 from the 5′end.

In other embodiments, the dsRNAi agent is a double blunt-ended of 20 nucleotides in length, wherein the sense strand contains at least one motif of three 2′-F modifications on three consecutive nucleotides at positions 8, 9, and 10 from the 5′end. The antisense strand contains at least one motif of three 2′-O-methyl modifications on three consecutive nucleotides at positions 11, 12, and 13 from the 5′end.

In yet other embodiments, the dsRNAi agent is a double blunt-ended of 21 nucleotides in length, wherein the sense strand contains at least one motif of three 2′-F modifications on three consecutive nucleotides at positions 9, 10, and 11 from the 5′end. The antisense strand contains at least one motif of three 2′-O-methyl modifications on three consecutive nucleotides at positions 11, 12, and 13 from the 5′end.

In certain embodiments, the dsRNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′end; the antisense strand contains at least one motif of three 2′-O-methyl modifications on three consecutive nucleotides at positions 11, 12, and 13 from the 5′end, wherein one end of the RNAi agent is blunt, while the other end comprises a 2 nucleotide overhang. In one embodiment, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In certain embodiments, every nucleotide in the sense strand and the antisense strand of the dsRNAi agent, including the nucleotides that are part of the motifs are modified nucleotides. In certain embodiments each residue is independently modified with a 2′-O-methyl or 3′-fluoro, e.g., in an alternating motif. Optionally, the dsRNAi agent further comprises a ligand (such as, GalNAc₃).

In certain embodiments, the dsRNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In certain embodiments, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein Dicer cleavage of the dsRNAi agent results in an siRNA comprising the 3′-end of the second strand, thereby reducing expression of the target gene in the mammal. Optionally, the dsRNAi agent further comprises a ligand.

In certain embodiments, the sense strand of the dsRNAi agent contains at least one motif of three identical modifications on three consecutive nucleotides, where one of the motifs occurs at the cleavage site in the sense strand.

In certain embodiments, the antisense strand of the dsRNAi agent can also contain at least one motif of three identical modifications on three consecutive nucleotides, where one of the motifs occurs at or near the cleavage site in the antisense strand.

For a dsRNAi agent having a duplex region of 19-23 nucleotides in length, the cleavage site of the antisense strand is typically around the 10, 11, and 12 positions from the 5′-end. Thus the motifs of three identical modifications may occur at the 9, 10, 11 positions; the 10, 11, 12 positions; the 11, 12, 13 positions; the 12, 13, 14 positions; or the 13, 14, 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the dsRNAi agent from the 5′-end.

The sense strand of the dsRNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In some embodiments, the sense strand of the dsRNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistries of the motifs are distinct from each other, and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the dsRNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In some embodiments, the wing modification on the sense strand or antisense strand of the dsRNAi agent typically does not include the first one or two terminal nucleotides at the 3′-end, 5′-end, or both ends of the strand.

In other embodiments, the wing modification on the sense strand or antisense strand of the dsRNAi agent typically does not include the first one or two paired nucleotides within the duplex region at the 3′-end, 5′-end, or both ends of the strand.

When the sense strand and the antisense strand of the dsRNAi agent each contain at least one wing modification, the wing modifications may fall on the same end of the duplex region, and have an overlap of one, two, or three nucleotides.

When the sense strand and the antisense strand of the dsRNAi agent each contain at least two wing modifications, the sense strand and the antisense strand can be so aligned that two modifications each from one strand fall on one end of the duplex region, having an overlap of one, two, or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In some embodiments, every nucleotide in the sense strand and antisense strand of the dsRNAi agent, including the nucleotides that are part of the motifs, may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′-hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′- or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′-end or ends can be phosphorylated.

It may be possible, e.g., to enhance stability, to include particular bases in overhangs, or to include modified nucleotides or nucleotide surrogates, in single strand overhangs, e.g., in a 5′- or 3′-overhang, or in both. For example, it can be desirable to include purine nucleotides in overhangs. In some embodiments all or some of the bases in a 3′- or 5′-overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

### DISCLOSURE

The iRNA may further comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In one embodiment, the dsRNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In certain embodiments, the dsRNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In certain embodiments, the nucleotide at the 1 position within the duplex region from the 5′-end in the antisense strand is selected from A, dA, dU, U, and dT. Alternatively, at least one of the first 1, 2, or 3 base pair within the duplex region from the 5′-end of the antisense strand is an AU base pair. For example, the first base pair within the duplex region from the 5′-end of the antisense strand is an AU base pair.

disclosuredisclosuredisclosuredisclosure In other embodiments, an RNAi agent of the disclosure may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric iRNAs that can be used in the methods of the disclosure. Such publications include WO2007/091269, U.S. Pat. No. 7,858,769, WO2010/141511, WO2007/117686, WO2009/014887, and WO2011/031520 the entire contents of each of which are hereby incorporated herein by reference.

In certain embodiments, the compositions and methods of the disclosure include a vinyl phosphonate (VP) modification of an RNAi agent as described herein. In exemplary embodiments, a 5′ vinyl phosphonate modified nucleotide of the disclosure has the structure:

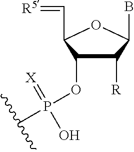

wherein X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphonate modifications are also contemplated for the compositions and methods of the instant disclosure. An exemplary vinyl phosphonate structure includes the preceding structure, where R5′ is ═C(H)—OP(O)(OH)2 and the double bond between the C5′ carbon and R5′ is in the E or Z orientation (e.g., E orientation).

As described in more detail below, the iRNA that contains conjugations of one or more carbohydrate moieties to an iRNA can optimize one or more properties of the iRNA. In many cases, the carbohydrate moiety will be attached to a modified subunit of the iRNA. For example, the ribose sugar of one or more ribonucleotide subunits of a iRNA can be replaced with another moiety, e.g., a non-carbohydrate (such as, cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g., fused rings. The cyclic carrier may be a fully saturated ring system, or it may contain one or more double bonds.

The ligand may be attached to the polynucleotide via a carrier. The carriers include (i) at least one “backbone attachment point,” such as, two “backbone attachment points” and (ii) at least one “tethering attachment point.” A “backbone attachment point” as used herein refers to a functional group, e.g., a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g., monosaccharide, disaccharide, trisaccharide, tetrasaccharide, oligosaccharide, or polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The iRNA may be conjugated to a ligand via a carrier, wherein the carrier can be cyclic group or acyclic group. In one embodiment, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a serinol backbone or diethanolamine backbone.

### E. Nucleic Acid Molecules Conjugated to Ligands

Another modification of the nucleic acid molecule, e.g., siRNA of the disclosure involves chemically linking to the nucleic acid molecule, e.g., siRNA one or more ligands, moieties or conjugates that enhance the activity, cellular distribution, or cellular uptake of the nucleic acid molecule, e.g., siRNA, into a cell. Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N. Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting, or lifetime of an iRNA agent into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. In some embodiments, ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxol, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present disclosure as ligands (e.g., as PK modulating ligands). In addition, aptamers that bind serum components (e.g., serum proteins) are also suitable for use as PK modulating ligands in the embodiments described herein.

Ligand-conjugated iRNAs of the disclosure may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present disclosure may be conveniently and routinely made through the well-known technique of solid-phase synthesis. Equipment for such synthesis is sold by several vendors including, for example, Applied Biosystems® (Foster City, Calif.). Any other methods for such synthesis known in the art may additionally or alternatively be employed. It is also known to use similar techniques to prepare other oligonucleotides, such as the phosphorothioates and alkylated derivatives.

In the ligand-conjugated iRNAs and ligand-molecule bearing sequence-specific linked nucleosides of the present disclosure, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present disclosure are synthesized by an automated synthesizer using phosphoramidites derived from ligand-nucleoside conjugates in addition to the standard phosphoramidites and non-standard phosphoramidites that are commercially available and routinely used in oligonucleotide synthesis.

Representative U.S. patents that teach the preparation of RNA conjugates include, but are not limited to, International PCT Publication No. WO 2009/073809; U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present disclosure also includes iRNA compounds that are chimeric compounds.

“Chimeric” iRNA compounds or “chimeras,” in the context of this disclosure, are iRNA compounds, such as, dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### F. Delivery of Nucleic Acid Molecules of the Disclosure

The method of delivery of a nucleic acid molecule to a large peritoneal macrophage (LPM) within a subject, such as a human subject (e.g., a subject in need thereof, such as a subject susceptible to or diagnosed with an inflammatory disease, an infectious disease, an autoimmune disease, and/or a cancer) can be achieved in a number of different ways. For example, delivery may be performed by contacting an LPM with a nanoparticle encapsulating a nucleic acid molecule of the disclosure either in vitro, ex vivo, or in vivo. In vivo delivery may also be performed directly by administering a nanoparticle encapsulating a nucleic acid molecule, e.g., an oligonucleotide, a DNA, an RNA, a ribozyme, an aptamer, and/or a DNAzyme, to a subject.

In general, any method of delivering a nanoparticle encapsulating a nucleic acid molecule of the disclosure (in vitro, ex vivo, or in vivo) can be adapted for use with a nucleic acid molecule of the disclosure (see e.g., Akhtar S. and Julian R L. (1992) Trends Cell. Biol. 2(5):139-144 and WO94/02595, which are incorporated herein by reference in their entireties). For in vivo delivery, factors to consider in order to deliver a nucleic acid molecule molecule include, for example, biological stability of the delivered molecule, prevention of non-specific effects, and accumulation of the delivered molecule in the target tissue. Nucleic acid molecules can be modified by chemical conjugation to lipophilic groups such as cholesterol to prevent degradation.

Products and systems, such as delivery vehicles, comprising the agents of the disclosure, especially those formulated as pharmaceutical compositions, as well as kits comprising such delivery vehicles and/or systems, as described herein, are also envisioned as being part of the present disclosure.

In certain embodiments, a therapeutic method of the disclosure includes the step of administering compositions of the disclosure, as described herein, with an implant or device. In certain embodiments, the device is bioerodible implant for treating a disease or condition described herein. The volume of composition administered according to the methods described herein is also dependent on factors such as the mode of administration, age of the patient, and type and severity of the disease being treated.

### G. Methods for Detecting Migration of Large Peritoneal Macrophages (LPMs) of the Disclosure

These present disclosure provides the potential to develop nucleic acid molecule based therapeutic modalities, such as RNAi therapies, targeted to LPMs without the need to remove them from the body and engineer them ex vivo; and utilizes these cells as a delivery modality.

Accordingly, in another aspect, the present disclosure provides a method of detecting migration of a large peritoneal macrophage (LPM) comprising a nanoparticle (for example, an LNP) encapsulating a nucleic acid molecule to an injured tissue in a subject, the method comprising administering the nanoparticle encapsulating the nucleic acid molecule to the subject, allowing the nanoparticle encapsulating the nucleic acid molecule to contact the LPM in the subject, thereby generating the LPM comprising the nanoparticle encapsulating the nucleic acid molecule, allowing the LPM comprising the nanoparticle encapsulating the nucleic acid molecule to migrate to the injured tissue and deliver the nucleic acid molecule to the injured tissue, thereby detecting the migration of the LPM comprising the nanoparticle encapsulating the nucleic acid molecule to the injured tissue in the subject. This broadens the opportunity to develop nucleic acid molecule therapies targeted to LPMs without the need to remove them from the body and engineer them ex vivo; and utilizes these cells as a delivery modality.

In some embodiments, the LPM comprising the nanoparticle encapsulating the nucleic acid molecule migrates to the injured tissue via systemic circulation in the subject.

In some embodiments, detecting the migration of the LPM comprising the nanoparticle encapsulating the nucleic acid molecule to the injured tissue in the subject is performed by Diffuse in vivo Flow Cytometry (DiFC).

The term “Diffuse in vivo Flow Cytometry (DiFC)” is used herein to refer to a technique of flow cytometry used for enumerating fluorescently labeled circulating cells noninvasively in the bloodstream. In particular, DiFC comprises use of laser-induced fluorescence and highly scattered photons to detect moving cells and fluorescent sensors in relatively large, deeply seated blood vessels. DiFC is non-invasive and does not require drawing blood, and can be performed continuously for extended periods of time and/or can be repeated at multiple timepoints to resolve the kinetics of the migration. Further, DiFC can be used to count events as they pass through systemic circulation in a live subject in real time (Tan X et al., 2019; and Pera V et al., 2017; each of which is incorporated in its entirety herein by reference). In some embodiments, DiFC is used for detecting fluorescent cells in blood, e.g., peripheral blood. In some embodiments, DiFC is used for detecting circulating tumor cells in blood, e.g., in a model of hematogenous metastasis.

In some embodiments, DiFC is performed about 0.5 hour, about 1 hour, about 2 hours, about 3 hours, about 6 hours, about 12 hours, about 24 hours or about 48 hours after administering a nanoparticle encapsulating a nucleic acid molecule to a subject.

In some embodiments, DiFC is performed about 0.5 hour after administering a nanoparticle encapsulating a nucleic acid molecule to a subject. In some embodiments, DiFC is performed about 1 hour after administering a nanoparticle encapsulating a nucleic acid molecule to a subject. In some embodiments, DiFC is performed about 2 hours after administering a nanoparticle encapsulating a nucleic acid molecule to a subject. In some embodiments, DiFC is performed about 3 hours after administering a nanoparticle encapsulating a nucleic acid molecule to a subject. In some embodiments, DiFC is performed about 6 hours after administering a nanoparticle encapsulating a nucleic acid molecule to a subject. In some embodiments, DiFC is performed about 12 hours after administering a nanoparticle encapsulating a nucleic acid molecule to a subject. In some embodiments, DiFC is performed about 24 hours after administering a nanoparticle encapsulating a nucleic acid molecule to a subject. In some embodiments, DiFC is performed about 48 hours after administering a nanoparticle encapsulating a nucleic acid molecule to a subject.

In some embodiments, the administering of the nanoparticle encapsulating the nucleic acid molecule to the subject is performed intraperitoneally.

In some embodiments, the nucleic acid molecule is labeled with a cy5.5 fluorophore.

In some embodiments, nucleic acid molecule is selected from the group consisting of an oligonucleotide, a DNA, an RNA, a ribozyme, an aptamer, and a DNAzyme.

In some embodiments, RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), Toll Like Receptor 4 (TLR4), and gLuc.

In some embodiments, the siRNA comprises at least one modified nucleotide. In some embodiments, the modified nucleotide is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In some embodiments, the nanoparticle is a lipid nanoparticle (LNP). In some embodiments, the LNP comprises cationic lipid C12-200, distearoylphosphatidylcholine (DSPC), cholesterol and Poly(ethylene) glycol (PEG)-C14. In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

### H. Methods for Inhibiting Expression of a Target Gene

The present disclosure also provides methods of inhibiting expression of a gene of interest, e.g., CD45, a HMGB1, a NFkB1, a TLR4, and/or a gLuc gene, in an LPM and/or in an injured tissue described herein. In some embodiments, the methods comprise contacting a nanoparticle encapsulating a nucleic acid molecule with an LPM, wherein the nucleic acid molecule is, for example, an siRNA, wherein the siRNA targets one or more genes selected from the group consisting of CD45, HMGB1, NFkB1, TLR4, and gLuc.

Contacting a nanoparticle encapsulating a nucleic acid molecule with an LPM includes contacting the LPM with the nanoparticle encapsulating the nucleic acid molecule in vivo; contacting the LPM with the nanoparticle encapsulating the nucleic acid molecule ex vivo; or contacting the LPM with the nanoparticle encapsulating the nucleic acid molecule in vitro. The contacting may be done directly or indirectly. Thus, for example, the nanoparticle encapsulating the nucleic acid molecule may be put into physical contact with the LPM by the individual performing the method, or alternatively, the nanoparticle encapsulating the nucleic acid molecule may be put into a situation that will permit or cause it to subsequently come into contact with the LPM.

Contacting an LPM in vitro or ex vivo may be done, for example, by incubating the LPM with the nanoparticle encapsulating the nucleic acid molecule. Contacting an LPM in vivo may be done, for example, by injecting the nanoparticle encapsulating the nucleic acid molecule into or near the tissue where the LPM is located, or by injecting the nanoparticle encapsulating the nucleic acid molecule into another area, e.g., the bloodstream or the subcutaneous space, such that the nanoparticle encapsulating the nucleic acid molecule will subsequently reach the tissue where the LPM to be contacted is located. For example, the nanoparticle encapsulating the nucleic acid molecule may contain or be coupled to a ligand, that directs the nanoparticle encapsulating the nucleic acid molecule to a site of interest, e.g., a peritoneal tissue or a non-peritoneal tissue. Combinations of in vitro, ex vivo and in vivo methods of contacting are also possible. For example, an LPM may also be contacted in vitro with a nanoparticle encapsulating the nucleic acid molecule and subsequently transplanted into a subject.

The term “inhibiting,” as used herein, is used interchangeably with “reducing,” “silencing,” “downregulating”, “suppressing”, and other similar terms, and includes any level of inhibition.

Inhibiting expression of a CD45, a HMGB1, a NFkB1, a TLR4, or a gLuc gene includes any level of inhibition of a CD45, a HMGB1, a NFkB1, a TLR4, or a gLuc gene, e.g., at least partial suppression of the expression of a CD45, a HMGB1, a NFkB1, a TLR4, or a gLuc gene. The expression of the CD45, HMGB1, NFKB1, TLR4, or gLuc gene may be assessed based on the level, or the change in the level, of any variable associated with CD45, HMGB1, NFKB1, TLR4, or gLuc gene expression, e.g., CD45, HMGB1, NFKB1, TLR4, or gLuc mRNA level or CD45, HMGB1, NFKB1, TLR4, or gLuc protein level.

The expression of a CD45, a HMGB1, a NFkB1, a TLR4, or a gLuc may also be assessed indirectly based on other variables associated with CD45, HMGB1, NFKB1, TLR4, or gLuc gene expression. Inhibition may be assessed by a decrease in an absolute or relative level of one or more variables that are associated with CD45, HMGB1, NFKB1, TLR4, or gLuc expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of a CD45, a HMGB1, a NFkB1, a TLR4, or a gLuc gene is inhibited by at least 50%, 55%, 60%, 65%, 70%, 75%, 80%, 85%, 90%, or 95%, or to below the level of detection of the assay. In some embodiments, expression of a CD45, a HMGB1, a NFkB1, a TLR4, or a gLuc gene is inhibited by at least 70%. It is further understood that inhibition of CD45, HMGB1, NFKB1, TLR4, or gLuc expression in certain tissues, e.g., in lung or liver, without a significant inhibition of expression in other tissues, may be desirable.

Inhibition of the expression of a CD45, a HMGB1, a NFkB1, a TLR4, or a gLuc gene may be manifested by a reduction of the amount of mRNA expressed by a first cell (e.g., an LPM and/or a cell of an injured tissue described herein) or group of cells (e.g., LPMs and/or cells of an injured tissue described herein) in which a CD45, a HMGB1, a NFkB1, a TLR4, or a gLuc gene is transcribed and which has or have been treated (e.g., by contacting the cell or cells with a nucleic acid molecule of the disclosure, or by administering a nanoparticle encapsulating a nucleic acid molecule of the disclosure to a subject in which the cells are or were present) such that the expression of a CD45, a HMGB1, a NFkB1, a TLR4, or a gLuc gene is inhibited, as compared to a second cell or group of cells substantially identical to the first cell or group of cells but which has not or have not been so treated (control cell(s) not treated with a nucleic acid molecule or not treated with a nucleic acid molecule targeted to the gene of interest). In some embodiments, the inhibition is assessed by determining the level of mRNA in treated cells as a percentage of the level of mRNA in control cells, using the following formula:

$$\frac{( m R N A in control cells ) - ( m R N A in treated cells )}{( m R N A in control cells )} 100 \%$$

In other embodiments, inhibition of the expression of a CD45, a HMGB1, a NFkB1, a TLR4, or a gLuc gene may be assessed in terms of a reduction of a parameter that is functionally linked to CD45, HMGB1, NFKB1, TLR4, or gLuc gene expression, e.g., CD45, HMGB1, NFKB1, TLR4, or gLuc protein level in blood or serum from a subject. CD45, HMGB1, NFKB1, TLR4, or gLuc gene silencing may be determined in any cell expressing CD45, HMGB1, NFKB1, TLR4, or gLuc, either endogenous or heterologous from an expression construct, and by any assay known in the art.

Inhibition of the expression of a CD45, a HMGB1, a NFkB1, a TLR4, or a gLuc protein may be manifested by a reduction in the level of the CD45, HMGB1, NFKB1, TLR4, or gLuc protein that is expressed by a cell or group of cells or in a subject sample (e.g., the level of protein in a blood sample derived from a subject). As explained above, for the assessment of mRNA suppression, the inhibition of protein expression levels in a treated cell (e.g., an LPM and/or a cell of an injured tissue described herein) or group of cells may similarly be expressed as a percentage of the level of protein in a control cell or group of cells, or the change in the level of protein in a subject sample, e.g., blood or serum derived therefrom.

A control cell, a group of cells, or subject sample that may be used to assess the inhibition of the expression of a CD45, a HMGB1, a NFkB1, a TLR4, or a gLuc gene includes a cell, group of cells, or subject sample that has not yet been contacted with an RNAi agent of the disclosure. For example, the control cell, group of cells, or subject sample may be derived from an individual subject (e.g., a human or animal subject) prior to treatment of the subject with a nucleic acid molecule of the disclosure or an appropriately matched population control.

The level of CD45, HMGB1, NFKB1, TLR4, or gLuc mRNA that is expressed by a cell or group of cells may be determined using any method known in the art for assessing mRNA expression. In one embodiment, the level of expression of CD45, HMGB1, NFKB1, TLR4, or gLuc in a sample is determined by detecting a transcribed polynucleotide, or portion thereof, e.g., mRNA of the CD45, HMGB1, NFKB1, TLR4, or gLuc gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™ Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression of CD45, HMGB1, NFKB1, TLR4, or gLuc is determined using a nucleic acid probe. The term “probe”, as used herein, refers to any molecule that is capable of selectively binding to a specific CD45, HMGB1, NFKB1, TLR4, or gLuc. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to CD45, HMGB1, NFKB1, TLR4, or gLuc mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of CD45, HMGB1, NFKB1, TLR4, or gLuc mRNA.

An alternative method for determining the level of expression of CD45, HMGB1, NFKB1, TLR4, or gLuc in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989) Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of CD45, HMGB1, NFKB1, TLR4, or gLuc is determined by quantitative fluorogenic RT-PCR (i.e., the TaqMan™ System).

The expression levels of CD45, HMGB1, NFKB1, TLR4, or gLuc mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of CD45, HMGB1, NFKB1, TLR4, or gLuc expression level may also comprise using nucleic acid probes in solution.

In some embodiments, the level of mRNA expression is assessed using branched DNA (bDNA) assays or real time PCR (qPCR). The use of these methods is described and exemplified in the Examples presented herein.

The level of CD45, HMGB1, NFKB1, TLR4, or gLuc protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the disclosure are assessed by a decrease in CD45, HMGB1, NFKB1, TLR4, or gLuc mRNA or protein level (e.g., in a liver biopsy). The inhibition of expression of CD45, HMGB1, NFKB1, TLR4, or gLuc may be assessed using measurements of the level or change in the level of CD45, HMGB1, NFKB1, TLR4, or gLuc mRNA or CD45, HMGB1, NFKB1, TLR4, or gLuc protein in a sample derived from fluid or tissue from the specific site within the subject (e.g., liver, lung, or blood).

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### III. COMPOSITIONS OF THE DISCLOSURE

A further aspect of the disclosure provides a composition comprising a large peritoneal macrophage (LPM), e.g., a GATA6+ LPM, comprising a nanoparticle encapsulating a nucleic acid molecule.

In some embodiments, the composition comprises a population of LPMs, e.g., GATA6+ LPMs, comprising a nanoparticle encapsulating a nucleic acid molecule. In some embodiments, the composition comprising a population of LPMs, e.g., GATA6+ LPMs, comprising a nanoparticle encapsulating a nucleic acid molecule comprises at least 1×10⁵, 1×10⁶, 1×10⁷, 1×10⁸, 1×10⁹, 1×10¹⁰, 1×10¹¹, or 1×10¹² LPMs comprising a nanoparticle encapsulating a nucleic acid molecule. In some embodiments, the composition comprising a population of LPMs comprising a nanoparticle encapsulating a nucleic acid molecule comprises at least 1×10⁵ LPMs comprising a nanoparticle encapsulating a nucleic acid molecule. In some embodiments, the composition comprising a population of LPMs comprising a nanoparticle encapsulating a nucleic acid molecule comprises at least 1×10⁶ LPMs comprising a nanoparticle encapsulating a nucleic acid molecule. In some embodiments, the composition comprising a population of LPMs comprising a nanoparticle encapsulating a nucleic acid molecule comprises at least 1×10⁷ LPMs comprising a nanoparticle encapsulating a nucleic acid molecule. In some embodiments, the composition comprising a population of LPMs comprising a nanoparticle encapsulating a nucleic acid molecule comprises at least 1×10⁸ LPMs comprising a nanoparticle encapsulating a nucleic acid molecule. In some embodiments, the composition comprising a population of LPMs comprising a nanoparticle encapsulating a nucleic acid molecule comprises at least 1×10⁹ LPMs comprising a nanoparticle encapsulating a nucleic acid molecule. In some embodiments, the composition comprising a population of LPMs comprising a nanoparticle encapsulating a nucleic acid molecule comprises at least 1×10¹⁰ LPMs comprising a nanoparticle encapsulating a nucleic acid molecule. In some embodiments, the composition comprising a population of LPMs comprising a nanoparticle encapsulating a nucleic acid molecule comprises at least 1×10¹¹ LPMs comprising a nanoparticle encapsulating a nucleic acid molecule. In some embodiments, the composition comprising a population of LPMs comprising a nanoparticle encapsulating a nucleic acid molecule comprises at least 1×10¹² LPMs comprising a nanoparticle encapsulating a nucleic acid molecule.

In some embodiments, the LPM is a GATA6+ LPM.

In some embodiments, the nucleic acid molecule is selected from the group consisting of an oligonucleotide, a DNA, an RNA, a ribozyme, an aptamer, and a DNAzyme.

In some embodiments, the RNA is selected from the group consisting of a sense RNA, an antisense RNA, a messenger RNA (mRNA), a transfer RNA (tRNA), a ribosomal RNA (rRNA), a small interfering RNA (siRNA), a double-stranded RNA (dsRNA), a short hairpin RNA (shRNA), a piwi-interacting RNA (piRNA), a micro RNA (miRNA), a small nucleolar RNA (snoRNA), a small nuclear RNA (snRNA), and a guide RNA (gRNA).

In some embodiments, the siRNA targets at least one gene selected from the group consisting of CD45, High Mobility Group Box 1 (HMGB1), Nuclear factor-kB1 (NFkB1), Toll Like Receptor 4 (TLR4), and gLuc.

In some embodiments, the siRNA comprises at least one modified nucleotide, as described herein.

In some embodiments, the nanoparticle is a lipid nanoparticle (LNP). In some embodiments, the LNP comprises cationic lipid C12-200, distearoylphosphatidylcholine (DSPC), cholesterol and Poly(ethylene) glycol (PEG)-C14. In some embodiments, the molar ratio of the C12-200, the DSPC, the cholesterol, and the PEG-C14 in the LNP is 50, 10, 38.5, and 1.5, respectively.

In some embodiments, the nanoparticle is a polymeric nanoparticle.

The present disclosure also includes pharmaceutical compositions and formulations which comprise the compositions of the disclosure, e.g., an LPM comprising a nanoparticle encapsulating a nucleic acid molecule. In some embodiments, the pharmaceutical compositions further comprise a pharmaceutically acceptable carrier. The pharmaceutical compositions containing the nucleic acid molecule described herein are useful for preventing or treating a disease, e.g., a disease selected from the group consisting of an inflammatory disease, an infectious disease, an autoimmune disease, and a cancer.

Such pharmaceutical compositions are formulated based on the mode of delivery. One example is compositions that are formulated for systemic administration via parenteral delivery, e.g., by peritoneal, subcutaneous (SC), intramuscular (IM), or intravenous (IV) delivery.

In some embodiments, the pharmaceutical compositions of the disclosure are sterile. In another embodiment, the pharmaceutical compositions of the disclosure are pyrogen free.

The pharmaceutical compositions of the disclosure may be administered in dosages sufficient to inhibit expression of a CD45, a HMGB1, a NFkB1, a TLR4, or a gLuc gene in an LPM and/or a cell of an injured tissue described herein. In general, a suitable dose of a nucleic acid molecule, e.g., an siRNA, encapsulated in a nanoparticle of the disclosure will be in the range of about 0.001 to about 200.0 milligrams per kilogram body weight of the recipient per day, generally in the range of about 1 to 50 mg per kilogram body weight per day. Typically, a suitable dose of a nucleic acid molecule, e.g., an siRNA, of the disclosure will be in the range of about 0.1 mg/kg to about 5.0 mg/kg, such as, about 0.3 mg/kg and about 3.0 mg/kg. A repeat-dose regimen may include administration of a therapeutic amount of a nanoparticle encapsulating the nucleic acid molecule on a regular basis, such as every month, once every 3-6 months, or once a year. In certain embodiments, the nanoparticle encapsulating the nucleic acid molecule is administered about once per month to about once per six months.

After an initial treatment regimen, the treatments can be administered on a less frequent basis. Duration of treatment can be determined based on the severity of disease.

In other embodiments, a single dose of the pharmaceutical compositions can be long lasting, such that doses are administered at not more than 1, 2, 3, or 4 month intervals. In some embodiments of the disclosure, a single dose of the pharmaceutical compositions of the disclosure is administered about once per month. In other embodiments of the disclosure, a single dose of the pharmaceutical compositions of the disclosure is administered quarterly (i.e., about every three months). In other embodiments of the disclosure, a single dose of the pharmaceutical compositions of the disclosure is administered twice per year (i.e., about once every six months).

The skilled artisan will appreciate that certain factors can influence the dosage and timing required to effectively treat a subject, including but not limited to mutations present in the subject, previous treatments, the general health or age of the subject, and other diseases present. Moreover, treatment of a subject with a prophylactically or therapeutically effective amount, as appropriate, of a composition can include a single treatment or a series of treatments.

The pharmaceutical compositions of the present disclosure can be administered in a number of ways depending upon whether local or systemic treatment is desired and upon the area to be treated. Administration may be parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The nanoparticle encapsulating the nucleic acid molecule can be delivered in a manner to target a particular tissue, such as a peritoneal tissue described herein.

In some embodiments, the disclosure features a pharmaceutical composition comprising a large peritoneal macrophage (LPM) comprising a nanoparticle encapsulating a nucleic acid molecule, e.g., an siRNA molecule, in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

The nucleic acid molecules, e.g., siRNA molecules, encapsulatd in a nanoparticle of the disclosure can be incorporated into pharmaceutical compositions. Such compositions typically include one or more species of the nucleic acid molecule, e.g., siRNA molecule, and a pharmaceutically acceptable carrier.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration to an LPM. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutical compositions of the present disclosure include, but are not limited to, solutions, emulsions, and liposome-containing formulations. These compositions can be generated from a variety of components that include, but are not limited to, preformed liquids, self-emulsifying solids, and self-emulsifying semisolids.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers.

### IV. METHODS OF TREATMENT OF THE DISCLOSURE

Methods of using a large peritoneal macrophage (LPM) comprising a nanoparticle encapsulating a nucleic acid molecule for treating various conditions in a subject in need thereof that may benefit from LPM-based therapies are described herein. The particular treatment regimen, route of administration, and any combination therapy will be tailored based on the particular condition, the severity of the condition, and the subject's overall health. The disclosure contemplates that administration of a composition comprising a nanoparticle encapsulating a nucleic acid molecule can be used to treat (including reducing the severity of the symptoms, in whole or in part) any of the conditions described herein.

In one aspect, the present disclosure provides a method of delivering a nucleic acid molecule to an injured tissue in a subject in need thereof, the method comprising administering a nanoparticle (for example, an LNP) encapsulating the nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, allowing the LPM comprising the nanoparticle encapsulating the nucleic acid molecule to migrate to the injured tissue, thereby delivering the nucleic acid molecule to the injured tissue in the subject.

The present disclosure also provides methods of therapeutically utilizing LPMs as delivery vehicles to carry the nanoparticle (for example, an LNP) encapsulated nucleic acid modalities to treat a disease, e.g., an inflammatory disease, an infectious disease, an autoimmune disease, or a cancer, in a subject in need thereof. Accordingly, the disclosure provides a method of treating a disease in a subject in need thereof, the method comprising administering a nanoparticle (for example, an LNP) encapsulating a nucleic acid molecule to the subject, allowing the nanoparticle encapsulating the nucleic acid molecule to contact a large peritoneal macrophage (LPM) in the subject, thereby generating an LPM comprising the nanoparticle encapsulating the nucleic acid molecule, allowing the LPM comprising the nanoparticle encapsulating the nucleic acid molecule to migrate to an injured tissue, thereby treating the disease in the subject.

In some embodiments, the disease is selected from the group consisting of an inflammatory disease, an infectious disease, an autoimmune disease, and a cancer.

The term “inflammatory disease” is used herein to refer to a disease, disorder, or condition characterized by having inflammation or an inflammatory component of body tissue. Inflammation may be localized or systemic. Inflammatory diseases notably include drug induced liver injury; peritoneal adhesions; inflammatory bowel disease; acute respiratory distress syndrome (ARDS); severe acute respiratory syndrome (SARS); idiopathic pulmonary fibrosis (IPF); hepatitis; graft rejection including skin graft rejection; chronic inflammatory diseases of the joint including arthritis, rheumatoid arthritis, osteoarthritis, acute gouty arthritis, and inflammatory bone diseases (e.g., associated with increased bone resorption); inflammatory lung diseases such as asthma, adult respiratory distress syndrome, and chronic obstructive airway disease; Behcet's disease; inflammatory diseases of the eye including corneal dystrophy, trachoma, onchocerciasis, uveitis, sympathetic ophthalmitis and endophthalmitis; chronic inflammatory diseases of the gums including gingivitis and periodontitis; tuberculosis; leprosy; inflammatory diseases of the kidney including uremic complications, glomerulonephritis and nephrosis; inflammatory disorders of the skin including scleroderma, psoriasis and eczema; inflammatory diseases of the central nervous system, including chronic demyelinating diseases of the nervous system, infectious meningitis, encephalomyelitis, Parkinson's disease, Huntington's disease, amyotrophic lateral sclerosis and viral or autoimmune encephalitis; autoimmune disorders, immune-complex vasculitis; systemic lupus erythematosus (SLE); and inflammatory diseases of the heart such as cardiomyopathy, coronary thrombosis, ischemic heart disease, hypercholesterolemia, atherosclerosis; as well as various other diseases with significant inflammatory components, including preeclampsia, schizophrenia, chronic liver failure, brain and spinal cord trauma, or endometriosis.

In some embodiments, the inflammatory disease is selected from the group consisting of arthritis, rheumatoid arthritis, osteoarthritis, acute gout arthritis, an inflammatory bone disease; an inflammatory lung disease, preferably asthma, adult respiratory distress syndrome, or chronic obstructive airway disease; Behcet's disease; an inflammatory disease of the eye preferably corneal dystrophy, trachoma, onchocerciasis, uveitis, sympathetic ophthalmitis or endophthalmitis; a chronic inflammatory disease of the gums preferably gingivitis or periodontitis; tuberculosis; leprosy; an inflammatory disease of the kidney preferably a uremic complication, glomerulonephritis or nephrosis; an inflammatory disorder of the skin preferably psoriasis; a chronic demyelinating diseases of the nervous system; infectious meningitis; encephalomyelitis; Parkinson's disease; Huntington's disease; amyotrophic lateral sclerosis; an immune-complex vasculitis; systemic lupus erythematosus (SLE); an inflammatory disease of the heart, preferably cardiomyopathy, coronary thrombosis, ischemic heart disease, hypercholesterolemia, or atherosclerosis; preeclampsia; schizophrenia; chronic liver failure, or brain or spinal cord trauma; endometriosis. Preferably, said inflammatory skin disease is selected from among acne (e.g., acne vulgaris or acne conglobate), rosacea, psoriasis, eczema, atopic dermatitis, scleroderma, seborrheic dermatitis, boils, carbuncles, pemphigus, cellulitis, Grover's disease, hidradenitis suppurativa, lichen planus, or any other inflammatory skin disease described herein. Said inflammatory bone disease is preferably selected from among osteoporosis, periodontal disease, ankylosing spondylitis, osteoarthritis, Paget's disease, Lumbar disc herniation (LDH, including e.g., bulging disc, protruded disc, extruded disc, and sequestrated disc), or rheumatoid arthritis, or any other bone disease in which inflammation mediates bone loss or inflammatory bone disease described herein.

In some embodiments, the inflammatory disease is drug induced liver injury. In some embodiments, the inflammatory disease is peritoneal adhesion. In some embodiments, the inflammatory disease is inflammatory bowel disease. In some embodiments, the inflammatory disease is acute respiratory distress syndrome (ARDS). In some embodiments, the inflammatory disease is severe acute respiratory syndrome (SARS). In some embodiments, the inflammatory disease is idiopathic pulmonary fibrosis (IPF). In some embodiments, the inflammatory disease is hepatitis. In some embodiments, the inflammatory disease is a chronic inflammatory disease. In some embodiments, the inflammatory disease is an inflammatory bone disease. In some embodiments, the inflammatory disease is an inflammatory lung disease. In some embodiments, the inflammatory disease is a chronic obstructive airway disease. In some embodiments, the inflammatory disease is Behcet's disease. In some embodiments, the inflammatory disease is an inflammatory disease of the eye. In some embodiments, the inflammatory disease is a chronic inflammatory diseases of the gums.

In some embodiments, the inflammatory disease is tuberculosis. In some embodiments, the inflammatory disease is leprosy. In some embodiments, the inflammatory disease is an inflammatory disease of the kidney. In some embodiments, the inflammatory disease is an inflammatory disease of the skin. In some embodiments, the inflammatory disease is an inflammatory disease of the central nervous system. In some embodiments, the inflammatory disease is a chronic demyelinating disease of the nervous system. In some embodiments, the inflammatory disease is infectious meningitis. In some embodiments, the inflammatory disease is encephalomyelitis. In some embodiments, the inflammatory disease is Parkinson's disease. In some embodiments, the inflammatory disease is Huntington's disease. In some embodiments, the inflammatory disease is amyotrophic lateral sclerosis. In some embodiments, the inflammatory disease is a viral or autoimmune encephalitis. In some embodiments, the inflammatory disease is immune-complex vasculitis. In some embodiments, the inflammatory disease is systemic lupus erythematosus. In some embodiments, the inflammatory disease is an inflammatory disease of the heart. In some embodiments, the inflammatory disease is preeclampsia. In some embodiments, the inflammatory disease is schizophrenia. In some embodiments, the inflammatory disease is chronic liver failure. In some embodiments, the inflammatory disease is brain trauma. In some embodiments, the inflammatory disease is spinal cord trauma. In some embodiments, the inflammatory disease is endometriosis

The term “infectious disease” is used herein to refer to any infection, disease or condition that can be caused by an organism such as a bacteria, a virus, a fungi or any other pathogenic microbial agent. In some embodiments, the infectious disease is selected from the group consisting of COVID-19, viral hepatitis, tetanus, typhoid fever, diphtheria, syphilis, bacterial vaginosis, Trichomonas vaginalis, meningitis, urinary tract infection, bacterial gastroenteritis, impetigo, cellulitis, pneumonia, lyme disease, and leprosy. In some embodiments, the infectious disease is an infection associated with one or more pathogens selected from the group consisting of coronavirus, Mycobacterium tuberculosis, Streptococcus, Pseudomonas, Shigella, Campylobacter, Salmonella, Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, and Vibrio cholera.

Viral hepatitis is an infection that causes liver inflammation and damage. Several different viruses cause hepatitis, including hepatitis A, B, C, D, and E. The hepatitis A and E viruses typically cause acute infections. The hepatitis B, C, and D viruses can cause acute and chronic infections. In some embodiments, the viral hepatitis described herein is selected from the group consisting of hepatitis A, hepatitis B, hepatitis C, hepatitis D, and hepatitis E.

In some embodiments, the infectious disease is a disease caused by a bacteria, a virus, or a fungi.

In some embodiments, the infectious disease is COVID-19. In some embodiments, the infectious disease is viral hepatitis. In some embodiments, the infectious disease is tetanus. In some embodiments, the infectious disease is typhoid fever. In some embodiments, the infectious disease is diphtheria. In some embodiments, the infectious disease is syphilis. In some embodiments, the infectious disease is bacterial vaginosis. In some embodiments, the infectious disease is Trichomonas vaginalis. In some embodiments, the infectious disease is meningitis. In some embodiments, the infectious disease is a urinary tract infection. In some embodiments, the infectious disease is bacterial gastroenteritis. In some embodiments, the infectious disease is impetigo. In some embodiments, the infectious disease is cellulitis. In some embodiments, the infectious disease is pneumonia. In some embodiments, the infectious disease is lyme disease. In some embodiments, the infectious disease is leprosy.

In some embodiments, the infectious disease is an infection associated with coronavirus. In some embodiments, the infectious disease is an infection associated with Mycobacterium tuberculosis. In some embodiments, the infectious disease is an infection associated with Streptococcus. In some embodiments, the infectious disease is an infection associated with Pseudomonas. In some embodiments, the infectious disease is an infection associated with Shigella. In some embodiments, the infectious disease is an infection associated with Campylobacter. In some embodiments, the infectious disease is an infection associated with Salmonella. In some embodiments, the infectious disease is an infection associated with Campylobacter jejuni. In some embodiments, the infectious disease is an infection associated with Enterococcus faecalis. In some embodiments, the infectious disease is an infection associated with Haemophilus influenza. In some embodiments, the infectious disease is an infection associated with Helicobacter pylori. In some embodiments, the infectious disease is an infection associated with Klebsiella pneumonia. In some embodiments, the infectious disease is an infection associated with Legionella pneumophila. In some embodiments, the infectious disease is an infection associated with Neisseria gonorrhoeae. In some embodiments, the infectious disease is an infection associated with Neisseria meningitides. In some embodiments, the infectious disease is an infection associated with Staphylococcus aureus. In some embodiments, the infectious disease is an infection associated with Streptococcus pneumonia. In some embodiments, the infectious disease is an infection associated with Vibrio cholera.

The term “autoimmune disease” is used herein to refer to any disease resulting from an immune response against a self-tissue or tissue component, including both self-antibody responses and cell-mediated responses. In some embodiments, the autoimmune disease is a condition that results from, or is aggravated by, the production of antibodies, e.g., by B cells, that are reactive with normal body tissues and antigens. In some embodiments, the autoimmune disease is one that involves secretion of an autoantibody that is specific for an epitope from a self-antigen (e.g., a nuclear antigen). The term autoimmune disease, as used herein, encompasses organ-specific autoimmune diseases, in which an autoimmune response is directed against a single tissue, and non-organ specific autoimmune diseases, in which an autoimmune response is directed against a component present in several or many organs throughout the body. Autoimmune diseases notably include rheumatologic autoimmune diseases, gastrointestinal and liver autoimmune diseases, vasculitis, renal autoimmune diseases, dermatological autoimmune diseases, hematologic autoimmune diseases, atherosclerosis, uveitis, autoimmune ear diseases, Raynaud's syndrome, diseases associated with organ transplantation and autoimmune endocrine diseases, such as diabetes.

In some embodiments, the autoimmune disease is a rheumatologic autoimmune disease. In some embodiments, the autoimmune disease is a gastrointestinal autoimmune disease. In some embodiments, the autoimmune disease is a liver autoimmune disease. In some embodiments, the autoimmune disease is vasculitis. In some embodiments, the autoimmune disease is a renal autoimmune disease. In some embodiments, the autoimmune disease is a dermatological autoimmune disease. In some embodiments, the autoimmune disease is a hematologic autoimmune disease. In some embodiments, the autoimmune disease is atherosclerosis. In some embodiments, the autoimmune disease is uveitis. In some embodiments, the autoimmune disease is an ear autoimmune disease. In some embodiments, the autoimmune disease is Raynaud's syndrome. In some embodiments, the autoimmune disease is an autoimmune endocrine disease. In some embodiments, the autoimmune disease is disease associated with organ transplantation.

In some embodiments, the rheumatologic autoimmune disease is selected from the group consisting of rheumatoid arthritis such as acute arthritis, chronic rheumatoid arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, chronic progredient arthritis, arthritis deformans, chronic primary polyarthritis, reactive arthritis, and ankylosing spondylitis, Sjogren's syndrome, scleroderma, lupus such as SLE and lupus nephritis, polymyositis/cryoglobulinemia dermatomyositis, antiphospholipid antibody syndrome, and psoriatic arthritis.

In some embodiments, the gastrointestinal and liver autoimmune is selected from the group consisting of autoimmune gastritis and pernicious anemia, autoimmune hepatitis, primary biliary cirrhosis, primary sclerosing cholangitis, and celiac disease.

In some embodiments, the vasculitis is selected from the group consisting of ANCA-associated vasculitis, Churg-Strauss vasculitis, Wegener's granulomatosis, and polyarteritis.

In some embodiments, the renal autoimmune diseases is selected from the group consisting of glomerulonephritis, syndrome Goodpasture, and Berger's disease.

In some embodiments, the dermatological autoimmune diseases is selected from the group consisting of psoriasis such as plaque psoriasis, guttate psoriasis, pustular psoriasis, and psoriasis of the nails, urticaria such as chronic allergic urticaria and chronic idiopathic urticaria, pemphigus vulgaris, bullous pemphigoid, lupus erythematosus, inflammatory hyperproliferative skin diseases, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non specific dermatitis, primary irritant contact dermatitis, and atopic dermatitis, eczema including allergic or atopic eczema, asteatotic eczema, dyshidrotic eczema, and vesicular palmoplantar eczema.

In some embodiments, the hematologic autoimmune diseases is selected from the group consisting of thrombocytopenic purpura, thrombotic thrombocytopenic purpura, post-transfusion purpura, and autoimmune hemolytic anemia.

In some embodiments, the autoimmune ear diseases is selected from the group consisting of inner ear disease and hearing loss.

In some embodiments, the autoimmune diseases associated with organ transplantation is selected from the group consisting of graft rejection and Graft vs Host disease (GvHD). In some embodiments, the organ transplant is selected from the group consisting of blood transplant, bone marrow transplant, stem cell transplant, kidney transplant, pancreas transplant, liver transplant, orthotopic liver transplant, lung transplant, heart transplant, intestine transplant, small intestine transplant, large intestine transplant, thymus transplant, allograft stem cells transplant, allograft of lesser intensity, bone transplant, tendon transplant, cornea transplant, skin transplant, cardiac valves transplant, veins transplant, arteries transplant, blood vessels transplant, stomach transplant, and testicle transplant.

In some embodiments, the autoimmune endocrine diseases is selected from the group consisting of juvenile onset (Type 1) diabetes mellitus, including pediatric insulin-dependent diabetes mellitus (IDDM), adult onset diabetes mellitus (Type II diabetes), autoimmune diabetes, idiopathic diabetes insipidus, and diseases related to diabetes (such as diabetic retinopathy, diabetic nephropathy, diabetic large-artery disorder) Addison's disease, and autoimmune thyroid disease (such as Graves' disease, Hashimoto's thyroiditis, subacute thyroiditis, idiopathic hypothyroidism).

In some embodiments, the autoimmune disease is selected from the group consisting of atherosclerosis, uveitis, and Raynaud's syndrome.

The term “cancer” used herein to refer to diseases caused by uncontrolled cell division, growth of cells in additional sites, and/or hyperproliferation of cells whose loss of normal controls results in unregulated growth, lack of differentiation, local tissue invasion, and/or metastasis. In some embodiments, the cancer is selected from the group consisting of hepatocellular carcinoma, acute lymphocytic cancer, acute myeloid leukemia, alveolar rhabdomyosarcoma, bladder cancer, bone cancer, brain cancer, breast cancer, cancer of the anus, anal canal cancer, rectum cancer, cancer of the eye, cancer of the intrahepatic bile duct, cancer of the joints, cancer of the neck, gallbladder cancer, pleura cancer, nose cancer, nasal cavity cancer, middle ear cancer, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumor, liver cancer, lung cancer, lymphoma, malignant mesothelioma, mastocytoma, melanoma, multiple myeloma, nasopharynx cancer, nonHodgkin lymphoma, ovarian cancer, pancreatic cancer, peritoneum or omentum, cancer, mesentery cancer, pharynx cancer, prostate cancer, colorectal cancer, renal cancer, skin cancer, small intestine cancer, soft tissue cancer, solid tumor, stomach cancer, testicular cancer, thyroid cancer, ureter cancer, and urinary bladder cancer. As used herein, the term “tumor” refers to an abnormal growth of cells or tissues, e.g., of malignant type or benign type.

In some embodiments, the cancer is hepatocellular carcinoma. In some embodiments, the cancer is acute lymphocytic cancer. In some embodiments, the cancer is acute myeloid leukemia. In some embodiments, the cancer is alveolar rhabdomyosarcoma. In some embodiments, the cancer is bladder cancer. In some embodiments, the cancer is bone cancer. In some embodiments, the cancer is brain cancer. In some embodiments, the cancer is breast cancer. In some embodiments, the cancer is cancer of the anus. In some embodiments, the cancer is anal canal cancer. In some embodiments, the cancer is rectum cancer. In some embodiments, the cancer is cancer of the eye. In some embodiments, the cancer is cancer of the intrahepatic bile duct. In some embodiments, the cancer is cancer of the joints. In some embodiments, the cancer is cancer of the neck. In some embodiments, the cancer is gallbladder cancer. In some embodiments, the cancer is pleura cancer. In some embodiments, the cancer is nose cancer. In some embodiments, the cancer is nasal cavity cancer. In some embodiments, the cancer is middle ear cancer. In some embodiments, the cancer is cancer of the oral cavity. In some embodiments, the cancer is cancer of the vulva. In some embodiments, the cancer is chronic lymphocytic leukemia. In some embodiments, the cancer is chronic myeloid cancer. In some embodiments, the cancer is colon cancer. In some embodiments, the cancer is esophageal cancer. In some embodiments, the cancer is cervical cancer. In some embodiments, the cancer is fibrosarcoma. In some embodiments, the cancer is gastrointestinal cancer. In some embodiments, the cancer is Hodgkin lymphoma. In some embodiments, the cancer is hypopharynx cancer. In some embodiments, the cancer is kidney cancer. In some embodiments, the cancer is larynx cancer. In some embodiments, the cancer is leukemia. In some embodiments, the cancer is liquid tumor. In some embodiments, the cancer is liver cancer. In some embodiments, the cancer is lung cancer. In some embodiments, the cancer is lymphoma, malignant mesothelioma. In some embodiments, the cancer is mastocytoma. In some embodiments, the cancer is melanoma. In some embodiments, the cancer is multiple myeloma. In some embodiments, the cancer is nasopharynx cancer. In some embodiments, the cancer is nonHodgkin lymphoma. In some embodiments, the cancer is ovarian cancer. In some embodiments, the cancer is pancreatic cancer. In some embodiments, the cancer is peritoneum or omentum cancer. In some embodiments, the cancer is mesentery cancer. In some embodiments, the cancer is pharynx cancer. In some embodiments, the cancer is prostate cancer. In some embodiments, the cancer is colorectal cancer. In some embodiments, the cancer is renal cancer. In some embodiments, the cancer is skin cancer. In some embodiments, the cancer is small intestine cancer. In some embodiments, the cancer is soft tissue cancer. In some embodiments, the cancer is solid tumor. In some embodiments, the cancer is stomach cancer. In some embodiments, the cancer is testicular cancer. In some embodiments, the cancer is thyroid cancer. In some embodiments, the cancer is ureter cancer. In some embodiments, the cancer is urinary bladder cancer.

In some embodiments, the injured tissue is a non-peritoneal tissue. In some embodiments, non-peritoneal tissue is a lung tissue.

In some embodiments, the lung tissue comprises an ablation or decrease in levels of tissue resident macrophages (TRMs) relative to an uninjured lung tissue.

In some embodiments, the ablation or decrease in levels of the TRMs comprises an ablation or decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured lung tissue.

In some embodiments, the ablation or decrease in levels of the TRMs comprises an ablation or decrease of at least 0.1-fold relative to an uninjured lung tissue. In some embodiments, the ablation or decrease in levels of the TRMs comprises an ablation or decrease of at least 0.2-fold relative to an uninjured lung tissue. In some embodiments, the ablation or decrease in levels of the TRMs comprises an ablation or decrease of at least 1-fold relative to an uninjured lung tissue. In some embodiments, the ablation or decrease in levels of the TRMs comprises an ablation or decrease of at least 2-fold relative to an uninjured lung tissue. In some embodiments, the ablation or decrease in levels of the TRMs comprises an ablation or decrease of at least 5-fold relative to an uninjured lung tissue. In some embodiments, the ablation or decrease in levels of the TRMs comprises an ablation or decrease of at least 10-fold relative to an uninjured lung tissue. In some embodiments, the ablation or decrease in levels of the TRMs comprises an ablation or decrease of at least 20-fold relative to an uninjured lung tissue. In some embodiments, the ablation or decrease in levels of the TRMs comprises an ablation or decrease of at least 50-fold relative to an uninjured lung tissue. In some embodiments, the ablation or decrease in levels of the TRMs comprises an ablation or decrease of at least 100-fold relative to an uninjured lung tissue. In some embodiments, the ablation or decrease in levels of the TRMs comprises an ablation or decrease of at least 200-fold relative to an uninjured lung tissue. In some embodiments, the ablation or decrease in levels of the TRMs comprises an ablation or decrease of at least 500-fold relative to an uninjured lung tissue. In some embodiments, the ablation or decrease in levels of the TRMs comprises an ablation or decrease of at least 1,000-fold relative to an uninjured lung tissue. In some embodiments, the ablation or decrease in levels of the TRMs comprises an ablation or decrease of at least 10,000-fold relative to an uninjured lung tissue.

In some embodiments, the TRMs are alveolar macrophages.

In some embodiments, the LPM comprising the LNP nanoparticle encapsulating the nucleic acid molecule migrates to the lung tissue in about 1 hour, about 2 hours, about 6 hours, about 12 hours, or about 24 hours after the ablation or decrease in levels of the TRMs in the lung tissue.

In some embodiments, the LPM comprising the LNP nanoparticle encapsulating the nucleic acid molecule migrates to the lung tissue in about 1 hour after the ablation or decrease in levels of the TRMs in the lung tissue. In some embodiments, the LPM comprising the LNP nanoparticle encapsulating the nucleic acid molecule migrates to the lung tissue in about 2 hours after the ablation or decrease in levels of the TRMs in the lung tissue. In some embodiments, the LPM comprising the LNP nanoparticle encapsulating the nucleic acid molecule migrates to the lung tissue in about 6 hours after the ablation or decrease in levels of the TRMs in the lung tissue. In some embodiments, the LPM comprising the LNP nanoparticle encapsulating the nucleic acid molecule migrates to the lung tissue in about 12 hours after the ablation or decrease in levels of the TRMs in the lung tissue. In some embodiments, the LPM comprising the LNP nanoparticle encapsulating the nucleic acid molecule migrates to the lung tissue in about 24 hours after the ablation or decrease in levels of the TRMs in the lung tissue.

In some embodiments, the injured tissue is a peritoneal tissue. In some embodiments, the peritoneal tissue is a liver tissue.

In some embodiments, the liver tissue comprises an increase in levels of tissue resident macrophages (TRMs) relative to an uninjured liver tissue.

In some embodiments, the increase in levels of the TRMs comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the increase in levels of the TRMs comprises an increase of at least 0.1-fold relative to an uninjured liver tissue. In some embodiments, the increase in levels of the TRMs comprises an increase of at least 0.2-fold relative to an uninjured liver tissue. In some embodiments, the increase in levels of the TRMs comprises an increase of at least 0.5-fold relative to an uninjured liver tissue. In some embodiments, the increase in levels of the TRMs comprises an increase of at least 1-fold relative to an uninjured liver tissue. In some embodiments, the increase in levels of the TRMs comprises an increase of at least 2-fold relative to an uninjured liver tissue. In some embodiments, the increase in levels of the TRMs comprises an increase of at least 5-fold relative to an uninjured liver tissue. In some embodiments, the increase in levels of the TRMs comprises an increase of at least 10-fold relative to an uninjured liver tissue. In some embodiments, the increase in levels of the TRMs comprises an increase of at least 20-fold relative to an uninjured liver tissue. In some embodiments, the increase in levels of the TRMs comprises an increase of at least 50-fold relative to an uninjured liver tissue. In some embodiments, the increase in levels of the TRMs comprises an increase of at least 100-fold relative to an uninjured liver tissue. In some embodiments, the increase in levels of the TRMs comprises an increase of at least 200-fold relative to an uninjured liver tissue. In some embodiments, the increase in levels of the TRMs comprises an increase of at least 500-fold relative to an uninjured liver tissue. In some embodiments, the increase in levels of the TRMs comprises an increase of at least 1000-fold relative to an uninjured liver tissue. In some embodiments, the increase in levels of the TRMs comprises an increase of at least 10000-fold relative to an uninjured liver tissue.

In some embodiments, the TRMs are F4/80+.

In some embodiments, the LPM comprising the nanoparticle encapsulating the nucleic acid molecule migrates to the liver tissue in about 1 hour, about 2 hours, about 6 hours, about 12 hours, or about 24 hours after the increase in levels of TRMs in the liver tissue.

In some embodiments, the LPM comprising the nanoparticle encapsulating the nucleic acid molecule migrates to the liver tissue in about 1 hour after the increase in levels of TRMs in the liver tissue. In some embodiments, the LPM comprising the nanoparticle encapsulating the nucleic acid molecule migrates to the liver tissue in about 2 hours after the increase in levels of TRMs in the liver tissue. In some embodiments, the LPM comprising the nanoparticle encapsulating the nucleic acid molecule migrates to the liver tissue in about 6 hours after the increase in levels of TRMs in the liver tissue. In some embodiments, the LPM comprising the nanoparticle encapsulating the nucleic acid molecule migrates to the liver tissue in about 12 hours after the increase in levels of TRMs in the liver tissue. In some embodiments, the LPM comprising the nanoparticle encapsulating the nucleic acid molecule migrates to the liver tissue in about 24 hours after the increase in levels of TRMs in the liver tissue.

In some embodiments, the serum of the subject comprises an increase in level of one or more enzymes selected from the group consisting of alanine transaminase (ALT), aspartate transaminase (AST), and bilirubin relative to serum of a subject with an uninjured liver tissue.

In some embodiments, the increase in level of the one or more enzymes comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to the serum of a subject with an uninjured liver tissue.

In some embodiments, the increase in level of the one or more enzymes comprises an increase of at least 0.1-fold relative to the serum of a subject with an uninjured liver tissue. In some embodiments, the increase in level of the one or more enzymes comprises an increase of at least 0.2-fold relative to the serum of a subject with an uninjured liver tissue. In some embodiments, the increase in level of the one or more enzymes comprises an increase of at least 0.5-fold relative to the serum of a subject with an uninjured liver tissue. In some embodiments, the increase in level of the one or more enzymes comprises an increase of at least 1-fold relative to the serum of a subject with an uninjured liver tissue. In some embodiments, the increase in level of the one or more enzymes comprises an increase of at least 2-fold relative to the serum of a subject with an uninjured liver tissue. In some embodiments, the increase in level of the one or more enzymes comprises an increase of at least 5-fold relative to the serum of a subject with an uninjured liver tissue. In some embodiments, the increase in level of the one or more enzymes comprises an increase of at least 10-fold relative to the serum of a subject with an uninjured liver tissue. In some embodiments, the increase in level of the one or more enzymes comprises an increase of at least 20-fold relative to the serum of a subject with an uninjured liver tissue. In some embodiments, the increase in level of the one or more enzymes comprises an increase of at least 50-fold relative to the serum of a subject with an uninjured liver tissue. In some embodiments, the increase in level of the one or more enzymes comprises an increase of at least 100-fold relative to the serum of a subject with an uninjured liver tissue. In some embodiments, the increase in level of the one or more enzymes comprises an increase of at least 200-fold relative to the serum of a subject with an uninjured liver tissue. In some embodiments, the increase in level of the one or more enzymes comprises an increase of at least 500-fold relative to the serum of a subject with an uninjured liver tissue. In some embodiments, the increase in level of the one or more enzymes comprises an increase of at least 1000-fold relative to the serum of a subject with an uninjured liver tissue. In some embodiments, the increase in level of the one or more enzymes comprises an increase of at least 10000-fold relative to the serum of a subject with an uninjured liver tissue.

In some embodiments, the liver tissue comprises an increase in level of pro-inflammatory macrophages relative to an uninjured liver tissue.

In some embodiments, the increase in level of the pro-inflammatory macrophages comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the increase in level of the pro-inflammatory macrophages comprises an increase of at least 0.1-fold relative to an uninjured liver tissue. In some embodiments, the increase in level of the pro-inflammatory macrophages comprises an increase of at least 0.2-fold relative to an uninjured liver tissue. In some embodiments, the increase in level of the pro-inflammatory macrophages comprises an increase of at least 0.5-fold relative to an uninjured liver tissue. In some embodiments, the increase in level of the pro-inflammatory macrophages comprises an increase of at least 1-fold relative to an uninjured liver tissue. In some embodiments, the increase in level of the pro-inflammatory macrophages comprises an increase of at least 2-fold relative to an uninjured liver tissue. In some embodiments, the increase in level of the pro-inflammatory macrophages comprises an increase of at least 5-fold relative to an uninjured liver tissue. In some embodiments, the increase in level of the pro-inflammatory macrophages comprises an increase of at least 10-fold relative to an uninjured liver tissue. In some embodiments, the increase in level of the pro-inflammatory macrophages comprises an increase of at least 20-fold relative to an uninjured liver tissue. In some embodiments, the increase in level of the pro-inflammatory macrophages comprises an increase of at least 50-fold relative to an uninjured liver tissue. In some embodiments, the increase in level of the pro-inflammatory macrophages comprises an increase of at least 100-fold relative to an uninjured liver tissue. In some embodiments, the increase in level of the pro-inflammatory macrophages comprises an increase of at least 200-fold relative to an uninjured liver tissue. In some embodiments, the increase in level of the pro-inflammatory macrophages comprises an increase of at least 500-fold relative to an uninjured liver tissue. In some embodiments, the increase in level of the pro-inflammatory macrophages comprises an increase of at least 1000-fold relative to an uninjured liver tissue. In some embodiments, the increase in level of the pro-inflammatory macrophages comprises an increase of at least 10000-fold relative to an uninjured liver tissue.

In some embodiments, the pro-inflammatory macrophages comprise one or more markers selected from the group consisting of iNOS-1, and TNF-a. In some embodiments, the pro-inflammatory macrophages comprise an iNOS-1 marker. In some embodiments, the pro-inflammatory macrophages comprise an TNF-a marker.

In some embodiments, the liver tissue comprises a decrease in level of anti-inflammatory macrophages relative to an uninjured liver tissue.

In some embodiments, the decrease in level of the anti-inflammatory macrophages comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the decrease in level of the anti-inflammatory macrophages comprises a decrease of at least 0.1-fold relative to an uninjured liver tissue. In some embodiments, the decrease in level of the anti-inflammatory macrophages comprises a decrease of at least 0.2-fold relative to an uninjured liver tissue. In some embodiments, the decrease in level of the anti-inflammatory macrophages comprises a decrease of at least 0.5-fold relative to an uninjured liver tissue. In some embodiments, the decrease in level of the anti-inflammatory macrophages comprises a decrease of at least 1-fold relative to an uninjured liver tissue. In some embodiments, the decrease in level of the anti-inflammatory macrophages comprises a decrease of at least 2-fold relative to an uninjured liver tissue. In some embodiments, the decrease in level of the anti-inflammatory macrophages comprises a decrease of at least 5-fold relative to an uninjured liver tissue. In some embodiments, the decrease in level of the anti-inflammatory macrophages comprises a decrease of at least 10-fold relative to an uninjured liver tissue. In some embodiments, the decrease in level of the anti-inflammatory macrophages comprises a decrease of at least 20-fold relative to an uninjured liver tissue. In some embodiments, the decrease in level of the anti-inflammatory macrophages comprises a decrease of at least 50-fold relative to an uninjured liver tissue. In some embodiments, the decrease in level of the anti-inflammatory macrophages comprises a decrease of at least 100-fold relative to an uninjured liver tissue. In some embodiments, the decrease in level of the anti-inflammatory macrophages comprises a decrease of at least 200-fold relative to an uninjured liver tissue. In some embodiments, the decrease in level of the anti-inflammatory macrophages comprises a decrease of at least 500-fold relative to an uninjured liver tissue. In some embodiments, the decrease in level of the anti-inflammatory macrophages comprises a decrease of at least 1000-fold relative to an uninjured liver tissue. In some embodiments, the decrease in level of the anti-inflammatory macrophages comprises a decrease of at least 10000-fold relative to an uninjured liver tissue.

In some embodiments, the anti-inflammatory macrophages comprise one or more markers selected from the group consisting of Arg-1, and CD206. In some embodiments, the anti-inflammatory macrophages comprise an Arg-1 marker. In some embodiments, the anti-inflammatory macrophages comprise a CD206 marker.

In some embodiments, the liver tissue comprises an increase in level of one or more pro-inflammatory cytokines selected from the group consisting of CXCL5, CCL11, CXCL1, IL-6, IL-9, IL-23, IL-28, CXCL10, CCL7, CCL3 and CCL5 relative to an uninjured liver tissue.

In some embodiments, the liver tissue comprises an increase in level of CXCL5 relative to an uninjured liver tissue. In some embodiments, the liver tissue comprises an increase in level of CCL11 relative to an uninjured liver tissue. In some embodiments, the liver tissue comprises an increase in level of CXCL1 relative to an uninjured liver tissue. In some embodiments, the liver tissue comprises an increase in level of IL-6 relative to an uninjured liver tissue. In some embodiments, the liver tissue comprises an increase in level of IL-9 relative to an uninjured liver tissue. In some embodiments, the liver tissue comprises an increase in level of IL-23 relative to an uninjured liver tissue. In some embodiments, the liver tissue comprises an increase in level of IL-28 relative to an uninjured liver tissue. In some embodiments, the liver tissue comprises an increase in level of CXCL10 relative to an uninjured liver tissue. In some embodiments, the liver tissue comprises an increase in level of CCL7 relative to an uninjured liver tissue. In some embodiments, the liver tissue comprises an increase in level of CCL3 relative to an uninjured liver tissue. In some embodiments, the liver tissue comprises an increase in level of CCL5 relative to an uninjured liver tissue.

In some embodiments, the increase in level of the one or more pro-inflammatory cytokines comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the increase in level of the one or more pro-inflammatory cytokines comprises an increase of 0.1-fold relative to an uninjured liver tissue. In some embodiments, the increase in level of the one or more pro-inflammatory cytokines comprises an increase of 0.2-fold relative to an uninjured liver tissue. In some embodiments, the increase in level of the one or more pro-inflammatory cytokines comprises an increase of 0.5-fold relative to an uninjured liver tissue. In some embodiments, the increase in level of the one or more pro-inflammatory cytokines comprises an increase of 1-fold relative to an uninjured liver tissue. In some embodiments, the increase in level of the one or more pro-inflammatory cytokines comprises an increase of 2-fold relative to an uninjured liver tissue. In some embodiments, the increase in level of the one or more pro-inflammatory cytokines comprises an increase of 5-fold relative to an uninjured liver tissue. In some embodiments, the increase in level of the one or more pro-inflammatory cytokines comprises an increase of 10-fold relative to an uninjured liver tissue. In some embodiments, the increase in level of the one or more pro-inflammatory cytokines comprises an increase of 20-fold relative to an uninjured liver tissue. In some embodiments, the increase in level of the one or more pro-inflammatory cytokines comprises an increase of 50-fold relative to an uninjured liver tissue. In some embodiments, the increase in level of the one or more pro-inflammatory cytokines comprises an increase of 100-fold relative to an uninjured liver tissue. In some embodiments, the increase in level of the one or more pro-inflammatory cytokines comprises an increase of 200-fold relative to an uninjured liver tissue. In some embodiments, the increase in level of the one or more pro-inflammatory cytokines comprises an increase of 500-fold relative to an uninjured liver tissue. In some embodiments, the increase in level of the one or more pro-inflammatory cytokines comprises an increase of 1,000-fold relative to an uninjured liver tissue. In some embodiments, the increase in level of the one or more pro-inflammatory cytokines comprises an increase of 10,000-fold relative to an uninjured liver tissue.

In some embodiments, the liver tissue comprises a decrease in level of one or more anti-inflammatory cytokines selected from the group consisting of IL-4, and IL-10 relative to an uninjured liver tissue.

In some embodiments, the liver tissue comprises a decrease in level of IL-4 relative to an uninjured liver tissue. In some embodiments, the liver tissue comprises a decrease in level of IL-10 relative to an uninjured liver tissue.

In some embodiments, the one or more anti-inflammatory cytokines comprises a decrease of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to an uninjured liver tissue.

In some embodiments, the one or more anti-inflammatory cytokines comprises a decrease of at least 0.1-fold relative to an uninjured liver tissue. In some embodiments, the one or more anti-inflammatory cytokines comprises a decrease of at least 0.2-fold relative to an uninjured liver tissue. In some embodiments, the one or more anti-inflammatory cytokines comprises a decrease of at least 0.5-fold relative to an uninjured liver tissue. In some embodiments, the one or more anti-inflammatory cytokines comprises a decrease of at least 1-fold relative to an uninjured liver tissue. In some embodiments, the one or more anti-inflammatory cytokines comprises a decrease of at least 2-fold relative to an uninjured liver tissue. In some embodiments, the one or more anti-inflammatory cytokines comprises a decrease of at least 5-fold relative to an uninjured liver tissue. In some embodiments, the one or more anti-inflammatory cytokines comprises a decrease of at least 10-fold relative to an uninjured liver tissue. In some embodiments, the one or more anti-inflammatory cytokines comprises a decrease of at least 20-fold relative to an uninjured liver tissue. In some embodiments, the one or more anti-inflammatory cytokines comprises a decrease of at least 50-fold relative to an uninjured liver tissue. In some embodiments, the one or more anti-inflammatory cytokines comprises a decrease of at least 100-fold relative to an uninjured liver tissue. In some embodiments, the one or more anti-inflammatory cytokines comprises a decrease of at least 200-fold relative to an uninjured liver tissue. In some embodiments, the one or more anti-inflammatory cytokines comprises a decrease of at least 500-fold relative to an uninjured liver tissue. In some embodiments, the one or more anti-inflammatory cytokines comprises a decrease of at least 1000-fold relative to an uninjured liver tissue. In some embodiments, the one or more anti-inflammatory cytokines comprises a decrease of at least 10000-fold relative to an uninjured liver tissue.

In some embodiments, the administering of the nanoparticle encapsulating the nucleic acid molecule to the subject is performed intraperitoneally.

In some embodiments, the nucleic acid molecule is selected from the group consisting of an oligonucleotide, a DNA, an RNA, a ribozyme, an aptamer, and a DNAzyme.

The in vivo methods of the disclosure include administering compositions comprising a nanoparticle encapsulating a nucleic acid molecule, e.g., an siRNA molecule, to a subject. The composition can be administered by any means known in the art including, but not limited to intraperitoneal, or parenteral routes, including intracranial (e.g., intraventricular, intraparenchymal, and intrathecal), intravenous, intramuscular, subcutaneous, transdermal, airway (aerosol), nasal, rectal, intraocular (e.g., periocular, conjunctival, subtenon, intracameral, intravitreal, intraocular, anterior or posterior juxtascleral, subretinal, subconjunctival, retrobulbar, or intracanalicular injection), intravenous, intramuscular, subcutaneous, transdermal, airway (aerosol), and topical (including buccal and sublingual) administration.

In certain embodiments, the compositions are administered intraperitoneally. In certain embodiments, the compositions are administered by intravenous infusion or injection. In certain embodiments, the compositions are administered by subcutaneous injection. In certain embodiments, the compositions are administered by intramuscular injection.

The mode of administration may be chosen based upon whether local or systemic treatment is desired and based upon the area to be treated. The route and site of administration may be chosen to enhance targeting.

In one aspect, the present disclosure also provides methods for inhibiting the expression of a CD45, a HMGB1, a NFkB1, a TLR4, or a gLuc gene in the subject. The methods include administering to the subject a nanoparticle encapsulating a nucleic acid molecule, e.g., an siRNA that targets a CD45, a HMGB1, a NFkB1, a TLR4, or a gLuc gene in an LPM or a cell of the injured tissue in the subject. Reduction in gene expression can be assessed by any methods known in the art and by methods, e.g., qRT-PCR. Reduction in protein production can be assessed by any methods known it the art, e.g., ELISA. In certain embodiments, a blood sample serves as the subject sample for monitoring the reduction in the CD45, HMGB1, NFKB1, TLR4, or gLuc expression.

In one aspect, the present disclosure provides methods of treating a subject having a disorder that would benefit from reduction in CD45, HMGB1, NFKB1, TLR4, or gLuc expression, e.g., an inflammatory disease, an infectious disease, an autoimmune disease, or a cancer.

In an embodiment, the method includes administering a composition featured herein such that expression of the target gene, e.g., a CD45, a HMGB1, a NFkB1, a TLR4, or a gLuc gene is decreased, such as for about 1, 2, 3, 4, 5, 6, 1-6, 1-3, or 3-6 months per dose. In certain embodiments, the composition is administered once every 3-6 months.

In some embodiments, the nucleic acid molecules, e.g., siRNAs, useful for the methods and compositions featured herein specifically target a CD45, a HMGB1, a NFkB1, a TLR4, or a gLuc gene. Compositions and methods for inhibiting the expression of these genes using nucleic acid molecules, e.g., siRNAs can be prepared and performed as described herein.

The disclosure further provides methods and uses of the pharmaceutical composition described herein for treating a subject that would benefit from reduction and/or inhibition of CD45, HMGB1, NFKB1, TLR4, or gLuc gene expression, e.g., a subject having an inflammatory disease, an infectious disease, an autoimmune disease, or a cancer, in combination with other pharmaceuticals and/or other therapeutic methods, e.g., with known pharmaceuticals and/or known therapeutic methods, such as, for example, those which are currently employed for treating these disorders.

Accordingly, in some aspects of the disclosure, the methods which include administration of the compositions of the disclosure, further include administering to the subject one or more additional therapeutic agents.

Exemplary additional therapeutics and treatments for treating cancer, may include surgery, chemotherapy, radiation therapy, or the administration of one or more additional anti-cancer agents, such as a chemotherapeutic agent, a growth inhibitory agent, an anti-angiogenesis agent and/or a anti-neoplastic composition. Nonlimiting examples of anti-cancer agents, chemotherapeutic agents, growth inhibitory agents, anti-angiogenesis agents, and anti-neoplastic compositions that can be used in combination with the iRNA of the present disclosure are as follows.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, the composition of the disclosure may be further administered with gemcitabine-based chemotherapy in which one or more chemotherapy agents including gemcitabine or including gemcitabine and nab-paclitaxel are administered. In some such embodiments, the composition of the disclosure may be administered with at least one chemotherapy agent selected from gemcitabine, nab-paclitaxel, leukovorin (folinic acid), 5-fluorouracil (5-FU), irinotecan, and oxaliplatin. FOLFIRINOX is a chemotherapy regime comprising leukovorin, 5-FU, irinotecan (such as liposomal irinotecan injection), and oxaliplatin. In some embodiments, the composition of the disclosure may be further administered with gemcitabine-based chemotherapy. In some embodiments, the iRNA of the disclosure may be further administered with at least one agent selected from (a) gemcitabine; (b) gemcitabine and nab-paclitaxel; and (c) FOLFIRINOX. In some embodiments, the at least one agent is gemcitabine. In some such embodiments, the cancer to be treated is pancreatic cancer.

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al., (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA, or VEGF receptor(s), and other bioactive and organic chemical agents, etc. Combinations thereof are also included in the disclosure.

The composition of the disclosure and one or more additional therapeutic agents may be administered at the same time and/or in the same combination, e.g., intraperitoneally, or the additional therapeutic agent can be administered as part of a separate composition or at separate times and/or by another method known in the art or described herein.

The composition of the disclosure and an additional therapeutic agent and/or treatment may be administered at the same time and/or in the same combination, e.g., intraperitoneally, or the additional therapeutic agent can be administered as part of a separate composition or at separate times and/or by another method known in the art or described herein.

### V. KITS

In certain aspects, the instant disclosure provides kits that include a suitable container containing a pharmaceutical formulation of a nanoparticle encapsulating a nucleic acid molecule, e.g., an siRNA molecule.

Such kits include one or more nanoparticles encapsulating the nucleic acid molecule or an LPM, e.g., a GATA6+ LPM, comprising a nanoparticle encapsulating a nucleic acid molecule, and instructions for use, e.g., instructions for administering a prophylactically or therapeutically effective amount of the nanoparticle encapsulating the nucleic acid molecule or the LPM, e.g., a GATA6+ LPM, comprising a nanoparticle encapsulating a nucleic acid molecule. The pharmaceutical formulation may be in a vial or a pre-filled syringe. The kits may optionally further comprise means for administering the nanoparticle encapsulating the nucleic acid molecule or the LPM, e.g., a GATA6+ LPM, comprising a nanoparticle encapsulating a nucleic acid molecule, e.g., an siRNA (e.g., an injection device, such as a pre-filled syringe), or means for measuring the inhibition of CD45, HMGB1, NFKB1, TLR4, or gLuc (e.g., means for measuring the inhibition of CD45, HMGB1, NFKB1, TLR4, or gLuc mRNA; CD45, HMGB1, NFKB1, TLR4, or gLuc protein; and/or CD45, HMGB1, NFKB1, TLR4, or gLuc activity). Such means for measuring the inhibition of CD45, HMGB1, NFKB1, TLR4, or gLuc may comprise a means for obtaining a sample from a subject, such as, e.g., a blood or a tissue sample. The kits of the disclosure may optionally further comprise means for determining the therapeutically effective or prophylactically effective amount.

In certain embodiments the individual components of the pharmaceutical formulation may be provided in one container, e.g., a vial or a pre-filled syringe. Alternatively, it may be desirable to provide the components of the pharmaceutical formulation separately in two or more containers, e.g., one container for a nucleic acid molecule compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

This disclosure is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1. Formulation of a Lipid Nanoparticle Encapsulating a Nucleic Acid Molecule

Synthesis of siRNA targeting CD-45 and conjugation of cy5.5: Double-stranded small-interfering RNA targeting CD-45 was synthesized by use of standard phosphor-amidite chemistry. Chemical modifications were applied to the siRNA template, involving —O-methyl groups at the 2′-positions, and 2′-fluoro-groups at positions 2, 6, 14 and 16 of the antisense strand, and 7, 8, 9 and 10 of the sense strand along with capping the ends with 6 phosphorothioates (PSs) for protection from endonuclease- and exonuclease-mediated siRNA cleavage, respectively (Novobrantseva T I et al., Systemic RNAi-mediated Gene Silencing in Nonhuman Primate and Rodent Myeloid Cells. Mol Ther Nucleic Acids. 2012; 1(1):e4-e; Love K T et al., Lipid-like materials for low-dose, in vivo gene silencing. 2010; 107(5):1864-9; and Semple S C et al., Rational design of cationic lipids for siRNA delivery. Nature Biotechnology. 2010; 28(2):172-6; each of which is incorporated in its entirety herein by reference). Deprotection and purification of the crude oligoribonucleotides by anion exchange high-performance liquid chromatography were carried out according to established procedures (Novobrantseva T I et al., 2012; Love K T et al., 2010; and Semple S C et al., 2010; each of which is incorporated in its entirety herein by reference). siRNA targeting CD-45 mRNA target site (Accession #NM_001111316.2) was generated by annealing equimolar amounts of complementary sense and antisense strands (Novobrantseva T I et al., 2012; Love K T et al., 2010; and Semple S C et al., 2010; each of which is incorporated in its entirety herein by reference). A cy5.5 fluorophore was labeled on the 5′-end of the sense strand before formulating the siRNA into NPs.

Synthesis and characterization of C12-200-based LNPs and siRNA encapsulation: LNP-based nanoparticle formulations were synthesized utilizing the cationic lipid C12-200. Lipids were dissolved in 90% ethanol solution and mixed with siRNA solution (25 mM citrate, pH 3 ratio) at fixed speed (1:1 ratio) and diluted immediately with PBS to final 25% ethanol. The ethanol was then removed, and the external buffer replaced with PBS (155 mM NaCl, 3 mM Na2HPO4, 1 mM KH2PO4, pH 7.5) by dialysis. The particles had a component molar ratio of ~50/10/38.5/1.5 (C12-200/distearoylphosphatidylcholine/cholesterol/PEG-C14). Particle size and zeta potential were determined using a Malvern Zetasizer NanoZS. siRNA content was determined by ion exchange high-performance liquid chromatography (Agilent) assay using DNAPac Pa200 column (Dionex Corporation Dionex, 260 nm, 55 °C run at 2 ml/min). siRNA entrapment efficiency was determined by the Quant-iT RiboGreen RNA assay (Invitrogen). Briefly, siRNA entrapment was determined by comparing the signal of the RNA-binding dye RiboGreen in formulation samples in the absence and presence of the detergent Triton-X100. In the absence of detergent, the signal comes from accessible (unentrapped) siRNA only. In the presence of detergent, the signal comes from total siRNA.

Formulations comprising an LNP encapsulating a nucleic acid molecule, e.g., an siRNA were generated to identify the potential of using these formulations as a tool to study LPM migration, and as a therapeutic modality for gene-silencing in LPMs. A CD-45-targeting siRNA was utilized since CD-45 is a pan-macrophage surface marker (Pilling D et al., Identification of markers that distinguish monocyte-derived fibrocytes from monocytes, macrophages, and fibroblasts. PloS one. 2009; 4(10):e7475-e; Holt M P et al., Identification and characterization of infiltrating macrophages in acetaminophen-induced liver injury. 2008; 84(6):1410-21; Streuli M et al., Characterization of CD45 and CD45R monoclonal antibodies using transfected mouse cell lines that express individual human leukocyte common antigens. 1988; 141(11):3910-4; each of which is incorporated in its entirety herein by reference). A main objective was to investigate whether LPMs have the ability to migrate to areas of injury or depletion of other local TRMs, and if so, whether they migrated via systemic circulation. Hence, in order to track these cells, the sense strand of the siRNA was labeled with a cy5.5 fluorophore.

C12-200 is an important excipient for siRNA entrapment and intracellular delivery of oligonucleotides. This LNP system comprises a four-compartment system with C12-200 along with helper lipids Distearoylphosphatidylcholine (DSPC), cholesterol and Poly(ethylene) glycol (PEG)-C14 and had a component molar ratio of ~50/10/38.5/1.5 (C12-200/distearoylphosphatidylcholine/cholesterol/PEG-C14) (Novobrantseva T I et al., 2012; Love K T et al., 2010; Semple S C et al., 2010; and Leuschner F et al., Therapeutic siRNA silencing in inflammatory monocytes in mice. Nature Biotechnology. 2011; 29(11):1005-10; each of which is incorporated in its entirety herein by reference) (Table 2). Synthesis of C12-200 and formulation of siRNA into the C12-200 was carried out as previously described (Novobrantseva T I et al., 2012; Love K T et al., 2010; Semple S C et al., 2010; and Leuschner F et al., 2011; each of which is incorporated in its entirety herein by reference). The final encapsulation efficiency (EE) of siRNA was 63% and the encapsulated siRNA concentration was 0.29 mg/ml. Average particle size was 70.93 nm, and the polydispersity index (PDI) of the final formulation was 0.048 (Table 2).

TABLE 2
C12-200 formulation characterization, siRNA
encapsulation efficiency and concentration

C12-200-
encapsulated

| siRNA | Results | | | | |
|---|---|---|---|---|---|
| | | C12-200 | DSPC | Cholesterol | PEG-C14 |
| Lipid | mol % | 50 | 10 | 38.5 | 1.5 |
| concen- | Concen- | 100 | 40 | 20 | 100 |
| tration | tration | | | | |
| | (mg/ml) | | | | |
| Particle Size | Z-Ave d | 70.93 | | PDI | 0.048 |
| | (nm) | | | | |
| EE (%) | 63 | | | | |
| Encapsulated | 0.29 | | | | |
| siRNA | | | | | |
| concentration | | | | | |
| (mg/ml) | | | | | |
| Volume of | 6.5 | | | | |
| formulation | | | | | |
| (ml) | | | | | |

(Abbreviations: siRNA—small interfering RNA; DSPC—distearoylphosphatidylcholine; and EE—encapsulation efficiency)

### Example 2. Large Peritoneal Macrophages Comprising Lipid Nanoparticles Encapsulating a Nucleic Acid Molecule Migrate to the Lung Via Systemic Circulation in a Model of Alveolar Macrophage Depletion

LPMs are a unique type of TRMs in the peritoneal cavity, and novel findings about their behavior in context of acute tissue injuries have elucidated their tissue-specific functions and responses to injury stimuli. Tissue-specific localization and functional polarization of LPMs can be driven by zinc-finger transcription factor GATA6, which is specifically expressed in LPMs among all TRMs (Cassado AdA et al., 2015; and Okabe Y and Medzhitov R. 2014; each of which is incorporated in its entirety herein by reference). Despite being tissue-resident, LPMs have a unique migratory ability and can move to injured tissues within the abdominal cavity and impart wound healing properties (Parayath N N et al., 2018; Honda M et al., 2021; Ito T et al., 2021; Wang J and Kubes P. 2016; and Zindel J et al., 2021; each of which is incorporated in its entirety herein by reference). The present study provides a further exploration of this phenomenon more broadly across different tissues including migration of LPMs to non-peritoneal injured tissue. It is demonstrated that LPMs are a mature macrophage population that can readily migrate even to distant tissues in response to ablation of the local TRM population, in particular, to non-peritoneally located organs, such as the lungs.

AMs residing in the alveolar lumen of the lungs form the first line of defense for the respiratory tract (Hetzel M et al., Beyond “Big Eaters”: The Versatile Role of Alveolar Macrophages in Health and Disease. 2021; 22(7):3308; Rubins J B. Alveolar Macrophages. 2003; 167(2):103-4; Hu G, and Christman J W. Editorial: Alveolar Macrophages in Lung Inflammation and Resolution. 2019; 10; Woo Y D, Jeong D, Chung D H. Development and Functions of Alveolar Macrophages. Mol Cells. 2021; 44(5):292-300; and Herold S et al., Acute Lung Injury: How Macrophages Orchestrate Resolution of Inflammation and Tissue Repair 2011; 2; each of which is incorporated in its entirety herein by reference). It is demonstrated that selective depletion of AMs in the lungs via internasal administration of clodronate liposomes makes the LPMs more tropic towards the lungs and results in their infiltration (Van Rooijen N, and Hendrikx E. Liposomes for Specific Depletion of Macrophages from Organs and Tissues. In: Weissig V, editor. Liposomes: Methods and Protocols, Volume 1: Pharmaceutical Nanocarriers. Totowa, NJ: Humana Press; 2010. p. 189-203; Van Rooijen N, and Sanders A. Liposome mediated depletion of macrophages: mechanism of action, preparation of liposomes and applications. Journal of immunological methods. 1994; 174(1-2):83-93; Rooijen Nv, and Kesteren-Hendrikx Ev. “In vivo” Depletion of Macrophages by Liposome-Mediated “Suicide”. Methods in Enzymology. 373: Academic Press; 2003. p. 3-16; Van Rooijen N et al., Apoptosis of macrophages induced by liposome-mediated intracellular delivery of clodronate and propamidine. Journal of immunological methods. 1996; 193(1):93-9; and Fleisch H. Bisphosphonates in Bone Disease: From the Laboratory to the Patient: Elsevier Science; 2000; each of which is incorporated in its entirety herein by reference). A small interfering RNA (siRNA) labeled with fluorescent dye cyanine5.5 (cy5.5) was specifically delivered to LPMs using a lipid nanoparticle (LNP)-based delivery modality in C12-200-based LNPs (Oza D, and Amiji M M. 2022; Novobrantseva T I et al., Systemic RNAi-mediated Gene Silencing in Nonhuman Primate and Rodent Myeloid Cells. Mol Ther Nucleic Acids. 2012; 1(1):e4-e; Love K T et al., Lipid-like materials for low-dose, in vivo gene silencing. 2010; 107(5):1864-9; Semple S C et al., Rational design of cationic lipids for siRNA delivery. Nature Biotechnology. 2010; 28(2):172-6; and Leuschner F et al., Therapeutic siRNA silencing in inflammatory monocytes in mice. Nature Biotechnology. 2011; 29(11):1005-10; each of which is incorporated in its entirety herein by reference). C12-200 is a novel ‘lipidoid’-like ionizable lipid that has been characterized and used as the lipid of choice for immune-cell delivery of oligonucleotides in the past (Novobrantseva T I et al., 2012; and Leuschner F et al., 2011; each of which is incorporated in its entirety herein by reference). C12-200-based LNPs have a primary mechanism of uptake by macrophage-induced phagocytosis. Owing to their success in achieving peritoneal macrophage selective uptake, these lipids were utilized to formulate an siRNA encapsulating LNP (Novobrantseva T I et al., 2012; Love K T et al., 2010; Semple S C et al., 2010; and Leuschner F et al., 2011; each of which is incorporated in its entirety herein by reference). From these studies, infiltration of labeled LPMs was observed to the lungs 12-24 hours following AM depletion, opening the question of how these macrophages residing in the peritoneum migrate to and infiltrate the lungs.

One of the key questions of the study was to explore whether LPMs migrate to the lungs via systemic circulation. To answer this, an emerging technique of Diffuse In vivo Flow Cytometry (DiFC) was used to track migration of siRNA-cy5.5 labeled LPMs to the lungs via the circulatory system (Tan X et al., In vivo Flow Cytometry of Extremely Rare Circulating Cells. Scientific Reports. 2019; 9(1):3366; and Pera V et al., Diffuse fluorescence fiber probe for in vivo detection of circulating cells. 2017; 22 Journal of Biomedical Optics (3):037004; each of which is incorporated in its entirety herein by reference). DiFC allows non-invasive enumeration of fluorescent circulating cells in peripheral blood without the need to draw blood samples (Tan X et al., 2019; and Pera V et al., 2017; each of which is incorporated in its entirety herein by reference). DiFC uses laser-induced fluorescence and highly scattered photons to detect moving cells and fluorescent sensors in relatively large, deeply seated blood vessels (Tan X et al., 2019; Pera V et al., 2017; Di W et al., Real-time particle-by-particle detection of erythrocyte-camouflaged microsensor with extended circulation time in the bloodstream. 2020; 117(7):3509-17; and Williams A L et al., Short-Term Circulating Tumor Cell Dynamics in Mouse Xenograft Models and Implications for Liquid Biopsy. 2020; 10; each of which is incorporated in its entirety herein by reference). DiFC surprisingly revealed for the first time that LPMs migrate via the systemic circulation in the 12-24 hours window following AM depletion, thus providing deeper insights into route and kinetics of LPM migration post AM ablation.

The present study confirms a robust uptake of siRNAs to LPMs utilizing the C12-200-based LNP formulation and further identifies that LPMs indeed show an inherent tropism towards the lungs and infiltrate them upon ablation of AMs. Thus, LPMs provide a therapeutic means to deliver siRNA payloads to injured tissue. Together, these findings shed more light on a unique property of a cavity-associated TRM like LPM to naturally migrate towards a non-peritoneally located resident-macrophage-ablated tissue.

### Materials and Methods

In vivo studies in mice: Balb/c mice were obtained from Charles River Laboratories. All mice were on the Balb/c background. Animals were maintained in a specific pathogen-free environment with ad libitum access to food and water. Mice were housed under standardized conditions of temperature (21-22 °C) and illumination (12/12 h light/dark cycle). Mice of 8-12 weeks of age were used for experiments. Mice were gender-matched for experiments and experimental/control mice were bred separately.

Antibodies and reagents: Antibodies against CD11b conjugated to PE (Monoclonal Antibody (M1/70), PE, eBioscience™, 12-0112-82) (1:100 dilution) and F4/80 conjugated to FITC (Monoclonal Antibody (BM8), FITC, eBioscience™, 11-4801-82) (1:100 dilution) (for both flow and immunocytochemistry) were obtained from eBioscience™, antibodies for Fc block (anti-CD16/CD32 Mouse BD Fc Block™; 2.4G2 clone; diluted 1-2:200, 0.5-1 ug) was obtained from BD Biosciences. Antibodies against GATA6 (D61E4 XP® Rabbit mAb #5851) (1:50 dilution) and secondary antibody against the GATA6 Rabbit mAb (Anti-rabbit IgG (H+L), F(ab′)2 Fragment (Alexa Fluor®555 Conjugate) (1:1000 dilution) #4413 were obtained from Cell Signaling Technologies. Antibodies against actin (Alexa Fluor™ 488 Phalloidin, A12379) (1:5000 dilution) were obtained from Invitrogen™. NucBlue™ Live ReadyProbes™ Reagent (Hoechst 33342) (1:10,000 dilution) was used for staining the nuclei.

Mouse model of clodronate-induced alveolar macrophage ablation: For all the studies involving clodronate liposomal administration, mice were briefly anaesthetized by 2% isoflurane, and under the influence of mild anesthesia, 5 mg/kg of clodronate liposomes (Liposoma BV, C-005) were intranasally administered (Parayath N N et al., 2018; Van Rooijen N, and Hendrikx E. 2010; Van Rooijen N, and Sanders A. 1994; and Van Rooijen N et al., 1996; each of which is incorporated in its entirety herein by reference). Likewise, 5 mg/kg of blank no clodronate liposomes (Liposoma BV, P-005) that were used as controls were similarly injected. Depending on the study paradigm, clodronate/no clodronate liposomes were administered for 6 h, 12 h, 24 h, 48 h and 72 h as per the study designs.

For all the studies involving administration of siRNA-encapsulated C12-200, 1 mg/kg of the siRNA concentration were administered intraperitoneally for the respective treatment periods as described in the study designs.

For the studies involving DiFC, mice were anaesthetized with 2% isoflurane to reduce motion and kept under nosecone anesthesia to achieve a steady state of anesthesia. After shaving off the tail hair, optical fiber probes were then placed on the surface of the tail's vascular bundle along with ultrasound gel to minimize index of refraction mismatch. Heating pads were used to preserve blood circulation to the extremities. Mice were scanned for 45 minutes, which, based on the flow rate of the tail vasculature, allowed us to interrogate the whole peripheral blood volume of the mouse several times.

DiFC study analysis: Diffuse in vivo Flow Cytometry (DiFC) is an emerging field in bio photonics that uses laser light coupled to two optical fiber-probes in series to non-invasively detect and count fluorescently labeled cells flowing in the vasculature of small animals without having to take blood draws. Each DiFC optical fiber probe acquires real-time data and is detected by photomultiplier tubes (PMTs) (Tan X et al., 2019; incorporated in its entirety herein by reference).

DiFC data was analyzed as described previously (Tan X et al., 2019; incorporated in its entirety herein by reference). Briefly, first, the data was preprocessed by background subtraction. Then, the noise was calculated, which was defined as the standard deviation of the data. Detections that were shown had intensity spikes five times greater than the calculated noise, which were referred to as “peaks”. Afterwards, some smoothing was performed to clean up the signal. To reduce artifacts caused by motion or instrument noise, a “matching” algorithm was employed. This consists of analyzing the peak's height and width and matching it with similar peaks appearing in the second probe. When DiFC detects a peak in one optical fiber probe and then a following peak is detected in the other probe, separated by a predetermined time, it was called a “matched peak” since it can be deduced as a cell traveling in either the arterial blood (from the heart to periphery), or the venous blood (from periphery to the heart). In this study, it was observed that only 40 to 50% of the total detected peaks in both fibers were matched. This relatively low matching count rate is most likely attributed to small misalignments between both fibers on the target blood vessel. Because of this, in this study the total mean count rate between both fiber probes was showed.

Peritoneal lavage isolation: Studies where peritoneal lavage was isolated, mice were sacrificed by respiratory depression under 5% isoflurane, followed by cervical dislocation following the IACUC guidelines, and peritoneal lavage was isolated following the procedure described previously (Ghosn E E B, et al., Two physically, functionally, and developmentally distinct peritoneal macrophage subsets. 2010; 107(6):2568-73; incorporated in its entirety herein by reference). After peritoneal lavage isolation, peritoneal macrophages (PMs) were enriched following the steps from the peritoneal macrophage isolation kit (Miltenyi Biotec, Cat number 130-110-434) by magnetically labeling and depleting non-macrophage cells from peritoneal lavage and enriching for PMs. Enriched PMs were washed in ice-cold sterile PBS twice and resuspended in ACK lysis buffer (Gibco™ A1049201) to lyse residual red blood cells. Peritoneal macrophages were finally resuspended in ice cold sterile PBS. On average, after enrichment, a yield of 1×10⁶ peritoneal macrophage cells was obtained in the end. These cells were then used for downstream assays.

Broncho-alveolar lavage fluid (BALF) isolation: Studies where BALF was isolated, mice were sacrificed by respiratory depression under 5% isoflurane, followed by puncturing the diaphragm following the IACUC guidelines, and broncho alveolar lavage was isolated following the procedure described previously (Sun F et al., Murine Bronchoalveolar Lavage. Bio-protocol. 2017; 7(10); incorporated in its entirety herein by reference). BALF cells were washed in ice-cold sterile PBS twice and resuspended in ACK lysis buffer to lyse residual red blood cells. Finally, cells were resuspended in cold sterile PBS and counted. On average, a yield of 5×10⁵ alive cells were obtained. These cells were then used for downstream assays.

PBMC isolation: Studies where PBMCs were isolated, mice were put under anesthesia under 5% isoflurane, and blood was harvested by retro-orbital (RO) bleeds. This was followed by increasing the isoflurane in the chamber until induction of respiratory depression, followed by puncturing the diaphragm following the IACUC guidelines. Blood was collected in K2 EDTA tubes to avoid any clotting and 1 ml of ACK lysis buffer was added per 100 μl of whole blood and washed for a couple of times. This step was repeated until the layer of RBCs was not seen. This was followed by washing with ice-cold sterile 1×PBS, making the PBMCs ready to be further prepared for flow cytometry staining and analysis.

Flow cytometry: Once the cells from the peritoneal lavage and BALF were harvested (as described above), a single cell suspension was generated by pressing with a plunger of a 5-ml syringe through a 70-μm nylon mesh filter into a 50 ml conical tube and washing the cells through with 5-10 ml of PBS/FCS buffer. Finally, the cells were washed in cold sterile PBS, and resuspended in FACS buffer (eBioscience™ Flow Cytometry Staining Buffer, 00-4222-57). 50 μl of cell suspension (equivalent to 105 cells) was aliquoted in wells of a sterile 96-well U-bottomed plate and mixed gently by pipetting. This was followed by an addition of 50 μl of Fc block (anti-CD16/CD32 #BE0307; 2.4G2 clone; Bio X Cell, diluted 1-2:200, 0.5-1 μg) and incubated for 5-10 minutes to eliminate all non-specific binding. Optimal concentration was pre-determined for each antibody by priorly run pilot studies. Desired antibodies (CD11b, F4/80) were diluted to 2× of the desired final concentration (1:200) in 100 μl of FACS buffer and added to the cell suspension previously added to the respective wells (final Ab dilution 1:100). This was incubated for 20 minutes at 40 C in the dark, followed by washes in sterile PBS, and finally, resuspended in 100 μl of FACS buffer and run on the BD FACSymphony™ A3 Flow cytometer (BD Biosciences). Later on, all the data generated on the flow cytometer was analyzed using the FlowJo™ v10.8 Software (BD Life Sciences).

Immunocytochemistry of peritoneal and broncho-alveolar lavage cells: Once the cells from the peritoneal lavage and BALF were harvested and processed into a single cell-suspension (as described above), cells were culture in low-glucose DMEM containing 2% FBS and 1% penicillin/streptomycin at a concentration of 5×10⁴ cells per well in a 96-well tissue-culture treated plate (Cell carrier-96 ultra, 6055300) and left in an incubator (5% CO2, 370C) overnight. Cell media was removed and 50 μl of fixative (4% PFA in PBS) (Paraformaldehyde Solution, 4% in PBS, Thermo Scientific™) was added to each well. After an incubation of 10 minutes at room temperature (RT), fixative was removed, and cells were washed with ice cold PBS (3×). Cells were then incubated in 50 μl per well of PTX permeabilization buffer (0.3% TritonX-100 in PBS) (diluted from Triton™ X-100, Sigma Aldrich, 9036-19-5) for 10 minutes at RT and again washed with ice cold PBS (3×) thereafter. Cells were then blocked in blocking buffer (5% NGS in PBS+0.1% Tween-20) for an hour at RT in dark. After aspirating the blocking solution, 50 μl per well of the primary antibody solutions were added. Primary antibody solutions were prepared by diluting Abs in PBST (PBS+0.1% Tween-20) (information on primary Abs used described earlier under ‘Antibodies and reagent’ section). Cells were incubated at 40 C overnight in the dark, followed by washes with ice cold PBST (3×), and addition of 50 μl of secondary antibody solution (diluted in PBST similarly) and incubation for 2 hours at RT in the dark. After aspiration of secondary antibody, cells were washed 2× in ice cold PBST, and during the last wash, NucBlue™ Live ReadyProbes™ Reagent (Hoechst 33342) was added and incubated for 5 minutes. This was aspirated, and cold PBS was added followed by sealing the plate. The plate was read on an Opera Phenix® High Content confocal microscope from Perkin Elmer®. Digital images were acquired using a 20× objective lens and quantification of imaged cells was carried out by the automated algorithms of the confocal microscope after optimization and background subtractions.

Data and statistical analyses: All the data was expressed as Mean+/−SEM. Statistical significance was determined by either unpaired student t-test (two tailed) whenever comparison between two groups was involved. In case of 4 or more groups, one way-ANOVA (two tailed), followed by Tukey's post hoc test was used to determine statistical significance. All the experimental findings were reproduced with biological replicates of 4 unless specified otherwise, and individual experiments were reproduced twice to confirm consistency of results. A p value of <0.05 was considered as statistically significant. All the statistical analysis was carried out using GraphPad Prism v7.01 (GraphPad by Dotmatics, © 2022 GraphPad Software).

### Results

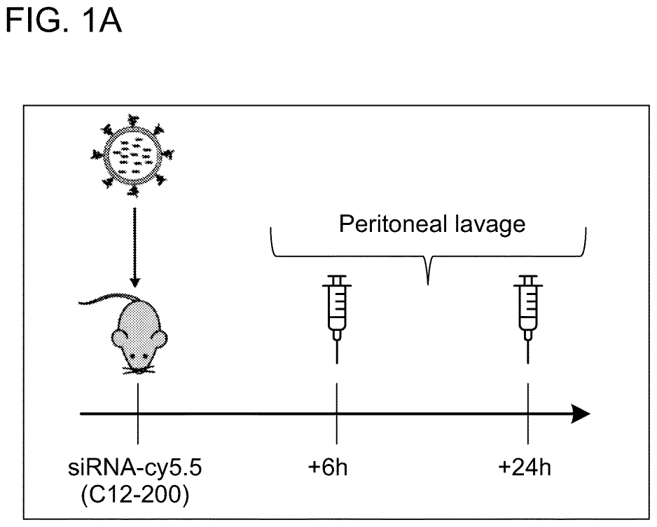

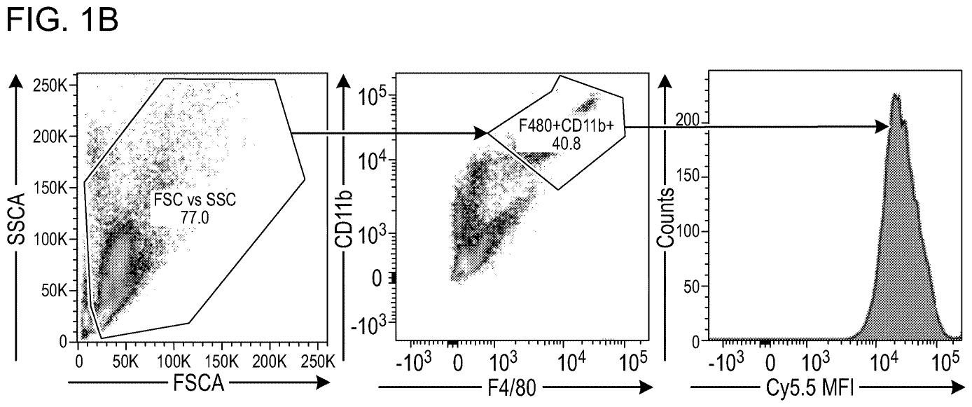

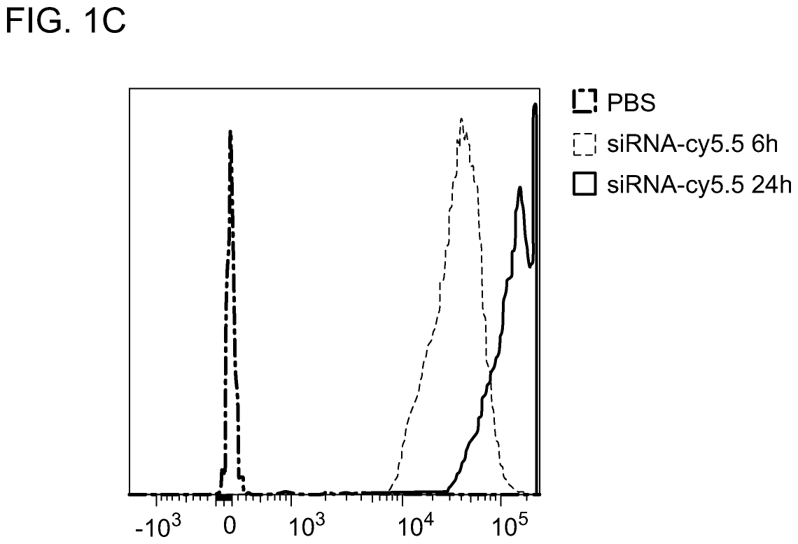

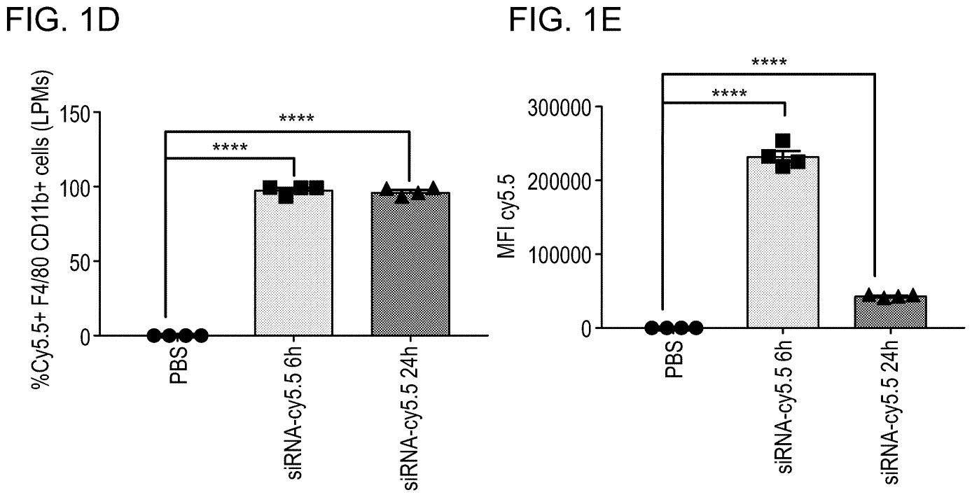

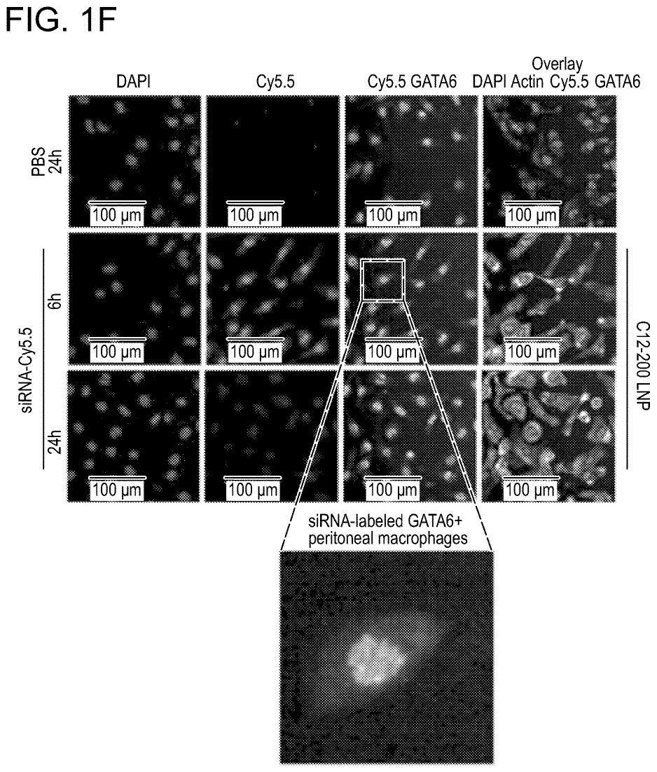

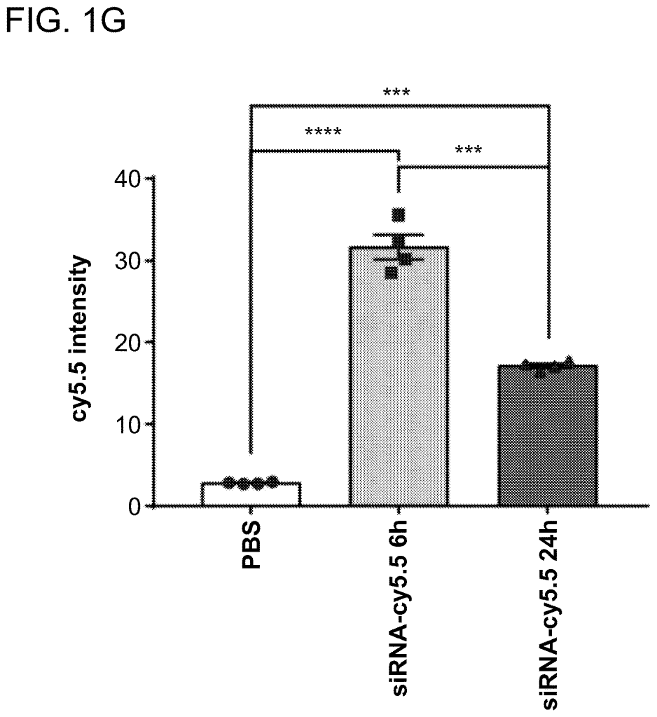

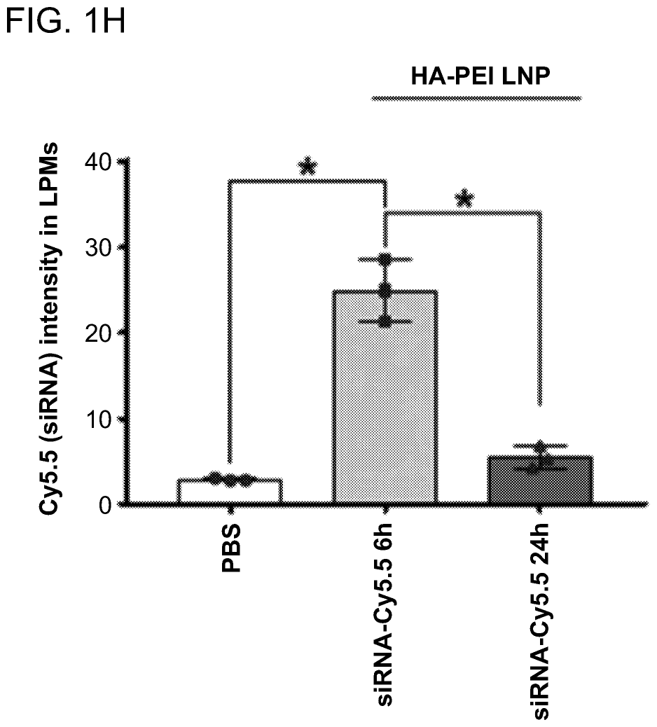

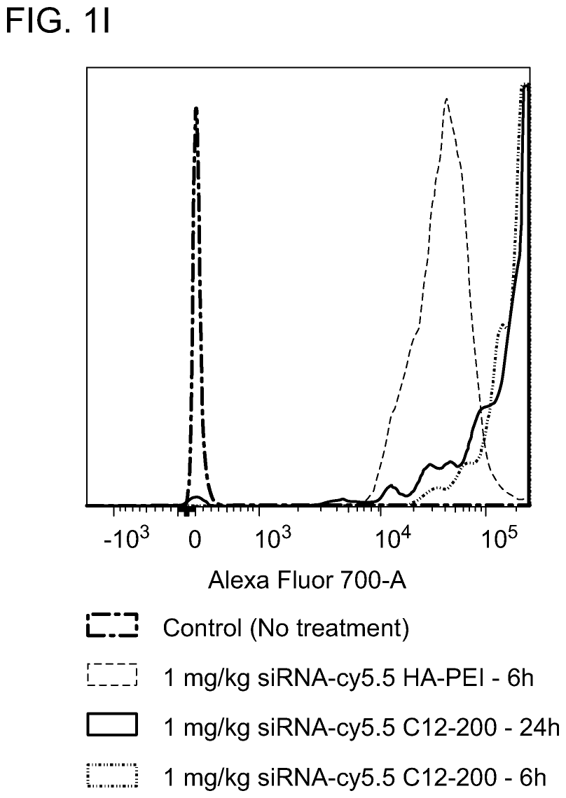

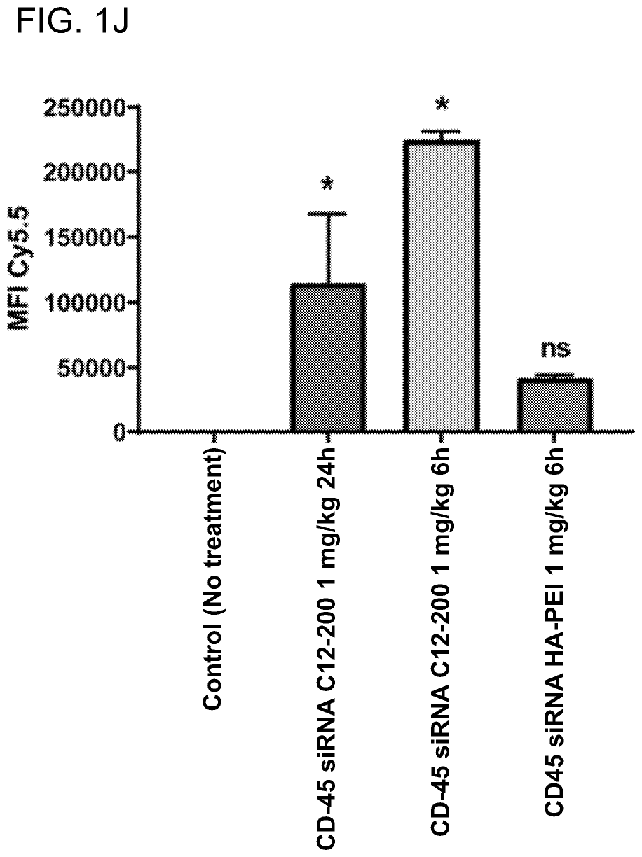

GATA6+ LPMs robustly take up C12-200 encapsulated siRNA: In order to characterize uptake of C12-200 encapsulated siRNA-cy5.5 in LPMs, peritoneal lavage was harvested post 6 h and 24 h intraperitoneal siRNA-cy5.5 administration, followed by enrichment of PMs by magnetically labeling and depleting non-macrophage cells (FIG. 1A). Flow cytometry analysis revealed a significant increase in the cy5.5 mean fluorescence intensity (MFI) upon treatment of siRNA-cy5.5 in the F4/80+CD11b+ gated population (FIGS. 1B-1E). Moreover, this was further validated by immunocytochemical analysis of the isolated PMs which suggested a robust uptake of cy5.5 labeled siRNA post 6 h treatment that decreased by 24 h in GATA6+ LPMs (FIGS. 1F-1G).

Delivery of HA-PEI and C12-200 encapsulated siRNA into LPMs: The primary goal of this aim was to compare the uptake efficiency, and also the mean fluorescence intensity of encapsulated cy5.5-labeled siRNA into peritoneal macrophages. CD44 receptor targeting hyaluronic acid (HA)-based nanoparticles (NP) have been extensively utilized as novel modalities to deliver oligonucleotide, as well as other nucleic acid payloads to macrophages, especially tissue resident peritoneal macrophages. Female Balb/c mice were dosed with 1 mg/kg of siRNAcy5.5-C12-200 and siRNA-cy5.5-HA-PEI NPs intraperitoneally, and peritoneal lavage was collected after 6 and 24 h. HA-PEI encapsulated cy5.5 labeled siRNA were taken up by LPMs at 6 h and 24 h (FIG. 1H).

Robust uptake of siRNA-cy5.5 encapsulated in C12-200 and HA-PEI was seen at 6 h treatment in LPMs. Uptake of siRNA-cy5.5-C12-200 at 6 h was higher than siRNA-cy5.5-HA-PEI dosed equivalently at 1 mg/kg of siRNA as evident by the cy5.5+ Mean Fluorescence Intensity (MFI) in macrophages (FIG. 1I-1J).

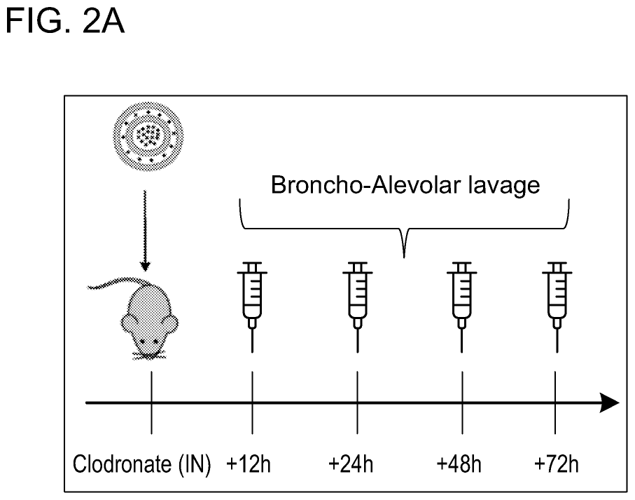

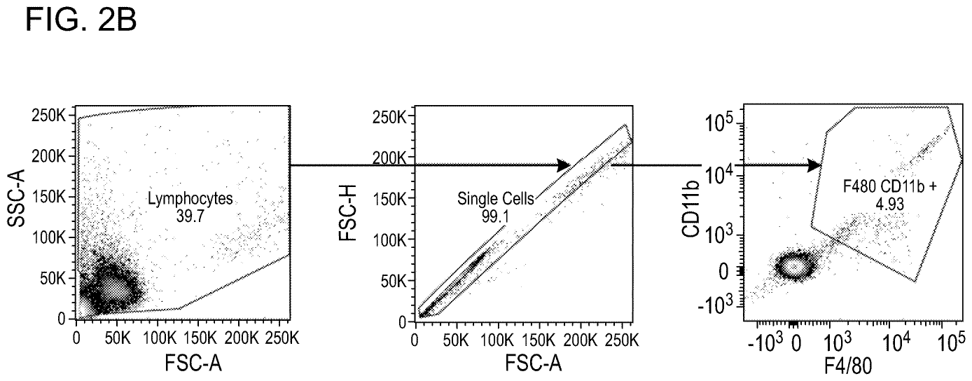

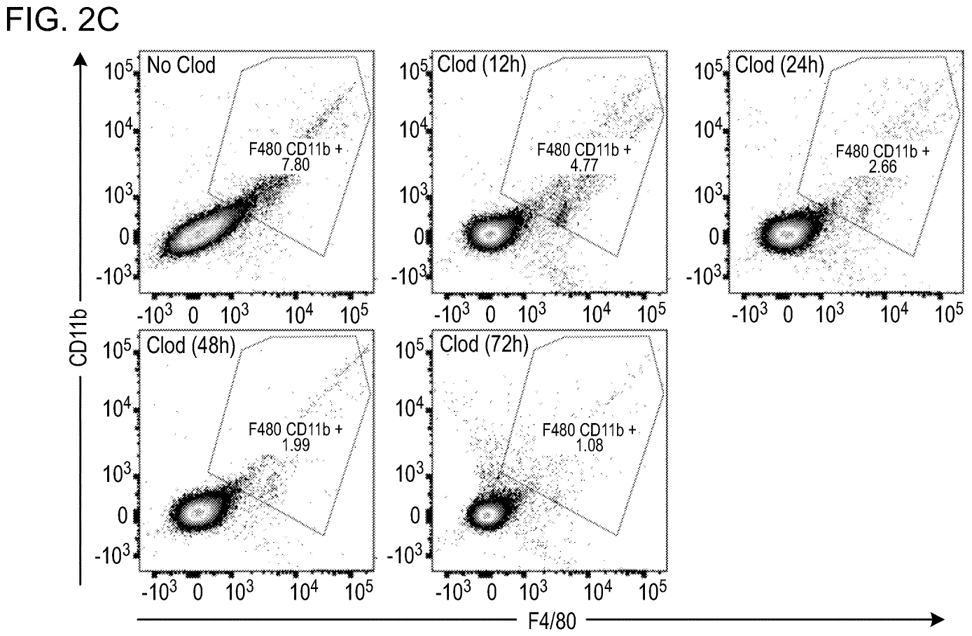

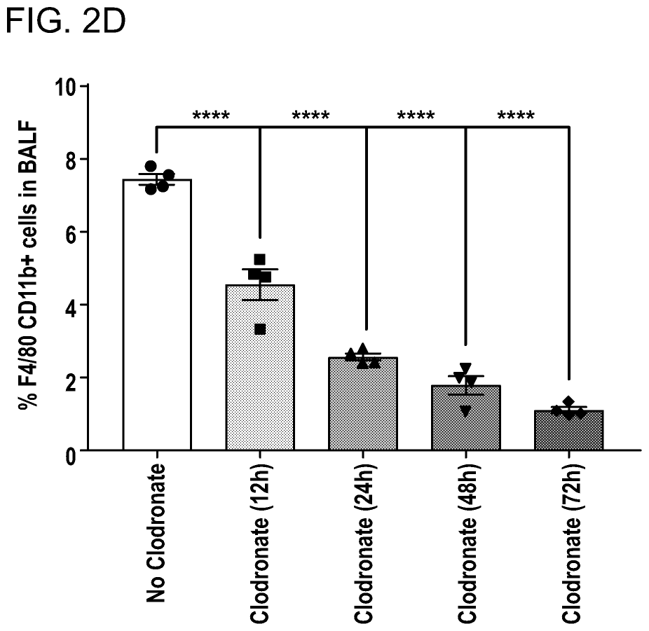

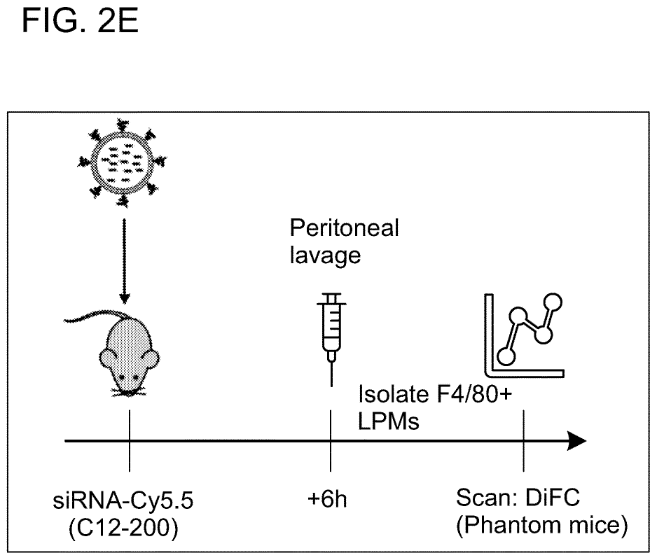

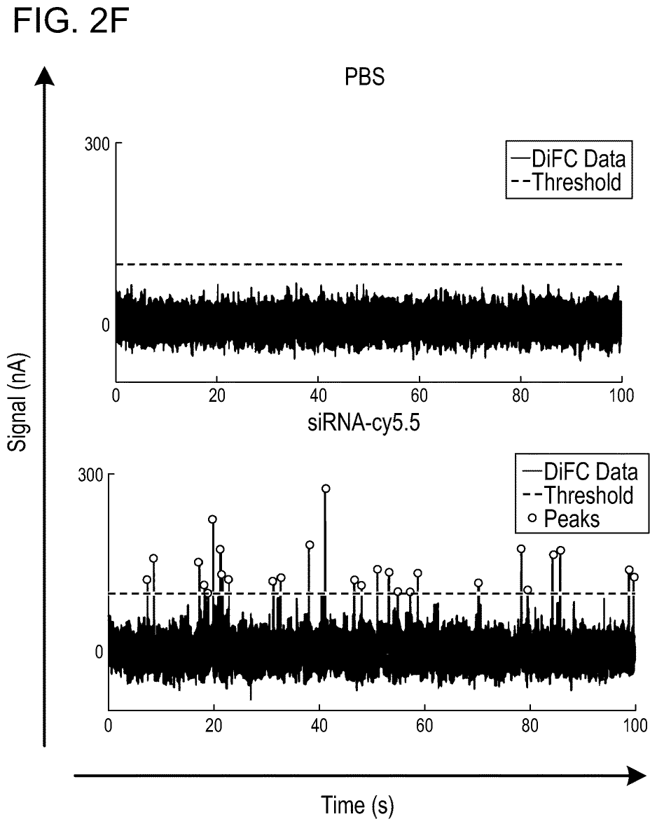

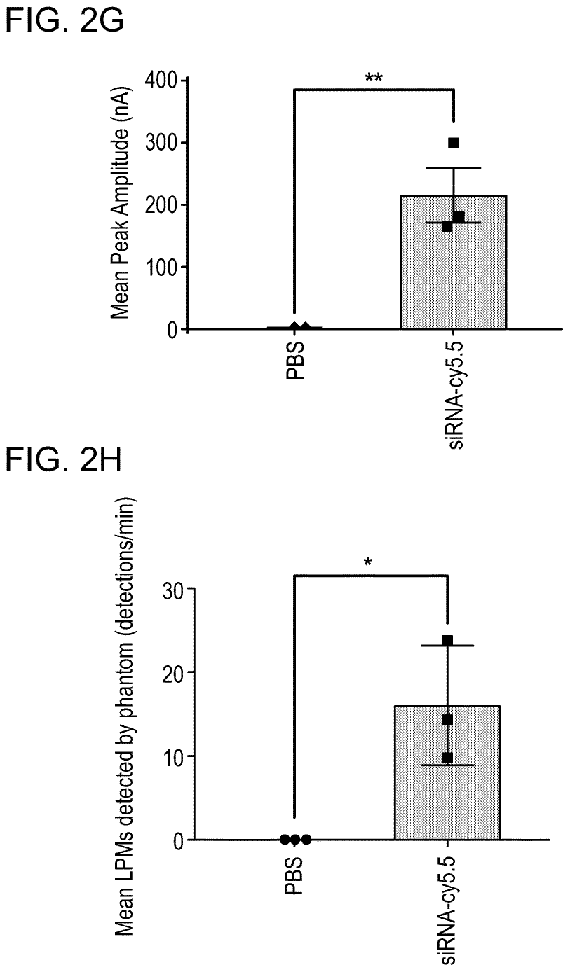

Intranasal administration of clodronate liposomes causes depletion of lung resident AMs: Intranasal administration of clodronate liposomes is well characterized to selectively deplete the lung resident AMs (Parayath N N et al., 2018; Van Rooijen N, and Hendrikx E. 2010; and Van Rooijen N, and Sanders A. 1994; each of which is incorporated in its entirety herein by reference). This study explored whether selective depletion of AMs led to LPM migration to the lungs, and aimed to selectively deplete the AMs by intranasally administering clodronate liposomes at a known cytotoxic dose of 5 mg/kg (Parayath N N et al., 2018; Van Rooijen N, and Hendrikx E. 2010; and Van Rooijen N, and Sanders A. 1994; each of which is incorporated in its entirety herein by reference). A dose of 5 mg/kg clodronate liposomes and an equivalent concentration of blank (no clodronate) liposomes were intranasally administered to mice, and broncho-alveolar lavage fluid (BALF) was isolated at 12 h, 24 h, 48 h and 72 h post clodronate administration to assess large macrophage depletion (FIG. 2A). Flow cytometry analysis revealed a time-dependent reduction in the percentage of F4/80+CD11b+ gated population with a significant reduction in the total number of large resident macrophage population at all time points (FIGS. 2B-2D). The results confirmed that there was a significant reduction in lung resident AMs.

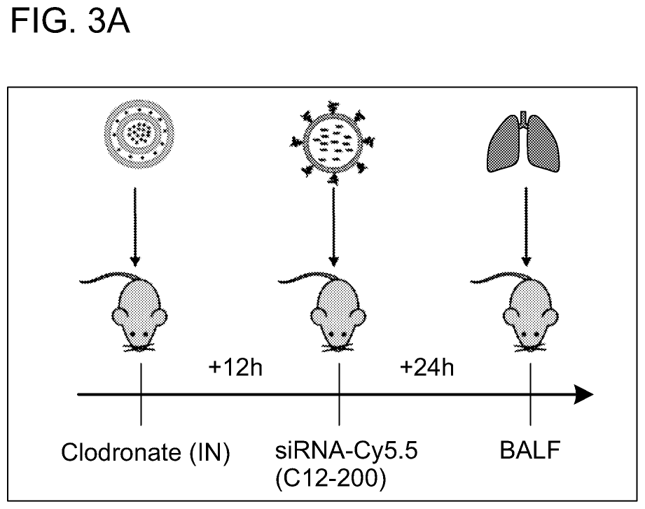

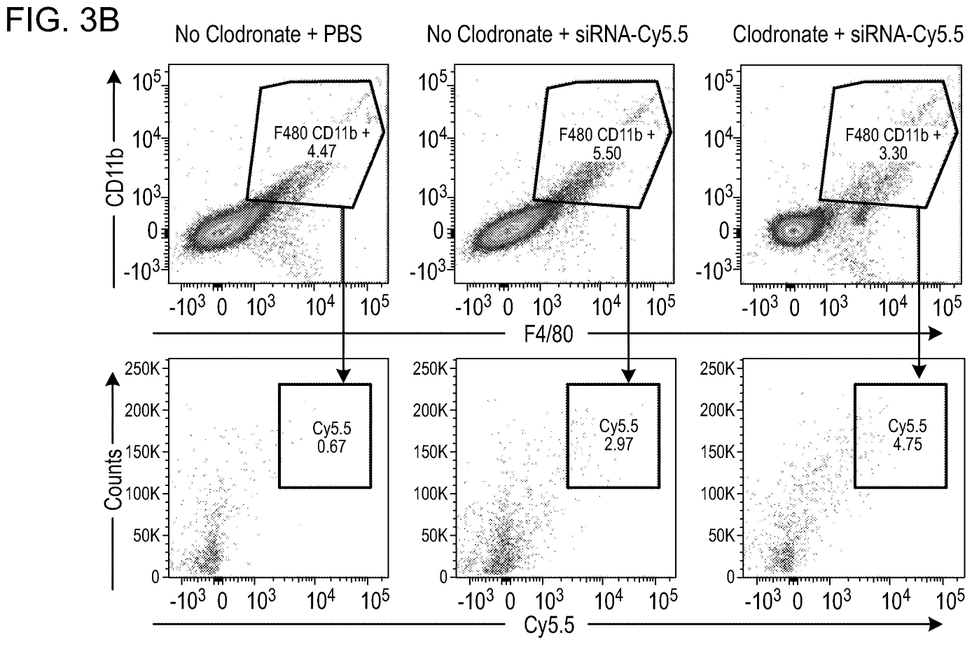

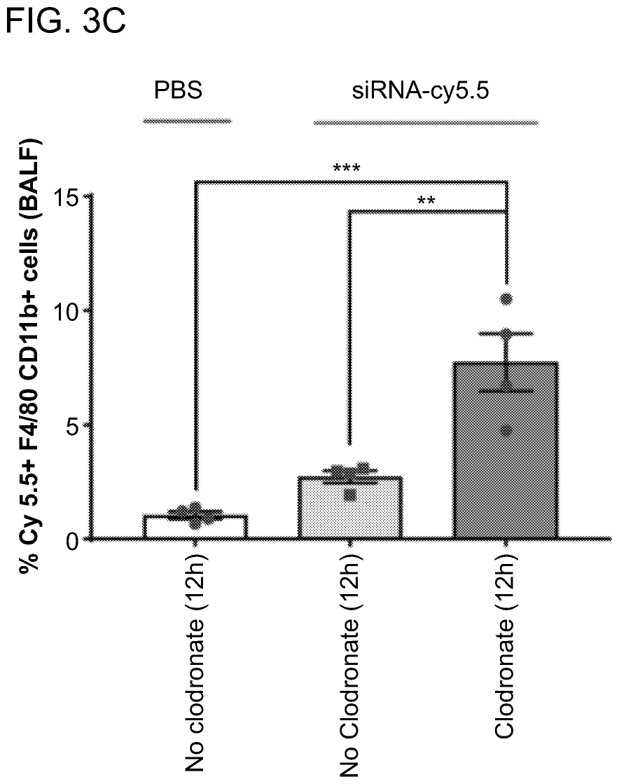

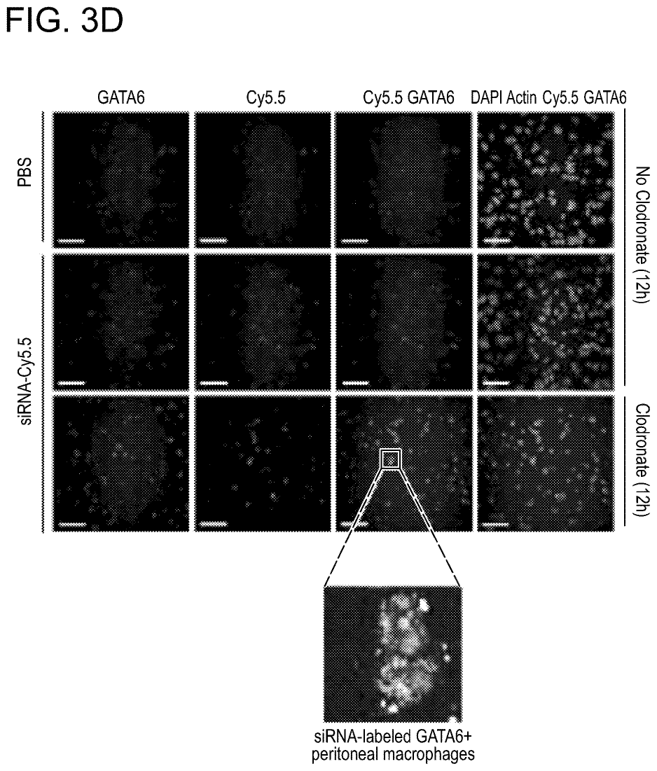

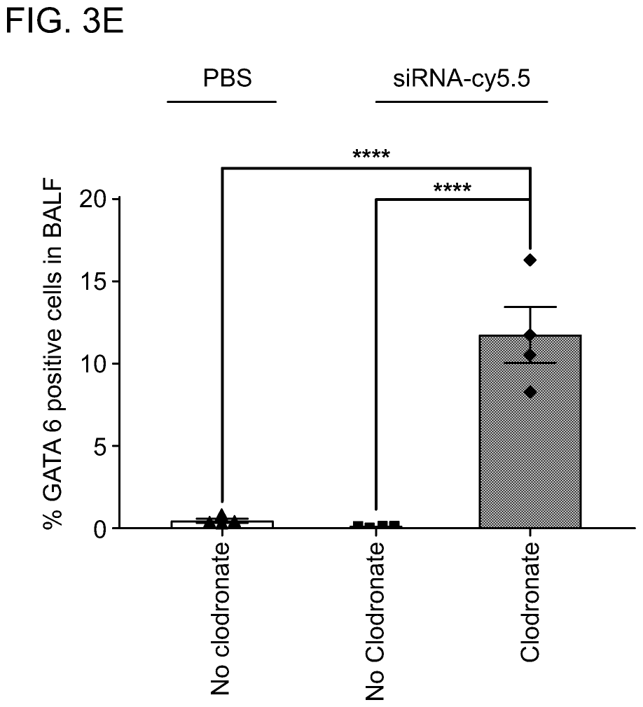

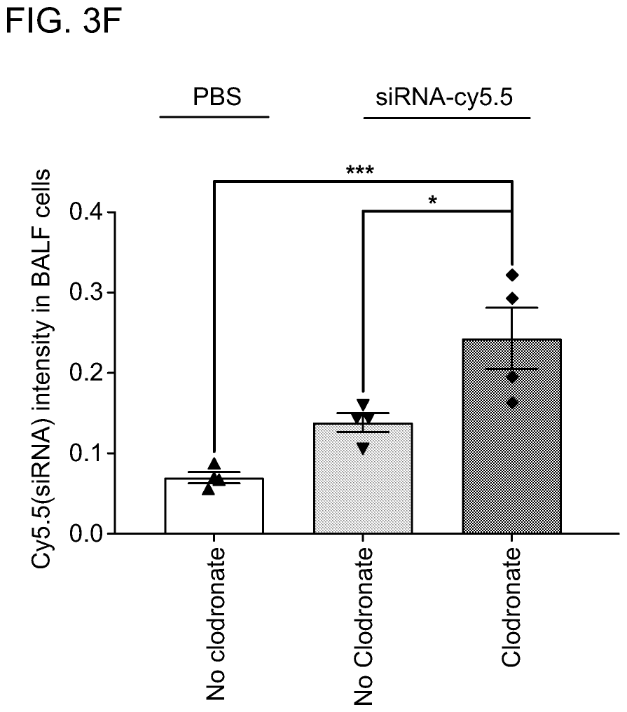

Increase in cy5.5 positive macrophages in the BALF after clodronate administration suggests an infiltration of a unique macrophage population to the lungs: Next, it was investigated whether right at an early time point after AM depletion, there was any infiltration of cy5.5-loaded mature F4/80 expressing macrophages into the lungs. Flow cytometry was used to phenotypically characterize macrophage cell populations in the BALF. Since it was previously observed that 12 h of clodronate treatment was enough to already reduce the AM population significantly, C12-200 encapsulated siRNA-cy5.5 was intraperitoneally dosed post 12 h following a 5 mg/kg intranasal administration of clodronate liposomes (FIG. 3A). Flow cytometry analysis revealed a significant increase in the percentage of cy5.5+ macrophages gated on the F4/80+CD11b+ population (FIGS. 3B-3C). This indicated that depleting AMs might be increasing infiltration of a unique cy5.5-loaded macrophage population, especially since the C12-200 encapsulated siRNA-cy5.5 was specifically administered intraperitoneally. Since it was previously confirmed that a robust and selective uptake of C12-200 encapsulated siRNA-cy5.5 to LPMs occurs as early as 6 h post siRNA administration (FIGS. 1B-1G), the next question raised was whether these infiltrating siRNA-cy5.5-loaded macrophages were indeed LPMs that had migrated to the lungs.

GATA6 expressing LPMs were detected in the BALF after 12 hours of clodronate administration: To identify whether LPMs were the source of the siRNA-cy5.5-loaded macrophages in the BALF, staining for the LPM expressing transcription factor GATA6 was performed. Since it has been well-known that GATA6 is selectively expressed by LPMs and not by AMs or any other monocyte derived or TRM populations, the present study aimed to utilize this marker to confirm the identity of this unique TRM population (Okabe Y and Medzhitov R. 2014; Honda M et al., 2021; Ito T et al., 2021; and Wang J and Kubes P., 2016; each of which is incorporated in its entirety herein by reference). Confocal imaging and immunocytochemical staining revealed a significant increase in the GATA6 stained cells in the BALF upon clodronate administration compared to the no-clodronate control (FIGS. 3D-3E). In fact, a strong nuclear staining of GATA6 was only seen in the BALF of clodronate-administered mice (FIG. 3D). Along with this, there was a clear colocalization of cy5.5+ GATA6+ cells (FIG. 3D). Additionally, there was also a further increase in cy5.5 intensity in the harvested BALF cells upon clodronate administration (FIG. 3F) demonstrating more migration of siRNA-cy5.5-loaded macrophages to the lungs after clodronate administration. In combination, these data supported the hypothesis that LPMs had migrated and infiltrated from the peritoneal cavity.

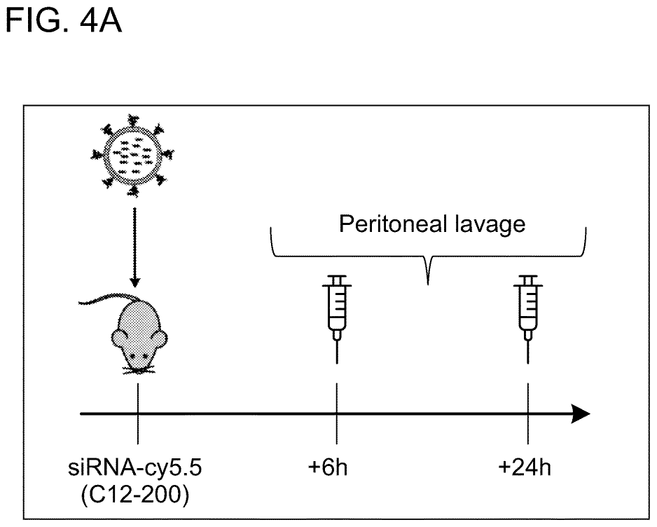

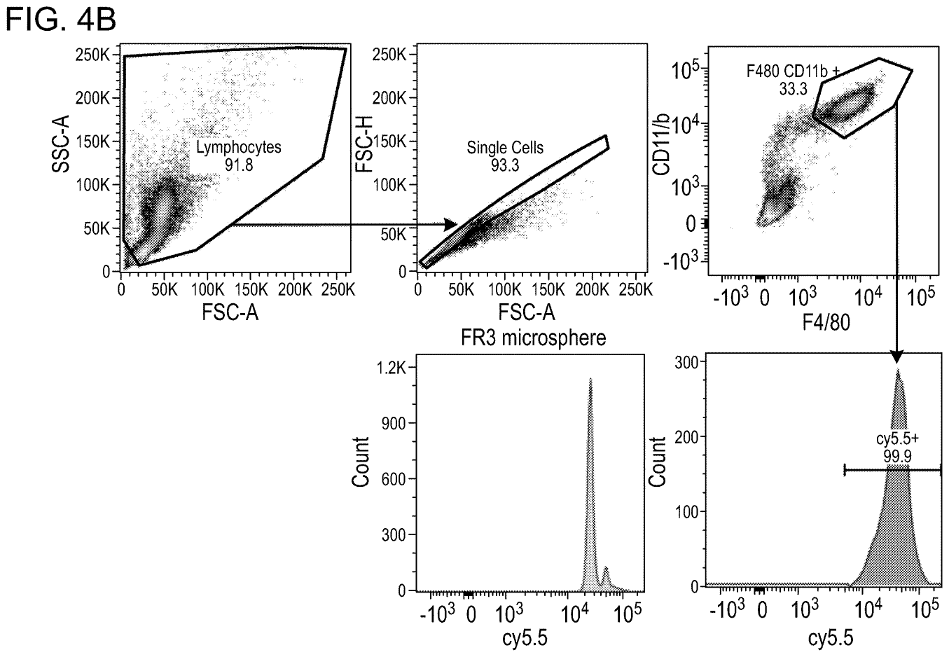

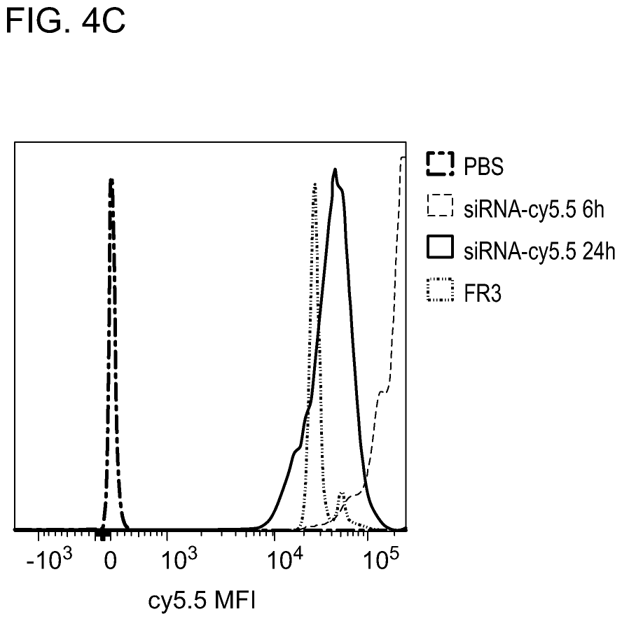

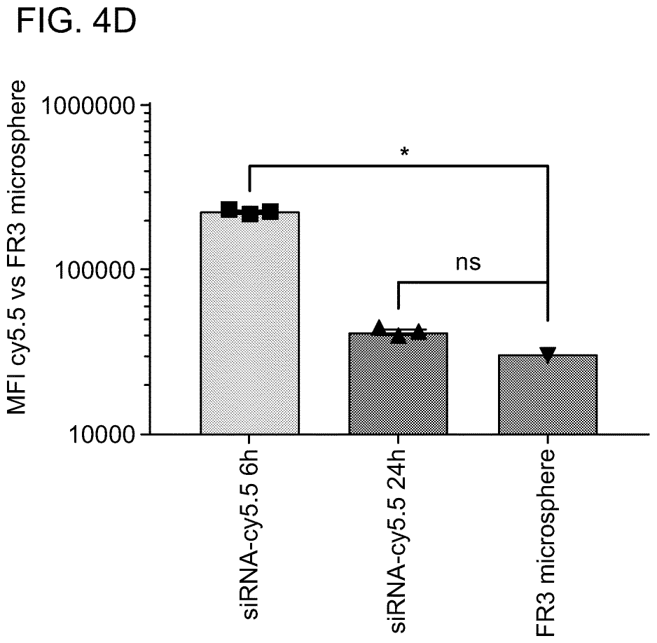

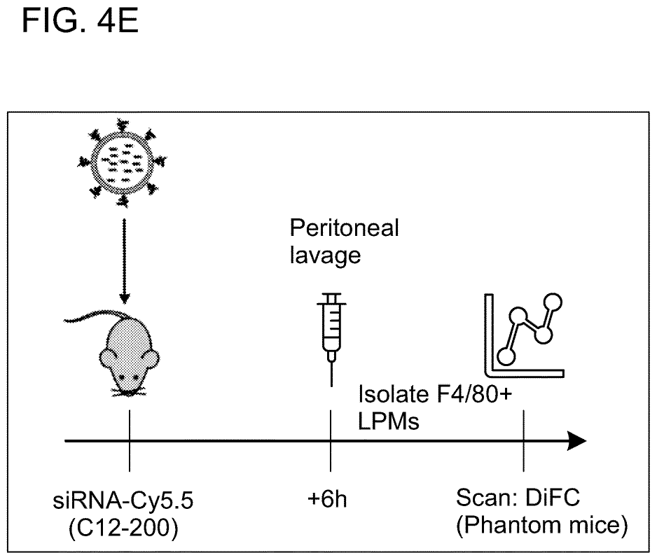

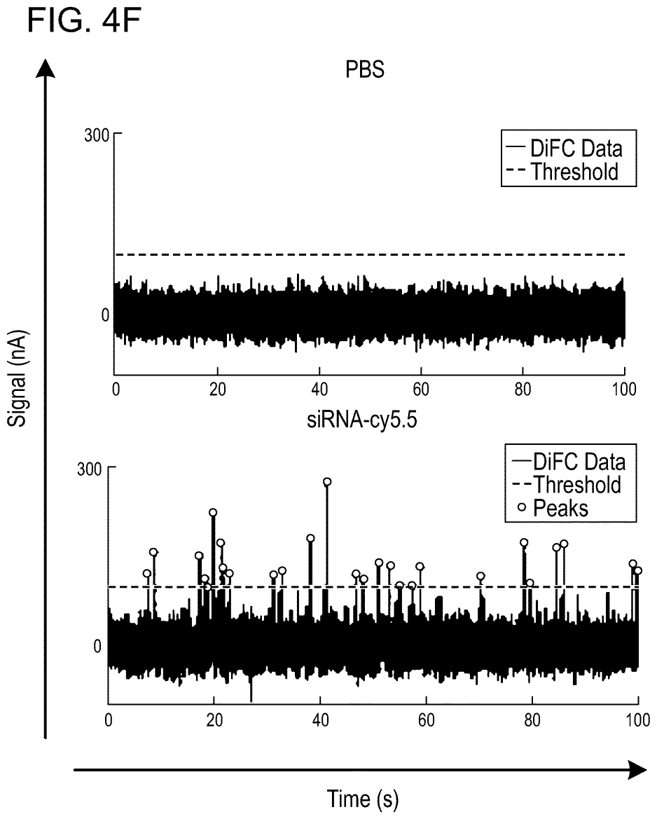

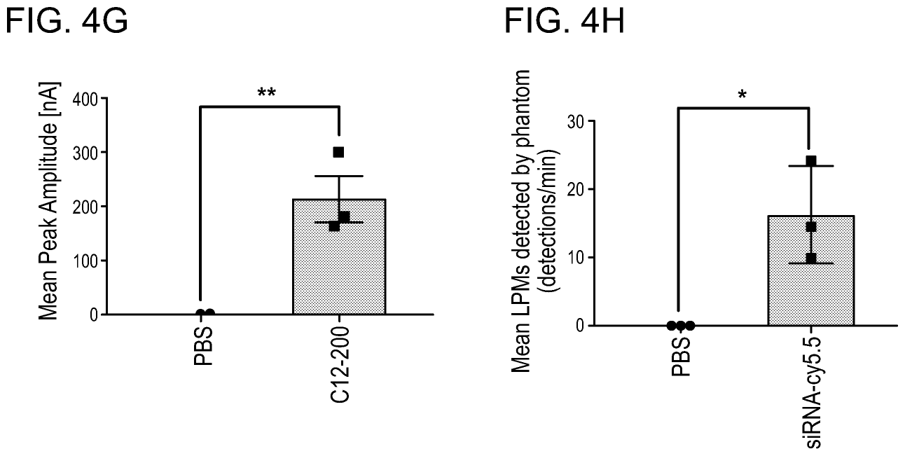

LPMs labeled with siRNA-cy5.5 in vivo were detected from peritoneal lavage by flow cytometry and DiFC scan in ‘phantom mouse’ model: The possibility of LPM intravasation into systemic circulation was next explored as one of the possible routes of how these mature TRMs migrate to the lungs upon AM depletion, and these fluorophore labeled cells were tracked in real time to further validate the initial findings. DiFC was used to detect and enumerate fluorescently labeled circulating macrophages in the vasculature. DiFC is a novel technique that utilizes the principles of near-infrared diffuse photons to detect and count fluorescently labeled cells flowing in arteries and veins (Tan X et al., 2019; and Pera V et al., 2017; each of which is incorporated in its entirety herein by reference). Hence, it makes it possible to count events as they pass through systemic circulation in a live mouse in real time (Tan X et al., 2019; and Pera V et al., 2017; each of which is incorporated in its entirety herein by reference). In a series of experiments as a follow up to DiFC, it was sought to confirm whether DiFC would indeed be detecting the cell-types of interest (cy5.5-labeled circulating LPMs) with sufficient accuracy. Hence, the fluorescence intensity of cy5.5-siRNA labeled LPMs was first tested against an internal reference standard microsphere Flash Red 3 (FR3) (Bangs Laboratories Inc.) (FIGS. 4A-4D). The study aimed to compare the Mean Fluorescence Intensity (MFI) of LPMs loaded with siRNA-cy5.5 with FR3 knowing that a higher MFI of cy5.5 in the LPMs on the red channel would label them brightly enough to be detected by DiFC later (Pera V et al., 2017; incorporated in its entirety herein by reference). After harvesting peritoneal lavage from mice dosed intraperitoneally with siRNA-cy5.5 for 6 h and 24 h, LPMs were enriched by magnetic bead depletion of non-target cells and MFI of cy5.5 was determined using flow cytometry (FIG. 4A). Flow cytometry analysis revealed that the MFI of cy5.5 of the F4/80+CD11b+ gated macrophages was higher than the FR3 microspheres at both 6 h and 24 h (FIGS. 4B-4D).

The study also confirmed detection of labeled LPMs isolated from the mouse peritoneal lavage harvested after 6 h of cy5.5 labeled siRNA treatment in a tissue simulating DiFC phantom model (Pera V et al., 2017; incorporated in its entirety herein by reference). This model was used as a proxy to further confirm detection of ‘peaks’ of cy5.5-loaded LPMs with accuracy as a prelude to the DiFC studies in vivo in mice. As previously described, phantom DiFC model incorporates a 3D-printed optical phantom which mimics the mouse tail vein. As has been shown before, the autofluorescence of the phantom approximates to that of biological tissue at near-infrared wavelengths. When isolated and enriched LPMs that were previously dosed with siRNA-cy5.5 were run through the phantom, not only was the mean peak amplitude sufficiently high in the siRNA-cy5.5 treated group (FIGS. 4F-4G) but there was also a significant increase in the number of ‘peaks’ that were detected (FIGS. 4F and 4H), pointing to detection of labeled LPMs in this pure sample set of harvested and enriched LPMs from mice. This confirmed that PMs were sufficiently labeled brightly enough for detection in mice in vivo with DiFC.

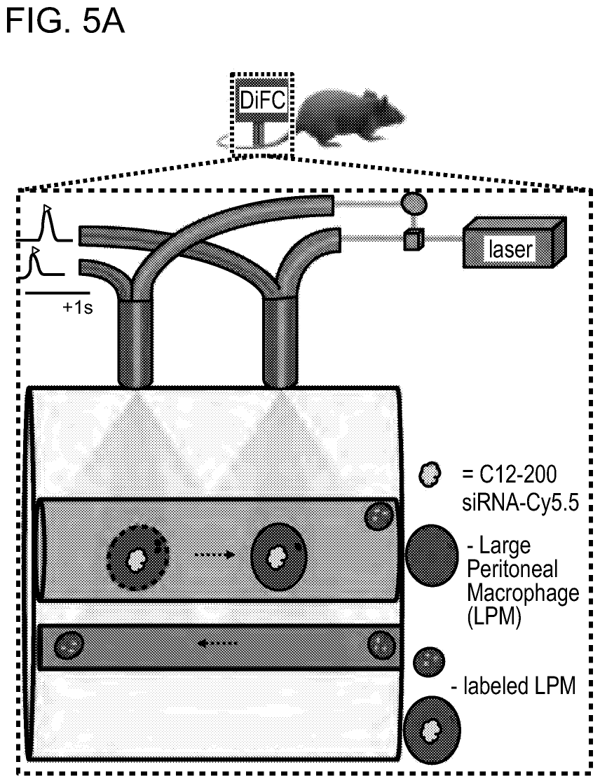

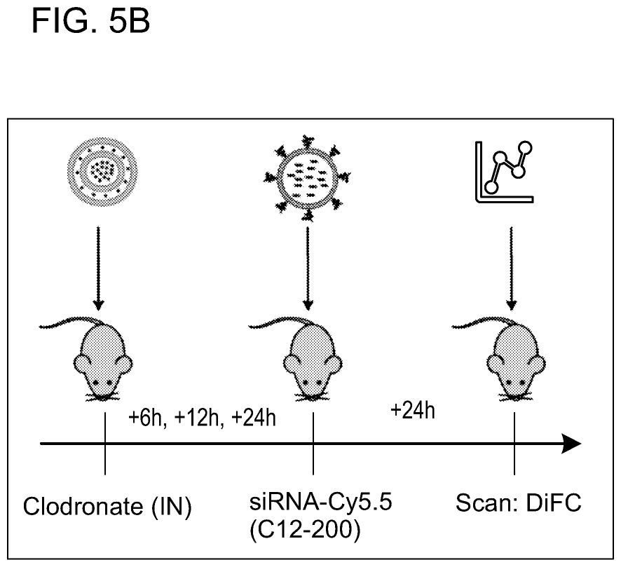

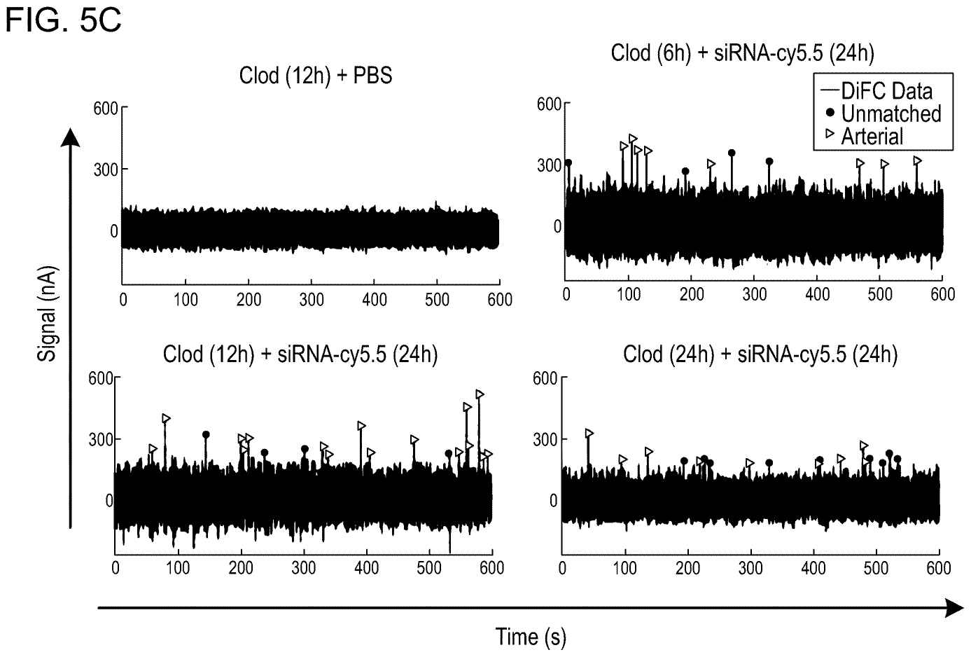

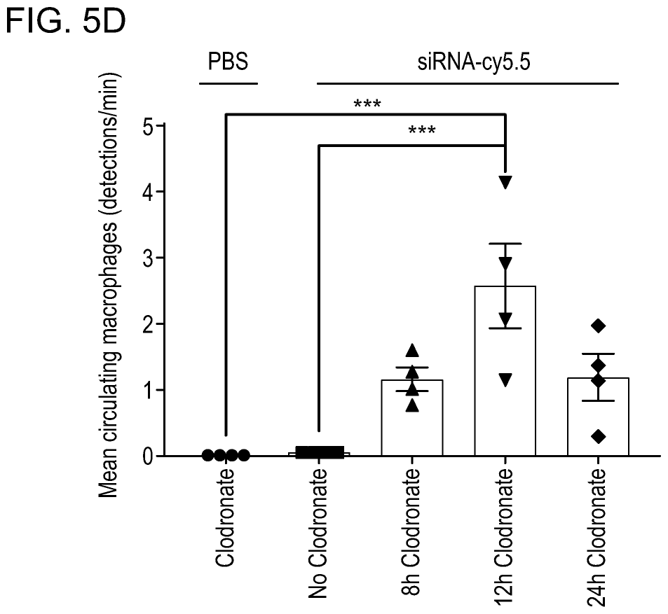

Systemically circulating labeled PMs were detected by DiFC upon clodronate-induced AM depletion: After the series of DiFC optimization experiments, siRNA-cy5.5-dosed mice were subjected to DiFC at earlier time points after intranasal clodronate administration. FIG. 5A depicts the schematics of the DiFC study. After either 6 h, 12 h and 24 h post clodronate administration, mice were injected with 1 mg/kg of the siRNA-cy5.5-C12-200 or a dose equivalent of 1×PBS controls intraperitoneally. DiFC was performed on live mice 24 h after siRNA administration (FIG. 5B) in quadruplicate for 45 minutes, allowing enumeration of labeled macrophages in systemic circulation. As shown in FIGS. 5C-5D, detection of cy5.5 encapsulated macrophages was indicated by a transient ‘peak’ as cells passed through the DiFC field of view, thereby indicating the presence of LPMs in the blood. These peaks were not observed in PBS only and no-clodronate liposome controls. The number of LPMs in circulation peaked 12 hours following clodronate administration (FIGS. 5C-5D). This strongly suggested that macrophage migration from the peritoneal cavity to lungs upon AM deletion was a time-dependent phenomenon peaking near 12 h, supporting hypothesis that this phenomenon is acutely driven.

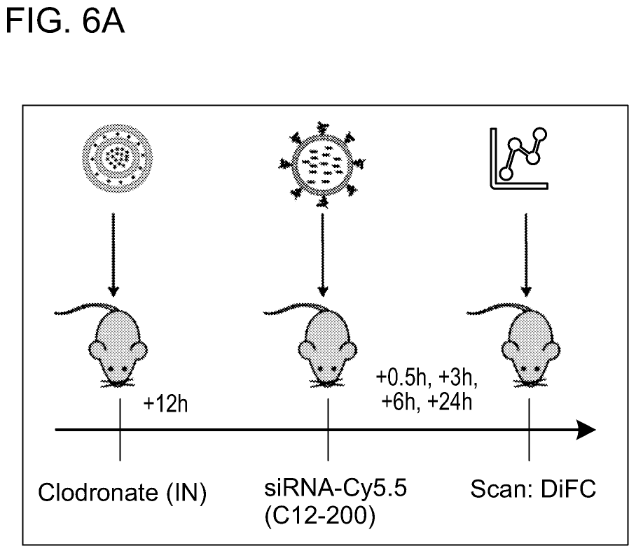

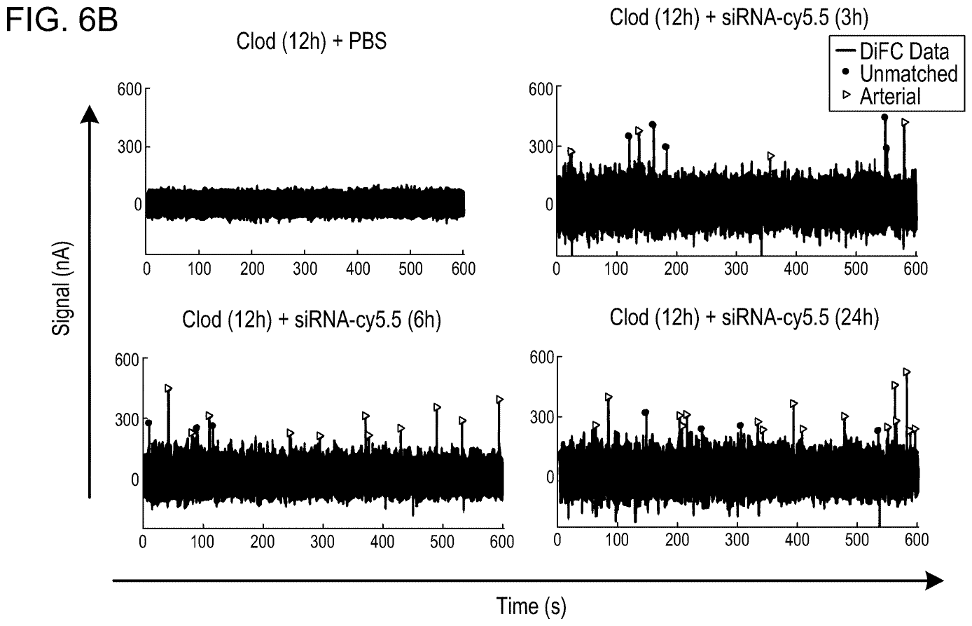

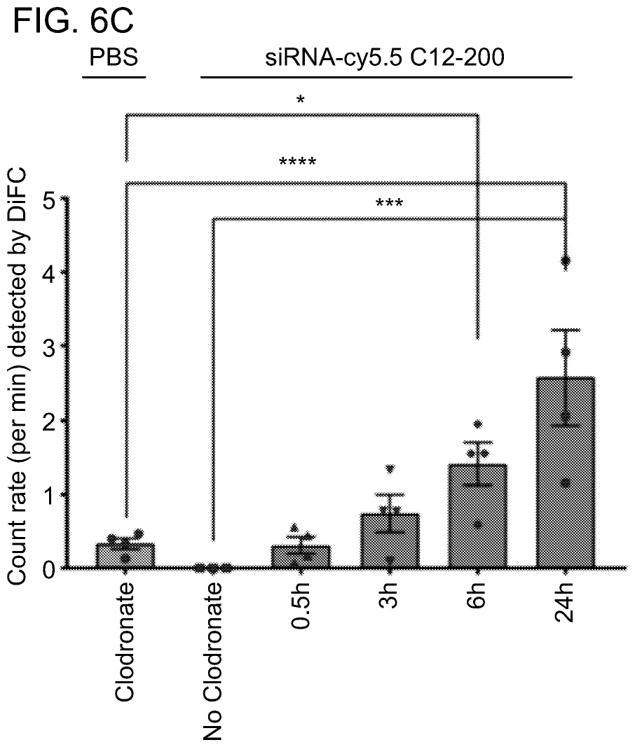

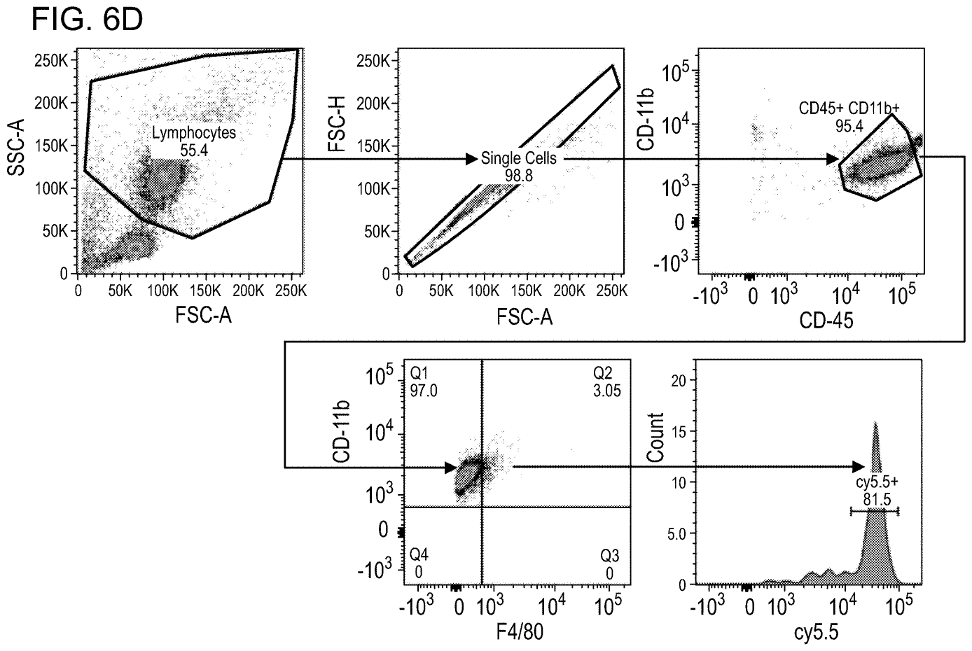

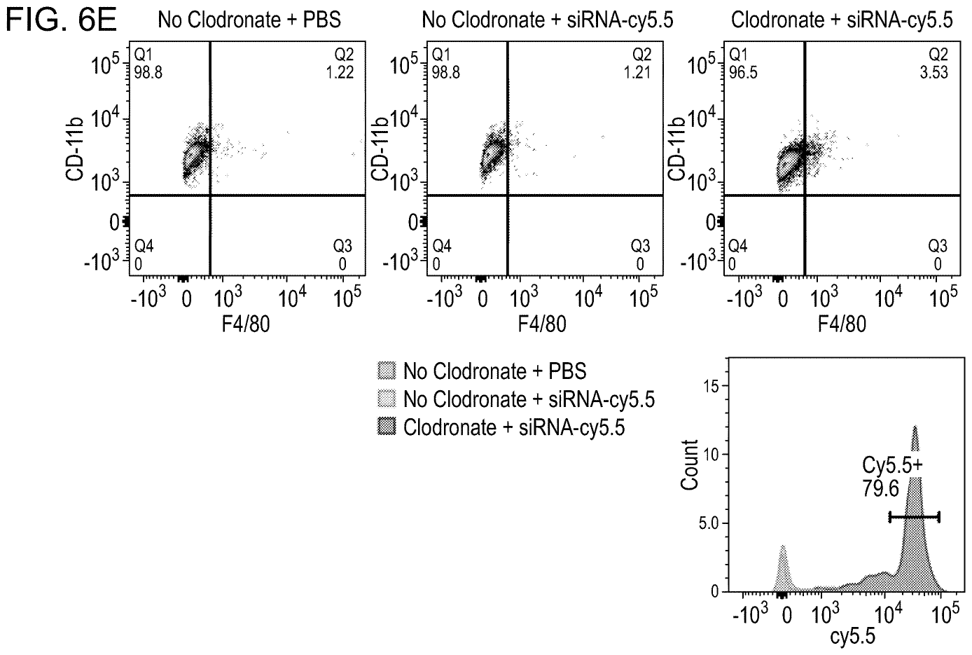

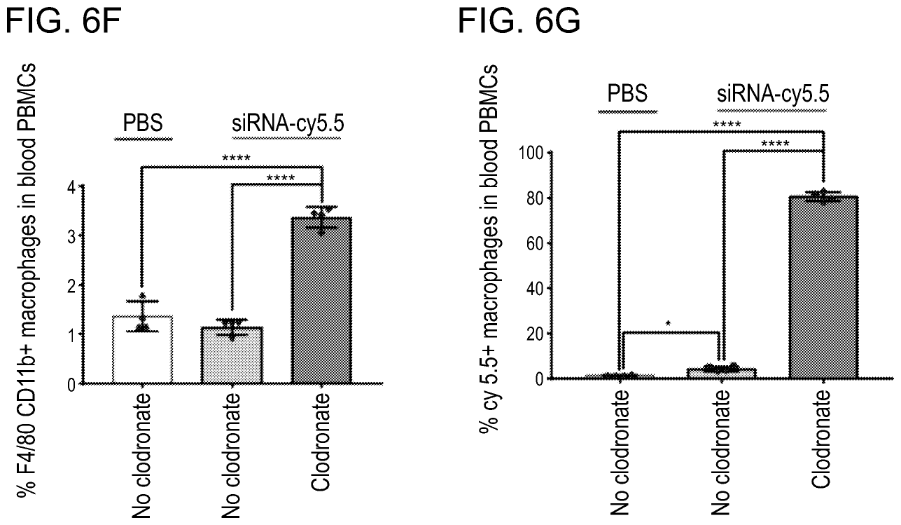

Increase in number of circulating LPMs was detected by DiFC scans across different time points over 24 h post 12 h clodronate administration: After confirming an active migration of LPMs to the lungs upon administration of clodronate liposomes, the kinetics of LPM numbers in circulation following clodronate administration were also considered. DiFC was performed at 30 minutes, 3 h, 6 h and 24 h after intraperitoneally injecting 1 mg/kg siRNA-cy5.5-C12-200 and (FIG. 6A). In all cases, the siRNA injection was performed 12 h after clodronate treatment (consistent with findings in FIG. 5 above). Each DiFC scan was performed for 45 minutes and repeated 3 to 4 times (FIG. 6B). As shown in FIG. 6C, a progressive increase in detected circulating LPMs was observed from 30 minutes to 24 h, with maximum observed 24 h post siRNA-cy5.5 injection. No clodronate-liposome treated mice produced negligible detections (FIGS. 6D-6G). In combination, these results demonstrate that intravasation of labeled PMs into systemic circulation followed clodronate-mediated AM depletion.

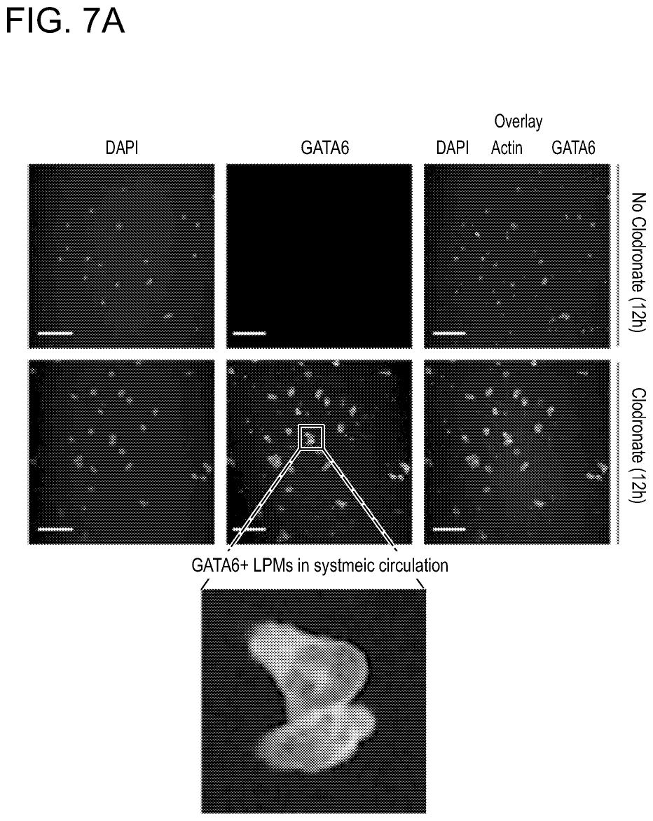

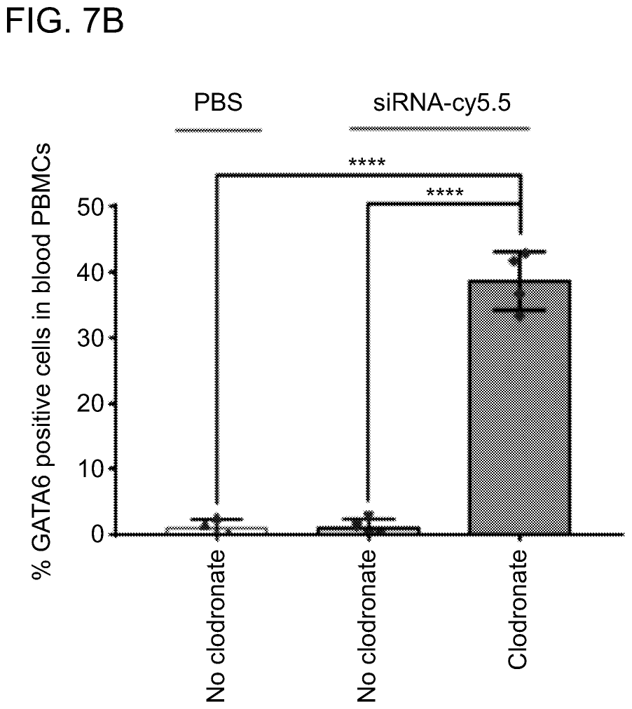

Circulating LPMs in whole blood were seen upon clodronate-induced AM depletion: Since DiFC results pointed out to the detection of LPMs in systemic circulation only when AM were depleted, the study further corroborated the DiFC results by further probing into whole blood circulating immune cells to detect the presence of mature F4/80+CD11b+ GATA6 expressing LPMs in whole blood. Hence, mice were dosed with C12-200 encapsulated siRNA-cy5.5, and isolated whole blood peripheral blood mononuclear cells (PBMCs) 24 h post siRNA-cy5.5 injection. Flow cytometry analysis revealed a significant increase in the mature macrophages in clodronate-treated versus no-clodronate control samples (FIGS. 6D and 6F). Although still a rare population observed in whole blood, there was a significant increase in the percentage of F4/80+CD11b+ cells seen only upon clodronate administration. Moreover, there was also a robust and significant increase in % cy5.5 cells in the F4/80+CD11b+ gated population after clodronate administration suggesting that most of the cy5.5-labeled mature macrophage population in circulation are dependent on AM depletion, eliminating the possibility of any non-specific cy5.5-labeled circulating cell population due to non-specific uptake of C12-200. This result further solidified the DiFC findings. Finally, immunofluorescence staining of harvested PBMCs revealed a significant increase in GATA6+ LPMs in blood after clodronate administration (FIGS. 7A and 7B). Since blood monocytes, or any other cells of the immune system do not express GATA6, the presence of GATA6 expressing cells resoundingly confirmed the presence of LPMs in systemic circulation which was directly dependent on clodronate mediated AM depletion. In combination, this data demonstrates that intravasation of siRNA-cy5.5 labeled LPMs into systemic circulation followed clodronate-mediated AM depletion.

The peritoneal cavity is a fluid-filled serous (Bain C C, and Jenkins S J. The biology of serous cavity macrophages. Cellular Immunology. 2018; 330:126-35; incorporated in its entirety herein by reference) cavity that is a source of naïve TRMs along with harboring a number of other immune cells including small PMs (monocyte-derived), B-cells and T-cells (Ray A, and Dittel B N. Isolation of mouse peritoneal cavity cells. J Vis Exp. 2010(35):1488; incorporated in its entirety herein by reference). It is only recently that the true heterogeneity of all the immune cell populations residing in the peritoneal cavity has been fully elucidated revealing unique transcriptomic profiles among TRMs belonging to the peritoneal cavity (Oza D and Amiji M M. 2022; Nobs S P and Kopf M. 2021; Okabe Y and Medzhitov R., 2014; Bain C C, and Jenkins S J, 2018; Okabe Y, and Medzhitov R. Tissue biology perspective on macrophages. Nature Immunology. 2016; 17(1):9-17; Jenkins S J, and Allen J E. The expanding world of tissue-resident macrophages. 2021; 51(8):1882-96; and 43. Lee C Z W, and Ginhoux F. Biology of resident tissue macrophages. Development. 2022; 149(8); each of which is incorporated in its entirety herein by reference). One such unique identifying marker of LPMs is the zinc-finger transcription factor GATA6 (Honda M et al., 2021; Ito T et al., 2021; Wang J and Kubes P., 2016; and Jin H et al., Genetic fate-mapping reveals surface accumulation but not deep organ invasion of pleural and peritoneal cavity macrophages following injury. Nature Communications. 2021; 12(1):2863; each of which is incorporated in its entirety herein by reference). GATA6-expressing LPMs have known tissue-specific roles and it has been established that they are unique in their ability to migrate to areas of injury within the peritoneum, a phenomenon not seen with other TRMs (Honda M et al., 2021; Ito T et al., 2021; Wang J and Kubes P., 2016; and Jin H et al., 2021; each of which is incorporated in its entirety herein by reference). The results suggest that this is an inherent property of this mature innate immune cell population, as they are readily available to sense injury elsewhere in the body and have an ability to migrate and influence the microenvironment and the reparative ability of the injured tissue.

The unique properties of DiFC were used to study whether LMs migrate into circulation following AM depletion in the lungs. Because DiFC is non-invasive and does not require drawing blood, it could be performed continuously for extended periods of time (for example, 45 minutes in this case) while mice are under anesthesia and could be repeated at multiple timepoints to resolve the kinetics of the migration (Tan X et al., 2019; Di W et al., 2020; and Williams A L et al., 2020; each of which is incorporated in its entirety herein by reference). DiFC is an optical technique that has been mainly used for cancer research, specifically detecting circulating tumor cells (CTCs) in mouse models of hematogenous metastasis. However, in this study, DiFC showed that there were fluorescent cells in the peripheral blood of mice only with AM depletion via clodronate and cy5.5 labeled siRNA-administration into the peritoneal cavity. This proved the need for a stimulus and implies some form of communication between the lungs and the peritoneum.

Macrophages are important therapeutic targets considering their multiple vital roles in inflammatory diseases, autoimmune diseases, and cancer (Oza D and Amiji M M. 2022; Zhang C et al., 2021; Xiao Y and Yu D. 2021; Wang H et al., 2021; and Tan Y et al., 2021; each of which is incorporated in its entirety herein by reference). Despite making significant progress with tissue-selective delivery with oligonucleotide therapies, there are still considerable roadblocks to selective delivery to immune cells (Setten R L et al., 2019; Wittrup A and Lieberman J. 2015; Aigner A. 2019; and Roberts T C et al., 2020; each of which is incorporated in its entirety herein by reference). The present study has directly demonstrated an ability to deliver a fluorescently (cy5.5)-labeled-siRNA to GATA6+ LPMs. Due to the success of NP-based delivery systems for delivering oligonucleotide therapies, the present study successfully utilized a novel approach of encapsulating modified double-stranded siRNA in a cationic lipid, C12-200 (Novobrantseva T I et al., 2012; Love K T et al., 2010; Whitehead K A et al., Degradable lipid nanoparticles with predictable in vivo siRNA delivery activity. Nature Communications. 2014; 5(1):4277; and Zhang Y et al., Lipids and Lipid Derivatives for RNA Delivery. Chemical Reviews. 2021; 121(20):12181-277; each of which is incorporated in its entirety herein by reference). This work therefore, demonstrates the potential of RNAi-mediated gene silencing as a therapeutic modality for macrophage delivery.

While it had been sufficiently established until now that LPMs are not necessarily ‘resident’ and can migrate and infiltrate peritoneally located organs like the liver and intestines via an avascular route, the results of the present study demonstrate the possibility of seeing this more broadly across non-peritoneally located organs and thus, suggest this migration as an inherent property of these unique TRM population (Honda M et al., 2021; Ito T et al., 2021; and Wang J and Kubes P., 2016; each of which is incorporated in its entirety herein by reference). These results therefore, demonstrate the potential to develop nucleic acid molecule based therapeutic modalities, such as RNAi therapies, targeted to LPMs without the need to remove them from the body and engineer them ex vivo; and utilizes these cells as a delivery modality.

### Example 3. Large Peritoneal Macrophages Comprising Lipid Nanoparticles Encapsulating a Nucleic Acid Molecule Migrate to the Liver Via Systemic Circulation in a Mouse Model of Acetaminophen-Induced Liver Injury

A mouse model of acetaminophen induced liver injury (AILI) was successfully developed as evident from the significant elevations in the circulating liver injury enzymes as well as necrosis observed by immunohistochemical staining of H&E. Injury phenotype was observed to be the worst from 6 to 24 hours, with evidence of wound healing and a reduction in inflammation observed by 48 hours. GATA6+ LPM infiltration was observed, with an increased infiltration seen from 6 to 24 hours. DiFC data demonstrated a clear detection of labeled peaks upon AILI, with the maximum number of peaks observed from 6 to 24 hours. Gene expression analysis and measuring secreted cytokine levels in peritoneal lavage and liver confirmed macrophage pro-inflammatory phenotype and an acute propagation of inflammation downstream to AILI.

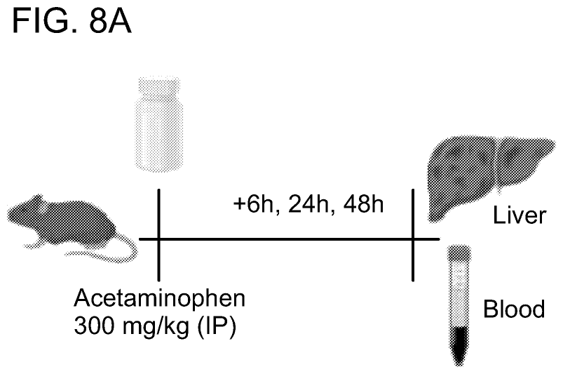

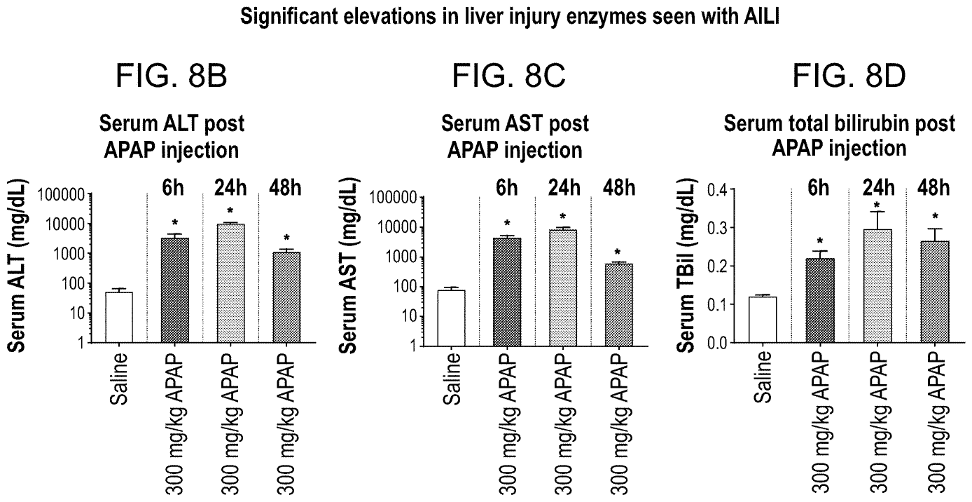

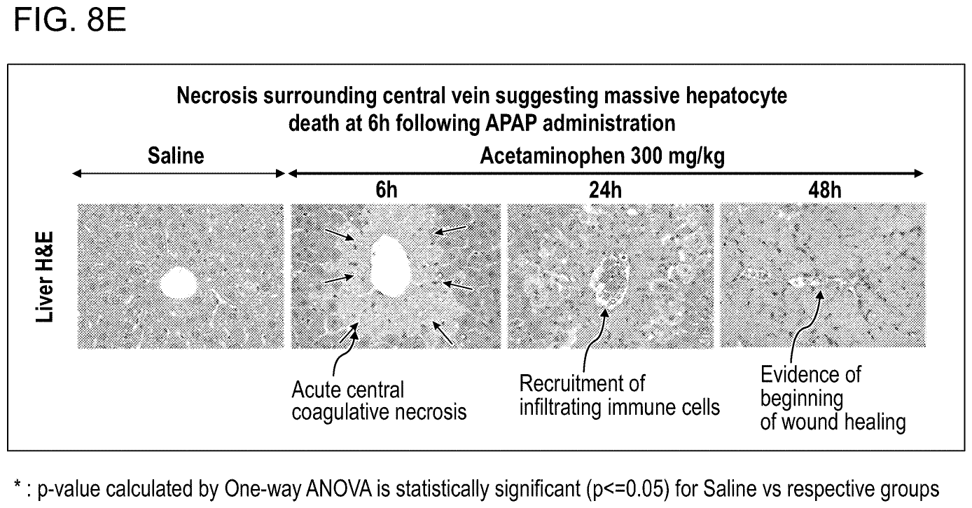

Development of a mouse model of AILI as observed from circulating biomarkers and immunohistochemical (IHC) evaluation of liver: Female Balb/c mice were used for all the APAP-induced liver injury studies. Mice were fasted for 12 h to deplete the glutathione reserves and then dosed with a 300 mg/kg dose of APAP via intraperitoneal injection. Blood, peritoneal lavage, and liver were isolated at 6 h, 24 h and 48 h post APAP injection. A study schematic can be seen in FIG. 8A. Livers were washed in ice-cold 1×PBS and left-lateral lobe of the liver was transferred to a formalin containing jar for fixation, to be further processed into paraffin-embedded tissue blocks for immunohistochemical staining. Remainder of the livers were snap frozen in liquid nitrogen to be further processed for downstream analysis.

The formalin containing left lobes of the livers was allowed to be fixed for 24 h, followed by dehydration of the tissues in ethanol solutions of increasing concentrations from 70% ethanol to 100% ethanol, until pure water-free alcohol reached. This was followed by tissue clearing by immersing in xylene+ethanol for 30 minutes, followed by immersion in pure xylene solution for 30 minutes. The thermostat was set to 560 °C, and filled with melted paraffin wax, and the tissue was immersed in it for 1 hour, followed by immersing into hard wax embedding cup for another hour for embedding the tissue. Paraffin blocks were trimmed to 5 um slice thickness and placed in water bath for 40-45 minutes. This was then followed by tissue mounting on a glass slide, dewaxing by placing the tissue in xylene for 5 minutes and rehydration of tissue by placing the slides in a decreasing ethanol concentration from 100% ethanol to 7-% ethanol for 3 minutes each. Finally, the tissues were stained with hematoxylin for 10 minutes at 300 C and washed in distilled water for 15 minutes followed again by dehydration in ethanol and xylene solution and then, staining for 0.5% eosin for 2 minutes followed by a quick water wash for 1 minute. This was followed by wiping off the extra xylene with a paper towel and sealing of the dry glass slide with a cover slip.

Additionally, blood samples were collected in the serum separator tubes by retro-orbital bleeding and were processed into serum by centrifuging them at 10,000 g for 10 min at 40 C and removal of supernatant serum samples to fresh protease/nuclease free tubes. Serum samples were run into a Beckman Coulter/Olympus AU480 clinical analyzer (Olympus, AU480) to measure some liver injury enzymes like serum ALT, AST and liver function biomarker like total bilirubin as per the manufacturer's instructions.

Serum liver injury circulating biomarkers like ALT and AST were overall, significantly elevated at all the time points especially at the earlier 6 h and 24 h time points, where there was a massive 10-100-fold increase in the transaminases possibly from the dying/injured hepatocytes. Along with this, there was also a significant increase in the total bilirubin at all the time points post APAP injection suggesting a reduction in the hepatocyte functional ability to clear out the bilirubin due to hepatocyte death/change in hepatocyte phenotype. This further validated that this is an acute injury model where in an earlier intervention to mitigate acute inflammation might be important (FIG. 8B-D).

From the H&E stain of the liver left lobe, it was observed that right at the 6 h time point, there was an ALI established with wide-spread acute central coagulative necrosis around the central vein (FIG. 8E). At the 24 h time point, an infiltration of recruited immune cells could be visualized, seen populating the central vein (FIG. 8E). By 48 h, there were signs of beginning of wound healing. FIG. 8 shows a representative image of all the groups, which is depicting the central vein region of the liver surrounded by pericentral pool of hepatocytes. This confirmed an acute injury phenotype in the liver seen at 6 h, followed by a rapid infiltration of immune cells and beginning of a wound healing phenotype as seen at 48 h.

Immunohistochemistry (IHC) staining of macrophage-specific protein F4/80 reveals an increase in F4/80+ macrophages from 6 to 48 hours: In order to track F4/80-expressing mature macrophages in the liver upon APAP-induced injury, previously embedded paraffin-embedded liver tissue blocks were cut into more 5 um slices, rehydrated as previously mentioned, and stained with a mature macrophage marker F4/80. The tissues were stained with the F4/80 antibody (#MA1-91124 Thermofisher) (1:500). The F4/80 antigen has been identified to be selectively expressed by cells of murine mononuclear phagocytic lineage. Since it is highly expressed in mouse mature and tissue resident macrophages, it was utilized to track macrophage expression.

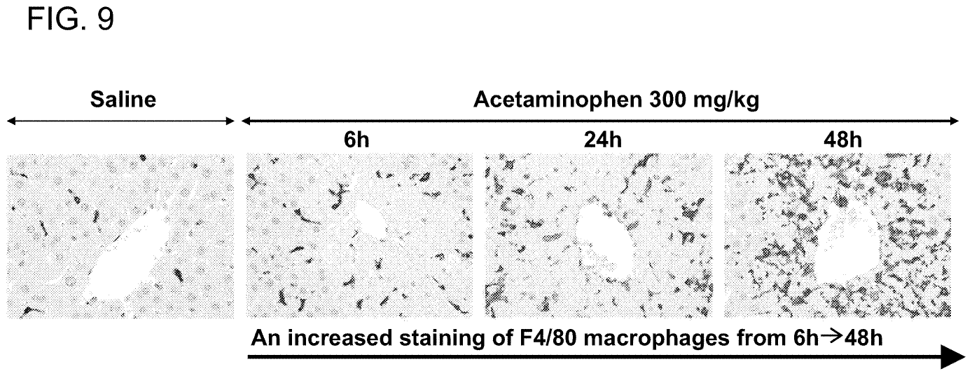

There was a progressive increase in the staining of mature macrophage population from 6 h to 48 h post APAP injection (FIG. 9). Also, the saline-treated group showed minimal number of stained F4/80+ cells showing the liver resident macrophages, Kupffer cells. Since it has been known that infiltrating myeloid cells are also important drivers of liver inflammation post AILI, an increase in F4/80 expressing cells from 6 h to 48 h suggested more and more infiltration of macrophages to the liver. Moreover, based on the IHC images, it was clear that the increase in expression was in the periportal region, in direct proximity to the portal vein. This raised the possibility of whether there was more infiltration of these myeloid cells extravasating from systemic circulation into the liver vasculature via portal circulation.

Staining for LPM specific nuclear GATA6 protein reveals infiltration of GATA6+ LPMs to the liver upon AILI: To confirm the infiltration of GATA6-expressing LPMs, the previously paraffin-embedded liver left lobes were stained with the GATA6 antibody (D61E4 XP® Rabbit mAb #5851) at a 1:50 dilution. GATA6 is an LPM-specific transcription factor that is not expressed by any other TRM or myeloid cell populations besides cavity-associated macrophages like LPMs. Since liver resident-Kupffer cells do not express it, it was used as a marker of LPM infiltration into the liver at 6 h, 24 h and 48 h post APAP-injury.

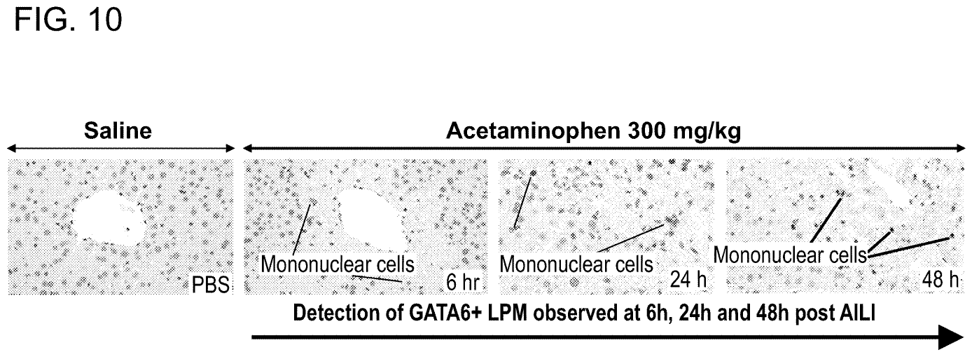

GATA6 IHC qualitatively confirmed the progressive increase in the GATA6+ positive cells from 6 h to 48. Saline treated group had no/minimal staining of nuclear GATA6 which progressively increased from 6 h APAP treatment to 24 h and 48 h treatment (FIG. 10). This finding validated the hypothesis of migration and infiltration of LPMs to the liver upon ALI, with an LPM infiltration seen in the liver as early as 6 h post APAP-injury.

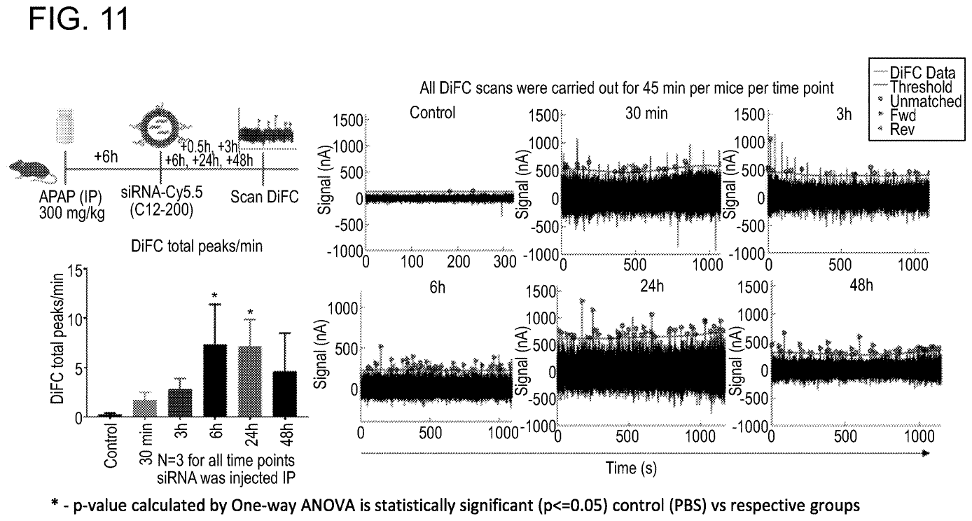

Systemically circulating rare labeled macrophages were detected by DiFC after inciting acute liver injury: Male Balb/c mice were injected intraperitoneally with 300 mg/kg of APAP. 6 h post APAP injection, 1 mg/kg of the siRNA-cy5 encapsulated in C12-200 LNP was intraperitoneally injected. Control mice were dosed with a dose-equivalent of 1×PBS. DiFC was performed on live mice and data was collected at 30 min, 3 h, 6 h, 24 h and 48 h post siRNA-cy5 administration to probe for circulating ‘peaks’ of cy5 labeled circulating cells. At every time point, mice were scanned for a total of 600 sec (10 min), which, based on prior DiFC studies has been known to be enough to scan the total volume of blood in mice. As shown in FIG. 11, detection of cy5-loaded macrophages by transient ‘peaks’ only upon APAP administration indicated the presence of these macrophages. The number of cy5-labeled macrophages in circulation appeared to peak between 6 h and 24 h. This suggested that macrophage migration from the peritoneal cavity into the liver upon liver injury might be via systemic circulation. These peaks were not observed in non-APAP-treated control mice, indicating that the circulating cy5-loaded macrophages are dependent on APAP-injury. Also, since there is an increase in number of circulating peaks from 3 h to 6 h, it is more likely that the LPM migration from the peritoneal cavity into the liver is more acutely driven with a peak migration seen from 6 h-48 h post APAP injury.

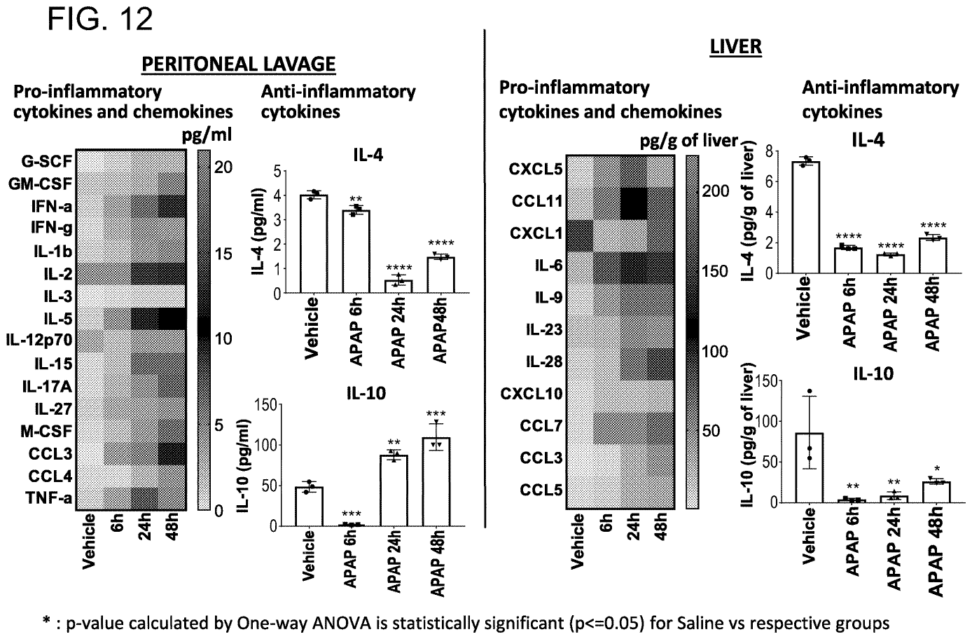

Circulating and secreted pro- and anti-inflammatory cytokine profile in peritoneal lavage and liver post APAP injection reveals a robust pro-inflammatory response to AILI: Frozen liver was ground into liver powder using a SPEX SamplePrep 2010 Geno/Grinder Tissue Homogenizer and total liver RNA was isolated using the Qiagen® 96w tissue extraction kit (74181). Following RNA extraction, total RNA was reversed transcribed into cDNA and subsequently qPCR was carried out to measure gene expression of some classic pro-inflammatory and anti-inflammatory macrophage markers. In order to isolate peritoneal lavage fluid, the harvested peritoneal lavage was spun at 300 g for 8 min and the cell supernatant was collected. The remaining peritoneal cells were subjected to magnetic separation of F4/80 macrophages using the Miltenyi Biotec peritoneal macrophage isolation kit as per the manufacturer's instructions. RNA extraction, cDNA reverse transcription and qPCR was carried out in enriched F4/80+ peritoneal macrophages to quantify gene expression of some pro- and anti-inflammatory macrophage markers. When the same macrophage marker expression was measured from the livers, a very similar trend again of a significant increase in the pro-inflammatory macrophage markers, and a reduction in the anti-inflammatory markers was observed (FIG. 12).

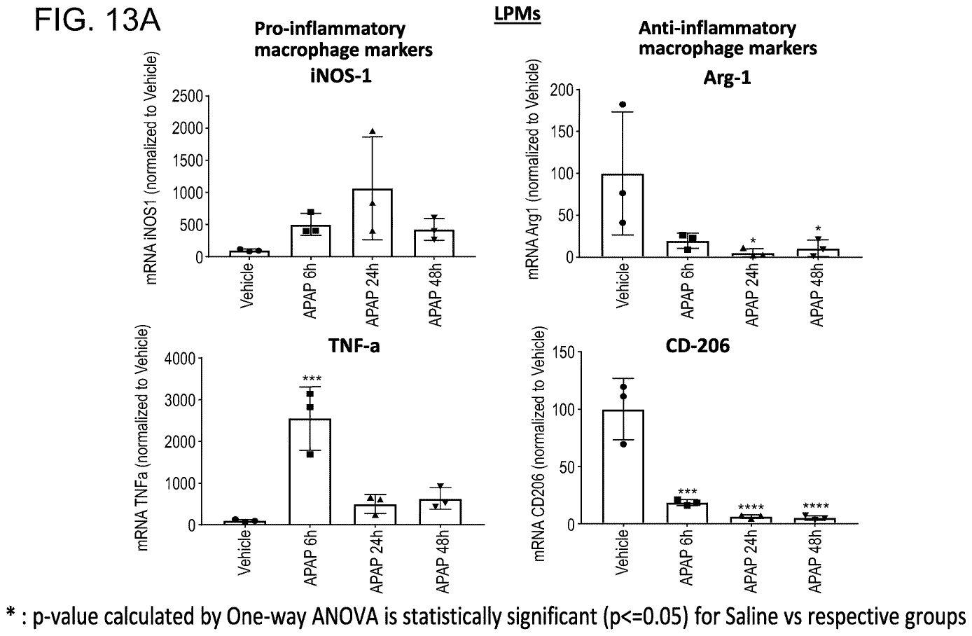

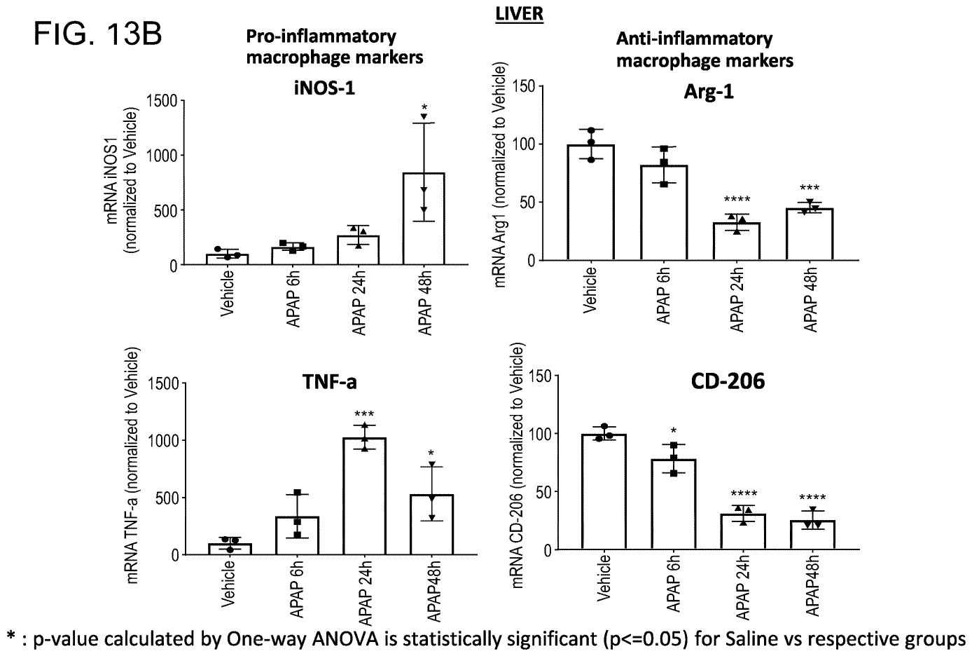

The Luminex Cytokine & Chemokine 36-Plex Mouse ProcartaPlex™ (EPX360-26092-901) kit was used to quantify the levels of 36 well characterized secreted cytokines and chemokines from both the liver lysate that was prepared as per the kit manufacturer's instructions, and the peritoneal lavage supernatant. Since the study's goal was to harness this migratory ability of LPMs to modulate their polarization state by RNAi-mediated gene silencing of some putative pro-inflammatory targets in LPMs, it was imperative to phenotypically characterize the polarization state of the isolated LPMs. Based on the results from the Luminex assay in the peritoneal lavage supernatant, it was found out that there was an increase in nearly all the pro-inflammatory cytokines (FIGS. 13A and 13B). Generally, all the cytokines were upregulated by 6 h, many of which were further upregulated by 24 h and 48 h. mRNA quantification data demonstrated a significant upregulation of some canonical pro-inflammatory macrophage markers like iNOS-1, TNF-a, IFN-g, IL-1b and CD-80. Additionally, similar to the observations in the secreted cytokines, within the LPMs as well, there was an initial significant reduction in some anti-inflammatory macrophage markers like Arg-1, CD-206 and CD-163 followed by beginning of recovery in expression by 48 h. This again confirmed that upon inciting injury to the liver, the macrophage polarization state in the peritoneal cavity changes to a more pro-inflammatory phenotype. Also, when the same macrophage marker expression was measured from the livers, a very similar trend was seen again of a significant increase in the pro-inflammatory macrophage markers, and a reduction in the anti-inflammatory markers. Further, there was a significant reduction seen in some of the putative anti-inflammatory cytokines like IL-4, IL-10, and IL-13 right at the 6 h time point decrease which was further reduced by 24 h. The same panel of cytokines was measured in the liver, and the same trend was observed of a significant increase in the pro-inflammatory cytokines from Vehicle to 6 h APAP treatment, and a further increase from 6 h to 48 h (FIG. 13). This was further validation of an acute inflammatory phenotype in the liver that was ensued due to hepatotoxicity and hepatocyte necrosis induced by APAP. The overall cytokine and peritoneal macrophage polarization data confirms the huge upregulation in the overall acute inflammatory phenotype in the liver as well as peritoneal cavity upon inciting injury by APAP. Since it was demonstrated that an siRNA modality can be efficiently delivered to LPMs using the C12-200-based LNPs, fully characterizing the model opens a great therapeutic opportunity to target the LPMs by an siRNA, and subsequently harness the LPM migration to the liver to carry these siRNA-encapsulated LNPs to the injured liver in order to mitigate the acute inflammation that ensues after APAP administration.

### Example 4. Robust In Vitro Silencing of HMGB1, NFKB1 and TLR4 in Large Peritoneal Macrophages was Observed with Lipid Nanoparticles Encapsulating Modified siRNAs

Synthesis and cy5 labeling of siRNAs were carried out as mentioned previously in Example 1, and the Accession numbers NM_008689.3, NM_021297.3, NM_001313894.1 were utilized as the mRNA transcript sequences for NF-κB, TLR-4 and HMGB1 respectively to design siRNAs against the targets. Double stranded 21/23-mer siRNAs were synthesized against these targets along with a control siRNA synthesized against gaussian luciferase (g-luc), since mouse genome does not express g-luc. Finally, a cy5 fluorophore was labeled on the 5-end of the sense strand before formulating the siRNAs into the C12-200 LNP.

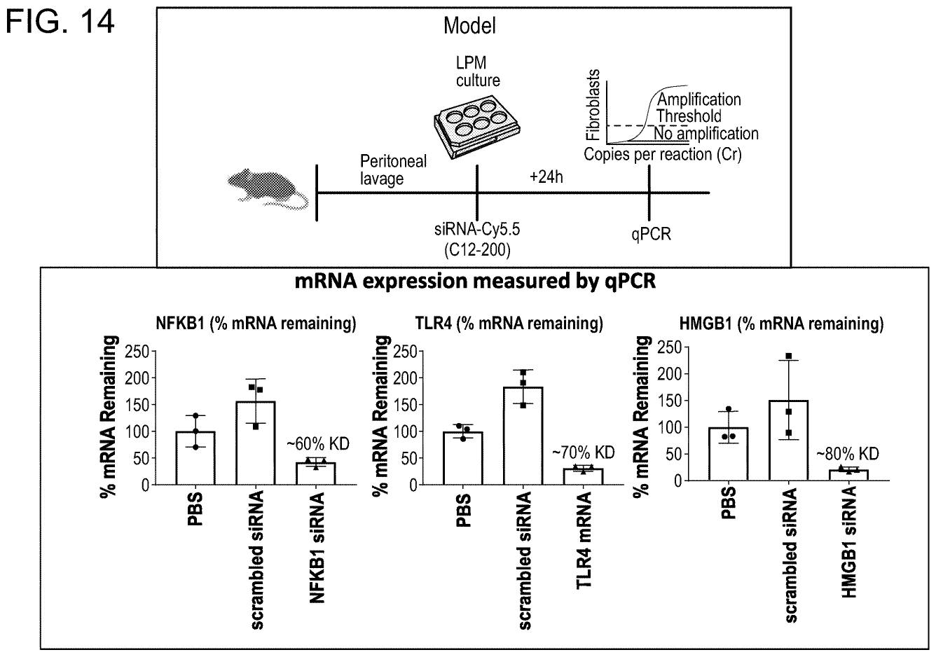

Formulation of all the siRNAs into C12-200 LNPs was carried out by the same steps as mentioned previously. In order to validate the siRNA sequences to assess target mRNA knockdown (KD) in vitro, knock-down of NF-κB, TLR-4 and HMGB1 KD was assessed in isolated primary mouse peritoneal macrophages. Female Balb/c mice were sacrificed, and peritoneal lavage was harvested. Isolated peritoneal lavage was plated and cultured in a 6-well plate at a density of 2×10⁶ cells/well in mouse peritoneal macrophage media. These cells were incubated in a 37 °C, 5% CO2 cell culture incubator overnight. The following morning, cells were transfected with the respective siRNA-cy5-C12-200 formulations for 24 h at a dose of 100 nM of siRNA per well and the cells were then harvested for RNA extraction and qPCR analysis of gene expression. RNA from the cells was isolated using the Qiagen® 96w tissue extraction kit (74181). Following RNA extraction, total RNA was reversed transcribed into cDNA and subsequently qPCR was carried out to measure gene expression of NF-κB, TLR-4 and HMGB1. For the data analysis, all the qPCR data was first normalized to a geo-mean of 2 housekeeping genes GAPDH and PPIB before being represented at mRNA expression relative to the respective Vehicle groups (FIG. 14). Based on gene expression analysis, there was a robust silencing of targets genes achieved, namely ~60%, ~70% and ~80% mRNA knockdown of NF-κB, TLR-4 and HMGB1 respectively relative to the PBS-treated group. The g-Luc control siRNA did not lead to any knock-down of any target mRNA.

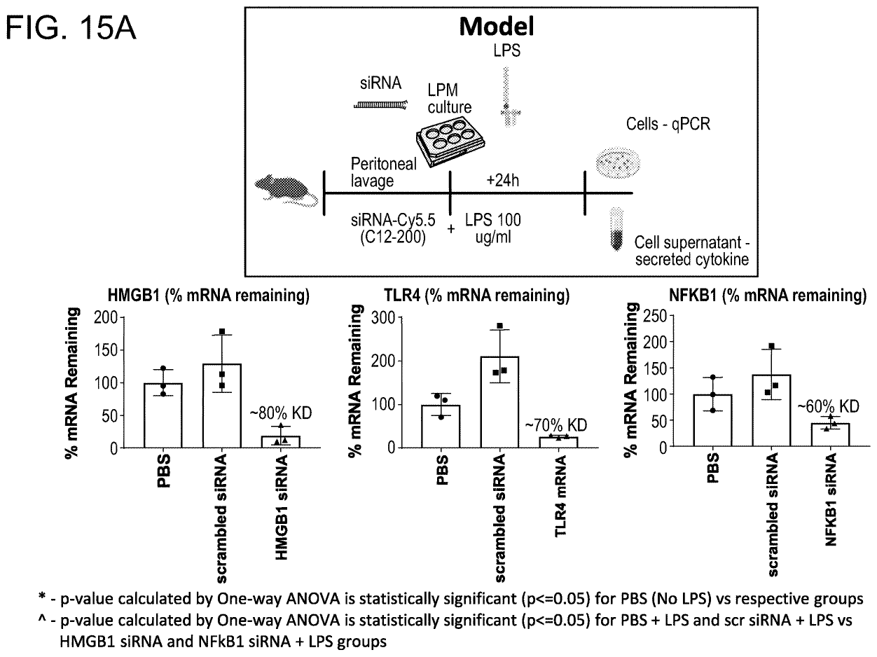

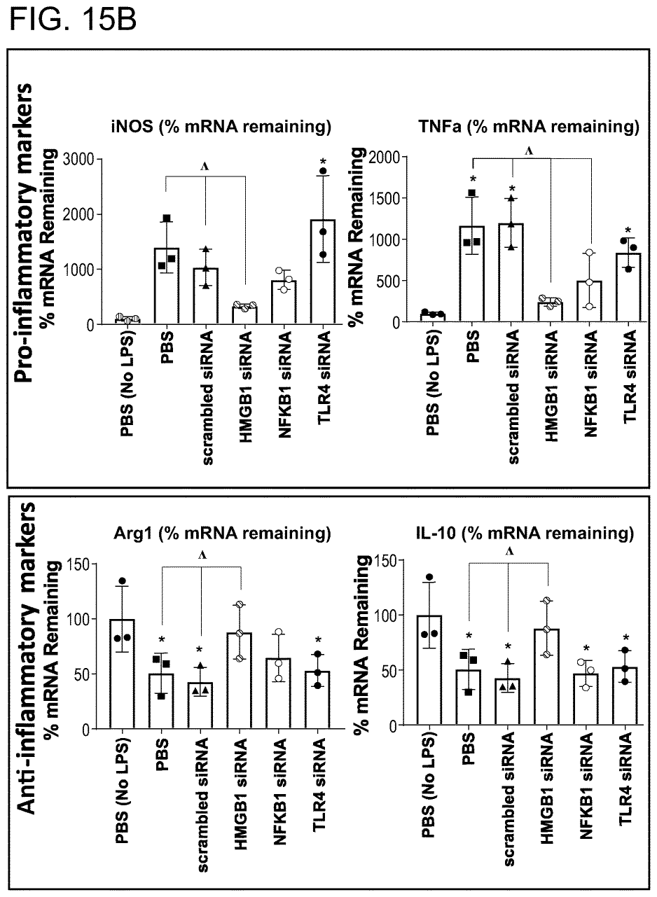

Further gene expression analysis of macrophage pro- and anti-inflammatory markers was performed in LPMs in an LPS-model of macrophage activation. Female Balb/c mice were sacrificed, and peritoneal lavage was harvested. Isolated peritoneal lavage was plated and cultured in a 6-well plate at a density of 2×10⁶ cells/well in mouse peritoneal macrophage media. These cells were incubated in a 37 °C, 5% CO2 cell culture incubator overnight. The following morning, cells were transfected with the respective siRNA-cy5-C12-200 formulations for 24 h at a dose of 100 nM of siRNA per well along with co-treating the cells with 100 μg/ml of LPS dissolved in 1×PBS. Cells were then harvested for RNA extraction and qPCR analysis of gene expression. RNA from the cells was isolated using the Qiagen® 96w tissue extraction kit (74181). Following RNA extraction, total RNA was reversed transcribed into cDNA and subsequently qPCR was initially carried out to confirm mRNA silencing of NF-κB, TLR-4 and HMGB1 (FIG. 15A). Along with that, gene expression was measured of a couple of well-known macrophage pro-inflammatory markers iNOS1 and TNF-α as well anti-inflammatory macrophage markers like Arg1 and IL-10. There was again a 60%, 70% and 80% mRNA KD seen in NF-κB, TLR-4 and HMGB1 mRNA expression relative to the PBS-treated group. Results demonstrated that HMGB1 silencing led to the most profound anti-inflammatory properties to the macrophages. Relative to the control siRNA and PBS treated groups, HMGB1 silencing led to significant reduction in pro-inflammatory markers iNOS-1 and TNF-α, as well as a significant increase in Arg1 and IL-10 anti-inflammatory markers (FIG. 15B). There was also some mitigation in inflammation upon NF-κB silencing, however, HMGB1 silencing led to the most significant protection from inflammation. Along with this, the siRNAs against HMGB1 also led to a higher KD of the target gene at ~80% KD compared to NF-κB and TLR-4 (FIG. 15A). Overall, this showed that silencing a DAMP like HMGB1 in LPMs might render a strong protection to the acute inflammation and downstream propagation to injury caused by APAP.

### Example 5. RNAi-Mediated Silencing of HMGB1 to Modulate LPM Polarization and Harnessing this as a Therapeutic Modality to Mitigate AILI

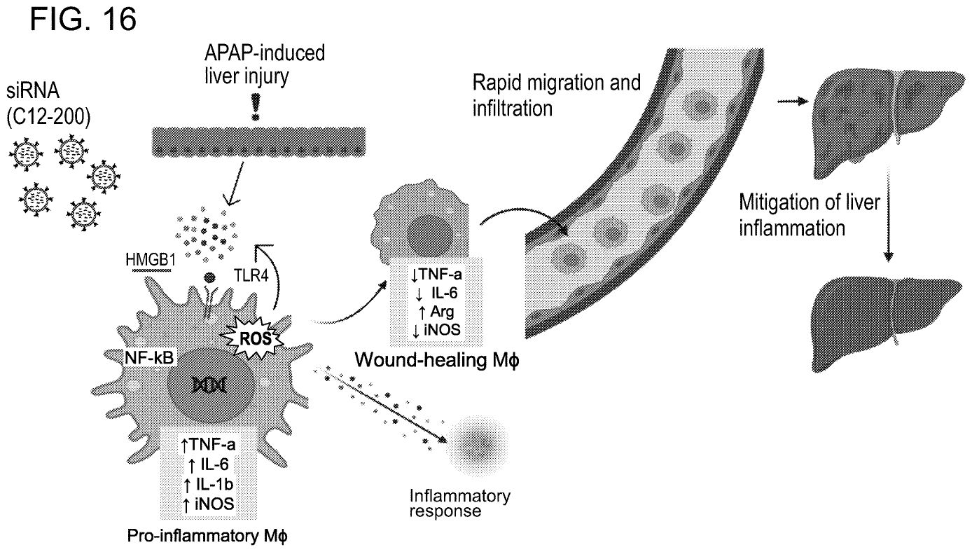

Macrophage polarization by siRNA-mediated silencing of HMGB1 in LPM are modulated and the migratory ability of LPMs to the liver is utilized to mitigate liver inflammation in a model of AILI (FIG. 16). PD properties of HMGB1 siRNA that is administered intravenously (IV) versus intraperitoneally (IP) is evaluated, and HMGB1 mRNA KD in LPM and liver is assessed in vivo. With the goal of silencing HMGB1 within the LPM cells and eventually the liver and harnessing the migratory ability of these LPMs to the liver in an APAP-induced hepatoxicity model, the study aimed to modulate macrophage polarization state to a more M2-like anti-inflammatory phenotype and use these LPMs to carry the siRNA into the liver to mitigate the massive inflammation downstream to the hepatocyte necrosis. C12-200 encapsulated siRNA targeting HMGB1 were used as described previously in the aforementioned Example(s). Initially, to confirm both the delivery and in vivo RNAi functionality of the C12-200 encapsulated HMGB1-siRNA in the LPMs and liver and secondly, the study carried out a target and hypothesis validation study in a mouse model of APAP-induced liver toxicity. Finally, a GalNAc-conjugated siRNA with the same sequence as the C12-200 encapsulated siRNA was utilized in order to assess benefit of silencing HMGB1 within the liver hepatocyte and compare both the therapeutic strategies of silencing a intracellular as well as secreted DAMP like HMGB1 in LPMs and myeloid cells versus the hepatocytes.

Confirmation of delivery of LPMs as carriers of C12-200 loaded siRNA-cy5 to the injured liver upon AILI: As described above, in vitro characterization revealed that HMGB1 silencing by siRNA led to the most robust protection of isolated mouse LPMs to LPS-induced injury and macrophage activation. The study aimed to initially validate the migration of LPMs carrying the C12-200 encapsulated HMGB1 siRNA to the liver upon inducing APAP injury to the liver, and furthermore, also confirm in vivo silencing of HMGB1 mRNA in the LPMs as well as in the liver. One of the main goals was to modulate LPM polarization towards a more wound-healing phenotype by muting the macrophage DAMP response upon silencing HMGB1, and furthermore use these cells as delivery modalities to also deliver these LNP-encapsulated siRNA to the liver and silence HMGB1 also within the liver. Since the hypothesis was based upon the inherent migratory property of LPMs to the liver, the study validated this migratory ability, and with it, confirmed whether these LPMs also bring in the C12-200 encapsulated siRNA to the liver. Flow cytometry was used to characterize macrophage populations within the liver non-parenchymal cells (NPCs), and immunofluorescence (IF) was used to look at GATA6 and cy5 expression in the NPCs.

Male Balb/c mice were administered vehicle (saline) or APAP at 300 mg/kg after being fasted for 12 h to deplete the glutathione levels along with administration of 1 mg/kg of HMGB1 targeting siRNA that was labeled with a cy5 fluorophore and encapsulated in a C12-200 LNP. At 6 h, 24 h and 48 h post siRNA and APAP administration, mice were sacrificed by CO2 asphyxiation, livers were perfused via the portal vein, and liver NPCs were isolated using the mouse liver dissociation kit from Miltenyi Biotec—Catalog number 130-105-807 following the kit manufacturers' protocol. Isolated liver NPCs were made into single cell suspensions and stained for antibodies against various cell markers: F4/80 and CD11b—mature macrophages, CD45—all lymphoid cells, CD31—endothelial cells, and CD38—other immune cells and hepatic stellate cells. Flow cytometry analysis revealed a significant increase in the number of F4/80+CD11b+ mature tissue macrophages for the population gated within the CD45+ lymphoid cell population post APAP induced injury. There was a progressive increase seen in this population from 6 h→24 h→48 h. This demonstrated the progressive infiltration and/or proliferation of liver mature macrophage population after AILI. Moreover, importantly, a time-dependent increase was seen in cy5+ cell population gated within these F4/80+CD11b+ macrophages reveling an increased infiltration of HMGB1 siRNA-cy5-loaded macrophages as well as an increase uptake of these LNP-encaulated siRNA within the liver macrophages post APAP. It was important to note here that even at 6 h post APAP injury, more than 60% of the F4/80+CD11b+ macrophages had already taken up the siRNA-cy5 which increased to >80% of macrophages taking up the siRNA by 48 h.

Since an increase in cy5-loaded mature macrophages was confirmed earlier by flow cytometry, the study further checked whether these infiltrating macrophages were LPMs. IF staining of LPM-specific marker GATA6 was used to look at liver-infiltrating LPMs. GATA6 is a zinc-finger transcription factor that is only expressed by LPMs, and the liver resident macrophages Kupffer cells, as well as any other infiltrating, and liver resident immune cells do not express it. IF imaging revealed the presence of GATA6-expressing LPMs in the liver NPCs at all the time points (6 h, 24 h and 48 h) after AILI. An overlay of F4/80 and GATA6 expressing cells with cy5, revealing that these LPMs were also carriers of the C12-200 encapsulated siRNA-cy5. Additionally, image quantification revealed a significant increase in the percentage of cy5+ cells within the liver NPCs with more than 30% of the macrophages carrying siRNA-cy5 as early as 6 h post AILI. These findings were encouraging in supporting our hypotheses of one, LPM migration to the liver upon acute injury, and secondly that these LPMs can also carry an LNP-loaded siRNA modality into the liver.

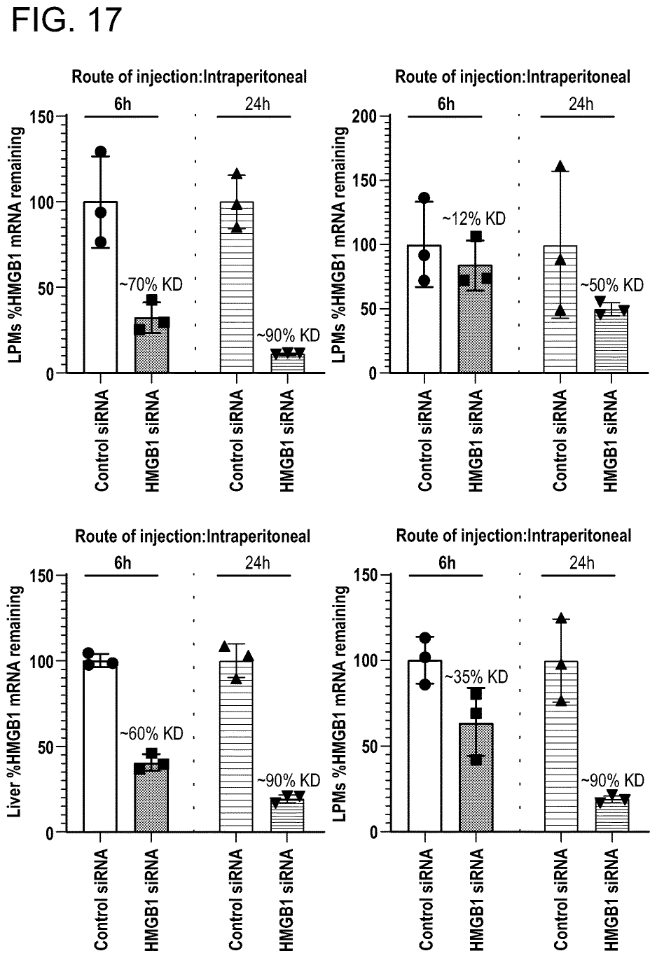

Assessment of pharmacodynamic properties of C12-200 encapsulated HMGB1 siRNA administered intravenously versus intraperitoneally on HMGB1 target mRNA silencing in LPM and liver in vivo: HMGB1 is a ubiquitously expressed protein, and is not specific to LPMs. In order to assess LPM specific KD of HMGB1 versus liver KD, two routes of administration were compared of the C12-200 encapsulated siRNA against HMGB1—intraperitoneal (IP) administration and intravenous (IV) administration of these LNP encapsulated siRNA. Male Balb/c mice were administered 1 mg/kg of C12-200 encapsulated siRNA-cy5 targeting HMGB1 mRNA and g-Luc (Gaussia luciferase control siRNA), and peritoneal lavage was harvested from mice at 6 h and 24 h post siRNA administration in order to assess HMGB1 KD. From the isolated peritoneal lavage, LPMs were magnetically enriched using the peritoneal macrophage isolation kit from Miltenyi Biotec, (Catalog 130-110-434) as previously described. RNA was isolated and reverse transcribed into cDNA, and qPCR analysis was carried out to assess HMGB1 KD. The probe used for measuring HMGB1 expression was ordered from Thermofisher (Catalog number: Mm00849805_gH). For the data analysis, all the qPCR data was first normalized to a geo-mean of 2 housekeeping genes GAPDH and PPIB before being represented at mRNA expression relative to the control siRNA group. Based on the gene expression analysis, there was significantly more KD of HMGB1 mRNA with the intraperitoneally administered C12-200 encapsulated siRNA (FIG. 17). When dosed intraperitoneally, there was already around 70% HMGB1 mRNA silencing seen at 6 h post siRNA administration, which was further increased to almost a 90% reduction in HMGB1 mRNA by 24 h in LPMs (FIG. 17). Compared to IP, IV administered siRNA led to a much lesser KD of the target mRNA as it barely silenced HMGB1 at 6 h, with about 50% mRNA silenced at 24 h (FIG. 17).

Evaluation of HMGB1 silencing in LPMs by C12-200 encapsulated HMGB1 siRNA on 30 mitigation of liver inflammation and ALI in a mouse model of AILI: After confirming the uptake as well as in vivo functionality of C12-200 encapsulated siRNAs targeting HMGB1 in LPMs, the study aimed to validate the KD of both the target, as well as a hypothesis of using LPM migration to the liver upon injury in a mouse APAP induced liver injury. For this purpose, 6-8-week-old male Balb/c mice were fasted for 12 hours to deplete and normalize the glutathione levels before APAP administration, and APAP was subsequently injected intraperitoneally the following morning at a sub-lethal dose of 300 mg/kg. To see whether silencing HMGB1 siRNA would lead to a benefit in the inflammatory phenotype and overall liver injury, the C12-200 encapsulated siRNA targeting HMGB1 was dosed in parallel with the APAP injection at 1 mg/kg of the siRNA concentration. Control siRNA targeting g-Luc was dosed for every HMGB1 targeting siRNA group at the same 1 mg/kg siRNA concentration, and a non-APAP saline treated group at the APAP-adjusted volume was also used. Along with this, in order to have a positive control for the study, the mice were dosed with a clinically validated antidote for APAP toxicity N-acetyl cysteine (NAC) by oral gavage at a dose of 150 mg/kg. After administration of the respective treatments along with APAP, mice were sacrificed at 6 h, 24 h and 48 h post administration of APAP and treatment. Livers for harvested from the mice, and left lateral lobes were separated and collected in formalin to be further processed into paraffin embedding for histopathological evaluation, and the remaining section was snap-frozen in liquid nitrogen, and then stored at −800 C for molecular analysis. Along with the livers, peritoneal lavage and blood were also collected. Blood was immediately processed into serum, which was then stored at −800 C until used for measuring circulating liver injury biomarkers. Peritoneal lavage was immediately processed to enrich LPMs following the Miltenyi Biotec peritoneal macrophages isolation protocol.

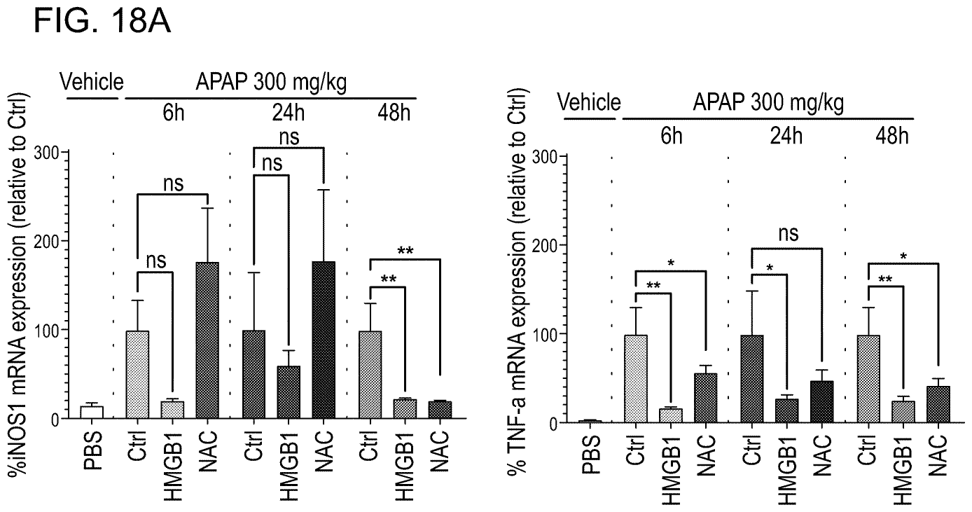

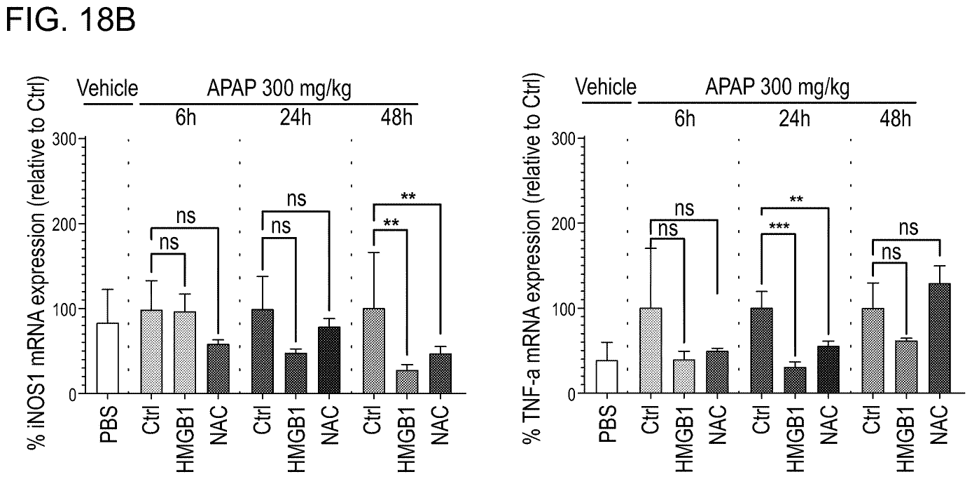

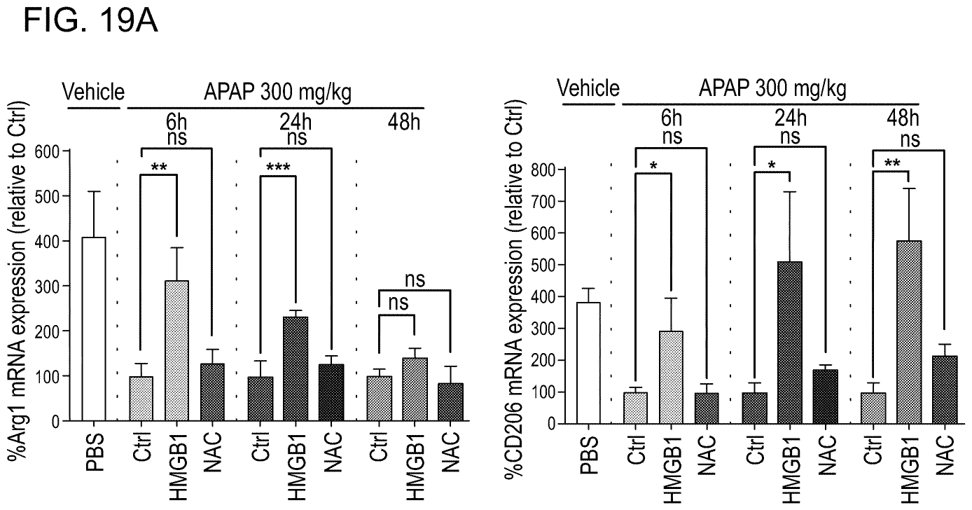

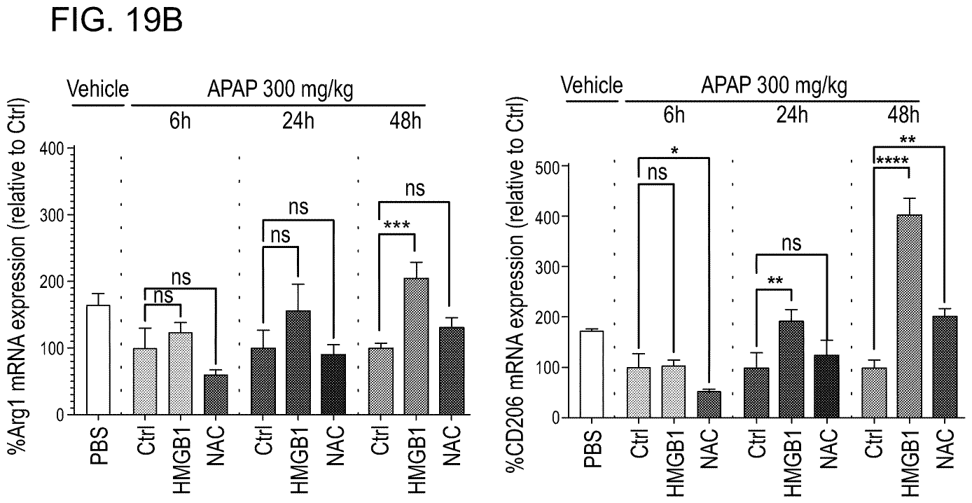

In order to first confirm the silencing of target HMGB1 mRNA in the LPMs as well as liver, RNA was extracted from the LPMs and frozen liver. qPCR analysis revealed significant silencing of HMGB1 in LPMs at all the time points (6 h, 24 h and 48 h). There was about 70%, 90% and 90% mRNA silencing observed at 6 h, 24 h and 48 h respectively. Further, to see whether HMGB1 knock-down leads to a modulation in macrophage phenotype to a more ‘M2-like’ macrophage with wound healing properties. In order to assess the state of macrophage polarization of the LPMs as well as within the liver, the study aimed to quantify expression of some of the pro-inflammatory and anti-inflammatory macrophage markers. Hence, from the RNA that was earlier isolated from the frozen livers and LPM samples, mRNA expression levels of a couple of classic ‘M1-like’ macrophage markers iNOS1 and TNF-α as well as a couple of putative ‘M2-like’ macrophage markers Arg1 and CD206 were also measured by qPCR. qPCR analysis revealed a significant reduction in the pro-inflammatory markers iNOS1 and TNF-αat all the 3 time points 6 h, 24 h and 48 h in the LPMs as well as within the liver (FIG. 18). Moreover, there was also an increase in the anti-inflammatory macrophage marker Arg1 and CD206, that was more prominently seen by 48 h (FIG. 19). This clearly demonstrated that upon silencing HMGB1, the macrophage polarization state was modulated to a more M2-like macrophage. Along with this, since it was previously demonstrated that a significant percentage of LPMs carrying the siRNA already migrate into the liver by 6 h, it was believed that this partly impacts the macrophage polarization state in the liver and nudges it to a more wound-healing anti-inflammatory phenotype, thus rendering an early protection from injury.

Utilizing a hepatocyte-targeting GalNAc conjugated siRNA against HMGB1 as control in an APAP-induced liver injury model: To further attribute findings of mitigation of inflammation elicited by AILI as well as a meaningful improvement in liver injury phenotype to silencing of HMGB1 in LPMs and the myeloid cellular component and not because of a hepatocyte-specific silencing of HMGB1, the same siRNA sequence against HMGB1 was conjugated to an N-acetyl galactosamine (GalNAc) ligand, which has been known to selectively be taken up by hepatocytes. By silencing hepatocyte specific HMGB1 and testing this in a mouse model of AILI, the study aimed to tease out the differences in phenotype improvement by silencing HMGB1 in hepatocyte versus LPMs r. For this purpose, male Balb/c mice were pre-treated a GalNAc-HMGB1 siRNA conjugate at a 3 mg/kg dose along with PBS dosed at an equivalent volume for 14 days by subcutaneous injection to silence the hepatocyte HMGB1 and then treated with a 300 mg/kg dose of APAP given intraperitoneally after fasting mice for 12 h. At 6 h, 24 h and 48 post siRNA and PBS administration, mice were euthanized, and livers were collected, left lateral lobe being fixed in formalin for 24 h, whereas the remaining section being snap frozen in liquid nitrogen and then stored at −800 C. Blood was also collected at each time point, and it was further processed into serum which was then analyzed in a clinical analyzer to measure liver injury circulating biomarkers.

Gene expression analysis revealed that HMGB1 mRNA silencing was robust at all the time points. There was about 90% mRNA silencing seen at 6 h, 24 h and 48 h in the whole liver tissue. For histopathological evaluation, the liver left lobes were allowed to fix in formalin for 24 h, and paraffin-embedded tissue blocks were made. Liver blocks were cut into 5 um slices and stained with hematoxylin and Eosin (H&E) for looking at the liver injury phenotype. H&E staining revealed a wide-spread hepatocellular degeneration/necrosis and all the three time points, with evidence of wound healing showing up at the 48 h time point. Importantly, unlike the robust protection that previously seen when HMGB1 siRNA was encapsulated in a C12-200 LNP and silenced in LPMs, hepatocyte HMGB1 silencing by a GalNAc-conjugated siRNA did not lead to any significant benefit in the liver injury phenotype. There were also other liver inflammation findings like neutrophil infiltrates and hepatocyte atrophy, which was consistent with APAP induced liver toxicity.

Serum samples were run into a Beckman Coulter/Olympus AU480 clinical analyzer (Olympus, AU480) to measure some liver injury enzymes like serum ALT, AST and liver function biomarker like TBil as per the manufacturer's instructions. Again, HMGB1 silencing in the liver hepatocytes using a GalNAc conjugated siRNA targeting HMGB1 did not lead to any meaningful benefit. There was a slight reduction seen in ALT, AST and TBil at 24 h post APAP injury, however, it was minimal and not significant. This further confirmed that the significant benefit could be mostly attributed to HMGB1 silencing in LPMs, and not to HMGB1 silencing in hepatocytes, which is an important observation validating the hypothesis of LPM modulation and migration to the liver upon injury, as well as being carriers of siRNA to the injured liver being an important contributing factor to the improvement in liver injury, and a significant protection from downstream inflammation induced by APAP induced liver injury.

### Example 6. RNAi-Mediated Silencing of HMGB1 in Liver Infiltrating GATA6+ Large Peritoneal Macrophages Prevents Acute Liver Injury

A study was designed to address whether tissue-resident macrophages (TRMs) like GATA6-expressing large peritoneal macrophages (GLPMs) migrate and infiltrate into an injured liver in an acute liver injury (ALI) model of AILI; and if so, whether they can intravasate from the peritoneal cavity into the systemic circulation. DiFC was employed as a means to probe for fluorophore labeled circulating cells in vivo in the vasculature. An n=4 biological replicates were used for all the mouse in vivo studies to address infiltration of GLPMs into the liver (Oza D et al., Theranostics. 2024; 14(6):2526-43; the entire contents of which are expressly incorporated herein by reference). All the GLPM liver migration assessment studies were independently replicated twice. All the in vitro studies done to assess pharmacodynamic (PD) and efficacy of targets were also independently replicated twice.

In the latter half of the study, an in vivo PD assessment study was performed to confirm silencing of target mRNA (HMGB1). Finally, proof-of-concept studies were performed in mice to assess the efficacy of a GLPM-selective C12-200 LNP-based delivery modality to silence pro-inflammatory gene HMGB1 using an siRNA and evaluate the therapeutic potential of such a modality and migratory property of GLPM to injured liver as a means to mitigate liver inflammation caused by acetaminophen (APAP). For these studies, the exact biological replicates per experiment have been described in individual figures. The sample size seemed ideal based on prior review of the model (APAP-induced hepatotoxicity), and the relatively tight and robust dataset confirmed the usage of the sample size as interpretable, and significantly meaningful (Yoon E et al., J Clin Transl Hepatol. 2016; 4(2):131-42; and Holt M P et al., J Leukoc Biol. 2008; 84(6):1410-21; the entire contents of each of which are expressly incorporated herein by reference).

All the data was expressed as Mean+/−SD. Statistical significance was determined by either unpaired student t-test (two tailed) whenever comparison between two groups was involved. In the case of 3 or more groups, one way-ANOVA (two tailed), followed by Tukey's post hoc test was used to determine statistical significance. All the experimental findings were reproduced with biological replicates of 4 unless specified otherwise, and individual experiments were reproduced twice to confirm consistency of results. A p value of <0.05 was considered as statistically significant. All the statistical analysis was carried out using GraphPad Prism v7.01 (GraphPad by Dotmatics, © 2022 GraphPad Software). For all the figures, *p<0.05, **p<0.01, ***p<0.001, **** p<0.0001, ns not significant.

GLPMs migrate to the liver in acute liver injury incited by APAP: In order to establish GLPM migration and infiltration into the injured liver in a physiologically relevant ALI mouse model, an APAP-induced hepatotoxicity model was used. GLPMs express a specific nuclear protein GATA6 which is neither expressed by liver resident Kupffer cells, nor by circulating monocytes and hence is the perfect GLPM-specific marker by which probing was intended for infiltrating GLPMs to the liver after AILI. Female BALB/c mice were intraperitoneally administered a 300 mg/kg dose of APAP for 6 hours, 24 hours and 48 hours. Significant upregulation of circulating liver injury enzymes as well as an increase in total bilirubin levels confirmed liver injury incited by APAP. Histopathological evaluation revealed wide-spread acute central coagulative necrosis by 6 hours. This confirmed an acute injury phenotype in the liver at 6 hours, followed by a rapid infiltration of immune cells and beginning of a wound healing phenotype as seen at 48 hours. Overall phenotype resonated well with AILI.

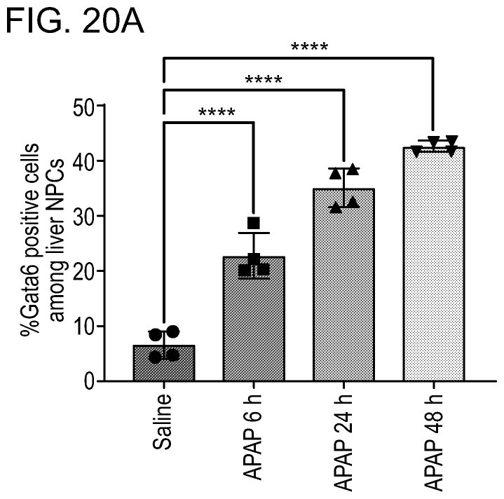

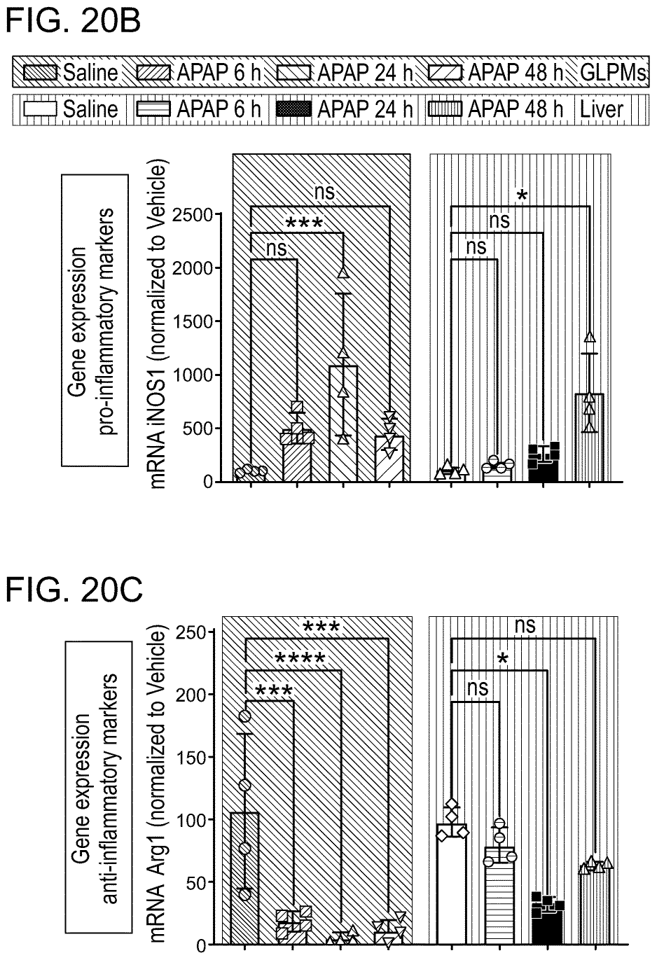

Immunofluorescence (IF) staining of GATA6 from isolated liver non-parenchymal cells (NPCs) was used to assess GLPM migration to injured liver. A clear co-staining of GATA6 with mouse macrophage marker F4/80 revealed a significant infiltration of GLPMs into the liver at all the time points measured (6 hours, 24 hours and 48 hours). Overall, there was a clear increase in GLPM infiltration to the liver that was driven by AILI (FIG. 20A). Additionally, immunohistochemical (IHC) staining of GATA6 protein also corroborated well with the IF data, confirming more GATA6 staining in the liver from 6 hours to 48 hours.

AILI drives GLPMs into a pro-inflammatory state: Gene expression of some well-known pro-inflammatory macrophage markers in the GLPMs enriched from the isolated peritoneal lavage as well as the liver NPCs was measured. There was a significant increase in the relative mRNA expression of classic macrophage pro-inflammatory marker like inducible nitric oxide synthase-1 (iNOS1) in not only the liver, but also in the GLPM population, with the most significant increase seen by 24 hours in the GLPMs, while in the liver, there was an increasing trend from 6 hours to 48 hours (FIG. 20B). Further, expression of the classic anti-inflammatory marker arginase-1 (Arg1) was significantly reduced in GLPMs (FIG. 20C).

Additionally, there was a significant increase in some secreted pro-inflammatory cytokines in GLPMs like tumor necrosis factor-alpha (TNF-α) and interferon-gamma (IFN-g), as well as a reduction in other anti-inflammatory cytokines like interleukin-4 (IL-4) (FIGS. 20D, 20E) that was measured in the peritoneal lavage supernatant. Further, a panel of putative circulating cytokines was measured where in pre-dominantly, a majority of them were upregulated by 6 hours, many of which were further increased by 24 hours and 48 hours post APAP administration. As expected, the majority of pro-inflammatory and pro-proliferative cytokines measured in the liver were significantly upregulated by 6 hours, with a further increase seen by 48 hours post APAP injection.

These results indicate that liver injury driven by APAP led to a significant change in the phenotypic state of GLPMs located in the surrounding abdominal cavity, and generally drove an inflammatory phenotype among them.

C12-200-based LNPs were utilized to deliver the Cy5-tagged-siRNA into GLPMs: Gaussia luciferase (gLuc) targeting siRNA was used initially as a fluorescent tag for all the GLPM tracking studies, and later on as a control siRNA. Since mouse genome does not contain gLuc, it served as the ideal control siRNA for the experiments. In order to fluorescently track the cells encapsulating the delivered siRNA in vivo, the sense strand of the gLuc siRNA was labelled to a Cy5 fluorophore. The C12-200 ionizable lipid was used as the delivery system to formulate the siRNA-Cy5 into an LNP system owing to its previous characterization on selectively delivering siRNAs into GLPMs (Oza D et al., Theranostics. 2024; 14(6):2526-43).

Systemically circulating siRNA-Cy5 (C12-200)-labeled GLPMs were detected by DiFC upon APAP-induced acute liver injury: Since a rapid infiltration of the peritoneally resident GLPMs into the liver upon AILI was observed, the possible route of this unique phenomenon was explored further. GLPMs intravasate and migrate to the lungs via systemic circulation upon depleting out the lung-resident AMs (Oza D et al., Theranostics. 2024; 14(6):2526-43). The earlier findings were expanded in context of an acute injury model within the peritoneally located liver tissue. These fluorophore-labeled GLPMs were tracked within the vasculature utilizing the same innovative live-cell tracking technique of DiFC that was previously used to track the migration of these cells to the lungs from the peritoneal cavity, as DiFC has been previously confirmed to accurately track siRNA-Cy5 (C12-200) labeled GLPMs (Oza D et al., Theranostics. 2024; 14(6):2526-43).

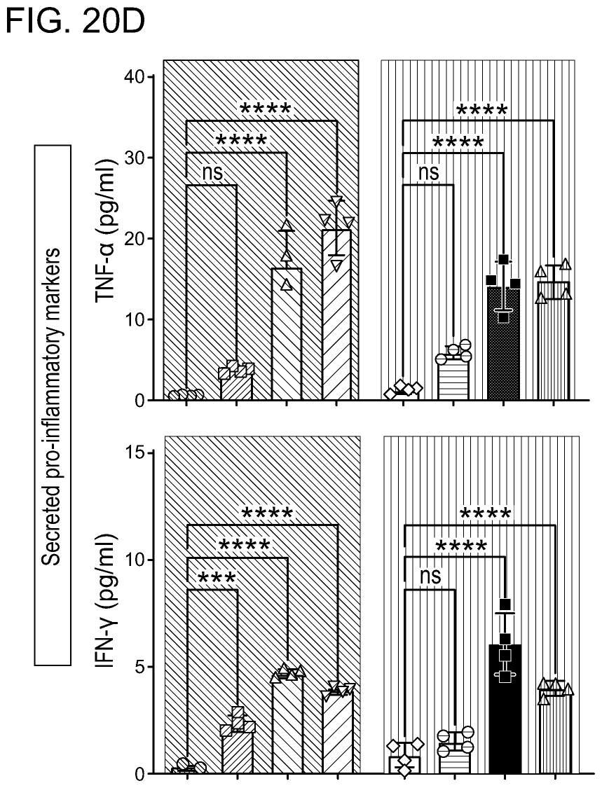

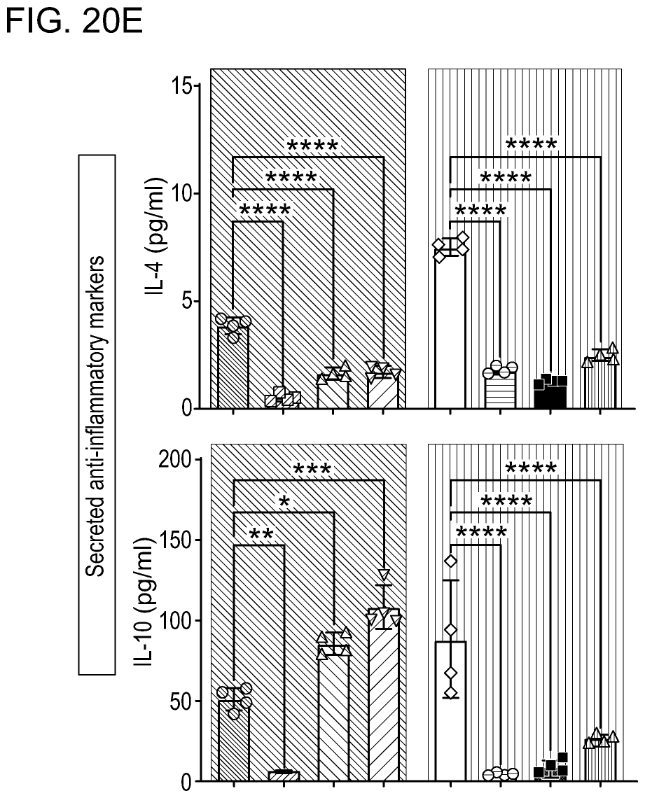

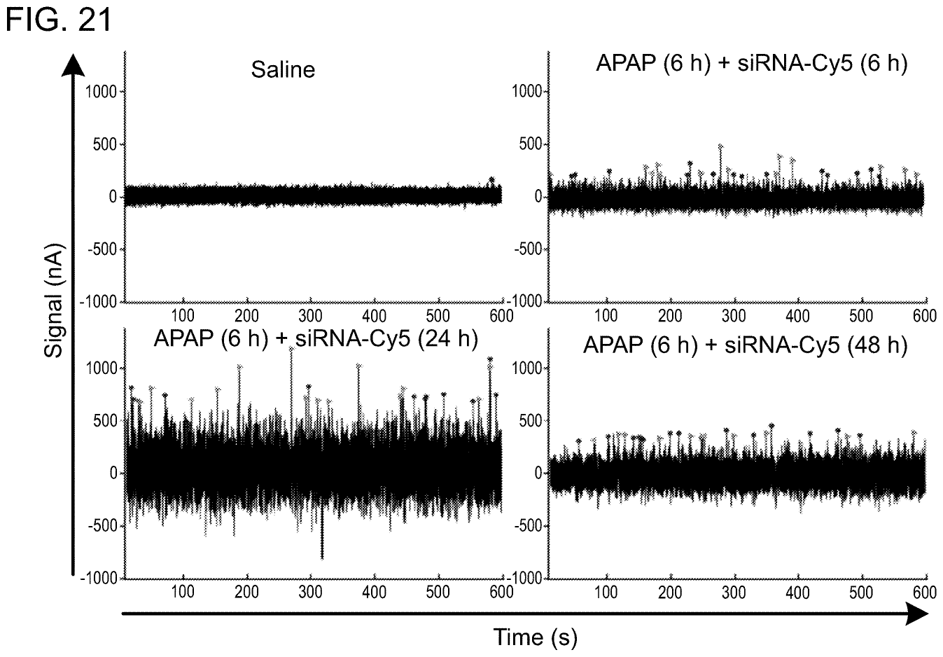

Female BALB/c mice were injected intraperitoneally with 300 mg/kg of APAP. Since a significant liver infiltration of GLPMs was seen at 6 hours, circulating siRNA-Cy5 (C12-200)-labeled GLPMs were probed at earlier time points. Hence, APAP was injected for 6 hours, followed by 1 mg/kg intraperitoneal administration of the siRNA-Cy5 (C12-200). Control mice were dosed with a weight-equivalent dose of 1×PBS. DiFC was performed on live mice and data was collected at 0.5 hour, 3 hours, 6 hours, 24 hours and 48 hours post siRNA-Cy5 (C12-200) administration to look for circulating ‘peaks’ of Cy5 labeled circulating cells. At every time point, mice were scanned for 45 minutes in quadruplicate, allowing for counting the labeled macrophages in systemic circulation in real time post APAP and siRNA-Cy5 (C12-200) injections. Detection of Cy5-labeled macrophages by transient ‘peaks’ only upon APAP administration indicated the presence of these macrophages in circulation (FIG. 21). The number of Cy5-labeled macrophages in circulation appeared to peak between 6 hours and 24 hours (FIG. 21). This provided an estimate on potential intravasation of fluorophore-labeled macrophages in real time, post APAP injury and showed a possible systemic route macrophage tropism into the liver upon APAP injury, especially since it was previously established that C12-200 encapsulated siRNA show a selective uptake into GLPMs at these earlier timepoints when dosed intraperitoneally (Oza D et al., Theranostics. 2024; 14(6):2526-43). DiFC data was indicative of maximum macrophage trafficking from the peritoneum into the vasculature at around 6 hours-48 hours post APAP administration which is also when more infiltration of GLPMs to the liver was observed (FIGS. 21, 20B).

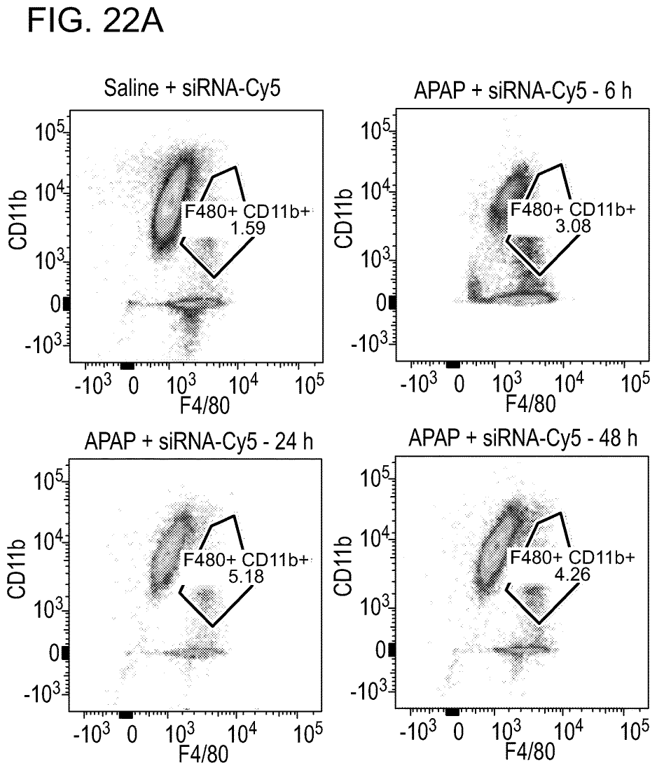

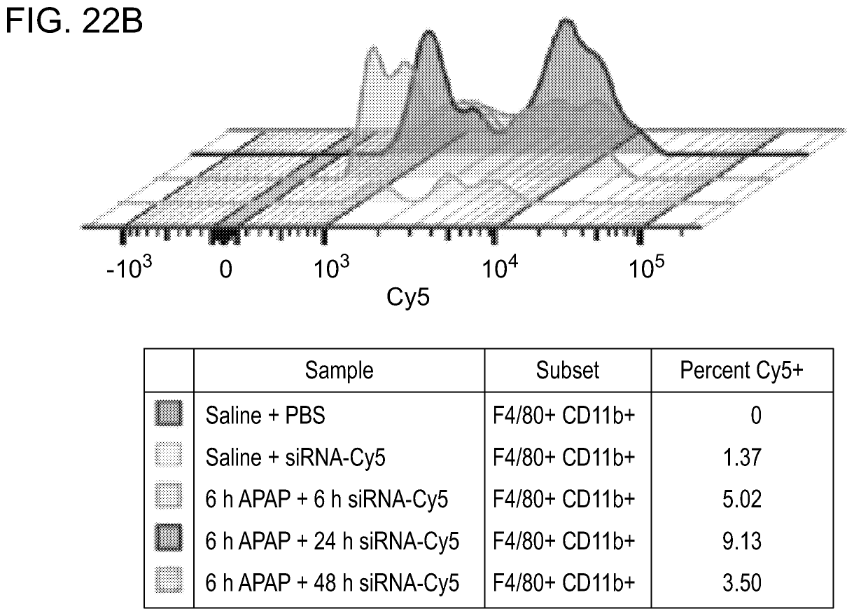

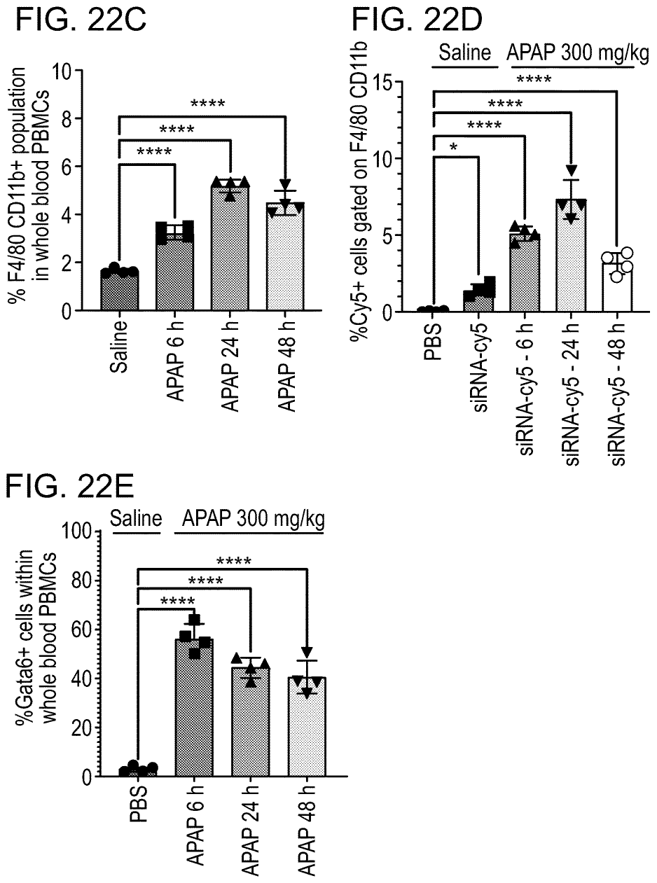

ALI by APAP-induced hepatotoxicity leads to intravasation of GLPMs into systemic circulation: Next, the DiFC findings were confirmed by specially probing for GATA6-expressing mature TRMs in whole blood following APAP administration. APAP was intraperitoneally injected again at 300 mg/kg dose along with a saline control, following which siRNA-Cy5 (C12-200) was administered, and blood was harvested at 6 hours, 24 hours and 48 hours post siRNA-Cy5 (C12-200) injection. Whole blood PBMCs were isolated after lysing the red blood cells (RBCs) from the blood and were only gated for F4/80+CD11b+ cells since those were the cell types of interest. Flow cytometry analysis revealed a significant increase in the F4/80+CD11b+ TRMs circulating in the APAP administered mice compared to saline-treated mice (FIG. 22A, 22C). Moreover, the percentage of Cy5+ cells gated within the F4/80+CD11b+ cell population significantly increased in the PBMCs from APAP-treated mice compared to saline controls (FIGS. 22B, 22D). The percentage of Cy5+ cells was the maximum at 6 hours and 24 hours, with a slight reduction by 48 hours (FIG. 22D). This corroborated well with the DiFC data where the maximum number of labeled macrophages seemed to be between 6 hours and 48 hours post AILI.

Additionally, IF staining of isolated PBMCs was carried out to look at GLPM-specific marker GATA6. Findings from IF revealed the detection of nuclear GATA6+ LPMs. Nuclear staining of GATA6+ also overlayed with DAPI stain confirming the presence of GLPMs in the blood only after AILI, since saline control only showed a DAPI stain without any evidence of GATA6 expressing cells, possibly simply other circulating leukocytes. This confirmed that the migration and intravasation of GLPMs into the vasculature was dependent on ALI incited by APAP. Furthermore, maximum percentage of GATA6+ cells was seen at 6 hours, followed by a slight reduction by 48 hours; however it was still significantly higher when compared to the saline control. This reflected a clear presence of GLPMs in the vasculature just hours after liver injury, with the maximum number of GLPMs in the circulation somewhere between 6 hours and 24 hours post AILI. Thus, the TRMs can indeed intravasate and migrate into the injured liver upon creating an acute injury to the liver.

In vitro mRNA silencing of pro-inflammatory genes HMGB1, NB-κB and TLR-4 in primary mouse GLPMs using siRNAs encapsulated into C12-200-based LNPs: Having confirmed the rapid migratory ability of siRNA carrying GLPMs to the injured liver via systemic circulation, it was next sought to harness this therapeutically by RNAi-mediated gene silencing of some well-characterized pro-inflammatory targets and eventually demonstrate whether this could be a meaningful therapy for acute liver injuries. For this purpose, a well characterized DAMP like high mobility group box-1 (HMGB1) protein, its putative receptor on macrophages toll-like receptor-4 (TLR-4), and the pro-inflammatory transcription factor nuclear factor-KB (NF-κB), were used as the targets from one of the most well-conserved central nodes of inflammation amplifying genes that could be silenced using an siRNA in these GLPMs (Yang R and Tonnesseen T I. Hepatol Int. 2019; 13(1):42-50; Yamamoto T and Tajima Y. Expert Rev Gastroenterol Hepatol. 2017; 11(7):673-82; Lotze M T and Tracey K J. Nat Rev Immunol. 2005; 5(4):331-42; Bianchi M G et al., Toxicology Research. 2015; 4(2):385-98; and Ganbold T et al., ACS Appl Mater Interfaces. 2020; 12(10):11363-74; the entire contents of each of which are expressly incorporated herein by reference). Chemically modified siRNAs targeting HMGB1, TLR-4, NF-κB, and gLuc (control) were synthesized as previously described and their sense strands were labeled with a Cy5 fluorophore (Oza D et al., Theranostics. 2024; 14(6):2526-43; and Novobrantseva T I et al., Mol Ther Nucleic Acids. 2012; 1(1):e4; the entire contents of each of which are expressly incorporated herein by reference). Synthesized siRNAs were then formulated into the C12-200-based LNPs (Brown C R et al., Nucleic Acids Res. 2020; 48(21):11827-44; the entire contents of which are expressly incorporated herein by reference). In order to validate the siRNA sequences to assess target mRNA knockdown (KD) in vitro, KD of HMGB1, TLR-4 and NF-κB transcripts were assessed in isolated primary mouse GLPMs enriched from harvested peritoneal lavage. Upon transfecting these primary GLPMs with 100 nM of the respective siRNA-Cy5 (C12-200) treatments along with an equal volume of 1×PBS control for 24 hours, robust mRNA silencing for all the targets was achieved, with an 80%, 70% and 60% mRNA KD of HMGB1, TLR-4 and NF-κB respectively seen when expression was normalized to PBS control. This validated the potency of the tested siRNA sequences. Also, no non-specific silencing of any of our target genes was seen with the control gLuc-targeting siRNA.

SiRNA-mediated silencing of HMGB1 renders protection from inflammation induced by LPS in vitro in GLPMs: Next, the lipopolysaccharide (LPS)-stimulated macrophage activation model was used as the in vitro model system which would closely recapitulate a pro-inflammatory macrophage phenotype in vivo (Rietschel E T et al., Faseb j. 1994; 8(2):217-25). This model was used to validate the inflammation-mitigating ability of RNAi-mediated silencing of HMGB1, TLR4 and NF-κB by measuring pro- and anti-inflammatory macrophage markers post LPS treatment and then selecting the better target to later prevent an inflammatory macrophage activation state in vivo.

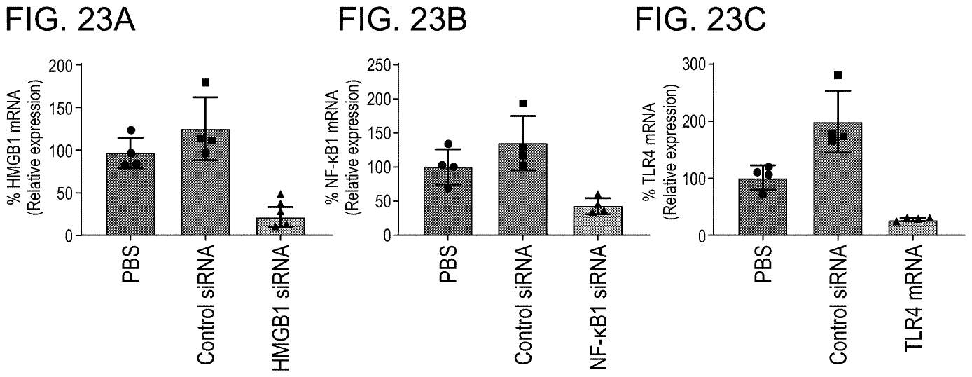

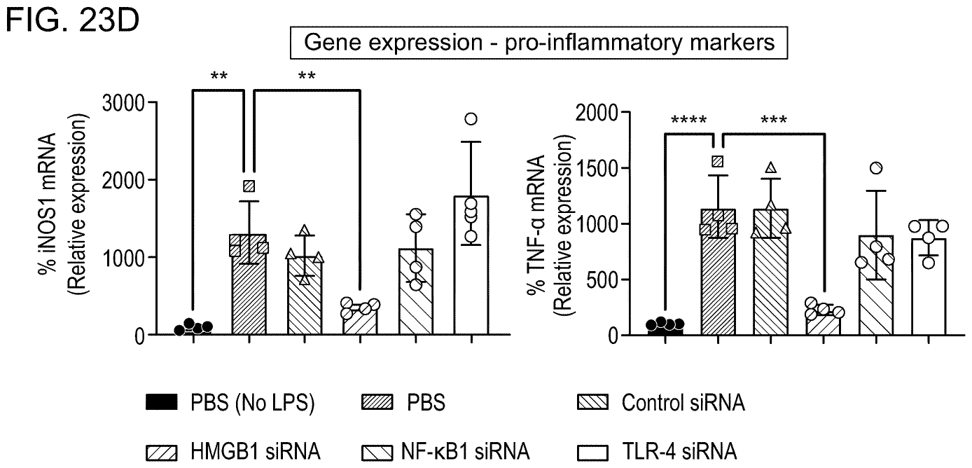

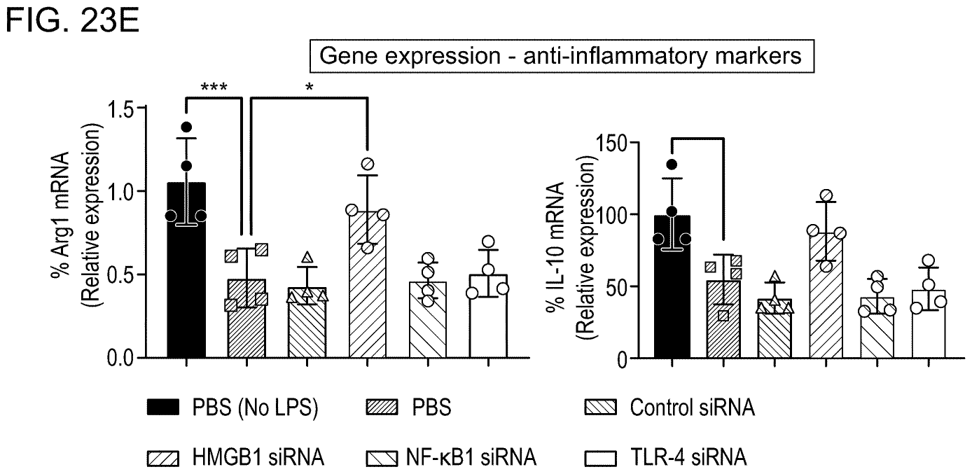

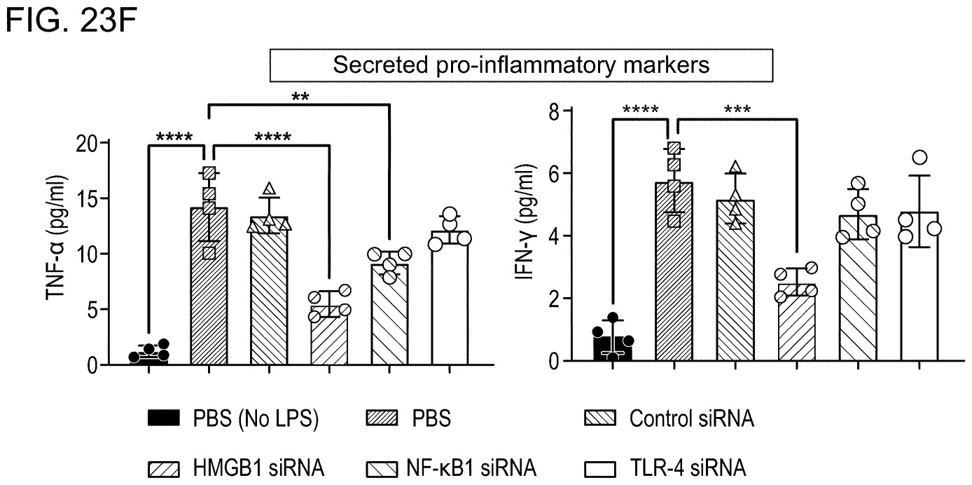

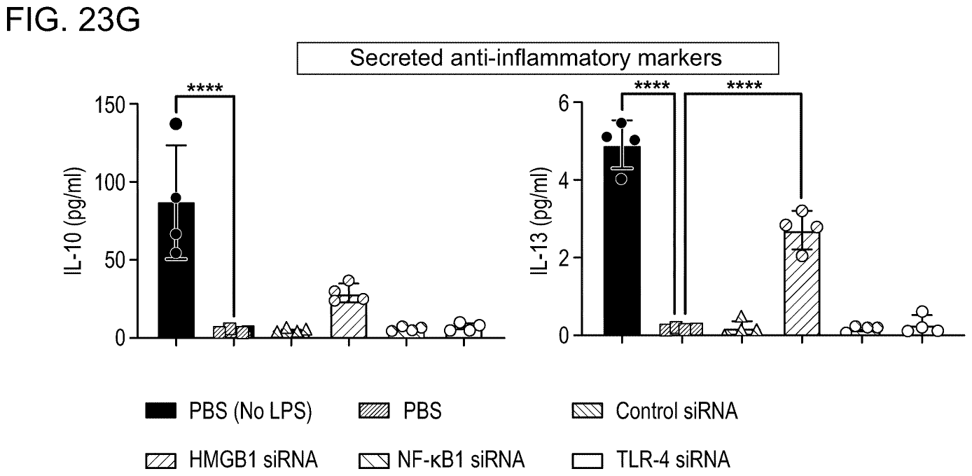

Hence, primary mouse GLPMs were subjected to 100 nM of the respective siRNA-Cy5 (C12-200) formulations for 24 hours along with co-treating the cells with 100 μg/ml of LPS dissolved in 1×PBS. There was again an 80%, 70% and 60% mRNA KD seen in HMGB1, TLR-4 and NF-κB mRNA expression relative to the PBS-treated group (FIGS. 23A-C). Relative to the PBS treated groups, HMGB1 silencing led to significant reduction in gene expression of macrophages pro-inflammatory markers iNOS-1 and TNF-α. Also, anti-inflammatory markers Arg1 and IL-10 were not as significantly downregulated in HMGB1 siRNA-treated group as the PBS-treated when compared to no-LPS control (FIGS. 23D, 23E).

Further, secreted cytokines measured from the cell supernatant also showed a similar trend where in there was a significant reduction in circulating pro-inflammatory cytokines TNF-α and interferon-gamma (IFN-γ). Levels of anti-inflammatory markers like IL-10 and IL-13 did not reduce as much in the HMGB1 siRNA-treated group as the PBS-treated group when compared to the no-LPS control (FIGS. 23F, 23G). Although NF-κB also led to a slight prevention in LPS-induced pro-inflammatory macrophage activation owing to some reduction in pro-inflammatory cytokine TNF-α, silencing of HMGB1 led to a clear prevention of macrophage activation to a pro-inflammatory phenotype by LPS (FIG. 23F). It was also notable that HMGB1 siRNA also led to the most KD of the target mRNA compared to other targets, which may also have factored into the more pronounced benefit seen upon HMGB1 silencing. Overall, this data suggested that inhibiting a more upstream DAMP like HMGB1 within the DAMP pro-inflammatory axis in GLPMs leads to a better prevention of LPS-induced macrophage activation and might potentially render protection against acute inflammation and downstream propagation to injury caused by APAP in vivo.

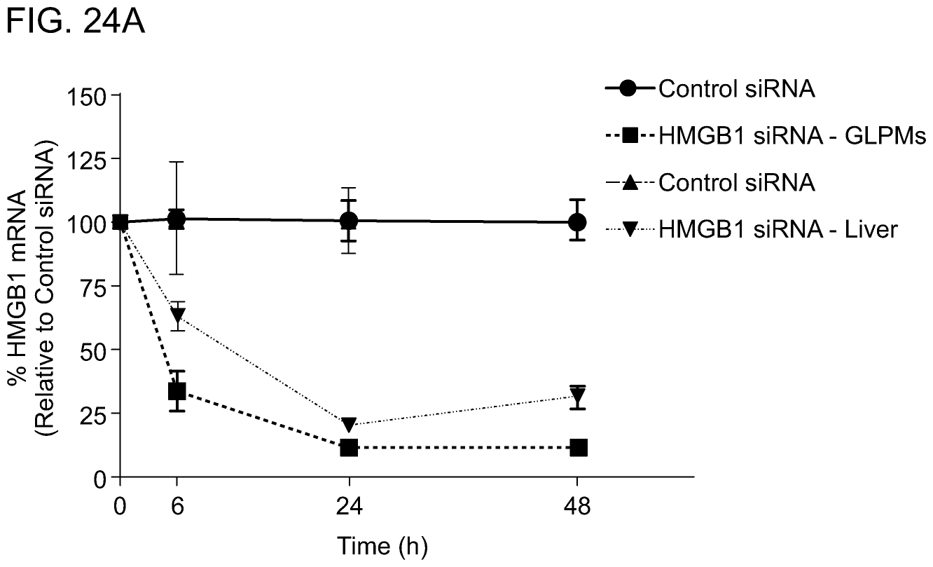

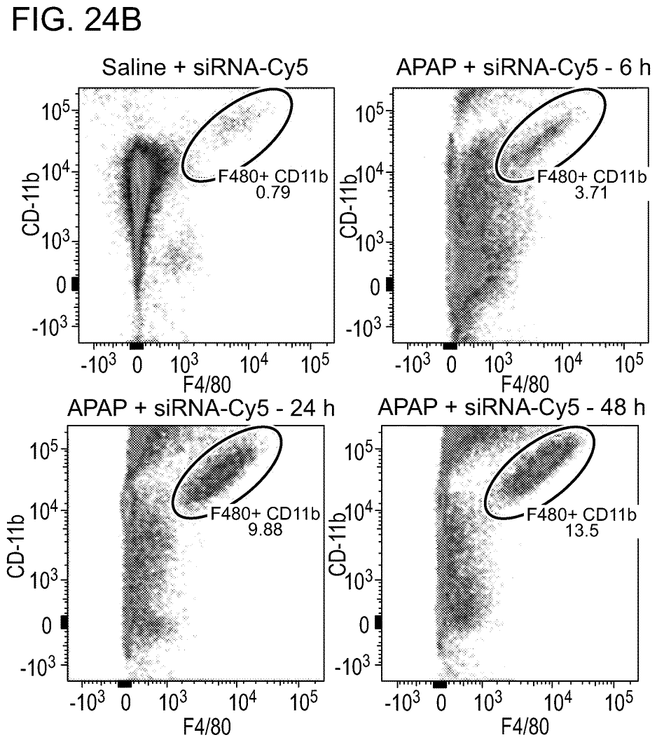

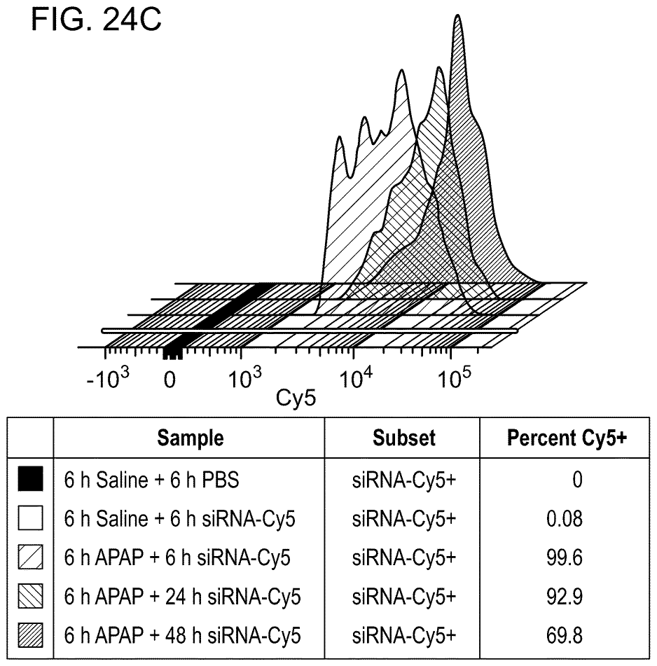

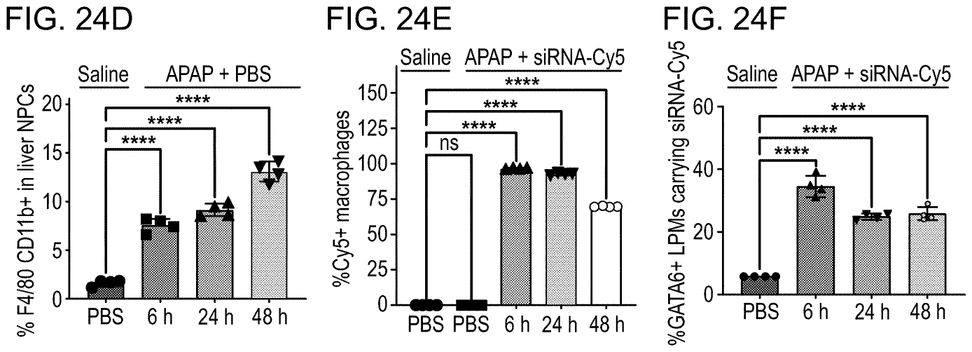

C12-200 encapsulated siRNA effectively silences HMGB1 in GLPMs in vivo: In order to 20 assess the in vivo pharmacodynamic (PD) properties of the siRNA-Cy5 (C12-200) targeting HMGB1, 1 mg/kg of HMGB1 siRNA-Cy5 (C12-200) was dosed intraperitoneally to female BALB/c mice along with a g-Luc control siRNA and peritoneal lavage and livers were isolated at 6 hours, 24 hours and 48 hours post injection. Isolated peritoneal lavage was enriched for GLPMs. Based on the gene expression analysis, there was already a significant mRNA KD of HMGB1 with the intraperitoneally administered siRNA-Cy5 (C12-200) by 6 hours, with only 30% HMGB1 mRNA remaining compared to the gLuc control siRNA where there was no reduction in expression (FIG. 24A). KD improved by 24 hours and was sustained to around 90% silencing of HMGB1 till 48 hours compared to gLuc control siRNA (FIG. 24A).

Along with GLPMs, PD in isolated liver NPCs was also measured since C12-200 LNPs may eventually be taken up by the liver and lead to a functional KD of HMGB1 transcripts within the liver. Liver was harvested and NPCs were isolated. KD in liver NPCs was minimal at 6 hours post injection, with a more significant KD of 75% seen by 24 hours, which was starting to recover by 48 hours (FIG. 23A). This was an important finding since it showed that intraperitoneally administered siRNA-Cy5 (C12-200) led to effective KD of target mRNA even at an earlier time point of 6 hours in GLPMs but not in the liver. Hence, these LNPs would help test the hypothesis of mitigating inflammation in the liver post AILI simply by silencing of HMGB1 in these liver infiltrating GLPMs. Since it was established earlier that there was significant infiltration of GLPMs into the liver already at 6 hours, upon preventing the release of HMGB1 from these cells within the first 6 hours of APAP administration, the inflammatory response could be muted or the downstream amplification of injury to the liver caused by APAP-induced hepatoxicity could be completely prevented.

GLPMs act as rapid carriers of siRNA to the injured liver post APAP-induced hepatotoxicity: Since both infiltration of GLPMs into the injured liver upon AILI as well as a robust RNAi-mediated silencing in vivo in GLPMs using C12-200-based LNPs were confirmed, the study aimed to determine whether the GLPMs carry fluorophore labeled siRNAs along with them when they migrate and infiltrate into the liver post AILI.

Female BALB/c mice were simultaneously administered a 300 mg/kg dose of APAP and a 1 mg/kg dose of gLuc-targeting control siRNA-Cy5 (C12-200) and livers were collected post 6 hours, 24 hours and 48 hours post injection. Saline dosed at the mouse equivalent weight along with the siRNA-Cy5 (C12-200) was an additional control along with a 1×PBS control, which were both dosed for 6 hours. Flow cytometry analysis of the harvested NPCs revealed a significant increase in the percentage of mature F4/80+CD11b+ TRM population (FIGS. 24B, 24D). Also, within this F4/80+CD11b+ population, there was a significant increase in the percentage of Cy5+ cells in the APAP-treated groups at all the time points of 6 hours, 24 hours and 48 hours compared to the saline+siRNA-Cy5 (C12-200) and saline+PBS controls (FIGS. 24C, 24E). This suggested that delivery of siRNA-Cy5 (C12-200) to the liver was clearly driven by the liver-infiltrating macrophage population contingent on liver injury incited, especially since there was no Cy5 signal seen in the liver NPCs in the saline treated group (C12-200)-treated group.

To further confirm these findings, IF imaging was carried out of the isolated NPCs and a significant increase in the percentage of GLPMs that were carrying the siRNA-Cy5 (C12-200) was observed at 6 hours, 24 hours and 48 hours when compared to the saline-treated control. Most infiltration of Cy5+ GLPMs was seen at 6 hours post siRNA-Cy5 (C12-200) administration, suggesting that GLPMs act as rapid carriers of siRNA to the injured liver post APAP-induced hepatotoxicity (FIG. 24F).

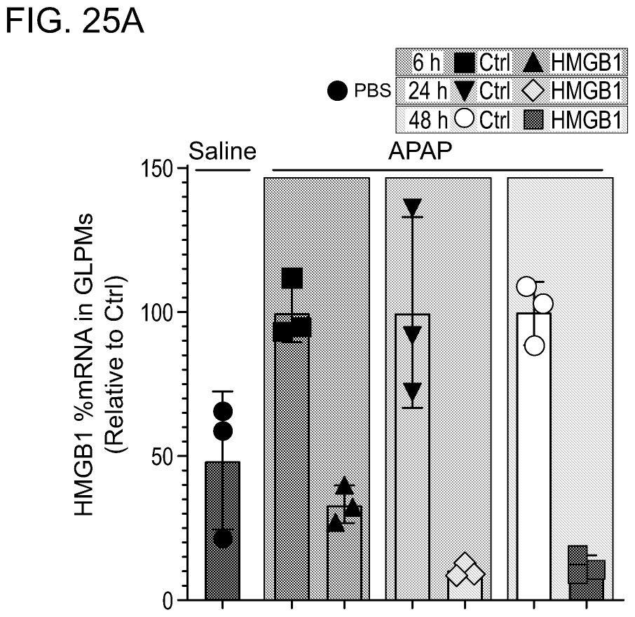

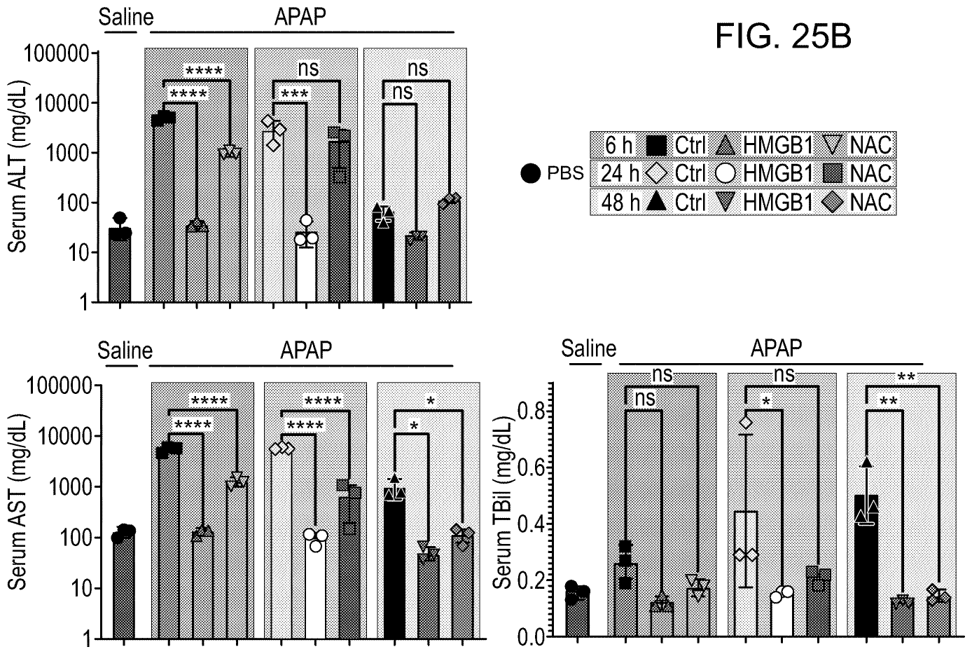

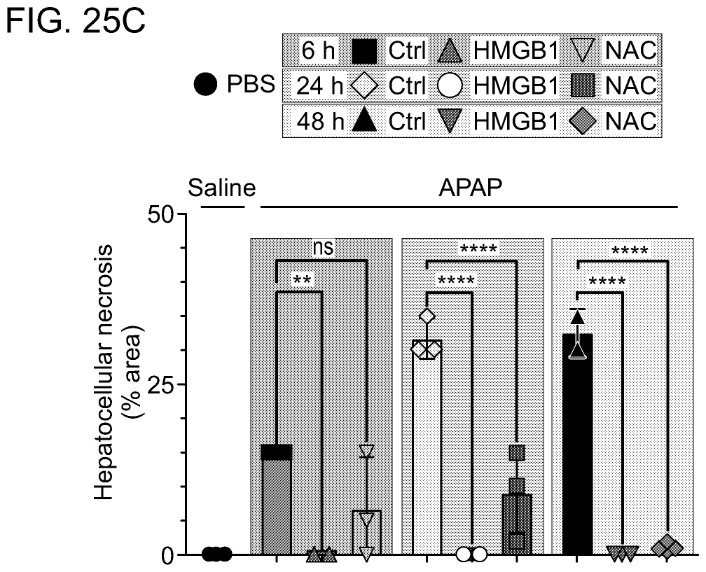

SiRNA-mediated silencing of HMGB1 in GLPMs renders a robust protection from APAP-induced hepatotoxicity: Female BALB/c mice were intraperitoneally administered a 300 mg/kg dose of APAP along with a 1 mg/kg dose HMGB1-targeting or gLuc (control)-targeting siRNA dosed intraperitoneally. A non-APAP saline treated group at the APAP-adjusted volume was also included as control. Along with this, in order to have a positive control for the study, the mice were dosed with a clinically validated antidote for APAP toxicity N-acetyl cysteine (NAC) by oral gavage at a dose of 150 mg/kg at the same time as APAP treatment (James L P et al., Toxicol Sci. 2003; 75(2):458-67; the entire contents of which are expressly incorporated herein by reference). Livers and peritoneal lavage were harvested at 6 hours, 24 hours and 48 hours for all the APAP and siRNA-Cy5 (C12-200) or NAC-treated groups, besides the saline control group, where they were collected at 48 hours post saline administration. Firstly, HMGB1 mRNA silencing was confirmed in the GLPMs with about 70%, 90% and 90% mRNA silencing observed at 6 hours, 24 hours, and 48 hours respectively compared to control siRNA (FIG. 25A). Serum liver injury biomarkers like ALT, AST and TBil revealed significant protection from liver injury in the HMGB1-treated groups at all the measured time points (FIG. 25B). Importantly, there seemed to be a robust response to HMGB1 siRNA treatment by the significantly lower serum ALT, AST and TBil levels at the early time point of 6 hours post HMGB1 treatment, which almost looked trending similarly to the saline-treated group (FIG. 25B). As expected, NAC-treatment also led to a significant reduction in the liver injury markers, generally at all the time points (FIG. 25B); however the effect of liver protection seemed smaller than the HMGB1-treated group.

Liver histopathological evaluation after hematoxylin and eosin (H&E) staining revealed hepatocellular degeneration/necrosis at 6 hours, 24 hours, and 48 hours post-dose for animals administered gLuc (control) siRNA (mild to moderate) or NAC (minimal to mild) but was not observed for animals administered HMGB1 siRNA. Additionally, hepatocellular atrophy was seen at 6 hours, 24 hours, and 48 hours post-dose for animals administered gLuc siRNA (mild) or NAC (minimal to mild) but was only observed at 6 hours post-dose for animals administered HMGB1 siRNA (minimal). Histopathological evaluation scores revealed almost a complete protection against APAP-induced liver injury in the livers of mice treated with HMGB1 siRNA at all the time points with a significant protection also seen with NAC-treatment (FIG. 25C), albeit not as much as HMGB1-treatment, especially at the earlier 6 hours and 24 hours time points.

HMGB1 silencing in GLPMs prevents macrophage activation and protection of sterile liver injury caused by downstream inflammation from AILI: Next, the study aimed to focus on the phenotypic state of the GLPMs, along with tracking the levels of some well-characterized pro- and anti-inflammatory secreted cytokines in the liver to check whether silencing HMGB1 led to the prevention of macrophage activation to a pro-inflammatory state.

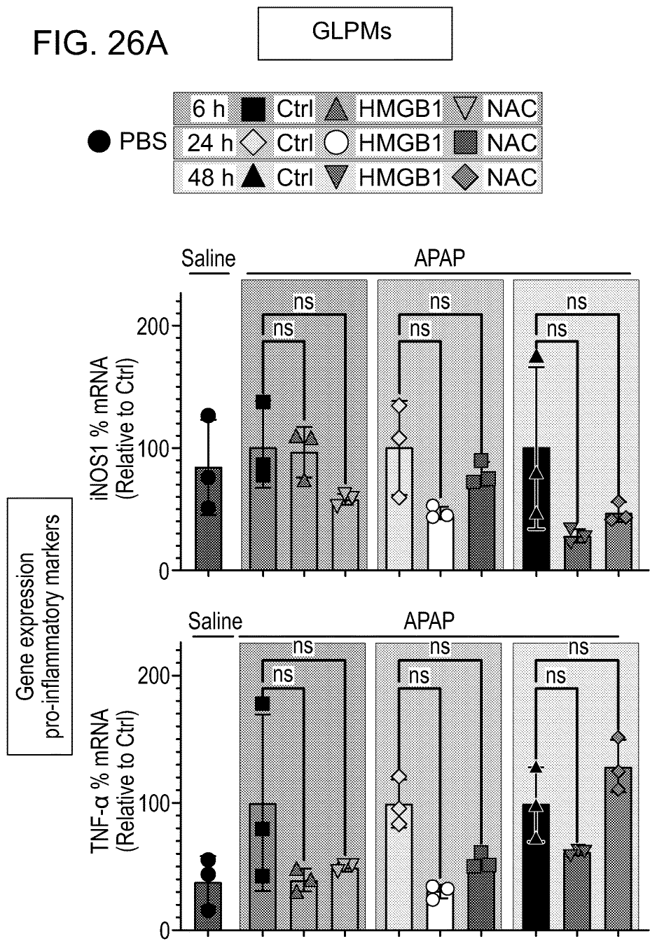

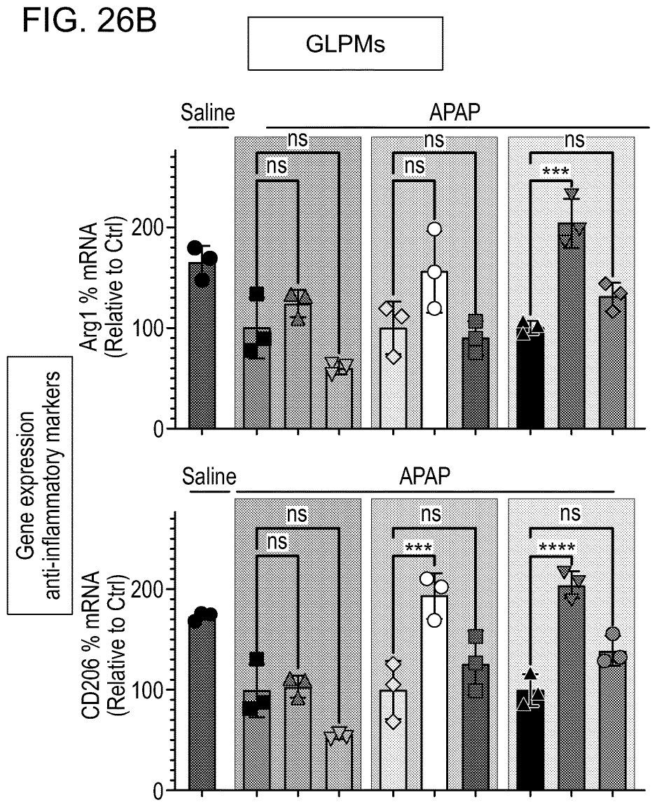

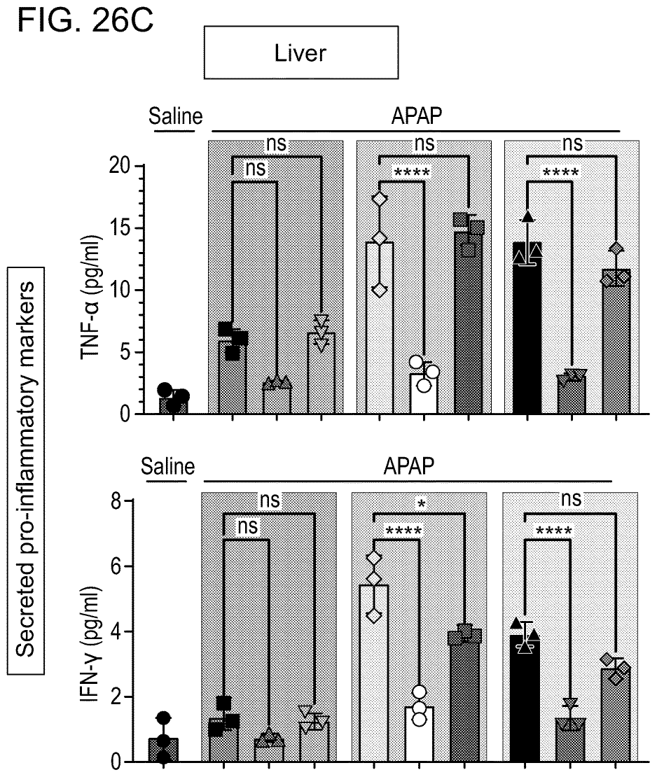

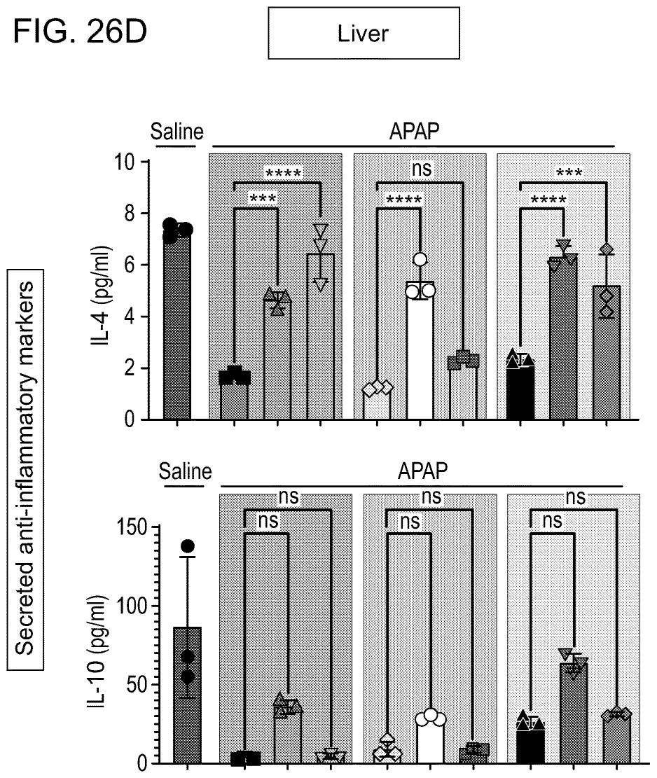

Upon measuring gene expression of some markers depicting state of macrophage modulation in the GLPMs, there was reduction in the classic pro-inflammatory macrophage markers iNOS1 and TNF-α, as well as normalization in expression of anti-inflammatory markers Arg1 and CD206 seen in the HMGB1 siRNA treated groups when compared to the gLuc control siRNA treated groups which, although not statistically significant by 6 hours, were almost trending similar to the saline-treated groups (FIGS. 26A, 26B). This pointed out to prevention of macrophage modulation to a pro-inflammatory state from AILI that was also seen earlier (FIGS. 20B, 20C). Similarly, in the liver, there were reductions in secreted pro-inflammatory cytokines TNF-α and IFN-γ as well as a normalization of anti-inflammatory cytokines IL-4 and IL-10 upon HMGB1 siRNA treatment when compared to the gLuc control siRNA treated groups (FIGS. 26C, 26D).

NAC-treated groups also minimally impacted the macrophage inflammatory state with slightly lower iNOS1 expression compared to gLuc siRNA, and a mild reduction in liver inflammatory cytokines TNF-α and IFN-γ, as well as some normalization of anti-inflammatory cytokine IL-4 (FIGS. 26A-D). However, these improvements were lesser compared to HMGB1 siRNA treated groups, and were typically only seen by 48 hours, not so much in the earlier 6 hours time point (FIGS. 26A-D). Hence, by preventing release of HMGB1 from the GLPMs, macrophage modulation, and in turn liver injury was prevented by delivery of these siRNA carrying GLPMs to the injured livers.

Silencing HMGB1 in hepatocyte by GalNAc-conjugated siRNA does not lead to any protection from acute liver injury: In order to further solidify the findings that silencing expression of HMGB1 in GLPMs, and not within the liver itself, led to a robust protection in liver, the same siRNA sequence against HMGB1 was conjugated to an N-acetyl galactosamine (GalNAc) ligand, which has been known to be selectively taken up by hepatocytes and lead to functional silencing of gene expression only in hepatocytes and not in any other infiltrating or liver-resident immune cells (Rajeev K G et al., ChemBioChem. 2015; 16(6):903-8; the entire contents of which are expressly incorporated herein by reference). By silencing hepatocyte specific HMGB1 transcript and testing this in a mouse model of AILI, the study aimed to tease out the differences in phenotype improvement by silencing HMGB1 in hepatocyte vs GLPMs that were observed earlier.

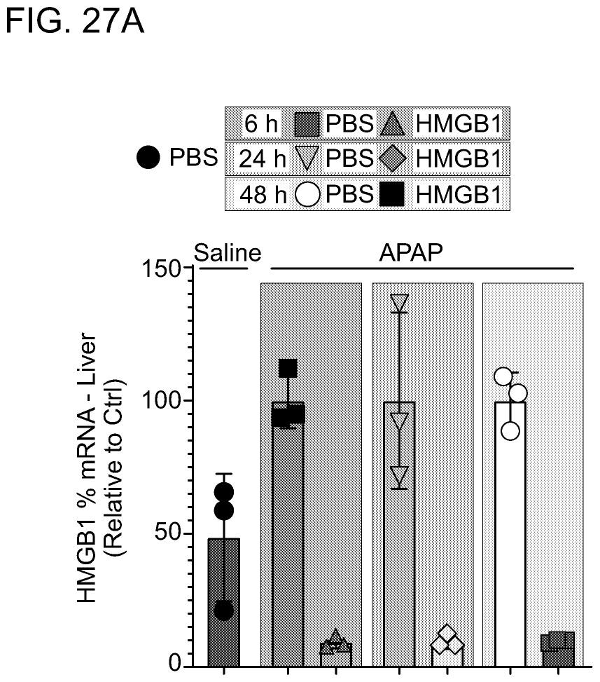

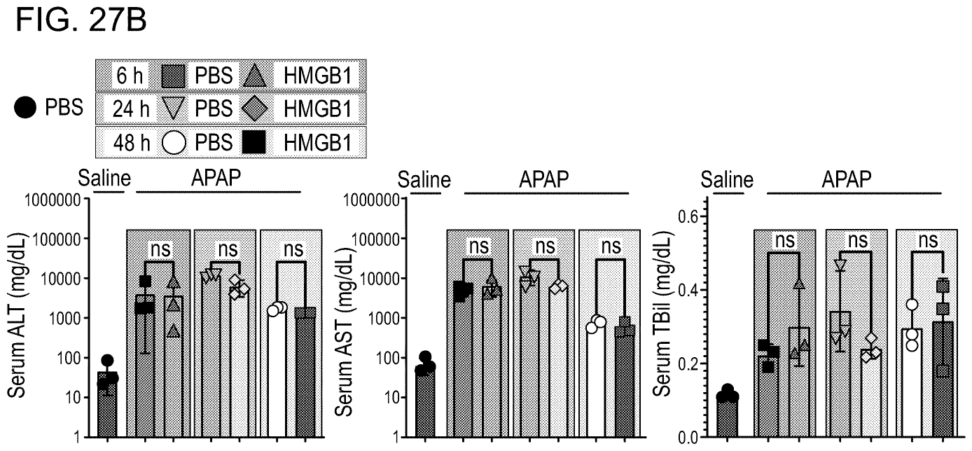

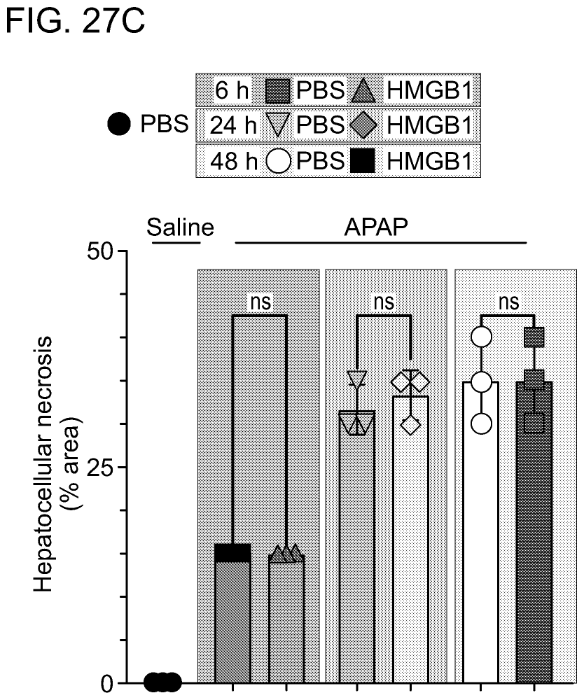

For this purpose, female BALB/c mice were pre-treated with a GalNAc-HMGB1 siRNA conjugate at a 3 mg/kg dose along with 1×PBS control dosed at an equivalent volume for 14 days by subcutaneous injection to silence the hepatocyte HMGB1 based on the relatively longer onset of action of a GalNAc-conjugated siRNA, and then treated with a 300 mg/kg dose of APAP given intraperitoneally for 6 hours, 24 hours, and 48 hours (Rajeev K G et al., ChemBioChem. 2015; 16(6):903-8). Firstly, a significant KD in HMGB1 expression was confirmed where in a sustained reduction in expression by 90% was seen in the liver samples collected at 6 hours, 24 hours, and 48 hours post AILI relative to PBS controls at the respective (FIG. 27A).

Serum chemistry findings revealed that HMGB1 silencing in the liver hepatocytes using a GalNAc conjugated siRNA did not lead to any meaningful benefit. There were slight reductions seen in ALT, AST and TBil at 24 hours post APAP injury compared to PBS control; however, they were minimal and not significant (FIG. 27B). Furthermore, liver H&E staining revealed that unlike the robust protection that was previously seen when HMGB1 siRNA was delivered in a C12-200 LNP and was silenced in GLPMs, GalNAc-HMGB1 did not lead to any significant benefit in the liver injury phenotype at any of the time points that were observed. The histopathological scores, too suggested that there was no benefit in the overall percentage of hepatocellular necrosis (FIG. 27C).

In view on the fast onset of action of siRNA-Cy5 (C12-200) targeting HMGB1 in GLPMs already by 6 hours, and their rapid migration to the injured liver, a near complete protection from injury as well as an anti-inflammatory phenotype (as seen from markers as early as 6 hours) was observed. This could be the primary initial mechanism of achieving protection of liver injury from the massive downstream inflammation propagated by APAP-driven liver injury and highlights the important translational potential of utilizing these siRNA-carrying GLPMs as delivery carriers of functional RNAi therapeutics to injured tissues, especially in more acutely driven injuries.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method of delivering a nucleic acid molecule to a large peritoneal macrophage (LPM), the method comprising contacting a nanoparticle encapsulating the nucleic acid molecule with the LPM, thereby delivering the nucleic acid molecule to the LPM.

2. (canceled)

3. (canceled)

4. The method of claim 1, wherein the LPM is a GATA6+ LPM.

5. The method of claim 1, wherein the nucleic acid molecule is selected from the group consisting of an oligonucleotide, a DNA, an RNA, a ribozyme, an aptamer, and a DNAzyme.

6.-0. (canceled)

11. The method of claim 1, wherein the nanoparticle is a lipid nanoparticle (LNP) or a polymeric nanoparticle.

12.-14. (canceled)

15. A method of delivering a nucleic acid molecule to an injured tissue in a subject in need thereof, the method comprising
administering a nanoparticle encapsulating the nucleic acid molecule to the subject,
allowing the nanoparticle encapsulating the nucleic acid molecule to contact a large peritoneal macrophage (LPM) in the subject, thereby generating an LPM comprising the nanoparticle encapsulating the nucleic acid molecule,
and allowing the LPM comprising the nanoparticle encapsulating the nucleic acid molecule to migrate to the injured tissue, thereby delivering the nucleic acid molecule to the injured tissue in the subject.

16. The method of claim 15, wherein the injured tissue is a non-peritoneal tissue or a peritoneal tissue.

17. The method of claim 16, wherein the non-peritoneal tissue is a lung tissue.

18.-41. (canceled)

42. The method of claim 15, wherein the administering of the nanoparticle encapsulating the nucleic acid molecule to the subject is performed intraperitoneally.

43. The method of claim 15, wherein the LPM is a GATA6+ LPM.

44. The method of claim 15, wherein the nucleic acid molecule is selected from the group consisting of an oligonucleotide, a DNA, an RNA, a ribozyme, an aptamer, and a DNAzyme.

45.-49. (canceled)

50. The method of claim 15, wherein the nanoparticle is a lipid nanoparticle (LNP) or a polymeric nanoparticle.

51.-53. (canceled)

54. A method of treating a disease in a subject in need thereof, the method comprising administering a nanoparticle encapsulating a nucleic acid molecule to the subject,
allowing the nanoparticle encapsulating the nucleic acid molecule to contact a large peritoneal macrophage (LPM) in the subject, thereby generating an LPM comprising the nanoparticle encapsulating the nucleic acid molecule,
and allowing the LPM comprising the nanoparticle encapsulating the nucleic acid molecule to migrate to an injured tissue, thereby treating the disease in the subject.

55. The method of claim 54, wherein the disease is selected from the group consisting of an inflammatory disease, an infectious disease, an autoimmune disease, and a cancer.

56.-61. (canceled)

62. The method of claim 54, wherein the injured tissue is a non-peritoneal tissue or peritoneal tissue.

63. (canceled)

64. The method of claim 62, wherein the injured tissue comprises an ablation or decrease in levels of tissue resident macrophages (TRMs) relative to an uninjured lung tissue.

65.-87. (canceled)

88. The method of claim 54, wherein the administering of the nanoparticle encapsulating the nucleic acid molecule to the subject is performed intraperitoneally.

89. The method of claim 54, wherein the LPM is a GATA6+ LPM.

90. The method of claim 54, wherein the nucleic acid molecule is selected from the group consisting of an oligonucleotide, a DNA, an RNA, a ribozyme, an aptamer, and a DNAzyme.

91.-95. (canceled)

96. The method of claim 54, wherein the nanoparticle is a lipid nanoparticle (LNP) or a polymeric nanoparticle.

97.-99. (canceled)

100. A method of detecting migration of a large peritoneal macrophage (LPM) comprising a nanoparticle encapsulating a nucleic acid molecule to an injured tissue in a subject, the method comprising administering the nanoparticle encapsulating the nucleic acid molecule to the subject,
allowing the nanoparticle encapsulating the nucleic acid molecule to contact the LPM in the subject, thereby generating the LPM comprising the nanoparticle encapsulating the nucleic acid molecule,
and allowing the LPM comprising the nanoparticle encapsulating the nucleic acid molecule to migrate to the injured tissue,
wherein detecting the migration of the LPM comprising the nanoparticle encapsulating the nucleic acid molecule to the injured tissue in the subject is performed by Diffuse in vivo Flow Cytometry (DiFC),
thereby detecting the migration of the LPM comprising the nanoparticle encapsulating the nucleic acid molecule to the injured tissue in the subject.

101.-132. (canceled)
